(19) United States
(12) Patent Application Publication
(10) Pub. No.: US 2026/0124217 A1
(43) Pub. Date: May 7, 2026

(54) COMPOSITIONS OF BIFUNCTIONAL INHIBITORS OF TETRACYCLINE INACTIVATING ENZYMES AND METHODS OF USE THEREOF

(71) Applicant: Washington University, St. Louis, MO (US)

(72) Inventors: Timothy Wencewicz, St. Louis, MO (US); Gautam Dantas, St. Louis, MO (US); Niraj Tolia, Bethesda, MD (US); Emily Williford, St. Louis, MO (US); Caitlin DeAngelo, St. Louis, MO (US); Kevin Blake, St. Louis, MO (US); Hirdesh Kumar, Bethesda, MD (US)

(73) Assignee: Washington University, St. Louis, MO (US)

(21) Appl. No.: 19/072,517

(22) Filed: Mar. 6, 2025

Related U.S. Application Data
(60) Provisional application No. 63/561,534, filed on Mar. 5, 2024.

(51) Int. Cl. A61K 31/65; A61P 31/04; C07C 237/26

(52) U.S. Cl. CPC A61K 31/65; A61P 31/04; C07C 237/26

(57) ABSTRACT

Among the various aspects of the present disclosure is the provision of bifunctional inhibitors of tetracycline inactivating enzymes and methods of use thereof. Herein, compositions for inhibitors of tetracycline inactivating enzymes are described. Additionally, a method of treatment for multidrug resistant bacterial infections using inhibitors of tetracycline inactivating enzymes is disclosed.

### CROSS-REFERENCE TO RELATED APPLICATIONS

This application claims the benefit of priority to U.S. Provisional Application Ser. No. 63/561,534 filed on Mar. 5, 2024, which is incorporated herein by reference in its entirety.

### STATEMENT REGARDING FEDERALLY SPONSORED RESEARCH OR DEVELOPMENT

This invention was made with government support under AI123394 awarded by the National Institutes of Health. The government has certain rights in the invention.

### MATERIAL INCORPORATED-BY-REFERENCE

Not applicable.

### FIELD OF THE INVENTION

The present disclosure generally relates to compositions of tetracycline destructase inhibitors and methods of use thereof.

### BACKGROUND OF THE INVENTION

Tetracycline (TC) antibiotics are a family of type-II polyketides originally isolated from Streptomyces aureofaciens. TCs have been in clinical use for >70 years as broad-spectrum antibiotics and continue to be used as frontline agents for treating a variety of infections caused by Gram-positive and Gram-negative bacteria. Until recently, it was thought that clinical TC resistance occurs primarily through the expression of efflux pumps and ribosome protection proteins. These resistance mechanisms have been largely overcome in the clinic by the development of last-generation TCs known as glycylcyclines including the FDA-approved drugs tigecycline, eravacycline, and omadacycline. Unfortunately, all known TC antibiotics are susceptible to an emerging third route of clinical resistance: enzymatic inactivation by tetracycline destructase (TDase) enzymes.

TDases are members of the class A flavin monooxygenase (FMO) enzyme family. TDases are FAD-dependent and use an NADPH/O2-coupled redox cycle to catalyze the inactivation of TC antibiotics. Oxidation of the bound TC substrate occurs via a C4a-peroxy-flavin intermediate resulting in substrate-dependent oxygen transfer (hydroxylation) and oxygen insertion (Baeyer-Villiger type) reactions. The resulting oxidized TC scaffolds lack antibacterial activity presumably due to a loss of binding affinity for the bacterial ribosome. TDases contain distinct substrate and FAD-binding domains connected via a C-terminal bridge helix. Two distinct types of TDases have <20% sequence homology and cluster by structural features, resistance phenotype, and ecological origin. Type 1 TDases have a constitutively open active site, provide resistance against all known TC antibiotic classes, and are found in human gut commensals and pathogens. Type 2 TDases contain an extra C-terminal helix that ‘gates’ the active site during the catalytic cycle, provide resistance to only first and second-generation TC antibiotics but not glycylcyclines, and are found primarily in environmental microbes. The structural and functional differences of Type 1 and 2 TDases have an important influence on substrate binding mode, flavin dynamics, mechanism of TC inactivation, and inhibition. At this point, Type 1 TDases appear to be the more likely clinical threat but the evolutionary connection between Type 1 and 2 TDases presents a unique opportunity to study TC resistance via enzymatic inactivation.

Enzymatic antibiotic inactivation is of particular concern given that this pathogen phenotype depletes the antibiotic challenge for the entire infection environment (including cells not expressing inactivating enzymes). The evolution of antibiotic inactivation enzymes under intense selective pressure is a potential gateway to pan resistance against entire drug classes. The clinical significance and global impact of this resistance mechanism have been fully realized for beta-lactam antibiotics where widespread dissemination of beta-lactamase encoding genes demands the co-administration of a beta-lactamase inhibitor to restore the clinical efficacy of beta-lactam antibiotics. Presumably, TDase inhibitors will be needed in the future given the mobilization and widespread distribution of TDase genes in the environment including clinical and agricultural settings. The emergence of TDases in clinical pathogens is on an upward trajectory and the deployment of new glycylcyclines could exacerbate this trend as has been observed following the release of new broad-spectrum beta-lactam antibiotics. Anhydrotetracycline (aTC), which differs from parent TC by dehydration of the C6 alcohol, is a pan TDase inhibitor and rescues whole cell activity of TC antibiotics in E. coli and Mycobacterium abscessus. The co-crystal structure of aTC bound to Tet(50) reveals a binding mode that is unique from the observed substrate binding mode leading to stabilization of the FAD-cofactor in an unproductive ‘OUT’ conformation that is stabilized through a π-π stacking interaction with the benzylic sidechain of Y267.

In addition to the TDase inhibitory activity, aTC alone has inherent antibacterial activity and some general toxicity at the effective concentrations (low μM) due in part to its ability to disrupt cellular membranes. Further, some Type 1 TDases can turnover aTC as a slow substrate indicating that aTC can sample the productive substrate binding mode with FAD in the ‘IN’ conformation. Simple modifications of the aTC scaffold including halogenation of C7 and C9 of the D-ring or demethylation of C6 of the C-ring were tolerated but failed to mitigate these potential liabilities.

### SUMMARY OF THE INVENTION

Among the various aspects of the present disclosure is the provision of compositions of bifunctional inhibitors of tetracycline inactivating enzymes and methods of use thereof.

Briefly, therefore, the present disclosure is directed to bifunctional inhibitors of tetracycline reductase and their use in the treatment of bacterial infections.

The present teachings include compositions for a bisubstrate Tetracycline Destructase Inhibitor compound. The composition can include a C9-substituted anhydrotetracycline (aTC) analog, which can be the structure:

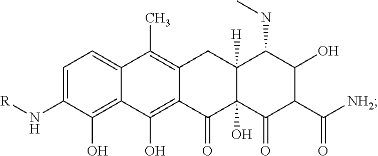

wherein R can be selected from Table 1.

In one aspect, the compound can inhibit both type 1 and type 2 tetracycline destructases (TDases). In another aspect, the compound is a competitive inhibitor of TDases. In another aspect, the compound binds sites comprising a substrate site and a nicotinamide site. In another aspect, the substrate site is a tetracycline (TC) binding site. In another aspect, the nicotinamide site is an NADPH binding site. In yet another aspect, the compound inhibits TDase reactions comprising a NADPH oxidation and a FAD reduction.

The present teachings also include a method of treating an infection with at least one of multidrug resistant (MDR) Gram-negative bacteria, extensively drug-resistant (XDR) Enterobacteriaceae species, and XDR Acinetobacter species. In one aspect, the method can include administering, to a patient in need thereof, a third-generation tetracycline in combination with a bisubstrate tetracycline destructase inhibitor compound.

In one aspect, the bisubstrate tetracycline destructase inhibitor compound comprises a C9-substituted anhydrotetracycline (aTC) analog, the C9-substituted anhydrotetracycline (aTC) analog comprising the structure:

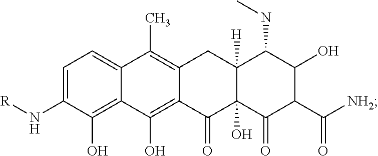

wherein R can be selected from Table 1.

In another aspect, the bisubstrate tetracycline destructase inhibitor compound inhibits both type 1 and type 2 tetracycline destructases (TDases). In another aspect, the bisubstrate tetracycline destructase inhibitor compound is a competitive inhibitor of TDases. In another aspect, the bisubstrate tetracycline destructase inhibitor compound binds sites on TDases comprising a substrate site and a nicotinamide site. In another aspect, the substrate site is a tetracycline (TC) binding site. In another aspect, the nicotinamide site is an NADPH binding site. In another aspect, the compound inhibits TDase reactions comprising a NADPH oxidation and a FAD reduction. In yet another aspect, the C9-substituted aTC analog comprises a TDase inhibitory activity and an antibacterial activity.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a schematic illustration of TDase-catalyzed hydroxylation of tetracycline (TC) at C11a to generate inactive TC-OH to achieve TC-resistance.

FIG. 1B is a ribbon model representation of the structure of Tet(50) with an inset of FAD-IN (chain A, PDB 5TUE) and FAD-OUT conformations (chain B, PDB 5TUE).

FIG. 1C is a graphical representation of the structure of Tet(50) bound to CTC with FAD-IN (PDB 5TUI).

FIG. 1D is a graphical representation of the structure of Tet(50) bound to a TC with FAD-OUT stabilized by Y267 (chain B, PDB 5TUF).

FIG. 1E is a graphical representation of the structure of compound 14 docked into the Tet(50) active site with FAD-OUT stabilized by the C9-benzamido substituent displacing Y267 rotated towards solvent. Docking performed using Glide and structures visualized using PyMOL.

FIG. 2 is a graph of apparent IC50 values measured through in vitro inhibition of Tet(X7) (blue) and Tet(50) (red) by compounds 1-21. Error bands depict standard deviations for three independent trials.

FIG. 3 is a graph of dose-dependent recovery of TC activity by aTC (1) and C9-aTC derivatives (4, 14, 16-20) against E. coli MegaX expressing Tet(X7) (solid lines) or Tet(50) (dashed lines) from a pZE21 inducible vector. The x-axis represents concentration of inhibitor (0-128 μg/mL) in the presence of constant TC (16 μg/mL). The y-axis represents growth rate generated from growth curve analysis as described in the methods section. The legend includes apparent IC50 values in μg/mL calculated for these data plots. Error bars represent standard deviation for three technical replicates.

FIG. 4A is a graphical representation of the compound TC (top) and the TC analogs compound 1 (middle) and compound 14 (bottom).

FIG. 4B is a set of graphs of TDase reactions with Tet(X7)+TC. The top graph shows time-dependent optical absorbance. The bottom graph shows relative percent LC-MS [M+H]+ ion counts for substrates and products normalized to an internal standard.

FIG. 4C is a set of graphs of TDase reactions with Tet(X7)+ compound 1. The top graph shows time-dependent optical absorbance. The bottom graph shows relative percent LC-MS [M+H]+ ion counts for substrates and products normalized to an internal standard

FIG. 4D is a set of graphs of TDase reactions with Tet(X7)+ compound 14. The top graph shows time-dependent optical absorbance. The bottom graph shows relative percent LC-MS [M+H]+ ion counts for substrates and products normalized to an internal standard.

FIG. 4E is a set of graphs of TDase reactions with Tet(50)+TC. The top graph shows time-dependent optical absorbance. The bottom graph shows relative percent LC-MS [M+H]+ ion counts for substrates and products normalized to an internal standard.

FIG. 4F is a set of graphs of TDase reactions with Tet(50)+ compound 1. The top graph shows time-dependent optical absorbance. The bottom graph shows relative percent LC-MS [M+H]+ ion counts for substrates and products normalized to an internal standard.

FIG. 4G is a set of graphs of TDase reactions with Tet(50)+ compound 14. The top graph shows time-dependent optical absorbance. The bottom graph shows relative percent LC-MS [M+H]+ ion counts for substrates and products normalized to an internal standard.

FIG. 4H is a graph of apparent steady-state kinetics of NADPH consumption in the presence of variable TDase inhibitor 1 or 14. Vmax was determined by fitting the observed reaction rates to the Michaelis-Menten equation. All TDase reactions were performed in at least duplicate as independent trials using pure NADPH.

FIG. 5A is a Lineweaver-Burke plot showing the competitive nature of Tet(50) inhibition by compound 14 with respect to TC.

FIG. 5B is a Lineweaver-Burke plot showing the competitive nature of Tet(50) inhibition by compound 14 with respect to NADPH.

FIG. 5C is a graphical representation of the overlay of the liganded x-ray crystal structures of Tet(50) bound to CTC (PDB: 5TUI) and aTC (PDB: 5TUF) highlight the distinct ligand binding modes and conformation of the FAD cofactor (FAD-IN for CTC; FAD-OUT for aTC). NADP+ was manually docked to highlight the presumed overlap of the space occupied by the Y267 sidechain or the NADP+/NADPH nicotinamide ring in stabilizing the FADOUT conformation through π-π stacking with the FAD isoalloxazine ring system.

FIG. 5D is a graphical representation of molecular docking of compound 14 captures the bifunctional binding mode where the aTC core scaffold overlaps with the original aTC binding mode with extended interactions of the C9-benzamide group across a solvent-exposed channel to allow for stabilization of the FAD isoalloxazine ring system through π-π stacking with overlap of the ligand space previously occupied by the Y267 sidechain or the NADP+/NADPH nicotinamide ring.

FIG. 6 is a schematic illustration of bisubstrate TDase inhibitors which block reduction of FAD by competitively binding to the TC and NADPH sites to prevent formation of peroxy flavin intermediate. aTC blocks the oxidation of TC and promotes the uncoupled formation of hydrogen peroxide by competitively binding to the TC site. aTC can be turned over as a competitive sacrificial substrate for Type 1 TDases.

FIG. 7A is a graphical representation of the synthesis of C9-aTC derivates 2-21.

FIG. 7B is an image of the R-groups 4-21 which can be used for the synthesis of C9-aTC derivates as shown in FIG. 7A.

FIG. 8A is a graphical representation of the mechanism of competitive TDase inhibitors.

FIG. 8B is a graphical representation of the mechanism of competitive bisubstrate TDase inhibitors.

FIG. 9A is a graphical representation of receptor preparation and validation of docking protocol. During receptor preparation, an alternate rotamer of Tyr267 was selected that orient away from the bound cofactor, FAD

FIG. 9B is a graphical representation of the docked (blue) and co-crystallized (white) binding poses of anhydrotetracycline (aTC). The docking score (in kcal/mol) is shown.

FIG. 9C is a graphical representation of the overlay of docked inhibitor #14 (blue) and co-crystallized aTC (white). The docking score (in kcal/mol) is shown.

FIG. 9D is a graphical representation of the overlay of docked inhibitor #20 (blue) and co-crystallized aTC (white). The docking score (in kcal/mol) is shown.

FIG. 9E is a graphical representation of the overlay of docked inhibitor #14 (white) and co-crystallized aTC (gray) showing inhibitor 14 extending into an open channel in the protein active site.

FIG. 10A is a graph of analytical LCMS analysis of TC optical absorbance trace. TC, Sigma, >95% purity. The y-axis represents optical absorbance at 263 nm. The x-axis represents retention time.

FIG. 10B is a graph of analytical LCMS analysis of Epi-TC optical absorbance trace. Epi-TC, Fluka, 99.5% purity. The y-axis represents optical absorbance at 263 nm. The x-axis represents retention time.

FIG. 10C is a graph of analytical LCMS analysis of TC and Epi-TC optical absorbance trace. TC, Sigma, >95% purity; Epi-TC, Fluka, 99.5% purity. The y-axis represents optical absorbance at 263 nm. The x-axis represents retention time.

FIG. 11A is a graph of analytical LCMS analysis of aTC optical absorbance trace. aTC, Chemodex, >98% purity. The y-axis represents optical absorbance at 263 nm. The x-axis represents retention time.

FIG. 11B is a graph of analytical LCMS analysis of Epi-aTC optical absorbance trace. Epi-aTC, Fluka, 88.6% purity. The y-axis represents optical absorbance at 263 nm. The x-axis represents retention time.

FIG. 11C is a graph of analytical LCMS analysis of aTC and Epi-aTC optical absorbance trace. aTC, Chemodex, >98% purity; Epi-aTC, Fluka, 88.6% purity. The y-axis represents optical absorbance at 263 nm. The x-axis represents retention time.

FIG. 12 is a set of graphs of Tet(X7) (top) and Tet(50) (bottom) degradation of epi-TC and TC. Apparent steady-state kinetic plots for TC and epi-TC are shown on the same scale to highlight apparent differences in substrate preference. Error bars represent standard deviations for three independent trials. Apparent steady state kinetic parameters from a fit to the Michaelis-Menten equation are: Tet(X7)+TC: Kapp=4.2±0.6 μM; vmax=0.049±0.001 min-1; Tet(X7)+epi-TC: Kapp=126±48 μM; vmax=0.065±0.016 min-1; Tet(50)+TC: Kapp=5.1±1.2 μM; vmax=0.069±0.004 min-1; Tet(50)+epi-TC: Kapp=50±10 μM; vmax=0.51±0.05 min-1.

FIG. 13 is a graph of the apparent velocity vs the concentration of epi-aTC indicating only trace consumption of epi-aTC in the presence of a TDase, indicating that Epi-aTC is not a substrate for Tet(X7) (red) or Tet(50) (blue). Error bars represent standard deviations for three independent trials.

FIG. 14 is a set of graphs of apparent TDase velocity at fixed [TC](25 μM) and variable [epi-aTC](0 or 150 μM). Epi-aTC is not a significant inhibitor of Tet(X7) (left) or Tet(50) (right). Error bars represent standard deviations for three independent trials. Tet(X7) p-value=0.0274; Tet(50) p-value=0.1061.

FIG. 15A is a set of graphs of half-maximal inhibitory concentrations (IC50) curves for the inhibition of Tet(X7) degradation of tetracycline. Inhibitors of Tet(X7) include aTC, compound 2, compound 3, compound 4, compound 5, and compound 6. Error bars represent standard deviations for three independent trials.

FIG. 15B is a set of graphs of half-maximal inhibitory concentrations (IC50) curves for the inhibition of Tet(X7) degradation of tetracycline by various inhibitors. Inhibitors of Tet(X7) include compound 7, compound 8, compound 9, compound 10, compound 11, and compound 12. Error bars represent standard deviations for three independent trials.

FIG. 15C is a set of graphs of half-maximal inhibitory concentrations (IC50) curves for the inhibition of Tet(X7) degradation of tetracycline in the presence of various inhibitors. Inhibitors of Tet(X7) include compound 13, compound 14, compound 15, compound 16, compound 17, and compound 18. Error bars represent standard deviations for three independent trials.

FIG. 15D is a set of graphs of half-maximal inhibitory concentrations (IC50) curves for the inhibition of Tet(X7) degradation of tetracycline in the presence of various inhibitors. Inhibitors of Tet(X7) include compound 19, compound 20, and compound 21. Error bars represent standard deviations for three independent trials.

FIG. 16A is a set of graphs of half-maximal inhibitory concentrations (IC50) curves for the inhibition of Tet(50) degradation of tetracycline in the presence of various inhibitors. Inhibitors of Tet(X7) include aTC, compound 2, compound 3, compound 4, compound 5, and compound 6. Error bars represent standard deviations for three independent trials.

FIG. 16B is a set of graphs of half-maximal inhibitory concentrations (IC50) curves for the inhibition of Tet(50) degradation of tetracycline in the presence of various inhibitors. Inhibitors of Tet(X7) include compound 7, compound 8, compound 9, compound 10, compound 11, and compound 12. Error bars represent standard deviations for three independent trials.

FIG. 16C is a set of graphs of half-maximal inhibitory concentrations (IC50) curves for the inhibition of Tet(50) degradation of tetracycline in the presence of various inhibitors. Inhibitors of Tet(X7) include compound 13, compound 14, compound 15, compound 16, compound 17, and compound 18. Error bars represent standard deviations for three independent trials.

FIG. 16D is a set of graphs of half-maximal inhibitory concentrations (IC50) curves for the inhibition of Tet(50) degradation of tetracycline in the presence of various inhibitors. Inhibitors of Tet(X7) include compound 19, compound 20, and compound 21. Error bars represent standard deviations for three independent trials.

FIG. 17 is a set of graphs of half-maximal inhibitory concentrations (IC50) curves for the aTC (1) (top) and compound 14 (bottom) inhibition of Tet(X7) degradation of tigecycline. Error bars represent standard deviations for three independent trials.

FIG. 18 is a set of graphs of bio-layer interferometry analysis of TC (top row), aTC (1, middle row), and 14 (bottom row) binding to Tet (X7) in the left column and Tet(50) in the right column. The y-axis represents ‘binding’ and the x-axis represents time. Each curve corresponds to the test compound concentration for Tet(X7) (left) and Tet(50) (right): top (TC), middle (aTC), bottom (compound 14).

FIG. 19A is a set of graphs of half-maximal inhibitory concentrations (IC50) curves for the inhibition of TC degradation by Tet(50) or Tet(X7). Error bars represent standard deviations for three technical replicates.

FIG. 19B is a set of graphs of half-maximal inhibitory concentrations (IC50) curves for the inhibition of TC degradation by Tet(50) or Tet(X7). Error bars represent standard deviations for three technical replicates.

FIG. 20A is an image of a petri dish showing antibacterial activity of compound 1, 14, and 20 determined by a modified Kirby-Bauer agar diffusion assay. Whole cell rescue of tigecycline (0.5 mg/mL) in E. coli BL21 cells expressing Tet(X7). Wells with C9-aTC analogs+tigecycline (wells 4, 5, and 6) show modest increase in zone size when compared to tigecycline alone (well 1). Wells: 1) Substrate alone; 2) aTC (1) alone (0.25 mg/mL); 3) Substrate+aTC (1) (0.25 mg/mL); 4) Substrate+14 (0.25 mg/mL); 5) Substrate+20 (0.25 mg/mL); 6) Substrate+14 (0.25 mg/mL)+20 (0.25 mg/mL); 7) 14 alone (0.25 mg/mL); 8) 20 alone (0.25 mg/mL).

FIG. 20B is an image of a petri dish showing antibacterial activity of compound 1, 14, and 20 determined by a modified Kirby-Bauer agar diffusion assay. Whole cell rescue of tetracycline (4 mg/mL) in E. coli BL21 cells expressing Tet(50). Wells with C9-aTC analogs+tetracycline (wells 4, 5, and 6) show larger zones of inhibition and clearer zones when compared to tetracycline alone (well 1). While aTC+tigecycline (well 3) shows a larger zone of inhibition in cells expressing Tet(X7) or Tet(50), aTC alone (well 2) has inherent antibacterial activity. Compounds 14 and 20 do not have any inherent antibacterial activity. Wells: 1) Substrate alone; 2) aTC (1) alone (0.25 mg/mL); 3) Substrate+aTC (1) (0.25 mg/mL); 4) Substrate+14 (0.25 mg/mL); 5) Substrate+20 (0.25 mg/mL); 6) Substrate+14 (0.25 mg/mL)+20 (0.25 mg/mL); 7) 14 alone (0.25 mg/mL); 8) 20 alone (0.25 mg/mL).

FIG. 21 is a graph quantifying H₂O₂ generation from TDase reactions. The concentration of H₂O₂ was determined for the TDase reactions shown in FIGS. 4B, 4C, 4D, 4E, 4F, and 4G by measuring the optical absorbance at 595 nm at 0, 30, 60, 90, and 120 min timepoints and comparing to a standard curve. Error bars represent standard deviations for three independent trials.

FIG. 22A is a Michaelis-Menten (MM) graph for compound 14 inhibition of Tet(50) degradation of tetracycline with varied [TC] concentrations. Reaction samples were prepared in TAPS buffer with 504 μM NADPH with varying concentrations of TC (0-30 μM) and contained a set amount of inhibitor (0 μM; blue or 32 μM; red) and 0.4 μM enzyme.

FIG. 22B is an MM graph for compound 14 inhibition of Tet(50) degradation of tetracycline with varied [NADPH]. Reaction samples were prepared in TAPS buffer with 25 μM TC with varying concentrations of NADPH (0-260 μM) and contained a set amount of inhibitor (0 μM; blue or 150 μM; red) and 0.4 μM enzyme.

FIG. 22C is an MM graph for compound 14 inhibition of Tet(X7) degradation of tetracycline with varied [TC]. Reaction samples were prepared in TAPS buffer with 504 μM NADPH with varying concentrations of TC (0-30 μM) and contained a set amount of inhibitor (0 μM; blue or 32 μM; red) and 0.4 μM enzyme.

FIG. 22D is an MM graph for compound 14 inhibition of Tet(X7) degradation of tetracycline with varied [NADPH]. Reaction samples were prepared in TAPS buffer with 25 μM TC with varying concentrations of NADPH (0-100 μM) and contained a set amount of inhibitor (0 μM; blue or 150 μM; red) and 0.4 μM enzyme.

FIG. 22E is a Lineweaver-Burk (LB) plot for compound 14 inhibition of Tet(X7) degradation of tetracycline with varied [TC].

FIG. 22F is a Lineweaver-Burk (LB) plot for compound 14 inhibition of Tet(X7) degradation of tetracycline with varied [NADPH].

FIG. 23 is a graph of tetracycline stability in Mueller-Hinton II Broth (MHII Broth) at 37° C. MHII Broth was prepared according to the instructions on the bottle. A solution of 64 μM tetracycline in MHII Broth was monitored by optical absorbance spectroscopy (280-550 nm, 1 nm and 30 min intervals) over 20 h at 37° C. The resulting raw data was normalized to an absorbance of 1.0.

FIG. 24 is a ribbon model representation of Tet(X6) which has conserved architecture of type 1 TDases. Structure of anhydrotetracycline free Tet(X6) (8ER1) includes the substrate binding domain (pink); FAD-binding domain (orange); and C-terminal bridge helix (blue). The Omit map for the bound FAD omitted from the Tet(X6) anhydrotetracycline free structure (8ER1) is green and density is contoured at 2.5 σ. The FAD is bound in an “IN” orientation consistent with previously solved type 1 TDase structures. The inset shows key residues in the Tet(X6) anhydrotetracycline free structure (8ER1) that interact with FAD are shown. Residues in the substrate binding pocket are also highlighted.

FIG. 25A is an image of a chemical structure of different tetracyclines and a table of R group modifications for the different tetracyclines (tetracycline, chlortetracycline, doxycycline, tigecycline, eravacycline, omadacycline.

FIG. 25B is a table showing Tet(X6) which confers minimum inhibitory concentrations (MIC). tetracycline (TET); doxycycline (DOX); chlortetracycline (CTC); omadacycline (OMA); tigecycline (TIG); eravacycline (ERA); anhydrotetracycline (aTC).

FIG. 25C is an MM graph for Tet(X6)-catalyzed degradation of tetracycline antibiotics. Note that the 65 μM data point for eravacycline is omitted for uniform scale on the x-axis. Error values represent standard deviations for three independent trials.

FIG. 25D is a table of apparent Km, kcat, and catalytic efficiencies (kcat/Km) of Tet(X6) against different tetracyclines.

FIG. 26A is a graph of whole cell inhibition of E. coli expressing tetracycline destructase enzymes. Anhydrotetracycline rescue Tet(X6)-mediated inactivation of tetracycline antibiotics. Tet(X6) (red), Tet(X7) (blue), pZE24 (gray).

FIG. 26B is a table of calculated fractional inhibitory concentration (FICI) for Tet(X6) and Tet(X7).

FIG. 26C is a set of graphs of in vitro aTC inhibition of Tet(X6) degradation of the tetracycline antibiotics TIG (top), OMA (middle), and ERA (bottom) as observed via an optical absorbance assay. Error values represent standard deviations for three independent trials.

FIG. 27A is a graphical representation of anhydrotetracycline which binds in a substrate-like orientation in Tet(X6). Polder maps (Fo-Fc map contoured at 3 σ) identify the substate like binding orientation of aTC in the two chains (chain A, left and chain B, right) of the Tet(X6) crystal structure.

FIG. 27B is a graphical representation of key interactions of anhydrotetracyclines with active site residues of Tet(X6).

FIG. 27C is a graphical representation of aligned crystal structures of Tet(X6)-anhydrotetracycline complex with Tet(X)-chlortetracycline (CTC) complex structure (PDB ID: 2Y6R). Aligned CTC and FAD from Tet(X) structures are shown in grey. The C11a of aTC is at 5.7 Å from C4a of the FAD isoalloxazine ring.

FIG. 28A is a schematic illustration of anhydrotetracycline oxidation catalyzed by type 1 TDases. C11a is a site of oxidation in aTC.

FIG. 28B is a graph showing the degradation of aTC as observed via optical absorbance spectroscopy.

FIG. 28C is a graph of extracted mass ion counts (normalized as relative %) for aTC and oxidized aTC-OH from LC-MS of Tet(X6) reaction from FIG. 29B.

FIG. 28D is a MM graph showing steady-state kinetic curve for Tet(X6)-catalyzed degradation of aTC. Error values represent standard deviations for three independent trials.

FIG. 29 is a set of images representing substrate and inhibitor binding modes in type 1 and type 2 TDases. In type 1 TDases (top panels), chlortetracycline (CTC) and anhydrotetracycline (aTC) bind in a similar substrate-like orientations. The bound co-factor, FAD (shown in blue) occupies an “IN” orientation. In contrast, CTC and aTC bind in distinct orientations in type 2 TDases (bottom panels). Anhydrotetracycline (aTC) locks FAD in an inactive “OUT” orientation.

FIG. 30A is an image of multiple sequence alignment of different TDases sequences. Protein sequences were retrieved from RCSB database in fasta format. Clustal omega was used to perform multiple sequence alignment. FAD-interacting residues are labeled as “F” and tetracycline substrate-interacting residues are labeled as “T”. PDB codes for different structures are as follows Tet(X2)-4A6N, Tet(X4)-7EPW, Tet(X6)-8ER1, Tet(X7)-6WG9, Tet(50)-5TUF, Tet(51)-5TUK, Tet(55)-5TUL, Tet(56)-5TUM.

FIG. 30B is a table of different TDase structures showing percentage sequence identity (lower half matrix; orange) and Cα-based r.m.s.d of different TDase structures. Total number of superimposed Cα atoms for each alignment are shown in parentheses (upper half matrix; blue).

FIG. 31 is a set of images showing the structural comparison of Tet(X6) (a type 1 TDase; PDB ID: 8ER1; left), Tet(50) (a type 2 TDase; PDB ID: 5TUF, chain A; middle), and merged (right). Tet(50) contains an additional “gatekeeper” α-helix at the C-terminus (red). This α-helix is a characteristic of type 2 TDases and is absent in type 1 TDase structures. Substrate-binding domain (pink), FAD-binding domain (orange), C-terminal bridge helix (blue).

FIG. 32 is a set of images showing the two-dimensional ligand interactions of ATC (top) and FAD (bottom) in chain A (left) and chain B (right) of the anhydrotetracycline bound Tet(X6). Residues are color-coded as per their physicochemical properties. H-bonds are shown in dotted arrows.

FIG. 33A is a ribbon model representation of crystal packing of an original anhydrotetracycline-free Tet(X6) structure (PDB ID: 8ER1; green) and the full image additional 5 Å symmetry mates (black).

FIG. 33B is a ribbon model representation of crystal packing of the original anhydrotetracycline-bound Tet(X6) structure (PDB ID: 8ER0; green) and full image contains additional 5 Å symmetry mates (black).

FIG. 34 is a set of graphs showing a Tet(X6)-catalyzed degradation of eravacycline (top) and omadacycline (bottom) under steady-state conditions. Velocity on the y-axis represents absorbance per minute measured continuously at 400 nm. The x-axis represents the working concentration of substrate, eravacycline or omadacycline, in micromolar. Error bars represent standard deviations for three independent trials.

FIG. 35A is a graphical representation of Polder maps (Fo-Fc map, σ=3) of bound anhydrotetracycline (ATC) and FAD in chain A (PDB ID: 8ER0).

FIG. 35B is a graphical representation of Polder maps (Fo-Fc map, σ=3) of bound anhydrotetracycline (ATC) and FAD in chain B (PDB ID: 8ER0).

FIG. 36 is a graph of 1H NMR (600 MHz, DMSO-d6) for compound 2.

FIG. 37 is a graph of 13C NMR (151 MHz, DMSO-d6) for compound 2.

FIG. 38 is a graph of COSY NMR (600 MHz, DMSO-d6) for compound 2.

FIG. 39 is a graph of HSQC NMR (600 MHz, DMSO-d6) for compound 2.

FIG. 40 is a graph of 1H NMR (600 MHz, DMSO-d6) for compound 3.

FIG. 41 is a graph of 13C NMR (151 MHz, DMSO-d6) for compound 3.

FIG. 42 is a graph of COSY NMR (600 MHz, DMSO-d6) for compound 3.

FIG. 43 is a graph of HSQC NMR (600 MHz, DMSO-d6) for compound 3.

FIG. 44 is a graph of HMBC NMR (600 MHz, DMSO-d6) for compound 3.

FIG. 45 is a graph of 1H NMR (500 MHz, DMSO-d6) for compound 4.

FIG. 46 is a graph of 13C NMR (125 MHz, DMSO-d6) for compound 4.

FIG. 47 is a graph of 1H NMR (500 MHz, DMSO-d6) for compound 5.

FIG. 48 is a graph of 13C NMR (125 MHz, DMSO-d6) for compound 5.

FIG. 49 is a graph of COSY NMR for compound 5.

FIG. 50 is a graph of HSQC NMR for compound 5.

FIG. 51 is a graph of HMBC NMR for compound 5.

FIG. 52 is a graph of 1H NMR (600 MHz, DMSO-d6) for compound 6.

FIG. 53 is a graph of 13C NMR (151 MHz, DMSO-d6) for compound 6.

FIG. 54 is a graph of COSY NMR (600 MHz, DMSO-d6) for compound 6.

FIG. 55 is a graph of HSQC NMR for compound 6.

FIG. 56 is a graph of HMBC NMR (600 MHz, DMSO-d6) for compound 6.

FIG. 57 is a graph of 1H NMR (600 MHz, DMSO-d6) for compound 7.

FIG. 58 is a graph of 13C NMR (151 MHz, DMSO-d6) for compound 7.

FIG. 59 is a graph of COSY NMR (600 MHz, DMSO-d6) for compound 7.

FIG. 60 is a graph of 1H NMR (500 MHz, DMSO-d6) for compound 8.

FIG. 61 is a graph of 13C NMR (125 MHz, DMSO-d6) for compound 8.

FIG. 62 is a graph of COSY NMR (500 MHz, DMSO-d6) for compound 8.

FIG. 63 is a graph of HSQC NMR (500 MHz, DMSO-d6) for compound 8.

FIG. 64 is a graph of HMBC NMR (500 MHz, DMSO-d6) for compound 8.

FIG. 65 is a graph of 1H NMR (600 MHz, DMSO-d6) for compound 9.

FIG. 66 is a graph of 13C NMR (151 MHz, DMSO-d6) for compound 9.

FIG. 67 is a graph of COSY NMR (600 MHz, DMSO-d6) for compound 9.

FIG. 68 is a graph of HSQC NMR (600 MHz, DMSO-d6) for compound 9.

FIG. 69 is a graph of HMBC NMR (500 MHz, DMSO-d6) for compound 9.

FIG. 70 is a graph of 1H NMR (500 MHz, DMSO-d6) for compound 10.

FIG. 71 is a graph of 13C NMR (151 MHz, DMSO-d6) for compound 10.

FIG. 72 is a graph of COSY NMR (500 MHz, DMSO-d6) for compound 10.

FIG. 73 is a graph of HSQC NMR (500 MHz, DMSO-d6) for compound 10.

FIG. 74 is a graph of HMBC NMR (500 MHz, DMSO-d6) for compound 10.

FIG. 75 is a graph of 1H NMR (500 MHz, DMSO-d6) for compound 11.

FIG. 76 is a graph of 13C NMR (125 MHz, DMSO-d6) for compound 11.

FIG. 77 is a graph of COSY NMR (500 MHz, DMSO-d6) for compound 11.

FIG. 78 is a graph of HSQC NMR (500 MHz, DMSO-d6) for compound 11.

FIG. 79 is a graph of HMBC NMR (500 MHz, DMSO-d6) for compound 11.

FIG. 80 is a graph of 1H NMR (500 MHz, DMSO-d6) for compound 12.

FIG. 81 is a graph of 13C NMR (125 MHz, DMSO-d6) for compound 12.

FIG. 82 is a graph of COSY NMR (500 MHz, DMSO-d6) for compound 12.

FIG. 83 is a graph of HSQC NMR (500 MHz, DMSO-d6) for compound 12.

FIG. 84 is a graph of HMBC NMR (500 MHz, DMSO-d6) for compound 12.

FIG. 85 is a graph of 1H NMR (500 MHz, DMSO-d6) for compound 13.

FIG. 86 is a graph of 1H NMR (600 MHz, DMSO-d6) for compound 14.

FIG. 87 is a graph of 13C NMR (151 MHz, DMSO-d6) for compound 14.

FIG. 88 is a graph of COSY NMR (600 MHz, DMSO-d6) for compound 14.

FIG. 89 is a graph of HSQC NMR (600 MHz, DMSO-d6) for compound 14.

FIG. 90 is a graph of HMBC NMR (600 MHz, DMSO-d6) for compound 14.

FIG. 91 is a graph of 1H NMR (600 MHz, DMSO-d6) for compound 15.

FIG. 92 is a graph of 1H NMR (600 MHz, DMSO-d6) for compound 16.

FIG. 93 is a graph of 1H NMR (151 MHz, DMSO-d6) for compound 16.

FIG. 94 is a graph of COSY NMR (600 MHz, DMSO-d6) for compound 16.

FIG. 95 is a graph of HSQC NMR (600 MHz, DMSO-d6) for compound 16.

FIG. 96 is a graph of HMBC NMR (600 MHz, DMSO-d6) for compound 16.

FIG. 97 is a graph of 1H NMR (600 MHz, DMSO-d6) for compound 17.

FIG. 98 is a graph of 13C NMR (151 MHz, DMSO-d6) for compound 17.

FIG. 99 is a graph of 1H NMR (600 MHz, DMSO-d6) for compound 18.

FIG. 100 is a graph of 13C NMR (151 MHz, DMSO-d6) for compound 18.

FIG. 101 is a graph of COSY NMR (600 MHz, DMSO-d6) for compound 18.

FIG. 102 is a graph of HSQC NMR (600 MHz, DMSO-d6) for compound 18.

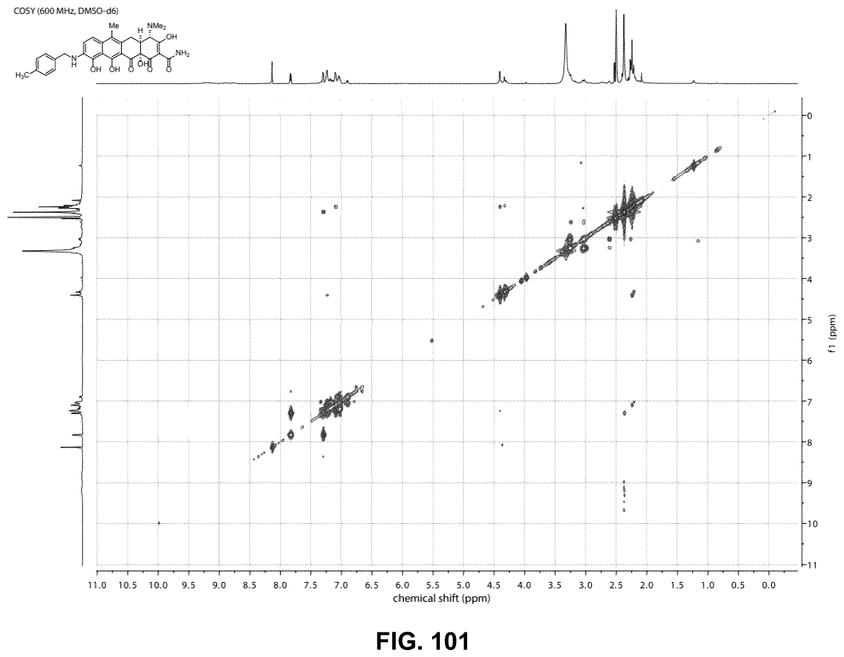

FIG. 101 is a graph of HMBC NMR (600 MHz, DMSO-d6) for compound 18.

FIG. 104 is a graph of 1H NMR (600 MHz, DMSO-d6) for compound 19.

FIG. 105 is a graph of 13C NMR (151 MHz, DMSO-d6) for compound 19.

FIG. 106 is a graph of COSY NMR (600 MHz, DMSO-d6) for compound 19.

FIG. 107 is a graph of HSQC NMR (600 MHz, DMSO-d6) for compound 19.

FIG. 108 is a graph of HMBC NMR (600 MHz, DMSO-d6) for compound 19.

FIG. 109 is a graph of 1H NMR (600 MHz, DMSO-d6) for compound 20.

FIG. 110 is a graph of 13C NMR (151 MHz, DMSO-d6) for compound 20.

FIG. 111 is a graph of COSY NMR (600 MHz, DMSO-d6) for compound 20.

FIG. 112 is a graph of HSQC NMR (600 MHz, DMSO-d6) for compound 20.

FIG. 113 is a graph of HMBC NMR (600 MHz, DMSO-d6) for compound 20.

FIG. 114 is a graph of 1H NMR (600 MHz, DMSO-d6) for compound 21.

FIG. 115 is a graph of 13C NMR (151 MHz, DMSO-d6) for compound 21.

FIG. 116 is a graph of COSY NMR (600 MHz, DMSO-d6) for compound 21.

FIG. 117 is a graph of HSQC NMR (600 MHz, DMSO-d6) for compound 21.

FIG. 118 is a set of graphs showing LCMS traces of purified compound 2. aTc-NO Crude—optical absorbance trace (top), aTC-NO Crude—mass trace (middle), aTC-NO Crude—aTC-NO [M+H]+ extracted mass (bottom).

FIG. 119 is a set of graphs showing LCMS traces of purified compound 3. aTc-NH2 Crude—optical absorbance trace (top), aTC-NH2 Crude—mass trace (middle), aTC-NH2 Crude—aTC-NH2 [M+H]+ extracted mass (bottom).

FIG. 120 is a set of graphs showing LCMS traces of purified compound 4. aTc-NH—CO—CH3 Prep-HPLC Purified—optical absorbance trace (top), aTC-NH—CO—CH3 Prep-HPLC Purified—mass trace (middle), aTC-NH—CO—CH3 Prep-HPLC Purified—aTC-NH—CO—CH3 [M+H]+ extracted mass (bottom).

FIG. 121 is a set of graphs showing LCMS traces of purified compound 5. aTc-NH—CO—(CH2)4-NH2 Prep-HPLC Purified—optical absorbance trace (top), aTC-NH—CO—(CH2)4-NH2 Prep-HPLC Purified—mass trace (middle), aTC-NH—CO—(CH2)4-NH2 Prep-HPLC Purified—aTC-NH—CO—(CH2)4-NH2 [M+H]+ extracted mass (bottom).

FIG. 122 is a set of graphs showing LCMS traces of purified compound 6. aTc-NH—CO-Ph Prep-HPLC Purified—optical absorbance trace (top), aTC-NH—CO-Ph Prep-HPLC Purified—mass trace (middle), aTC-NH—CO-Ph Prep-HPLC Purified—aTC-NH—CO-Ph [M+H]+ extracted mass (bottom).

FIG. 123 is a set of graphs showing LCMS traces of purified compound 7. aTc-NH—CO-Ph-2-NO2 Prep-HPLC Purified—optical absorbance trace (top), aTC-NH—CO-Ph-2-NO2 Prep-HPLC Purified—mass trace (middle), aTC-NH—CO-Ph-2-NO2 Prep-HPLC Purified—aTC-NH—CO-Ph-2-NO2 [M+H]+ extracted mass (bottom).

FIG. 124 is a set of graphs showing LCMS traces of purified compound 8. aTc-NH—CO-Ph-2-F Prep-HPLC Purified—optical absorbance trace (top), aTC-NH—CO-Ph-2-F Prep-HPLC Purified—mass trace (middle), aTC-NH—CO-Ph-2-F Prep-HPLC Purified—aTC-NH—CO-Ph-2-F [M+H]+ extracted mass (bottom).

FIG. 125 is a set of graphs showing LCMS traces of purified compound 9. aTc-NH—CO-Ph-2-Cl Prep-HPLC Purified—optical absorbance trace (top), aTC-NH—CO-Ph-2-Cl Prep-HPLC Purified—mass trace (middle), aTC-NH—CO-Ph-2-Cl Prep-HPLC Purified—aTC-NH—CO-Ph-2-Cl [M+H]+ extracted mass (bottom).

FIG. 126 is a set of graphs showing LCMS traces of purified compound 10. aTc-NH—CO-Ph-2-I Prep-HPLC Purified—optical absorbance trace (top), aTC-NH—CO-Ph-2-1 Prep-HPLC Purified—mass trace (middle), aTC-NH—CO-Ph-2-I Prep-HPLC Purified—aTC-NH—CO-Ph-2-I [M+H]+ extracted mass (bottom).

FIG. 127 is a set of graphs showing LCMS traces of purified compound 11. aTc-NH—CO-Ph-3-F Prep-HPLC Purified—optical absorbance trace (top), aTC-NH—CO-Ph-3-F Prep-HPLC Purified—mass trace (middle), aTC-NH—CO-Ph-3-F Prep-HPLC Purified—aTC-NH—CO-Ph-3-F [M+H]+ extracted mass (bottom).

FIG. 128 is a set of graphs showing LCMS traces of purified compound 12. aTc-NH—CO-Ph-3-Cl Prep-HPLC Purified—optical absorbance trace (top), aTC-NH—CO-Ph-3-Cl Prep-HPLC Purified—mass trace (middle), aTC-NH—CO-Ph-3-Cl Prep-HPLC Purified—aTC-NH—CO-Ph-3-Cl [M+H]+ extracted mass (bottom).

FIG. 129 is a set of graphs showing LCMS traces of purified compound 13. aTc-NH—CO-Ph-4-Br Prep-HPLC Purified—optical absorbance trace (top), aTC-NH—CO-Ph-4-Br Prep-HPLC Purified—mass trace (middle), aTC-NH—CO-Ph-4-Br Prep-HPLC Purified—aTC-NH—CO-Ph-4-Br [M+H]+ extracted mass (bottom).

FIG. 130 is a set of graphs showing LCMS traces of purified compound 14. aTc-NH—CO-Ph-4-NO2 Prep-HPLC Purified—optical absorbance trace (top), aTC-NH—CO-Ph-4-NO2 Prep-HPLC Purified—mass trace (middle), aTC-NH—CO-Ph-4-NO2 Prep-HPLC Purified—aTC-NH—CO-Ph-4-NO2 [M+H]+ extracted mass (bottom).

FIG. 131 is a set of graphs showing LCMS traces of purified compound 15. aTc-NH—CO—CH2-Ph Prep-HPLC Purified—optical absorbance trace (top), aTC-NH—CO—CH2-Ph Prep-HPLC Purified—mass trace (middle), aTC-NH—CO—CH2-Ph Prep-HPLC Purified—aTC-NH—CO—CH2-Ph [M+H]+ extracted mass (bottom).

FIG. 132 is a set of graphs showing LCMS traces of purified compound 16. aTc-NH—CH2-Ph Prep-HPLC Purified—optical absorbance trace (top), aTC-NH—CH2-Ph Prep-HPLC Purified—mass trace (middle), aTC-NH—CH2-Ph Prep-HPLC Purified—aTC-NH—CH2-Ph [M+H]+ extracted mass (bottom).

FIG. 133 is a set of graphs showing LCMS traces of purified compound 17. aTc-NH—CH2-Ph-4-NO2 Prep-HPLC Purified—optical absorbance trace (top), aTC-NH—CH2-Ph-4-NO2 Prep-HPLC Purified—mass trace (middle), aTC-NH—CH2-Ph-4-NO2 Prep-HPLC Purified—aTC-NH—CH2-Ph-4-NO2 [M+H]+ extracted mass (bottom).

FIG. 134 is a set of graphs showing LCMS traces of purified compound 18. aTc-NH—CH2-Ph-4-CH3 Prep-HPLC Purified—optical absorbance trace (top), aTC-NH—CH2-Ph-4-CH3 Prep-HPLC Purified—mass trace (middle), aTC-NH—CH2-Ph-4-CH3 Prep-HPLC Purified—aTC-NH—CH2-Ph-4-CH3 [M+H]+ extracted mass (bottom).

FIG. 135 is a set of graphs showing LCMS traces of purified compound 19. aTc-NH—CH2-Ph-4-Br Prep-HPLC Purified—optical absorbance trace (top), aTC-NH—CH2-Ph-4-Br Prep-HPLC Purified—mass trace (middle), aTC-NH—CH2-Ph-4-Br Prep-HPLC Purified—aTC-NH—CH2-Ph-4-Br [M+H]+ extracted mass (bottom).

FIG. 136 is a set of graphs showing LCMS traces of purified compound 20. aTc-NH—CH2-Ph-4-CF3 Prep-HPLC Purified—optical absorbance trace (top), aTC-NH—CH2-Ph-4-CF3 Prep-HPLC Purified—mass trace (middle), aTC-NH—CH2-Ph-4-CF3 Prep-HPLC Purified—aTC-NH—CH2-Ph-4-CF3 [M+H]+ extracted mass (bottom).

FIG. 137 is a set of graphs showing LCMS traces of purified compound 21. aTc-NH—CH2-CH2-Ph Prep-HPLC Purified—optical absorbance trace (top), aTC-NH—CH2-CH2-Ph Prep-HPLC Purified—mass trace (middle), aTC-NH—CH2-CH2-Ph Prep-HPLC Purified—aTC-NH—CH2-CH2-Ph [M+H]+ extracted mass (bottom).

### DETAILED DESCRIPTION OF THE INVENTION

The present disclosure is based, at least in part, on the discovery that by appending a nicotinamide isostere to the C9-position of the aTC D-ring, bisubstrate tetracycline reductase (TDase) inhibitors are generated. The bisubstrate inhibitors can have extended interactions with TDases by spanning both the TC and presumed NADPH binding pockets of the TDase. This can simultaneously block TC binding and reduction of FAD by NADPH while ‘locking’ TDases in an unproductive FAD ‘out’ conformation. As shown herein, the structure-based design of bisubstrate TDase inhibitors, as well as their mechanism of action are described.

One aspect of the present disclosure provides the synthesis and biological characterization of bifunctional inhibitors and pro-drug inhibitors of tetracycline inactivating enzymes. These compounds can be used in combination therapy with traditional tetracycline antibiotics to overcome resistance via enzymatic inactivation.

### Tetracycline Destructase (TDase) Modulation Agents

As described herein, TDase expression has been implicated in various diseases, disorders, and conditions, including tetracycline-resistant bacterial infections. As such, modulation of TDase (e.g., modulation of its ability to degrade tetracycline) in concert administration of a tetracycline compound can be used for treatment of such conditions. A TDase modulation agent can modulate TDase response or induce or inhibit TDase. TDase modulation can comprise modulating the expression of TDase in, on, and outside cells, modulating the quantity of cells that express TDase, or modulating the quality of the TDase-expressing cells.

TDase modulation agents can be any composition or method that can modulate TDase expression on cells (e.g., by binding TDase). For example, a TDase modulation agent can be an activator, an inhibitor, an agonist, or an antagonist. As another example, TDase modulation can be the result of gene editing.

A TDase modulation agent can be a TDase antibody (e.g., a monoclonal antibody to TDase).

A TDase modulating agent can be an agent that induces or inhibits progenitor cell differentiation into TDase expressing cells. For example, TDase small molecule inhibitors can be used to block TDase.

TDase Signal Reduction, Elimination, or Inhibition by Small Molecule Inhibitors, shRNA, siRNA, or ASOs

As described herein, a TDase modulation agent can be used for use in tetracycline-resistant bacterial infection therapy. A TDase modulation agent can be used to reduce/eliminate or enhance/increase TDase signals. For example, a TDase modulation agent can be a small molecule inhibitor of TDase. As another example, a TDase modulation agent can be a short hairpin RNA (shRNA). As another example, a TDase modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### TDase Inhibiting Agent

One aspect of the present disclosure provides for targeting of TDase, its substrate, or its downstream signaling. The present disclosure provides methods of treating or preventing bacterial infections based on the discovery that by appending a nicotinamide isostere to the C9-position of the aTC D-ring, bisubstrate tetracycline reductase (TDase) inhibitors are generated. The bisubstrate inhibitors can have extended interactions with TDases by spanning both the TC and presumed NADPH binding pockets. This can simultaneously block TC binding and reduction of FAD by NADPH while ‘locking’ TDases in an unproductive FAD ‘out’ conformation.

As described herein, inhibitors of TDase (e.g., antibodies, fusion proteins, small molecules) can reduce or prevent TC-resistant bacterial infections. A TDase inhibiting agent can be any agent that can inhibit TDase, downregulate TDase, or knockdown TDase.

As an example, a TDase inhibiting agent can inhibit TDase signaling.

For example, the TDase inhibiting agent can be an anti-TDase antibody. Furthermore, the anti-TDase antibody can be a murine antibody, a humanized murine antibody, or a human antibody.

As another example, the TDase inhibiting agent can be an anti-TDase antibody, wherein the anti-TDase antibody prevents binding of TDase to its receptor or prevents activation of TDase and downstream signaling.

As another example, the TDase inhibiting agent can be a fusion protein. For example, the fusion protein can be a decoy receptor for TDase. Furthermore, the fusion protein can comprise a mouse or human Fc antibody domain fused to the ectodomain of TDase.

As another example, a TDase inhibiting agent can be any of the small molecules described in the present disclosure, which have been shown to be a potent and specific inhibitor of TDase signaling.

As another example, a TDase inhibiting agent can be an inhibitory protein that antagonizes TDase. For example, the TDase inhibiting agent can be a viral protein, which has been shown to antagonize TDase.

As another example, a TDase inhibiting agent can be a short hairpin RNA (shRNA) or a short interfering RNA (siRNA) targeting TDase.

As another example, a TDase inhibiting agent can be an sgRNA targeting TDase.

Methods for preparing a TDase inhibiting agent (e.g., an agent capable of inhibiting TDase signaling) can comprise the construction of a protein/Ab scaffold containing the natural TDase receptor as a TDase neutralizing agent; developing inhibitors of the TDase substrate “down-stream”; or developing inhibitors of the TDase production “up-stream”.

Inhibiting TDase can be performed by genetically modifying TDase in a subject or genetically modifying a subject to reduce or prevent expression of the TDase gene, such as through the use of CRISPR-Cas9 or analogous technologies, wherein, such modification reduces or prevents a bacterial infection.

### Chemical Agent:

Non-limiting examples of TDase inhibiting agents are described herein. In some aspects, the TDase inhibiting agents comprise compounds with structure (I):

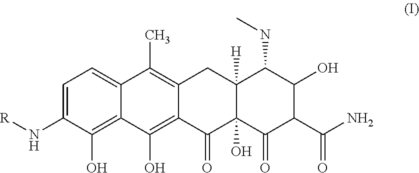

wherein R is selected from:

TABLE 1
R groups for formula I.

| R Group | |
|---|---|
| 4 | 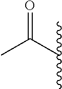 [SMILES: *C(C)=O] |
| 5 | 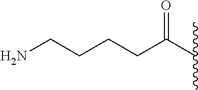 [SMILES: *C(=O)CCCCN] |
| 6 | 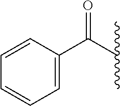 [SMILES: *C(=O)c1ccccc1] |
| 7 | 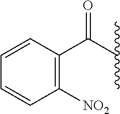 [SMILES: *C(=O)c1ccccc1[N+](=O)[O-]] |
| 8 | 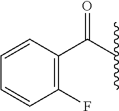 [SMILES: *C(=O)c1ccccc1F] |
| 9 | 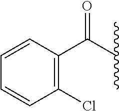 [SMILES: *C(=O)c1ccccc1Cl] |
| 10 | 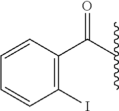 [SMILES: *C(=O)c1ccccc1I] |
| 11 | 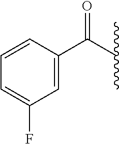 [SMILES: *C(=O)c1cccc(F)c1] |
| 12 | 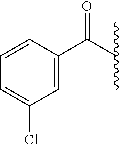 [SMILES: *C(=O)c1cccc(Cl)c1] |
| 13 | 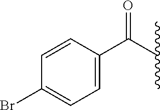 [SMILES: *C(=O)c1ccc(Br)cc1] |
| 14 | 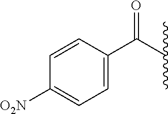 [SMILES: *C(=O)c1ccc([N+](=O)[O-])cc1] |
| 15 | 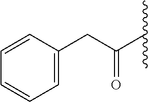 [SMILES: *C(=O)Cc1ccccc1] |
| 16 | 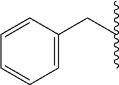 [SMILES: *Cc1ccccc1] |
| 17 | 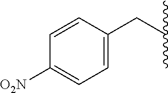 [SMILES: *Cc1ccc([N+](=O)[O-])cc1] |
| 18 | 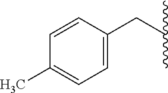 [SMILES: *Cc1ccc(C)cc1] |
| 19 | 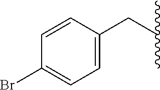 [SMILES: *Cc1ccc(Br)cc1] |
| 20 | 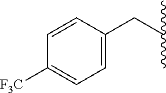 [SMILES: *Cc1ccc(C(F)(F)F)cc1] |
| 21 | 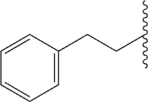 [SMILES: *CCc1ccccc1] |

R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C1-10alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C1-10alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C1-10alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C1-10 alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above, and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH2-cyclopropyl, —O—CH2-cyclobutyl, —O—CH2-cyclopentyl, —O—CH2-cyclohexyl, —O—CH2-cycloheptyl, —O—CH2-cyclooctyl, —O—CH2-cyclononyl, —O—CH2-cyclodecyl, —O—(CH2)2-cyclopropyl, —O—(CH2)2-cyclobutyl, —O—(CH2)2-cyclopentyl, —O—(CH2)2-cyclohexyl, —O—(CH2)2-cycloheptyl, —O—(CH2)2-cyclooctyl, —O—(CH2)2-cyclononyl, or —O—(CH2)2-cyclodecyl. An alkoxyl can be saturated, partially saturated, or unsaturated. The “alkoxyl” can be optionally substituted.

The term “cycloalkyl”, as used herein, unless otherwise indicated, can include an aromatic, non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, -CH2-cyclopropyl, -CH2-cyclobutyl, -CH2-cyclopentyl, -CH2-cyclopentadienyl, -CH2-cyclohexyl, -CH2-cycloheptyl, or -CH2-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with the formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatography. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing a bacterial infection in a subject in need of administration of a therapeutically effective amount of a TDase inhibitor in combination with a therapeutically effective amount of tetracycline antibiotic compound, so as to treat a tetracycline-resistant bacterial infection.

Methods described herein are generally performed on a subject in need thereof. A subject in need of the therapeutic methods described herein can be a subject having, diagnosed with, suspected of having, or at risk for developing a bacterial infection, including tetracycline resistant bacterial infections. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a TDase inhibitor is, for example, an amount that would cause the desired therapeutic effect in a subject while minimizing undesired side effects. In various embodiments, an effective amount of a TDase inhibitor described herein can substantially inhibit bacterial TDase activation associated with tetracycline treatment of a bacterial infection, thereby slowing the progress of a bacterial infection, or limiting the development of a bacterial infection.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a TDase inhibitor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a bacterial infection.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a TDase inhibitor can occur as a single event or over a time course of treatment. For example, a TDase inhibitor can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for bacterial infections.

A TDase inhibitor can be administered simultaneously or sequentially with another agent, such as a tetracycline antibiotic, an anti-inflammatory, or another agent. For example, a TDase inhibitor can be administered simultaneously with another agent, such as a tetracycline antibiotic or an anti-inflammatory. Simultaneous administration can occur through the administration of separate compositions, each containing one or more of a TDase inhibitor, a tetracycline antibiotic, an anti-inflammatory, or another agent. Simultaneous administration can occur through the administration of one composition containing two or more of a TDase inhibitor, a tetracycline antibiotic, an anti-inflammatory, or another agent. A TDase inhibitor can be administered sequentially with a tetracycline antibiotic, an anti-inflammatory, or another agent. For example, a TDase inhibitor can be administered before or after the administration of a tetracycline antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a TDase inhibitor, a tetracycline antibiotic, and solubilizing agents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 2—Structure of Anhydrotetracycline-Bound Tet(X6) Reveals the Mechanism for Inhibition of Type 1 Tetracycline Destructases

Inactivation of tetracycline antibiotics by tetracycline destructases (TDases) remains a significant clinical and agricultural threat. TDases can be classified as type 1 Tet(X)-like TDases and type 2 soil-derived TDases. Type 1 TDases are widely identified in clinical pathogens. A combination therapy of tetracycline and a TDase inhibitor is much needed to rescue the clinical efficacy of tetracyclines. Anhydrotetracycline is a pan-TDase inhibitor that inhibits both type 1 and type 2 TDases. In this example, structural, biochemical, and cellular evidence that anhydrotetracycline binds in a substrate-like orientation and competitively inhibits the type 1 TDase Tet(X6) as a sacrificial substrate is presented. Anhydrotetracycline interacting residues in Tet(X6) are conserved within type 1 TDases indicating a conserved binding mode and mechanism of inhibition. This mode of binding and inhibition is distinct from the inhibition of type 2 TDases by anhydrotetracycline. This study forms the framework for the development of next-generation therapies to counteract enzymatic tetracycline resistance.

### Introduction

Tetracyclines are a class of broad-spectrum antibiotics widely used in clinical and agricultural settings and considered one of the big four antibiotics for human use. Intensive use for more than eight decades has given rise to high abundance and diversity of tetracycline resistance genes in clinical pathogens. Historically, the two main mechanisms of tetracycline resistance have been ribosomal protection and drug efflux. To maintain the efficacy of this drug class against antibiotic resistance by these mechanisms, third-generation tetracyclines have been recently developed by chemical modification of the tetracycline moiety. Of these third-generation drug molecules, tigecycline is a last resort antibiotic used to treat infections with multidrug resistant (MDR) gram-negative bacteria and extensively drug-resistant (XDR) Enterobacteriaceae and Acinetobacter species.

Although tigecycline and other last-generation tetracyclines circumvent resistance by ribosomal protection or efflux pumps, these molecules are inactivated by a group of enzymes called tetracycline destructases (TDases). In recent years, we and others have identified and characterized a plethora of TDases found in commensal, environmental, and pathogenic bacteria. TDases are class A flavin-dependent monooxygenases that covalently modify and inactivate the core tetracycline moiety. Enzymatic inactivation of tetracyclines is unique in comparison to canonical tetracycline-resistance mechanisms because inactivation renders the tetracycline molecule incapable of further activity. TDases are broadly classified into two types based on sequence-structure-function characteristics: Tet(X)-like TDases and soil-derived TDases. Tet(X)-like TDases, which include the prototypical Tet(X) enzyme, have been identified in human gut metagenomes and pathogens, and can inactivate tetracyclines of all generations, including tigecycline and recently FDA-approved drugs sarecycline, eravacycline and omadacycline. In contrast, soil-derived TDases have been primarily identified in soil metagenomes and can inactivate first- and second-generation tetracyclines but show limited activity against last-generation tetracyclines. However, this soil-derived group of TDases can also be identified from other ecosystems. Tet(X)-like TDases and soil-derived TDases share only ˜20% amino acid similarity and they have broadly similar structures. Therefore, we propose naming these two classes of TDases as type 1 TDases for Tet(X)-like TDases; and type 2 TDases for those originally identified in soil metagenomes. A potent TDase inhibitor is needed for use in combination therapy to rescue the efficacy of the tetracycline group of antibiotics against pathogens expressing TDases.

Anhydrotetracycline (aTC) is the first broad spectrum TDase inhibitor that blocks type 1 and type 2 TDases in vitro and in bacterial phenotypic assays. We previously reported the crystal structure of anhydrotetracycline bound to the type 2 TDase Tet(50), showing it is a competitive inhibitor that binds in a distinct mode in the substrate binding cavity; these structural insights enabled development of anhydrotetracycline derivatives as additional type 2 TDase inhibitors. However, the binding and inhibition mode of anhydrotetracycline to type 1 TDases are unknown.

Here, we investigate Tet(X6), a type 1 TDase first discovered in a Proteus genomospecies, by determining anhydrotetracycline-free and anhydrotetracycline-complexed X-ray crystal structures. Tet(X6) inactivates all classes of tetracyclines demonstrated by in vitro enzyme assays and phenotypic studies in E. coli. The whole cell growth inhibitory activity of tetracycline against E. coli expressing Tet(X6) is rescuable with anhydrotetracycline. The structures revealed that anhydrotetracycline binds in a substrate-like orientation in Tet(X6) and serves as a competitive inhibitor. In contrast, type 2 TDases are unable to metabolize anhydrotetracycline, and anhydrotetracycline binds to type 2 TDases in a distinct orientation as a mechanistic and competitive inhibitor. Michaelis-Menten steady-state kinetic studies confirmed that anhydrotetracycline is a substrate for Tet(X6). Direct detection of the anhydrotetracycline Tet(X6) oxidation product by LC-MS further supports a model for competitive inhibition as a sacrificial substrate. The current study explains the differences in structural and functional characteristics of anhydrotetracycline between the two classes of TDases.

### Results

### Tet(X6) Contains a Conserved Architecture of Type 1 Tetracycline Destructases.

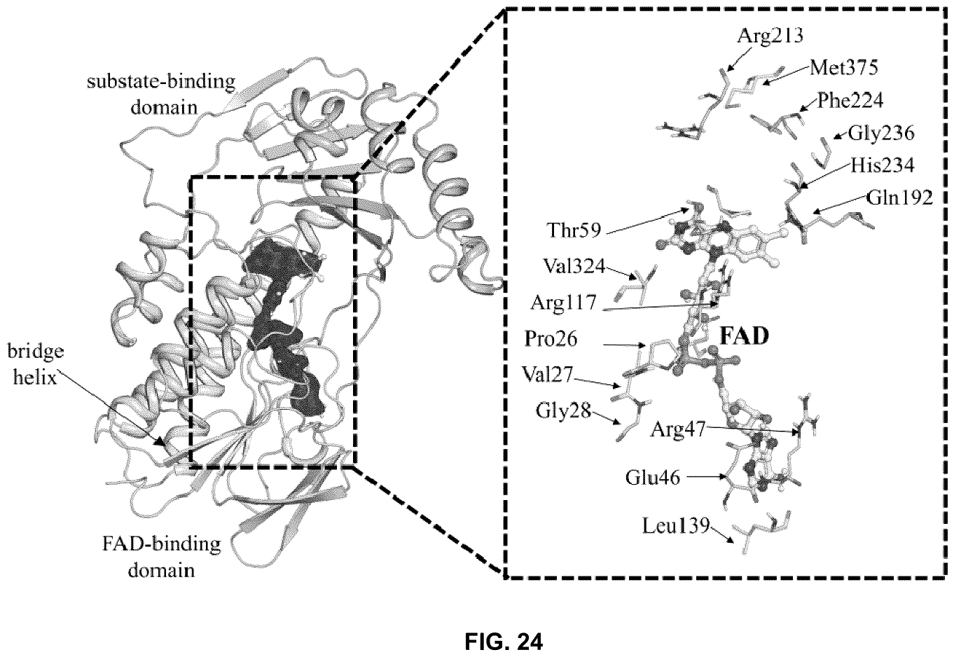

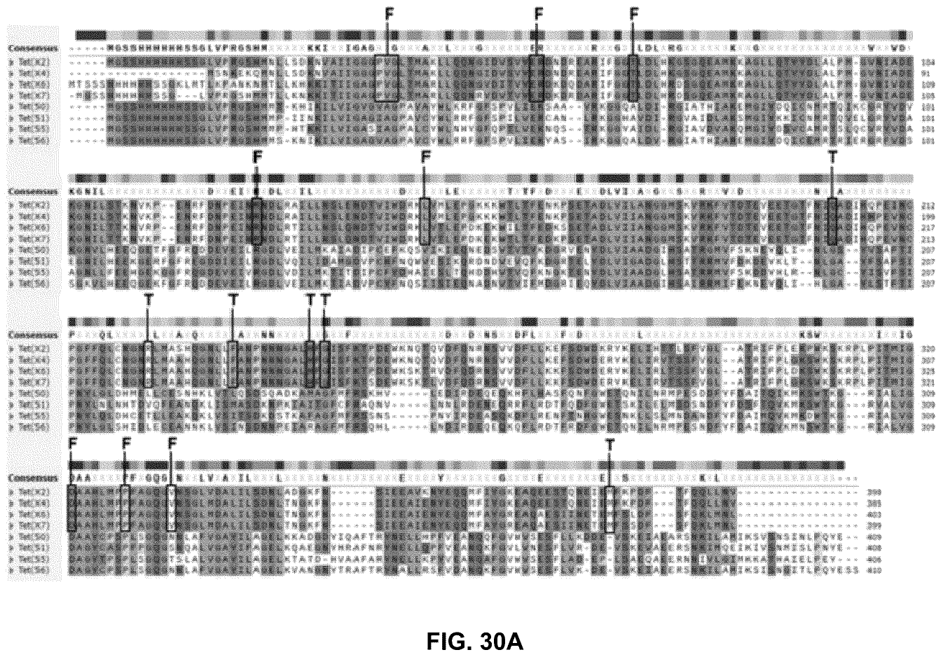

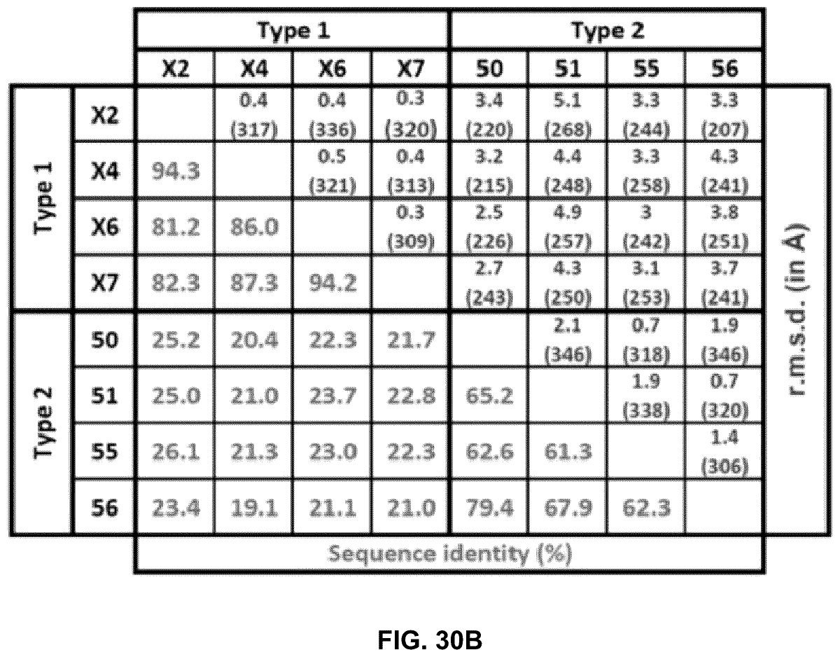

We determined the X-ray structure of Tet(X6) at 2.2 Å resolution with Rfree/Rwork of 0.22/0.19 (Table 3). This FAD complexed, anhydrotetracycline-free enzyme structure consists of a conserved architecture for type 1 TDases: a characteristic Rossman fold containing an FAD-binding domain, a substrate binding domain, and a C-terminal bridge helix that connects the two domains (FIG. 24). The 393 amino acid long polypeptide chain alternates four times between the substrate-binding domain and FAD-binding domain. The Tet(X6) holo-enzyme resembles previously reported type 1 TDases with high structural similarity of <0.5 Å r.m.s.d. on C^{α} atoms, and high sequence identity of >85% (FIG. 30A).

TABLE 3
Each structure was solved from a single crystal. Values in
parentheses are for highest-resolution shell.
Data Reduction Statistics for the Tet(X6) and Tet(X6) in
complex with anhydrotetracycline structures

| | |
|---|---|
| | Tet(X6) + Anhydro- |
| Tet(X6) (8ER1) | tetracycline (8ER0) |

Data collection

| | | |
|---|---|---|
| Space group | P 1 21 1 | P 1 21 1 |

Cell dimensions

| | | |
|---|---|---|
| a, b, c (Å) | 43.77, 52.49, 95.17 | 87.09, 52.21, 94.87 |
| α, β, γ (°) | 90, 95.88, 90 | 90, 95.44, 90 |

| | | | | |
|---|---|---|---|---|
| Resolution (Å) | 19.9-1.9 | (1.94-1.9) | 19.84-2.2 | (2.28-2.2) |
| R_{merge} | 0.031 | (0.234) | 0.095 | (0.592) |
| I/σI | 18.1 | (2.8) | 9.8 | (5.1) |
| Completeness (%) | 98.8 | (98.0) | 98.8 | (98.0) |
| Redundancy | 3.5 | (3.3) | 3.5 | (3.4) |

Refinement

| | | | | |
|---|---|---|---|---|
| Resolution (Å) | 19.9-1.9 | (1.94-1.9) | 19.84-2.2 | (2.28-2.2) |
| No. reflections | 33,536 | (3273) | 42,923 | (4215) |

| | | |
|---|---|---|
| R_{work}/R_{free} | 0.20/0.23 | 0.19/0.23 |

No. atoms

| | | |
|---|---|---|
| Protein | 2952 | 5877 |
| Ligands | 84 | 268 |
| Water | 152 | 263 |

B-factors

| | | |
|---|---|---|
| Protein | 41.1 | 33.1 |
| Ligands | 33.6 | 38.7 |
| Water | 41.8 | 32.9 |

R.m.s. deviations

| | | |
|---|---|---|
| Bond lengths (Å) | 0.011 | 0.003 |
| Bond angles (°) | 1.09 | 0.57 |

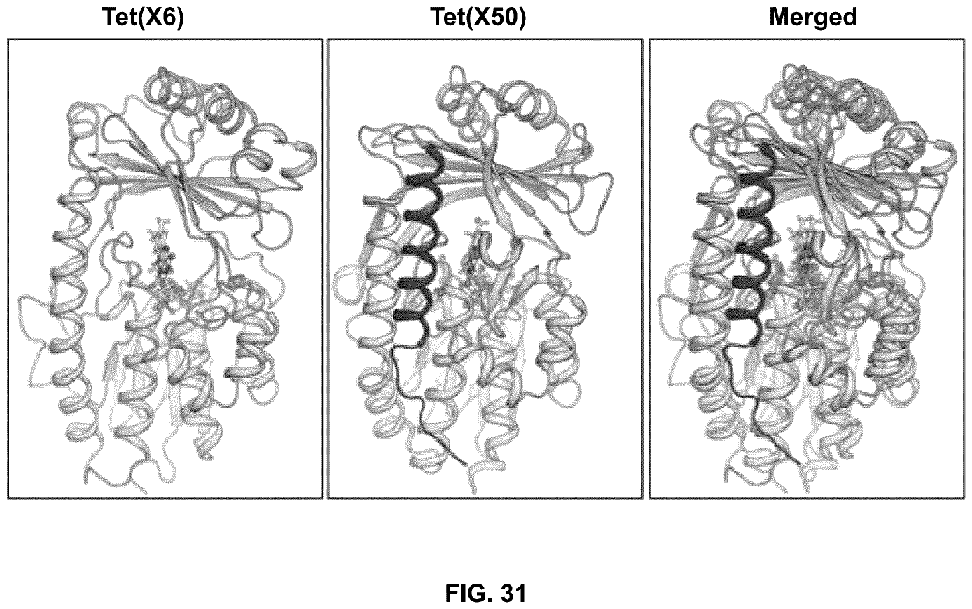

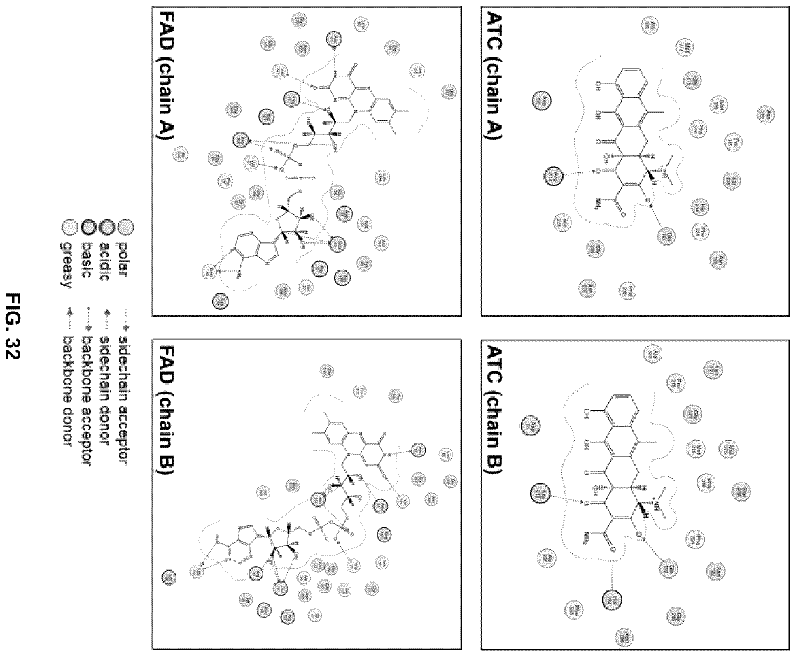

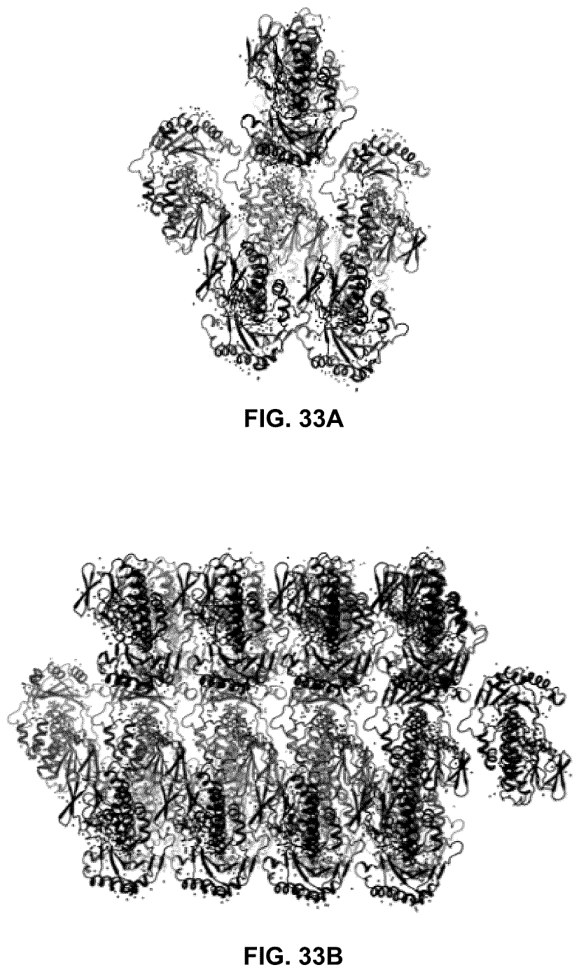

Tet(X6) contains a single C-terminal bridge helix similar to other type 1 TDase structures (Tet(X2)—PDB 2Y6R, Tet(X4)—PDB 7EPV, and Tet(X7)—PDB 6WG9) (FIG. 30B) and this constitutes a major distinction from type 2 TDases that contain a 2nd α-helix at the C-terminus (FIG. 24, FIG. 31). The FAD cofactor is bound in an “IN” orientation in Tet(X6). Thus far, the FAD conformation in structures of type 1 TDases have only been captured in this “IN” orientation, while structures of type 2 TDases have been solved for both “IN” and “OUT” orientations. It is assumed, but not yet experimentally validated, that the FAD cofactor present in the type 1 TDases share this two-state binding mode. The FAD-binding pocket and the substrate-binding pocket are conserved among all four of the solved type 1 TDase structures (FIG. 24, FIG. 30B). The conserved FAD-interacting residues include Pro26, Val27, Gly28, Glu46, Arg47, Thr59, Arg117, Leu139, and Val324. The conserved substrate-binding residues are Gln192, Arg213, Phe224, His234 and Gly236. Met375 of the C-terminal bridge helix also interacts with the bound tetracycline substrate and is conserved among the type 1 TDases (FIG. 24, FIG. 30B, FIG. 32). The Tet(X6) X-ray crystal structures have asymmetric units and packing of crystal lattices (FIG. 33A, FIG. 33B). Despite structural similarities, type 1 TDases can show significant differences in enzyme kinetics with up to -10-fold difference in apparent catalytic efficiency. Mutations outside the TDase active site generated via directed evolution and natural selection have been shown to enhance enzyme efficiency and resistance levels in whole cell assays. It is likely that surface-exposed residues affect both conformational dynamics and active site environments of different TDases, leading to differences in protein stability and enzyme efficiency that influence resistance phenotypes. Additional putative binding sites have been observed at the entrance of the active site in the Tet(X)-minocycline complex crystal structure (PDB ID: 4A99) that could plausibly guide the substrate into the active site.

### Tet(X6) Confers Pan-Tetracycline-Resistance

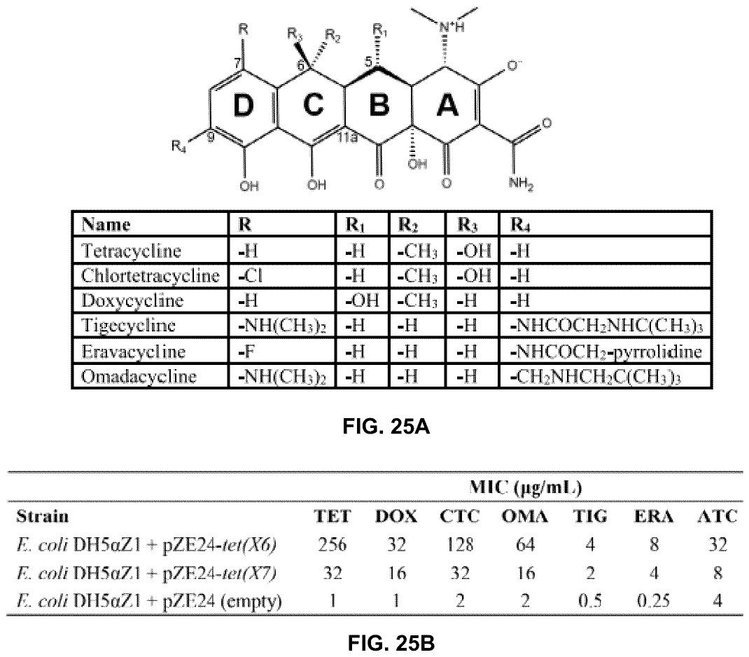

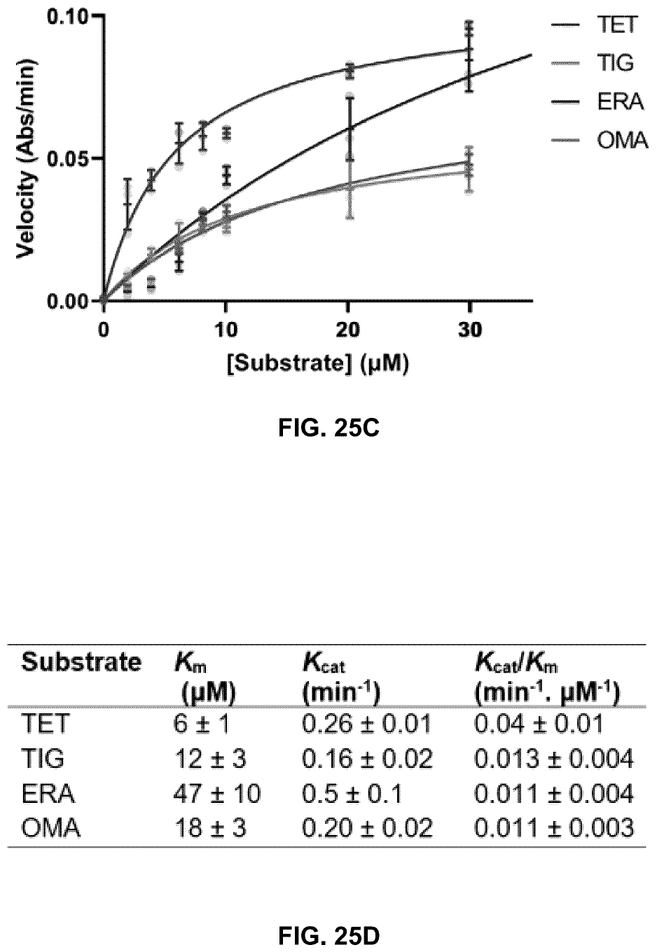

We validated the activity of Tet(X6) in the E. coli DH5αZ1+pZE24 system using microbroth dilution antibiotic susceptibility tests (FIG. 25A, FIG. 25B). For positive and negative controls, we also characterized the resistance profiles of E. coli strains producing Tet(X7) or containing the empty pZE24 vector (i.e., with no tetracycline resistance gene). In accordance with the initial report on this enzyme, heterologous expression of Tet(X6) in E. coli conferred high minimal inhibitory concentrations (MICs) against tetracycline antibiotics from all three generations (FIG. 25B). The highest MICs were against first-generation drug molecules tetracycline and chlortetracycline (256 and 128 μg/mL, respectively). Consistent with other type 1 TDases, Tet(X6) also conferred resistance to doxycycline, a second-generation tetracycline, as well as third-generation tetracyclines tigecycline, omadacycline, and eravacycline. Notably, the Tet(X6) strain's MICs are 2- to 8-fold higher than the Tet(X7) strain, which had been previously considered one of the most active TDases (FIG. 25B).

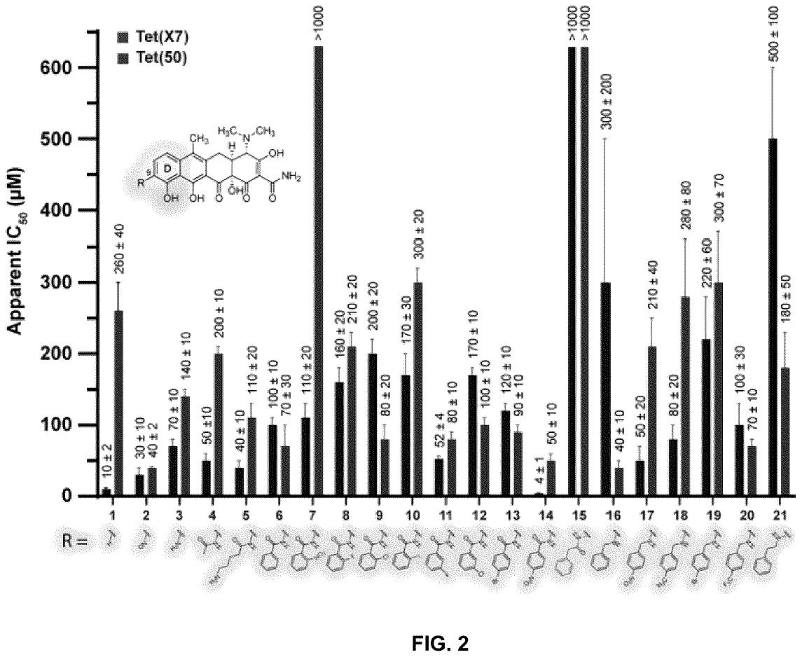

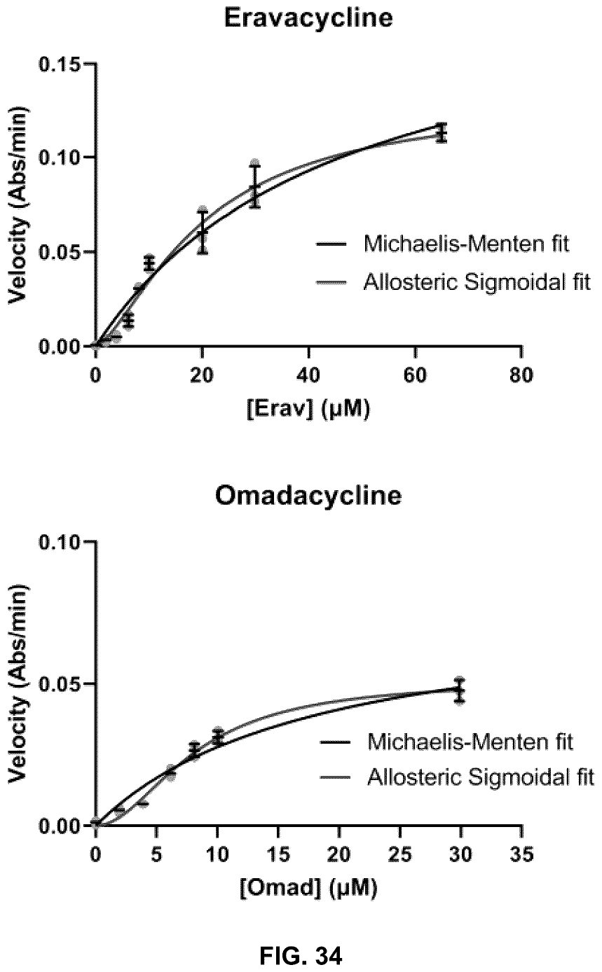

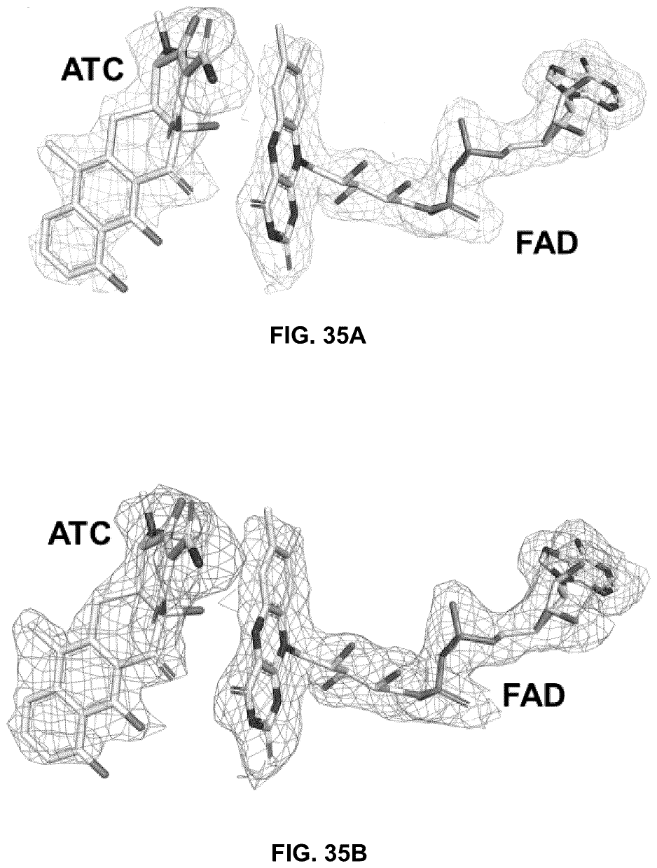

To study substrate binding and catalytic efficiency of Tet(X6) under steady state conditions, we continuously monitored the change in absorbance at 400 nm (unique Amax for tetracyclines) to observe direct enzymatic inactivation of tetracycline substrates: tetracycline, tigecycline, omadacycline, and eravacycline by Tet(X6) (FIG. 25C, FIG. 25D). The velocity versus substrate concentration curves for tetracycline and tigecycline were hyperbolic with good fit to the standard Michaelis-Menten equation. The curves for eravacycline and omadacycline appeared sigmoidal in nature, indicating the potential for allostery or multiple binding orientations for the substrate, the latter of which has been observed for the TDase family. Curve fitting to Michaelis-Menten and allosteric sigmoidal models in GraphPad prism both produced acceptable fits (R²>0.95) (FIG. 34). Based on the Michaelis-Menten fits for all antibiotic substrates, the range of apparent Kₘ values was 5-57 within the error of uncertainty ranging from 17-25% for these data fits (FIG. 2D). Among the five substrates studied, tetracycline showed the lowest apparent Kₘ value of 6±1 μM, suggesting the highest apparent binding affinity, followed by tigecycline (apparent Kₘ=12±3 μM), omadacycline (apparent Kₘ=18±3 μM), and eravacycline (apparent Kₘ=47±10 μM). The apparent catalytic efficiency of Tet(X6) was greatest for tetracycline as the substrate (k_{cat}/Kₘ=0.04±0.01 min⁻¹ μM⁻¹) compared to third-generation tetracycline substrates, and is mainly driven by Kₘ. The apparent catalytic efficiencies (k_{cat}/Kₘ) for Tet(X6)-catalyzed oxidation of eravacycline (k_{cat}/Kₘ=0.011±0.004 min⁻¹ μM⁻¹), tigecycline (k_{cat}/Kₘ=0.013±0.004 min⁻¹ μM⁻¹), and omadacycline (k_{cat}/Kₘ=0.011±0.003 min⁻¹ μM⁻¹) were similar.

### Anhydrotetracycline Rescues Tet)X6)-Mediated Inactivation of Tetracycline Antibiotics

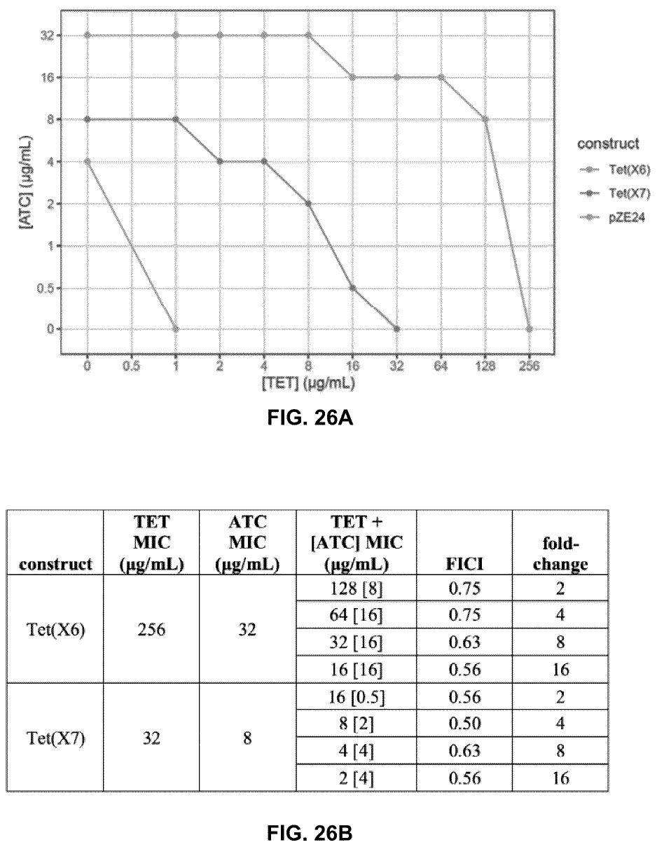

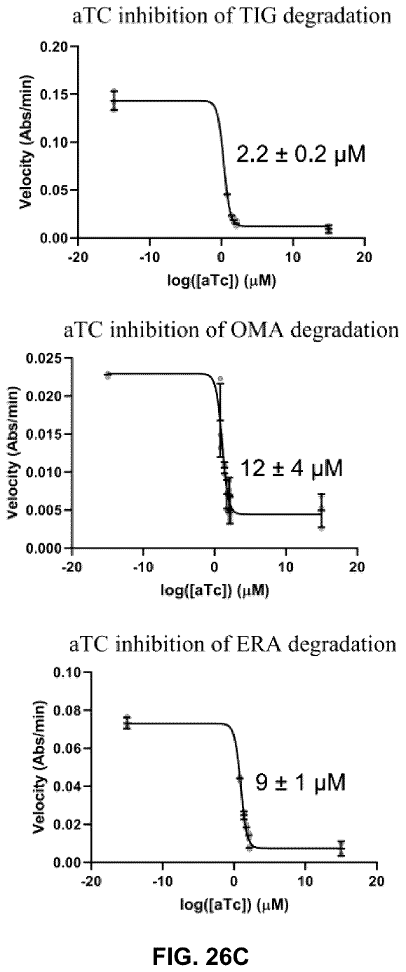

We have previously established that anhydrotetracycline inhibits a wide range of TDases and therefore can be classified as a pan-TDase inhibitor. Here, we study if anhydrotetracycline can rescue tetracycline activity against E. coli producing Tet(X6). We identified anhydrotetracycline concentrations that result in a tetracycline MIC lower than that of tetracycline alone, using checkerboard broth microdilution antibiotic susceptibility assays which test for cell growth in multiple tetracycline-anhydrotetracycline combinations. The addition of 16 μg/mL anhydrotetracycline reduced by 16-fold the concentration of tetracycline required to inhibit growth of Tet(X6)-producing E. coli, from 256 μg/mL to 16 μg/mL (FIG. 26A). The calculated fractional inhibitory concentration (FICI) index is provided for reference (FIG. 26B).

While anhydrotetracycline has antibiotic activity on its own against E. coli, we used concentrations below the MIC (32 μg/mL; FIG. 25B), then evaluated the inhibitory activity of anhydrotetracycline against Tet(X6)-mediated degradation of tetracycline antibiotics (FIG. 26C). The apparent half-maximal inhibitory concentrations (IC50s) observed were in the low micromolar range (2-12 μM), consistent with concentrations used in the whole cell rescue assays. Together these results suggest that, as with other TDases, anhydrotetracycline inhibition is a promising combination therapy against bacteria producing Tet(X6).

### Anhydrotetracycline Binds in a Substrate-Like Orientation in Type 1 TDases

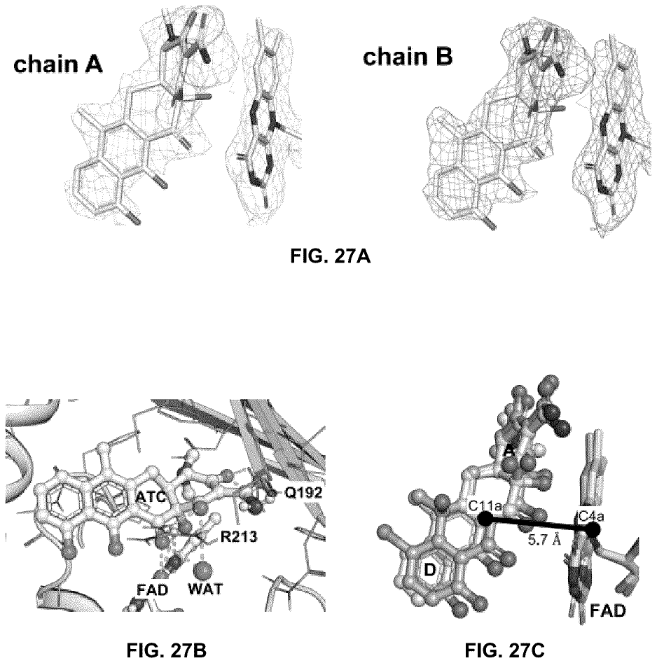

An X-ray co-crystal structure of anhydrotetracycline in complex with Tet(X6) was determined at 2.2 Å resolution (Table 3). A clear non-protein density was observed in the substrate-binding cavity, consistent with the size and shape of anhydrotetracycline (FIG. 27A). This structure shows that anhydrotetracycline binds in a substrate-like orientation in Tet(X6). The isoalloxazine group of bound FAD occupies an ‘IN’ orientation in the Tet(X6)-anhydrotetracycline complex structure, as observed for the anhydrotetracycline-free structure (FIG. 27A). The anhydrotetracycline binds to Tet(X6) in a substrate-like orientation, placing the A-ring close to the FAD and the D-ring close to the C-terminal bridge helix. The orientation of anhydrotetracycline in Tet(X6) remains conserved in structures of tetracycline substrates in complex with other type 1 TDases and is likely driven by the shared planarity of rings B, C, and D between anhydrotetracycline and other tetracycline substrates.

To compare polar and non-polar interactions of anhydrotetracycline in Tet(X6) with previously studied interactions in other type 1 TDase-substrate complexes, we aligned the Tet(X6)-anhydrotetracycline complex structure to a previously solved X-ray crystal structure of Tet(X) in complex with chlortetracycline (PDB ID: 2Y6R). The protein-ligand interactions between the tetracycline moieties and the residues of type 1 TDases remain conserved in the Tet(X6)-anhydrotetracycline and Tet(X)-chlortetracycline structures (FIG. 32). For example, the substitution of the 2,3-enol hydroxyl group at the A-ring of the bound anhydrotetracycline forms a hydrogen bond with side chains of Gln192 (FIG. 27B). The carboxamide carbonyl oxygen at the 2-position of anhydrotetracycline forms a hydrogen bond with Arg213. A water molecule serves as a hydrogen-bonding bridge between the carboxamide substitution of anhydrotetracline and Thr59. In addition, the Phe224-side chain stabilizes the bound anhydrotetracycline through π-cation interaction with the 7-dimethylamino substitution of anhydrotetracycline. Further, the isoalloxazine-ring system of FAD forms two additional H-bonds with anhydrotetracycline. The O4 and N5 atoms of FAD form H-bonds to the keto-enol moiety (012) and nearby hydroxyl group (012a) of bound anhydrotetracycline. These interactions contribute to orienting the anhydrotetracycline scaffold for hydroxylation by a putative C4a-peroxy-flavin reactive intermediate at site C11a, with at an appropriate short distance of -5.6 Å from the C4a of FAD-isoalloxazine heterocycle. Anhydrotetracycline also makes two H-bonds with aromatic amino acids: hydrogen atom at the C-7 position in the D-ring of anhydrotetracycline with the backbone oxygen atom of Phe319; and oxygen of the carboxamide moiety attached to the C-2 position of the A-ring of anhydrotetracycline with the aromatic hydrogen atom of His234 (FIG. 27B). An alignment of the Tet(X6)-anhydrotetracycline complex with previously solved Tet(X)-chlortetracycline complex (PDB ID: 2Y6R) confirmed a conserved distance of -5.7 Å between C11a of anhydrotetracycline and C4a of the FAD isoalloxazine ring, suggesting a correlation of this binding mode with hydroxylation of C11a across substrate classes (FIG. 27C).

### Anhydrotetracycline Oxidation is Catalyzed by Type 1 TDases

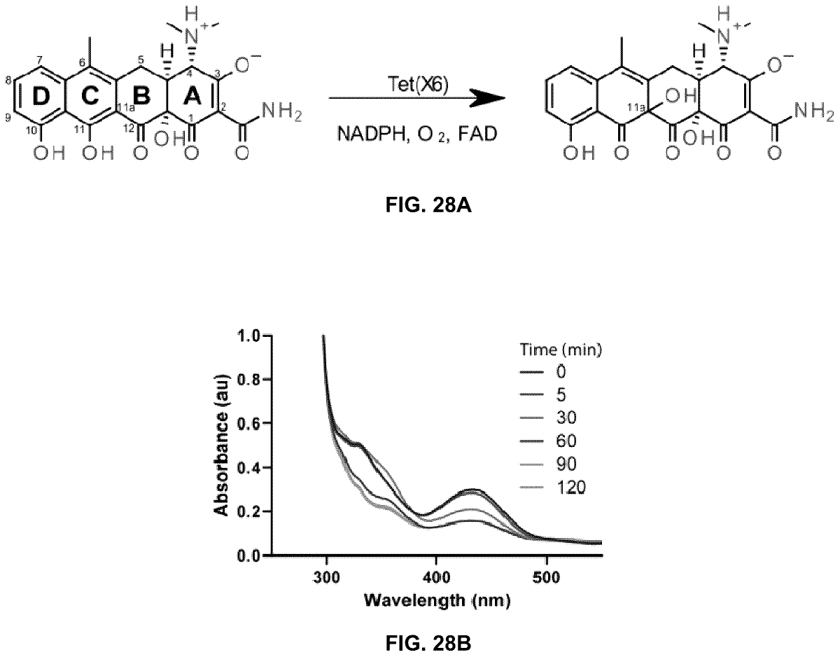

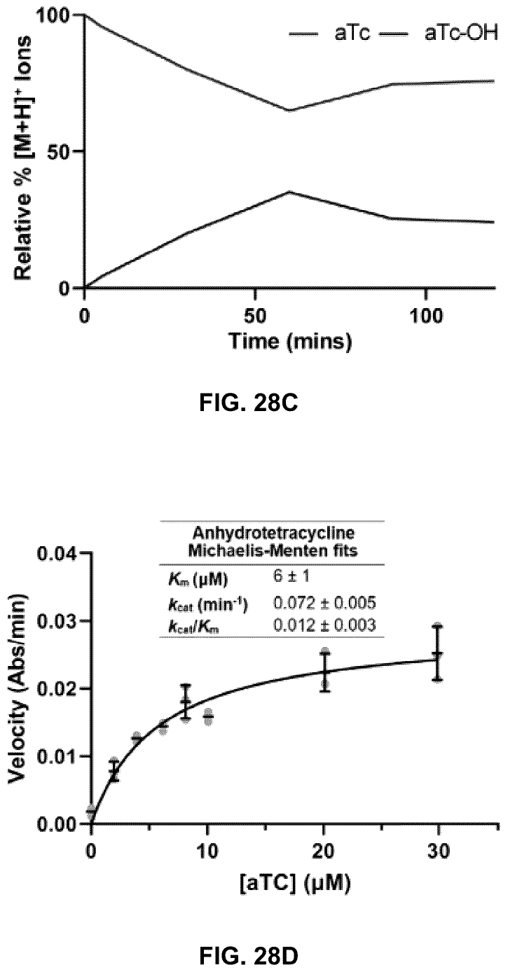

We have previously established that anhydrotetracycline is a pan destructase inhibitor that inhibits diverse type 1 and type 2 TDases. While type 2 TDases cannot metabolize anhydrotetracycline, type 1 TDases such as Tet(X) are capable of slowly turning over anhydrotetracycline as a substrate. The Tet(X6)-anhydrotetracycline complex structure shows a substrat-e-like binding mode of anhydrotetracycline in Tet(X6). —Therefore, we speculated that Tet(X6) can oxidize anhydrotetracycline at the C11a atom (FIG. 28A). To assess the potential for anhydrotetracycline to serve as a substrate for Tet(X6), we performed an in vitro optical absorbance kinetic assay and LC-MS analysis, as previously reported. We observed degradation of anhydrotetracycline as indicated by the time-dependent decrease at 440 nm (unique Amax for anhydrotetracycline under assay conditions) in the presence of Tet(X6), NADPH, and O₂ (FIG. 28B). We analyzed the same reaction mixtures by LC-MS and identified ions corresponding to the predicted mass for the [M+O+H]⁺ molecular ion of oxidized anhydrotetracycline that appeared with stoichiometric loss of the [M+H]⁺ ion corresponding to the parent anhydrotetracycline predicted mass (FIG. 28C). To evaluate the catalytic efficiency of Tet(X6) degradation of anhydrotetracycline, Michaelis-Menten steady-state kinetics were determined (FIG. 28D). The observed apparent binding affinity (Kₘ) was 6±1 μM, similar to tetracycline, but lower than tigecycline (apparent Kₘ=12±3 μM), omadacycline (apparent Kₘ=18±3 μM), and eravacycline (apparent Kₘ=47±10 μM) (FIG. 25D). The apparent k_{cat} for Tet(X6) with anhydrotetracycline as the substrate, however, was significantly lower than tetracycline antibiotic substrates by up to a factor of 7, at 0.072±0.005 μM (FIG. 28D). This implies that the reduced catalytic efficiency of Tet(X6) for turning over anhydrotetracycline (k_{cat}/Kₘ=0.012±0.003 min⁻¹ μM⁻¹) compared to tetracycline (k_{cat}/Kₘ=0.04±0.01 min⁻¹ μM⁻¹) is driven by an apparent reduction in k_{cat}. This is consistent with the model that anhydrotetracycline binds rapidly in the active site of Tet(X6) and serves as a competitive substrate for Tet(X6) with a much lower rate of catalysis (k_{cat}). The binding orientation and enzyme kinetics of anhydrotetracycline in Tet(X6) open new avenues for rational drug design to develop potent anhydrotetracyline-based type 1 TDase inhibitors that further reduce k_{cat} or eliminate catalysis.

### Discussion

In this study, we determined X-ray structures of the Type 1 TDase Tet(X6) as a holo-enzyme (FAD-bound) and in complex with anhydrotetracycline. The X-ray structure of the Tet(X6)-anhydrotetracycline complex demonstrates a substrate-like binding orientation of anhydrotetracycline, supported by biochemical and cellular studies which indicate a substrate-like metabolism. We established that anhydrotetracycline competitively inhibits Tet(X6)-mediated inactivation of tetracycline antibiotics despite the ability of Tet(X6) to turnover anhydrotetracycline as a substrate. This type of sacrificial substrate inhibition of enzymes serves as the basis of clinically useful β-lactamase inhibitor/β-lactam antibiotic combination therapies. The inhibition of β-lactamases is achieved through an initial hydrolysis of the bound inhibitor leading to covalent adduct formation (clavulanic acid and sulbactam) or reversible hydrolysis (avibactam). Here, the inhibition of Tet(X6) by anhydrotetracycline is reversible and the oxidized anhydrotetracycline product is released. Hence, the apparent inhibition of Tet(X6) arises due to the ability of anhydrotetracycline to outcompete tetracycline antibiotics for binding combined with a slower rate of enzymatic turnover of anhydrotetracycline relative to tetracyclines. The sacrificial nature of anhydrotetracycline occupies TDases in bacterial cells to reduce the rate and likelihood of TDase-mediated degradation of tetracycline antibiotics when used in combination therapies. The type 2 TDases cannot metabolize anhydrotetracycline in this manner. Type 1 TDases from clinical pathogens contain conserved residues in the FAD-binding pocket and in the substrate-binding pocket, suggesting a shared mechanism of anhydrotetracycline binding and enzyme inhibition. Our X-ray crystal structures provide insights into the binding mode of anhydrotetracycline in type 1 TDases and may guide design and development of more potent TDase inhibitors that do not act as sacrificial substrates.

Multiple recent studies have suggested combination therapy consisting of a tetracycline antibiotic with a TDase inhibitor is also feasible. Park et al. reported that anhydrotetracycline can rescue tetracycline efficacy in pathogens expressing Tet(56), a type 2 TDase. Markley et al. generated several analogs of anhydrotetracycline with halogenation of the D-ring to extend the spectrum of inhibition against type 1 and type 2 TDases. Liu et al. demonstrated that combining antiviral agent azidothymidine (AZT) with tigecycline decreased the survival of E. coli expressing Tet(X4). Xu et al. established that plumbagin, a natural naphthoquinone isolated from plants, shows synergistic effects with tetracycline antibiotics against Tet(X3)-/Tet(X4)-producing bacteria. Deng et al. confirmed a combination of Bi(NO₃)₃ and tigecycline can prevent development of resistance in bacteria expressing Tet(X). Most recently, Williford et al. reported a series of C9-benzamide and C9-benzylamine anhydrotetracycline analogs that act as bisubstrate inhibitors of type 1 and type 2 TDases. Despite these recent investigational studies, the structural characteristics of an inhibitor in clinically relevant TDases (i.e., type 1 TDases), remain unknown. Structural insights into TDase-inhibitor complexes are imperative to design potent and effective TDase inhibitors.

The structural architecture of a TDase is composed of three conserved, key features: (i) a Rossman-fold containing FAD-binding domain; (ii) a substrate-binding domain; (iii) and a C-terminal bridge helix (FIG. 24). TDases are broadly classified into two main classes: type 1 TDases (also known as Tet(X)-like TDases) and type 2 TDases (also known as soil-derived TDases). A key structural difference between type 1 and type 2 TDases is an additional C-terminal, ‘gatekeeper’ α-helix present in type 2 TDases (FIG. 31). This ‘gatekeeper’ helix regulates substrate loading and catalysis and may plausibly clash with D-ring substituted tetracyclines that include third-generation tetracyclines. Therefore, type 2 TDases remain inactive against third-generation tetracyclines. In contrast, type 1 TDases that lack the ‘gatekeeper’ C-terminal helix can accommodate D-ring substitutions on the tetracycline moiety and inactivate all available tetracyclines. The dynamics of the bound FAD cofactor also play a critical role in enzyme catalysis/inhibition. During the substrate oxidation step of the catalytic cycle, the FAD occupies an ‘IN’ orientation to position the presumed C4a-peroxy-flavin for oxygen transfer to C11a of the bound substrate. The oxidized FAD transitions to the ‘OUT’ state for subsequent regeneration (presumably reduction by NADPH) and availability for the next round of catalysis.

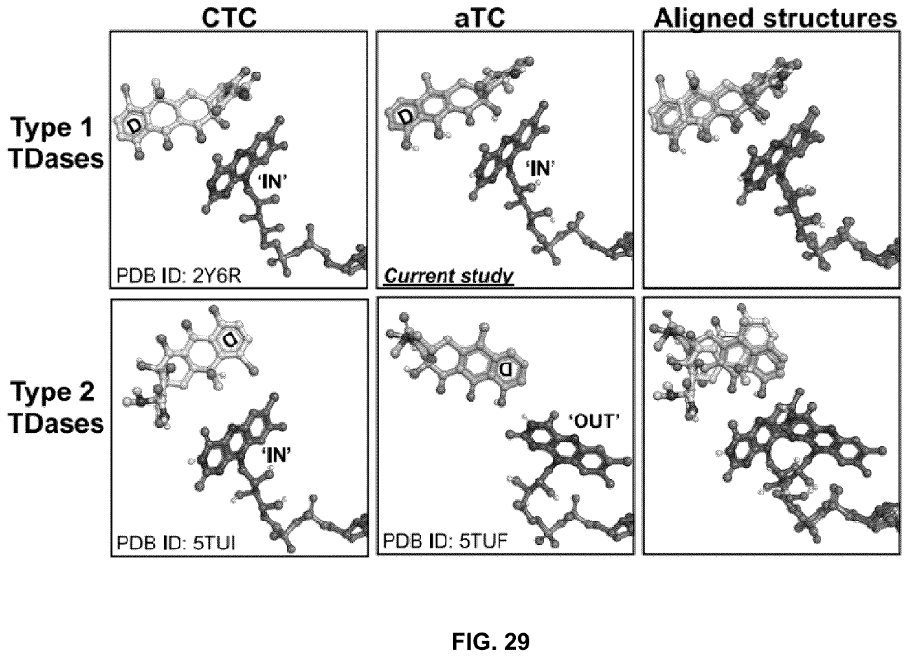

Prior to this report, no structural details have been available for type 1 TDases in complex with an inhibitor. Anhydrotetracycline is a pan-TDase inhibitor that acts against both classes of TDases in different biochemical and cellular assays. Here, we have solved the X-ray structure of a type 1 TDase, Tet(X6), in complex with anhydrotetracycline. The Tet(X6)-anhydrotetracycline complex shows the FAD cofactor bound in an ‘IN’ conformation that is distinct from the ‘OUT’ conformation observed for FAD in X-ray structures of type 2 TDases complexed with anhydrotetracycline. The anhydrotetracycline binds in a substrate-like orientation in the Tet(X6) active site and would compete for binding with diverse tetracycline substrates (FIG. 29). Steady-state kinetics suggest anhydrotetracycline is a good binding ligand but a poor substrate for catalysis relative to tetracycline antibiotics. The extra site of dehydration at the C5′-C6 bond of anhydrotetracycline creates a more stable aromatic naphthalene moiety compared to the styrene moiety present in tetracyclines. Anhydrotetracycline also benefits from extended conjugation across the sensitive 1,3-diketo/enol system formed by atoms C11-C11a-C12 at the C,D-ring juncture. Oxidation at C11a of anhydrotetracycline and tetracycline presumably occurs via hydroxyl group transfer from the reactive C4a-peroxy-flavin intermediate. The extra stabilization of anhydrotetracycline is likely to reduce the nucleophilicity of the enol tautomer at C11a and slow the rate of oxidation at C11a compared to more reactive tetracycline substrates.

Type 1 TDases are more commonly found in pathogens. Despite conserved residues in the FAD-binding pocket and in substrate-binding pockets, different type 1 TDases show distinct catalytic efficiencies towards different substrates. Mutations in regions distant from ligand binding cavities may plausibly contribute to the difference in structural dynamics and influence local active site structures resulting in different catalytic efficiencies. Indeed, a surface-localized, single point mutation, Thr280Ala in Tet(X2) reduces the apparent Kₘ for minocycline by two-fold.

Anhydrotetracycline-based inhibitors have been shown to inhibit diverse TDases in enzymatic and cellular studies. This study provides structural insights into the binding mode of anhydrotetracycline complexed with type 1 TDase Tet(X6) and reveals key pharmacophoric features in the active site cavity, which can be explored further to develop improved anhydrotetracycline-based inhibitors with enhanced binding affinity and enzyme inhibition. Specifically, side chains of Asp61, His63, Asn112, Gln322, Glu367, Asn371 in the vicinity of the D-ring of bound anhydrotetracycline may be targeted to form additional H-bonds with the designed inhibitor through substituents on the D-ring of the anhydrotetracycline scaffold. Similarly, side chains of Asp61 and Arg213 are positioned favorably to form additional H-bonds with polar group substitutions at the C-ring of the anhydrotetracycline scaffold. Substituents on the B- and A-rings of tetracycline scaffold could possibly form additional H-bonds with the side chains of Ser238, Asn190, His234, Gln192. These findings establish a foundation for rational structure-based design of anhydrotetracycline-based inhibitors to combat antibiotic resistance conferred by type 1 TDases.

### Methods

### Cloning and Protein Expression

The coding region of tet(X6) (QHN11884.1) was cloned in a pET28 vector (cleavage sites: Age1, Kpn1) with a 6-His tag at the C-terminus. The cloned construct was then transformed into E. coli BL21 (DE3) to express the protein. Cells were cultured at 37° C. in LB media, containing 0.03 mg/ml kanamycin, until the OD₆₀₀ (optical density at λ=600 nm) reached 0.6-0.8. At that point, the temperature was lowered to 18° C. and expression was induced with 1 mM Isopropyl β-D-1-thiogalactopyranoside (Sigma-Aldrich, St. Louis, MO). The induced culture was grown overnight (˜20 h) at 18° C. and centrifuged at the maximum speed (20 min, 4° C.) to pellet down the cells.

### Protein Purification

The cell pellet was resuspended in the lysis buffer [50 mM Tris (pH 8.0), 100 mM NaCl, 10 mM imidazole (pH 8.0) 10 mM beta-mercaptoethanol (BME), Pierce™ protease inhibitor tablet (catalog #A32963; Thermo Scientific)] and stored at −80° C. To purify the protein, the frozen pellet was thawed in the presence of 0.25 mg/mL lysozyme. The cells were disrupted using a sonicator (ON/OFF/total-time: 0.5/0.5/120 s). The cell lysate was centrifuged at 25,000 g for 20 min and the supernatant was loaded on nickel rapid run agarose beads (Goldbio) that were previously equilibrated with the wash buffer [50 mM Tris (pH 8.0), 100 mM NaCl, 10 mM imidazole (pH 8.0), 5 mM BME and Pierce™ protease inhibitor tablet (catalog #A32963; Thermo Scientific). The beads were washed three times with five-column volume of wash buffer and finally eluted with 3-column volume of the elution buffer [50 mM Tris (pH 8.0), 100 mM NaCl, 500 mM imidazole (pH 8.0)]. The eluted protein sample was further purified by gel purification using HiLoad 16/600 Superdex 200 μg column (GE Healthcare) equilibrated with 10 mM Hepes (pH 7.4), 100 mM NaCl, 5 mM DTT. The fractions containing the protein of interest were pooled and concentrated using a 10 K MWCO Amicon centrifugal filter (Millipore). During all steps, the sample was kept at 4° C.

### Crystallization, Data Collection, and Structure Refinement

Tet(X6) was concentrated to 20 mg/mL and crystallized by vapor diffusion in hanging drop at 18° C. in 0.2 M potassium thiocyanate and 20% (w/v) PEG 3350. Crystals were transferred into 0.2 M potassium thiocyanate, 20% (w/v) PEG 3350 and 20% PEG 400 for 15-30 s and flash-cooled in liquid nitrogen. 4 mM anhydrotetracycline was added to 15 mg/mL Tet(X6), centrifuged at 10,000 g for 10 min at 4° C., and the complex crystallized by vapor diffusion in hanging drop at 18° C. in 0.2 M potassium thiocyanate and 20% (w/v) PEG 3350. The co-crystals were transferred into 0.2 M potassium thiocyanate, 20% (w/v) PEG 3350 and ethylene glycol for 15-30 s and flash-cooled in liquid nitrogen. Diffraction data were collected at 100 K on beamline 22-ID (APS). 900 frames were collected with the oscillation step of 0.2 degrees. Sample to detector distance was set to 235 mm for anhydrotetracycline-free Tet(X6) crystal and to 250 mm for Tet(X6)+anhydrotetracycline crystal. All data processing and structure analysis were performed using SBGrid. Diffraction data was reduced and scaled using XDS. Tet(X6) structure was solved by molecular replacement using Phaser with the Tet(X7) structure (PDB ID: 6WG9; sequence identity: ˜94%) as a starting model. The protonation pattern of anhydrotetracycline was defined as previously described. Structure refinement was performed in Phenix and Coot. The final model was validated using the Molprobity server.

### Antibiotic Susceptibility and Checkerboard Inhibition Assays

Tetracycline resistance genes were cloned into the KpnI and MluI sites of the pZE24 plasmid (Expressys); this plasmid is maintained using kanamycin and its P_{lac/ara-1} promoter can be regulated with IPTG and arabinose. For high expression, 1 mM IPTG fully relieves repression by Lac thus only IPTG was used in whole-cell tests. Chemically competent E. coli DH5αZ1 (Expressys) was transformed with these pZE24 constructs by heat shock. Minimum inhibitory concentrations (MICs) were measured as per Clinical and Laboratory Standards Institute (CLSI) guidelines. Substrates and inhibitors were dissolved in DMSO (20 mg/mL) then diluted to working concentrations in cation-adjusted Mueller-Hinton II broth supplemented with 50 μg/mL kanamycin. Antibiotic susceptibility testing panels were prepared in 96-well flat-bottom microplates (Corning) by two-fold serial dilution of the antibiotic of interest. For checkerboard whole cell inhibition assays, anhydrotetracycline was two-fold serially diluted in a constant concentration of tetracycline. Liquid cultures of each strain were grown to exponential phase then diluted to a standard concentration (OD₆₀₀=0.0015, which is equivalent to double ˜5×10⁵ CFU/mL) and inoculated into each panel at a 1:1 ratio. Thus, each well had a final concentration of 50 μg/mL kanamycin, 1 mM IPTG, ˜5×10⁵ CFU/mL (0.5 MacFarland) cells, and variable concentrations of the antibiotic of interest or anhydrotetracycline. Each strain-antibiotic/inhibitor combination was tested in triplicate, along with no-drug and no-cell controls. Inoculated panels were sealed with Breathe-Easy membranes (Sigma-Aldrich) and incubated at 37° C. for 20 h. MICs were scored by absorbance measurements at 600 nm (OD₆₀₀) using the Synergy H1 microplate reader (Biotek Instruments, Inc). Synergy of inhibitor and tetracycline combinations was determined using the fractional inhibitory concentration index (FICI) method where FICI>1 indicates antagonism, FICI=1 indicates additivity, and FICI<1 indicates synergy:

$$FICI = \frac{MIC {A}_{combo}}{MIC {A}_{alone}} + \frac{MIC {B}_{combo}}{MIC {B}_{alone}}$$

### Characterization of Substrate Degradation by Scanning Optical Absorbance Spectroscopy and LC-MS

All in vitro kinetic assays were prepared open to air in non-degassed buffer solutions at room temperature. TDase reactions were prepared in 100 mM TAPS buffer (pH 8.5) with an NADPH regenerating system (40 mM glucose-6-phosphate, 4 mM NADP+, 1 mM MgCl₂, 4 U/mL glucose-6-phosphate dehydrogenase), 20 μM substrate and 0.24 μM Tet(X6) (all concentrations represent final working concentrations). In vitro reactions were monitored by optical absorbance spectroscopy on an Agilent Cary 50 UV-visible spectrophotometer using polystyrene cuvettes. Reaction progress was monitored by optical absorbance spectroscopy (280-550 nm, 1 nm and 5 min intervals) over 2 h. Aliquots of reaction sample (150 μL) were removed and quenched (600 μL of 1:1 acetonitrile/0.25 M aqueous HCl) immediately after enzyme was added (0 min) and at 5, 30, 60, 90, and 120 min intervals. The quenched samples were centrifuged (5000 rpm, 4° C.) for 5 min, and 600 μL of the resulting supernatant was mixed with an Fmoc-Ala internal standard (3.12 μM final concentration) and analyzed by LC-MS in positive ion mode (single trial). LC-MS was acquired using an Agilent 6130 single quadrupole instrument (ESI+) with G1313 autosampler, G1315 diode array detector, and 1200 series solvent module with separation on a Phenomenex Gemini C18 column, 50×2 mm (5 um) fit with a guard column cassette. LCMS solvents were 0.1% formic acid in H₂O (A) and 0.1% formic acid in acetonitrile (B). Solvent gradient was linear starting from 0% B to 95% B over 20 min at a flow rate of 0.5 mL/min. LCMS data were processed using ChemStation software version B.04.02 SP1. Extracted ion chromatograms (EICs) for the expected [M+H]⁺ molecular ions corresponding to substrate and mono-hydroxylated product were normalized to the [M+H]⁺ counts for an Fmoc-Ala internal standard.

### Characterization of Steady-State Kinetics for Substrate Inactivation

All experiments were prepared open to air in non-degassed buffer solutions at room temperature. Reactions were prepared in 100 mM TAPS buffer at pH 8.5 with 0-30 μM substrate (a 65 μM concentration was included for eravacycline to reach vₘₐₓ), 504 μM NADPH, 5.04 mM MgCl2, and 0.4 μM Tet(X6) (final working concentrations). Reactions were initiated by the addition of Tet(X6) and were monitored continuously via optical absorbance spectroscopy at 400 nm (440 nm for aTC inactivation) for 2 min (performed in triplicate as independent trials). Initial enzyme velocities were determined by linear regression using Agilent Cary WinUV Software over the linear range of the reaction (typically between 0 to 1 min), plotted against the concentration of the substrate, and fitted to the Michaelis-Menten or allosteric sigmoidal nonlinear regression equations using GraphPad Prism 6.

### Determination of Apparent Tet(X6) Inhibitor IC50 Values

All experiments were prepared open to air in non-degassed buffer solutions at room temperature. Half-maximal inhibitory concentrations (IC50) for the inhibition of Tet(X6) were determined from the velocities of substrate degradation in the presence of varying concentrations of inhibitor. Reaction samples were prepared in 100 mM TAPS buffer (pH 8.5) with 504 μM NADPH, 5.04 mM MgCl₂, 25.3 μM TC, varying concentrations of inhibitor (typically 0-146 μM), and 0.4 μM Tet(X6) (final working concentrations). Reactions were initiated by the addition of Tet(X6) and were monitored continuously via optical absorbance spectroscopy at 400 nm for 2 min (performed in triplicate as independent trials). Initial enzyme velocities were determined by linear regression using Agilent Cary WinUV Software over the linear range of the reaction (typically between 0 to 1 min). The velocities were plotted against the logarithm of inhibitor concentration, and apparent IC₅₀ values were determined using nonlinear regression analysis in GraphPad Prism v6. Each set of experiments included a no-TDase control reaction which was used as the full enzyme inhibition velocity and assigned to an inhibitor concentration of 1×10¹⁵, as well as a no-inhibitor control which was assigned an inhibitor concentration of 1×10⁻¹⁵ Å no-TC control was also performed to search for potentially competitive background signals generated from the enzymatic degradation of the inhibitor itself. For all inhibitor-enzyme combinations, the initial velocities of the no-TC controls were negligible.

### Statistics and Reproducibility

X-ray structural analysis statistics including number of crystals used per data set are include in Table 3. Standard statistical analysis for X-ray diffraction data processing and analysis were adhered to and presented in Table 3. Enzyme assays were performed in two or three independent trials and whole cell inhibition assays were performed in triplicate.

### Example 1—Structure-Based Design of Bisubstrate Tetracycline Destructase Inhibitors that Block Flavin Redox Cycling

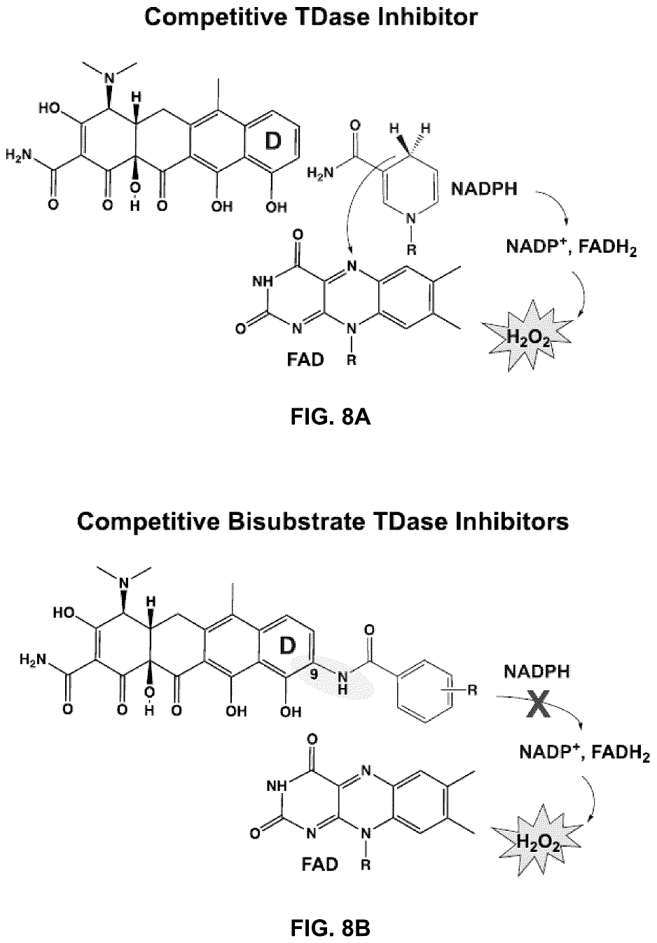

Tetracyclines (TCs) are an important class of antibiotics threatened by an emerging new resistance mechanism—enzymatic inactivation. These tetracycline-inactivating enzymes, also known as tetracycline destructases (TDases), inactivate all known TC antibiotics including drugs of last resort. Combination therapies consisting of a TDase inhibitor and TC antibiotic represent an attractive strategy for overcoming this type of antibiotic resistance. Here, the structure-based design, synthesis, and evaluation of bifunctional TDase inhibitors derived from anhydrotetracycline (aTC) are reported. By appending a nicotinamide isostere to the C9-position of the aTC D-ring, we generated bisubstrate TDase inhibitors. The bisubstrate inhibitors have extended interactions with TDases by spanning both the TC and presumed NADPH binding pockets. This simultaneously blocks TC binding and reduction of FAD by NADPH while ‘locking’ TDases in an unproductive FAD ‘out’ conformation (FIG. 8A, FIG. 8B).

### Introduction

Tetracycline (TC) antibiotics are a family of type-II polyketides originally isolated from Streptomyces aureofaciens. TCs have been in clinical use for >70 years as broad-spectrum antibiotics and continue to be used as frontline agents for treating a variety of infections caused by Gram-positive and Gram-negative bacteria. Until recently, it was thought that clinical TC resistance occurs primarily through the expression of efflux pumps and ribosome protection proteins. These resistance mechanisms have been largely overcome in the clinic by the development of last-generation TCs known as the glycylcyclines including the FDA-approved drugs tigecycline, eravacycline, and omadacycline. Unfortunately, all known TC antibiotics are susceptible to an emerging third route of clinical resistance: enzymatic inactivation by tetracycline destructase (TDase) enzymes.

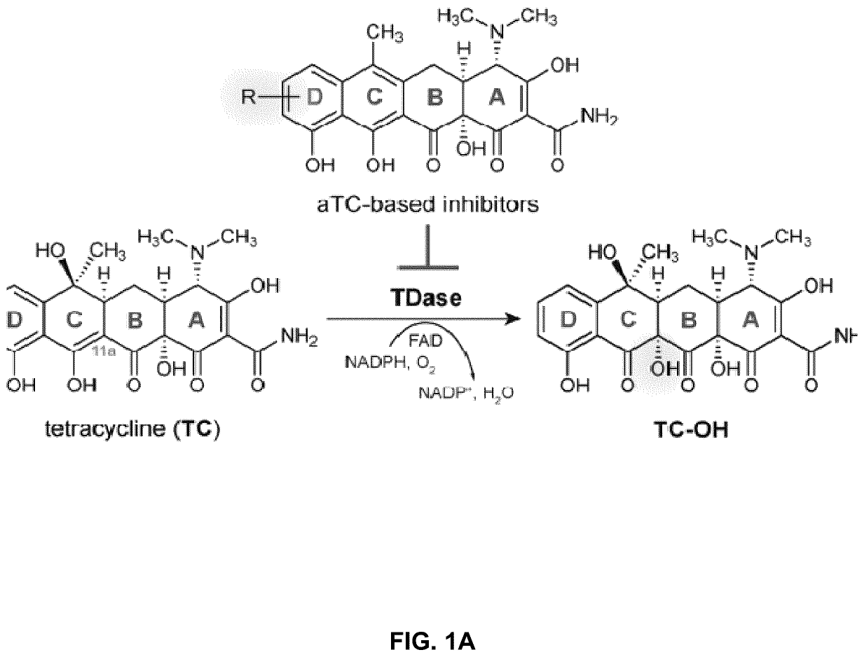

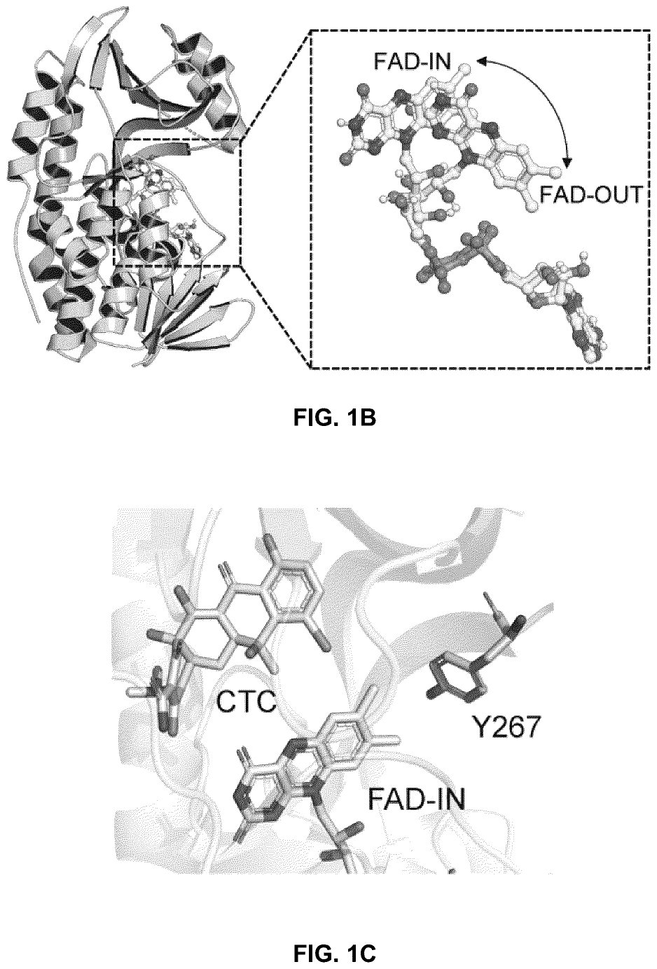

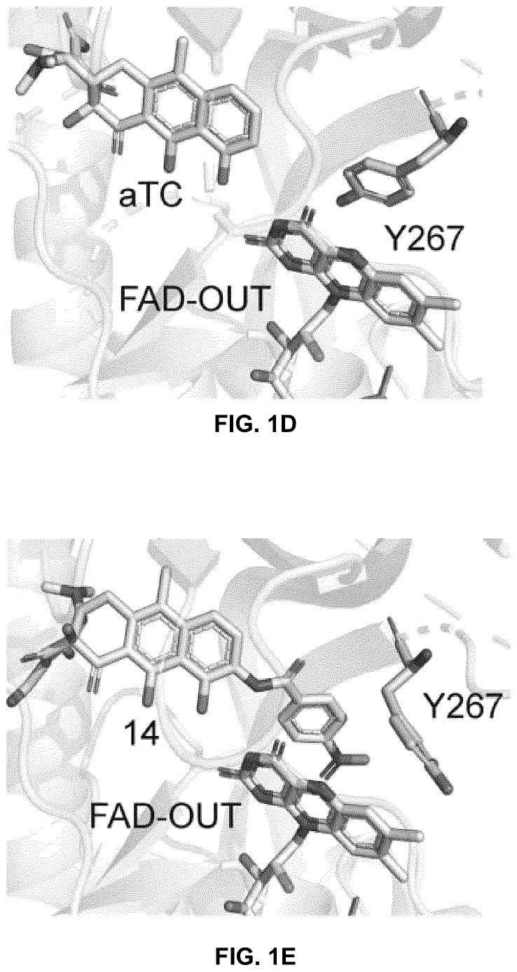

TDases are members of the class A flavin monooxygenase (FMO) enzyme family. TDases are FAD-dependent and use an NADPH/O2-coupled redox cycle to catalyze the inactivation of TC antibiotics. Oxidation of the bound TC substrate occurs via a C4aperoxy-flavin intermediate resulting in substrate-dependent oxygen transfer (hydroxylation) and oxygen insertion (Baeyer-Villiger type) reactions (FIG. 1A). The resulting oxidized TC scaffolds lack antibacterial activity presumably due to a loss of binding affinity for the bacterial ribosome. TDases contain distinct substrate and FAD-binding domains connected via a C-terminal bridge helix (FIG. 1A). Two distinct types of TDases have and are found in human gut commensals and pathogens. Type 2 TDases (represented in this study by Tet(50)) contain an extra C-terminal helix that ‘gates’ the active site during the catalytic cycle, provide resistance to only first and second-generation TC antibiotics but not glycylcyclines, and are found primarily in environmental microbes. The structural and functional differences of Type 1 and 2 TDases have an important influence on substrate binding mode, flavin dynamics, mechanism of TC inactivation, and inhibition. At this point, Type 1 TDases appear to be the more likely clinical threat but the evolutionary connection between Type 1 and 2 TDases presents a unique opportunity to study TC resistance via enzymatic inactivation.

Enzymatic antibiotic inactivation is of particular concern given that this pathogen phenotype depletes the antibiotic challenge for the entire infection environment (including cells not expressing inactivating enzymes). Evolution of antibiotic inactivation enzymes under intense selective pressure is a potential gateway to pan resistance against entire drug classes. The clinical significance and global impact of this resistance mechanism has been fully realized for beta-lactam antibiotics where widespread dissemination of betalactamase encoding genes demands the co-administration of a beta-lactamase inhibitor to restore clinical efficacy of beta-lactam antibiotics. Presumably, TDase inhibitors will be needed in the future given the mobilization and widespread distribution of TDase genes in the environment including clinical and agricultural settings. The emergence of TDases in clinical pathogens is on an upward trajectory and the deployment of new glycylcyclines could exacerbate this trend as has been observed following the release of new broad-spectrum beta-lactam antibiotics. Anhydrotetracycline (aTC), which differs from parent TC by dehydration of the C6 alcohol, is a pan TDase inhibitor and rescues whole cell activity of TC antibiotics in E. coli and Mycobacterium abscessus. The co-crystal structure of aTC bound to the Type 2 TDase Tet(50) reveals a binding mode that is unique from the observed substrate binding mode leading to stabilization of the FAD-cofactor in an unproductive ‘OUT’ conformation that is stabilized through a π-π stacking interaction with the benzylic sidechain of Y267 (FIG. 1C, FIG. 1D).

In addition to the TDase inhibitory activity, aTC alone has inherent antibacterial activity and some general toxicity at the effective concentrations (low μM) due in part to its ability to disrupt cellular membranes. Further, some Type 1 TDases can turnover aTC as a slow substrate indicating that aTC can sample the productive substrate binding mode with FAD in the ‘IN’ conformation. Simple modifications of the aTC scaffold including halogenation of C7 and C9 of the D-ring or demethylation of C6 of the C-ring were tolerated but failed to mitigate these potential liabilities. Thus, we were motivated in this work to rationally design more effective TDase inhibitors with improved selectivity, potency, and stability by using structure-guided molecular modeling (FIG. 1E). We identified a solvent exposed channel between the aTC D-ring and Y267 in the aTC-Tet(50) co-crystal structure (PDB: 5TUF) that appeared to be accessible by benzamide and benzylamine substituents appended to the C9-position of the aTC D-ring. Hence, we designed and synthesized a series of ‘bisubstrate’ TDase inhibitors predicted to span the aTC binding site and occupy the space filled by Y267 used to stabilize FAD in the ‘OUT’ conformation through π-π stacking of the aryl substituent with the FAD isoalloxazine heterocycle. This is also the space proposed to be occupied by the NADPH nicotinamide ring during FAD reduction. The rationally designed bisubstrate inhibitors were stable towards TDase-catalyzed oxidation eliminating the slow catalytic turnover observed for aTC and showed dose dependent recovery of TC activity in whole cell assays. We report informative structure-activity relationships and mechanistic studies to guide future inhibitor optimization and identify synergistic inhibitor-antibiotic pairs to overcome TC resistance via enzymatic inactivation. Our approach to bisubstrate mechanism-based inhibitor design exploits flavin dynamics, perturbs redox couples, and may be broadly applicable across the class A FMO enzyme family.

### Results and Discussion

### Structure-Based Design of Bisubstrate TDase Inhibitors

The structure of the aTC-Tet(50) inhibition complex (PDB: 5TUF) shows aTC bound in the active site adjacent to and overlapping with the substrate binding site. The A-ring of aTC points towards the entrance of the active site and the D-ring extends toward a solvent exposed channel. The FAD cofactor is trapped in the ‘OUT’ conformation sterically occluded from accessing the ‘IN’ conformation by the bound aTC inhibitor. The FAD ‘OUT” conformation is stabilized through a hydrogen bond between the flavin N3-H and the aTC C10-OH and a π-π stacking interaction with the 4-hydroxy-benzyl sidechain of Y267 (FIG. 1D). A similar Tyr-mediated π-π stacking interaction has been observed for other FMOs including kynurenine-3-monooxygenase (KMO). A group at GlaxoSmithKline (GSK) proposed that this Tyr residue occupies the space that must be filled by the nicotinamide portion of NADPH during the reductive step. No co-crystal structure of a class A FMO liganded with NAD(P)H or NAD(P)+ in a catalytically relevant conformation has been reported and the functional binding site for this cosubstrate remains elusive. The GSK group used X-ray structures of KMO with competitive inhibitors occupying the substrate binding site combined with molecular docking of NADP+ to guide the design of bisubstrate inhibitors composed of known benzoxazolidinone inhibitor scaffolds fused with nicotinamide isosteres. We adapted the GSK scaffold hybridization approach to design a series of benzamide and benzylamine-substituted aTC analogs as potential bisubstrate inhibitors of TDases.

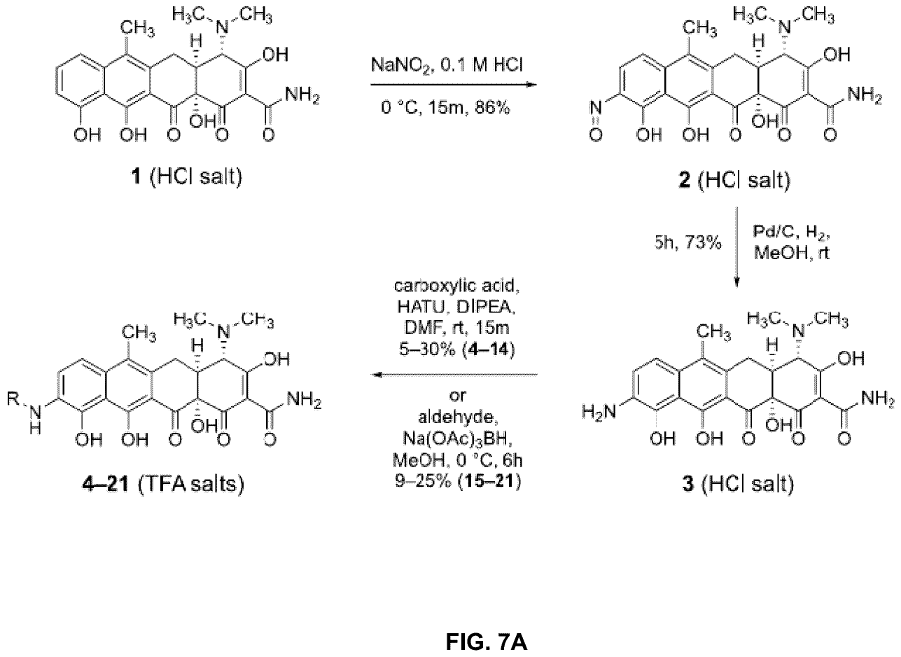

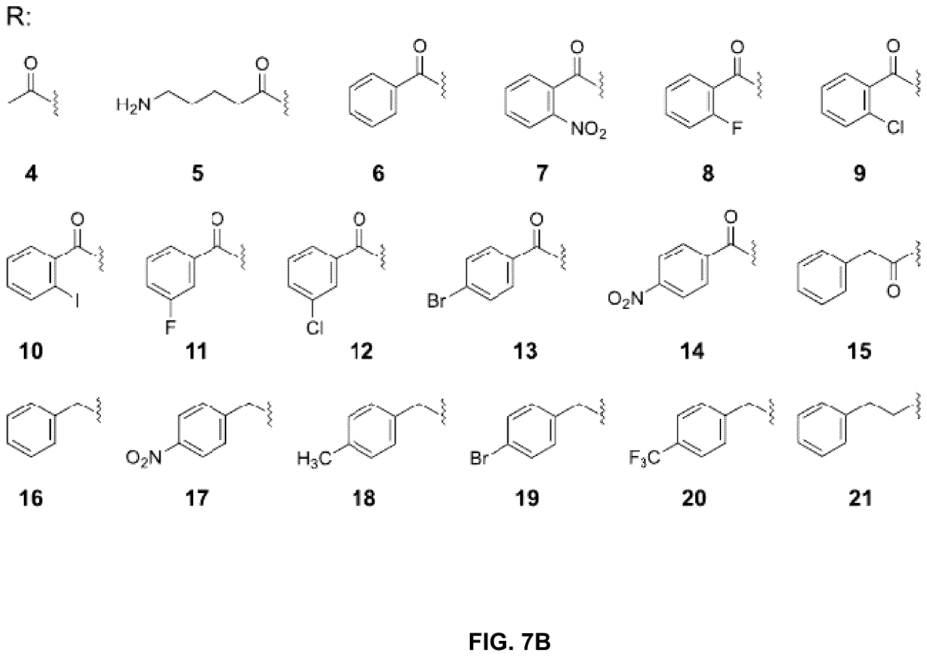

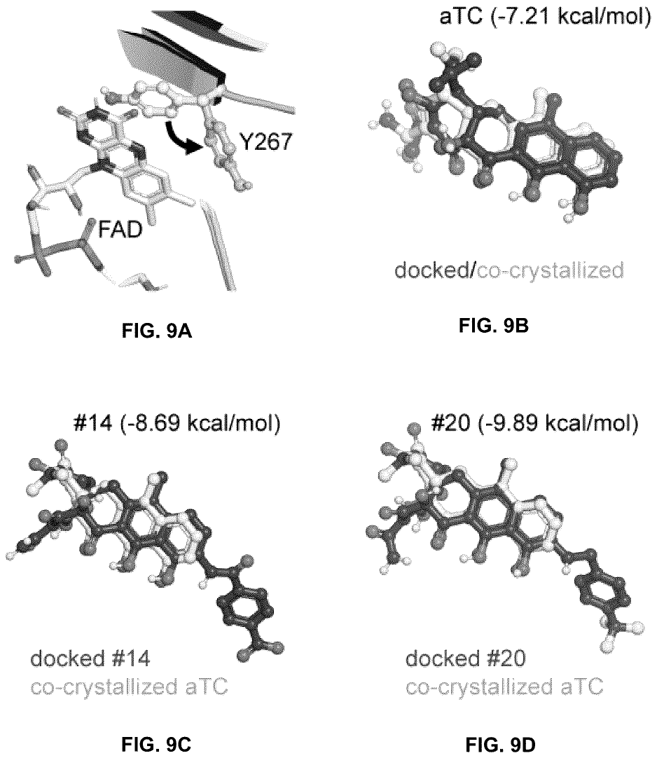

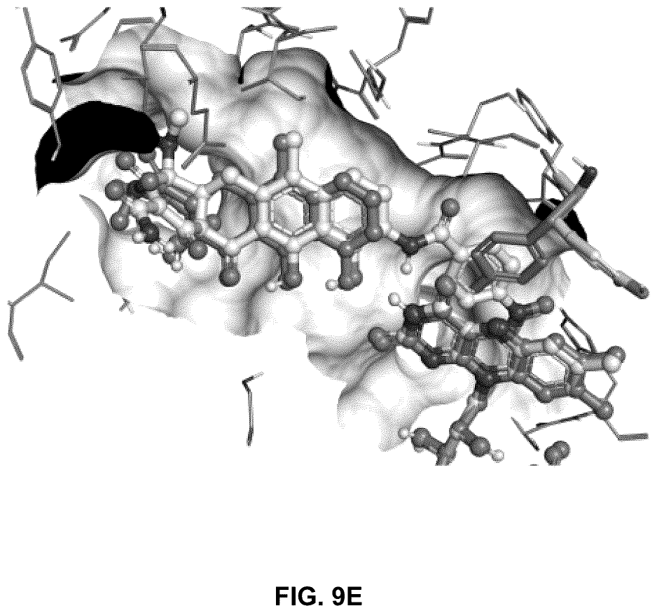

We used the co-crystal structure of aTC in complex with the Type 2 TDase Tet(50) (PDB: 5TUF) as a template for molecular modeling of bisubstrate inhibitors (FIG. 1D). We considered a variety of substituents at C9 of the bound aTC which is ˜4.8 Å from the nearest carbon atom of the Y267 phenyl ring (compounds 1-21; FIG. 7A, FIG. 7B). We hypothesized that positioning an aryl substitution from the aTC D-ring at the C9-position could mimic the FAD isoalloxazine-Y267 π-π stacking interaction and potentially improve inhibitor binding relative to the parent aTC scaffold. We used the Schrodinger platform to prepare the Tet(50) receptor using an alternate rotamer of Y267 allowing for accommodation of C9-substituents (FIG. 9A). Molecular docking was performed using Glide and validated through the docking of aTC which overlapped in good agreement with the experimentally observed binding mode (docking score=−7.1 kcal/mol; FIG. 9B). We predicted the binding modes for p-NO2-benzamide-C9-aTC (14) (docking score=−8.69 kcal/mol) and p-CF3-benzylamine-C9-aTC (20) (docking score=−9.89 kcal/mol) analogs. The docked structures aligned well with the crystallographically observed aTC binding conformation and adopt an apparent π-π stacking interaction with the FAD isoalloxazine ring. C9 of aTC is ˜3.8 Å and ˜5.0 Å from C1 and C2 of the phenyl substituents, respectively. These distances are in good agreement with the experimentally observed distance of ˜4.87 Å from C9 of aTC and the Y267 phenyl ring (FIG. 1E, FIG. 9C, FIG. 9D). The top binding poses support the bisubstrate inhibitor design where amide and amine linked C9 aryl substituents on the aTC scaffold have the proper geometry needed to span the solvent accessible channel without disturbing the aTC inhibitor binding mode.

### Synthesis of Bisubstrate TDase Inhibitors

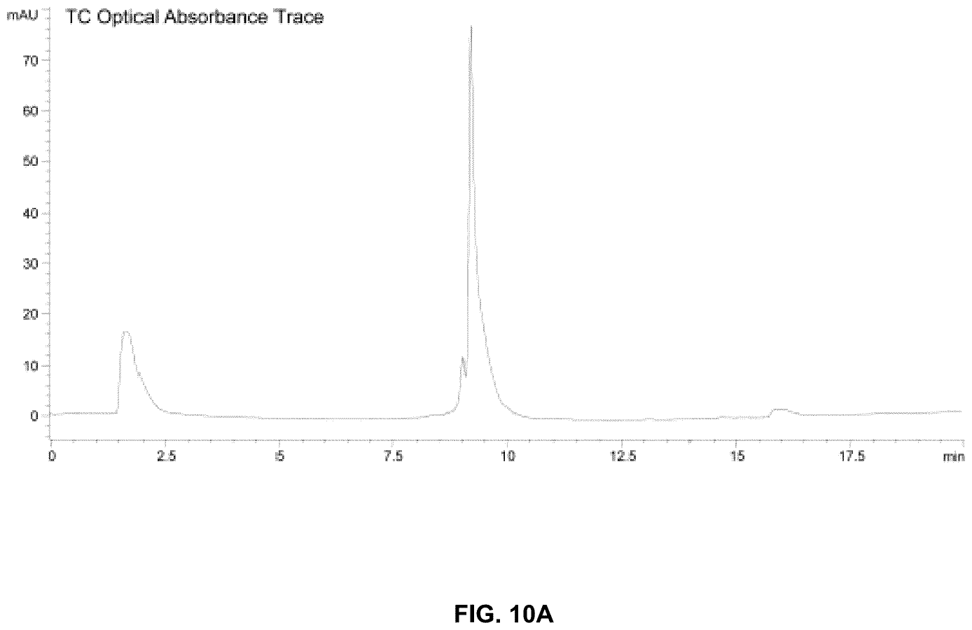

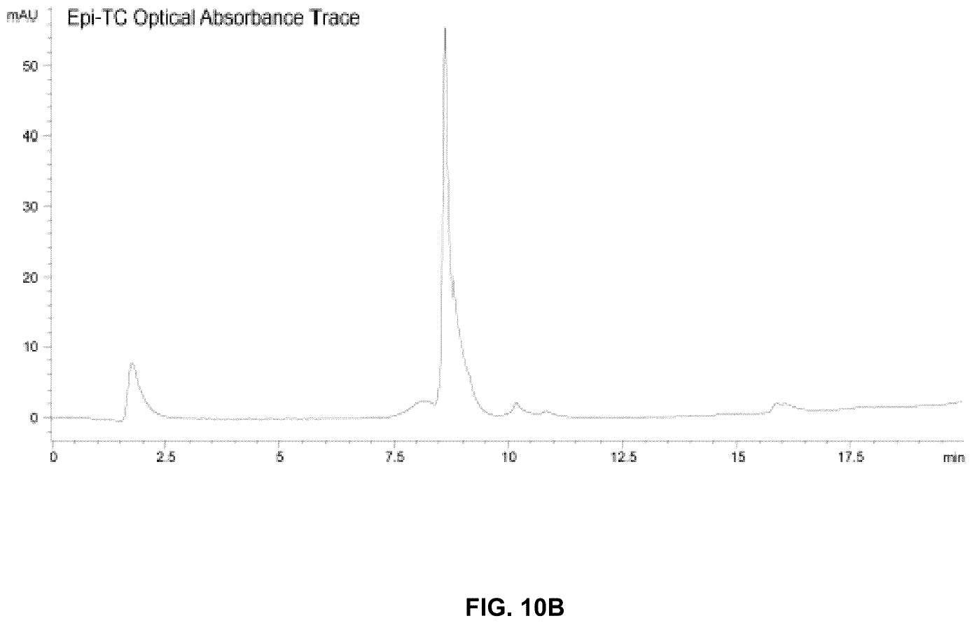

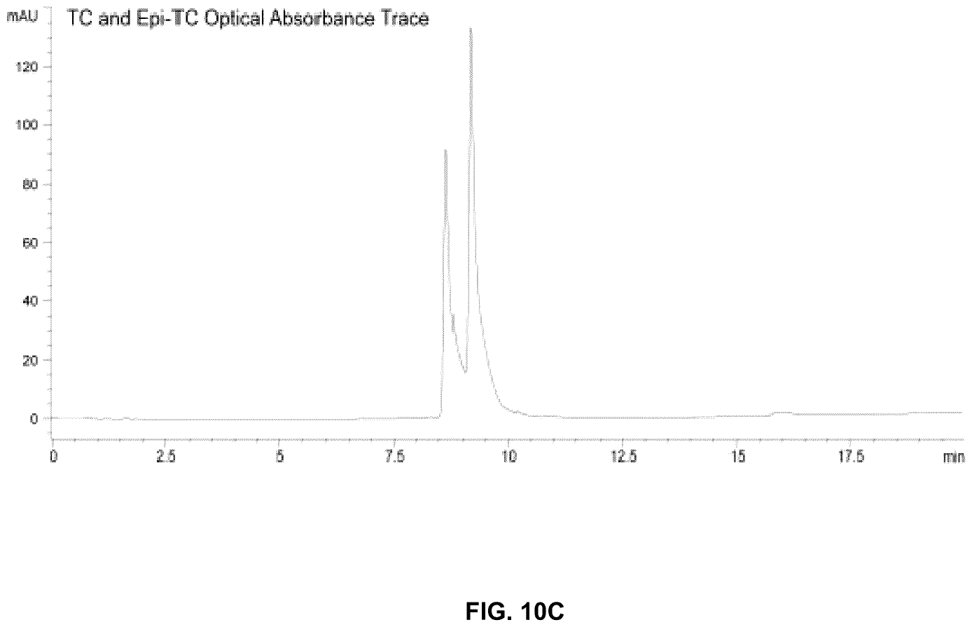

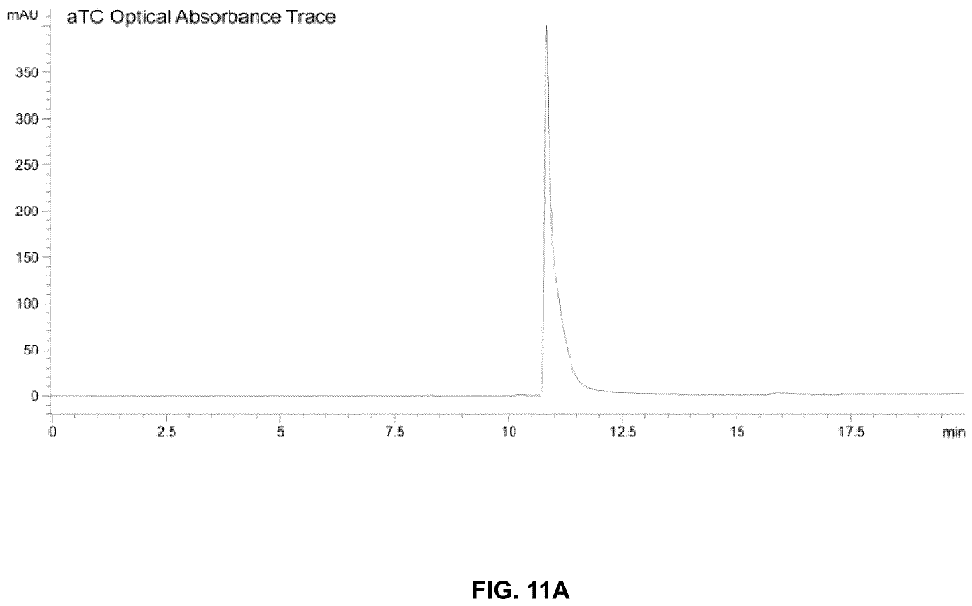

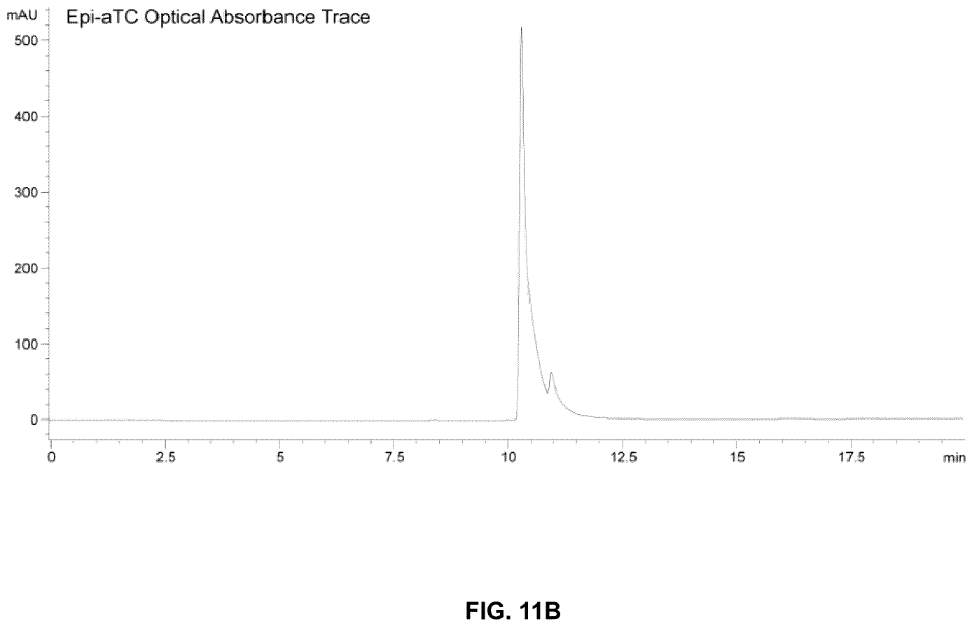

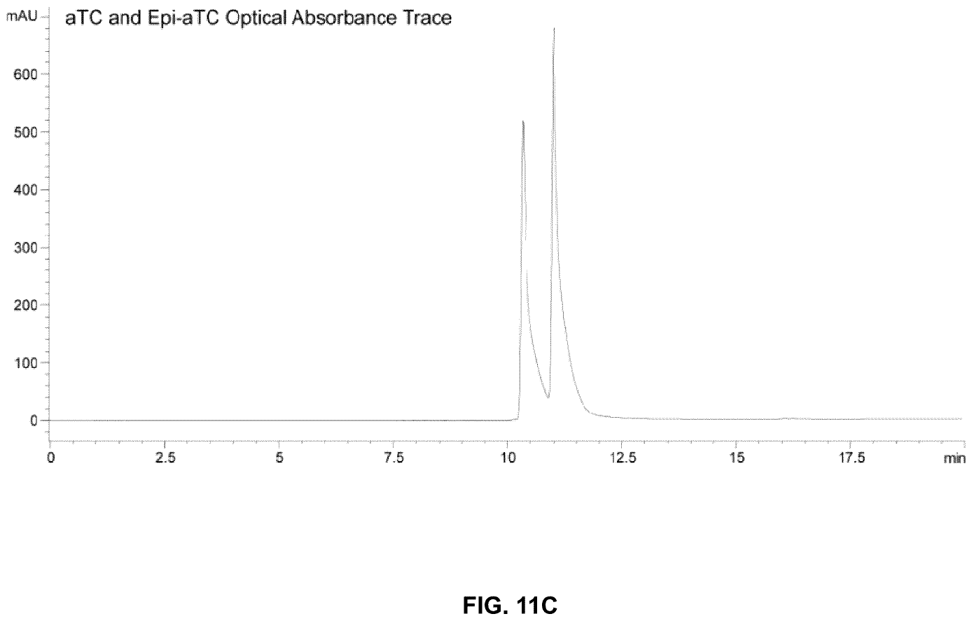

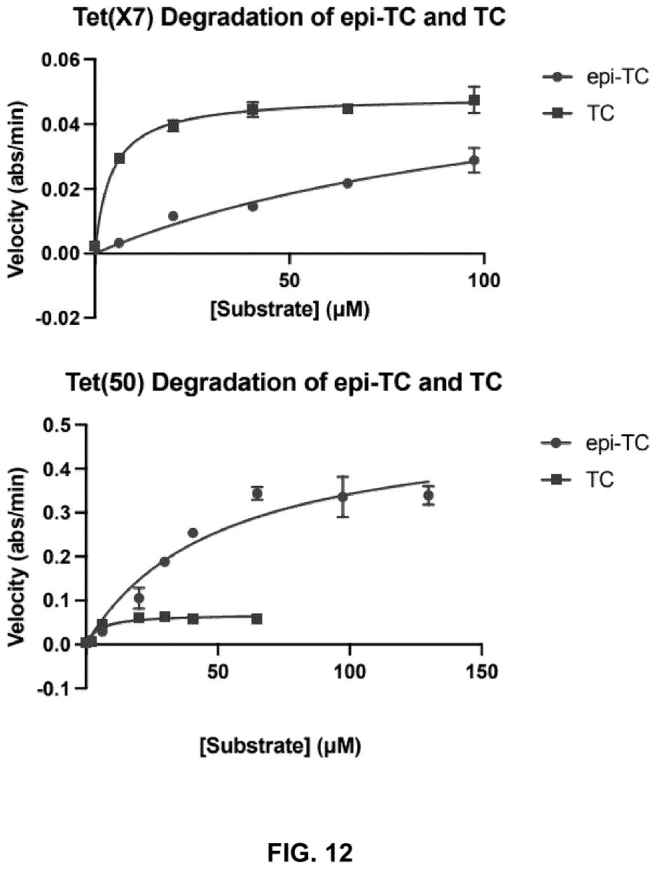

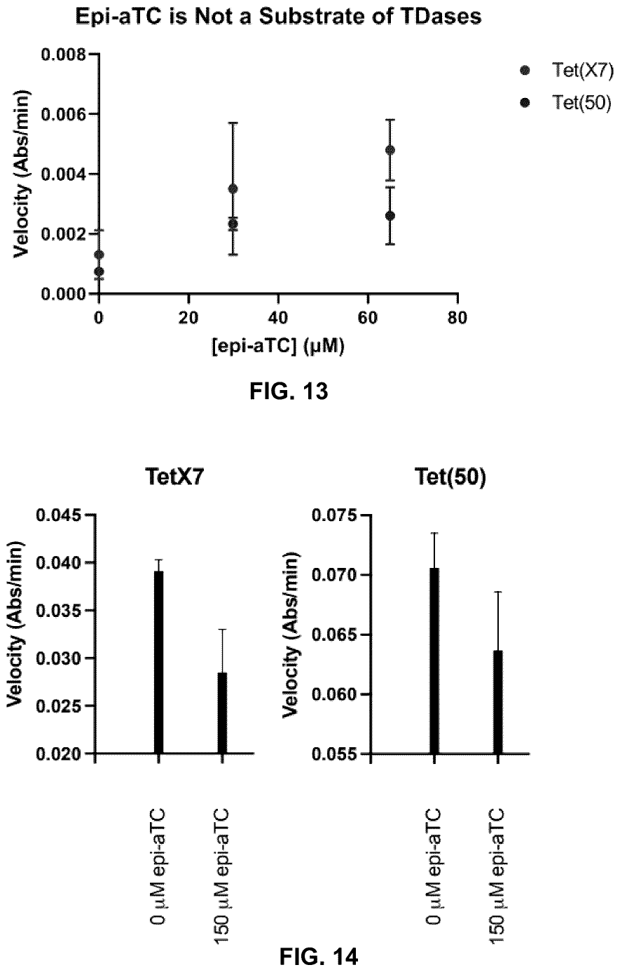

We designed a simple and scalable semi-synthetic route to prepare aTC analogs via a C-9 amination strategy (FIG. 7A, FIG. 7B). Initial attempts to nitrate aTC (1) under standard nitration conditions (HNO3/H2SO4) produced mixtures of C-9 and C-7 nitro-aTC analogs. To our surprise, we serendipitously discovered that nitrosylation of aTC (1) proceeded smoothly with high regioselectivity for C-9 substitution. Treatment of aTC (1) with sodium nitrite under acidic conditions provided C9-nitroso-aTC (2) as the corresponding HCl salt in 86% yield. This stable nitroso compound was converted to the corresponding C9-amino-aTC (3) via Pd-catalyzed hydrogenolysis resulting in 73% yield of the desired product. This simple two-step procedure can be accomplished on gram scale to provide C9-amino-aTC (3) as the corresponding HCl salt with no chromatographic steps. We used C9-amino-aTC (3) as a central intermediate to access C9-aTC analogs with an amide or amine linkage via nucleophilic acyl substitutions with carboxylic acid substrates or reductive aminations with aldehyde substrates, respectively. Treatment of C9-amino-aTC (3) with various carboxylic acids under optimized amide coupling conditions (HATU, DIPEA, DMF, rt, 15 min) provided amides 4-15 as the corresponding TFA salts in 5-30% yield after final purification by prep-HPLC. Treatment of C9-amino-aTC (3) with various aldehydes under optimized reductive amination conditions (Na(OAc)3BH, MeOH, 0° C., 6 h) provided amines 16-21 as the corresponding TFA salts in 9-25% yield after final purification by prep-HPLC. All compounds were fully characterized by NMR and HRMS with purity analysis by LC-MS conducted prior to biological testing. Some samples contained varying amounts of C4-epimers as reported in the Methods section. Equilibration of C4-epimers is common for TC, aTC, and related compounds and is pH dependent. C4-epi-TC has reduced affinity for the ribosome and Tet repressor proteins presumably due to the disruption of essential interactions between the A-ring substituents and conserved binding site residues. We demonstrated that C4-epi-TC also has reduced apparent binding affinity to Tet(X7) and Tet(50) and C4-epi-aTC is not an inhibitor or substrate of these two enzymes (FIG. 10A, FIG. 10B, FIG. 10C, FIG. 11A, FIG. 11B, FIG. 11C, FIG. 12, FIG. 13, FIG. 14). Hence, we assumed that only the C4-(S) epimers of compounds 1-21 contributed significantly to the observed biological activities.

### In Vitro SAR of Bifunctional TDase Inhibitors

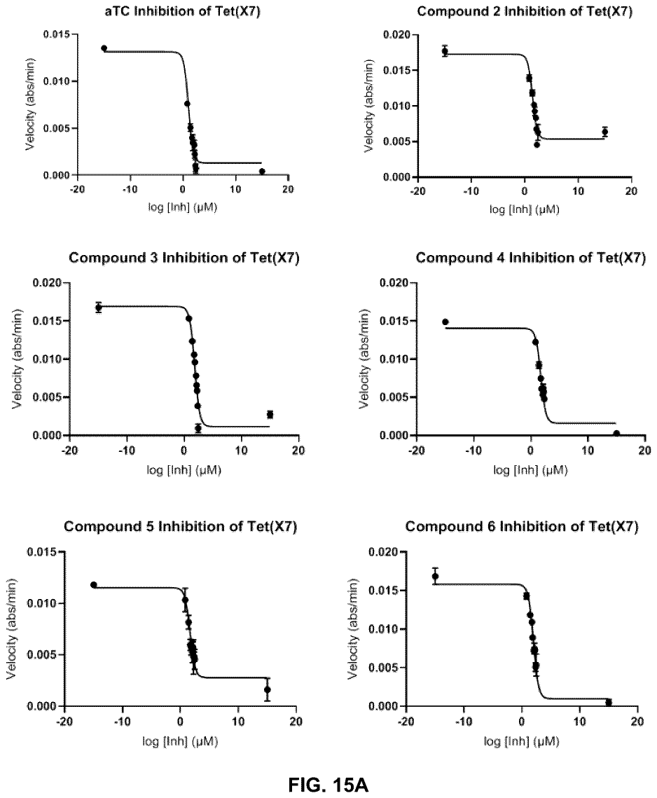

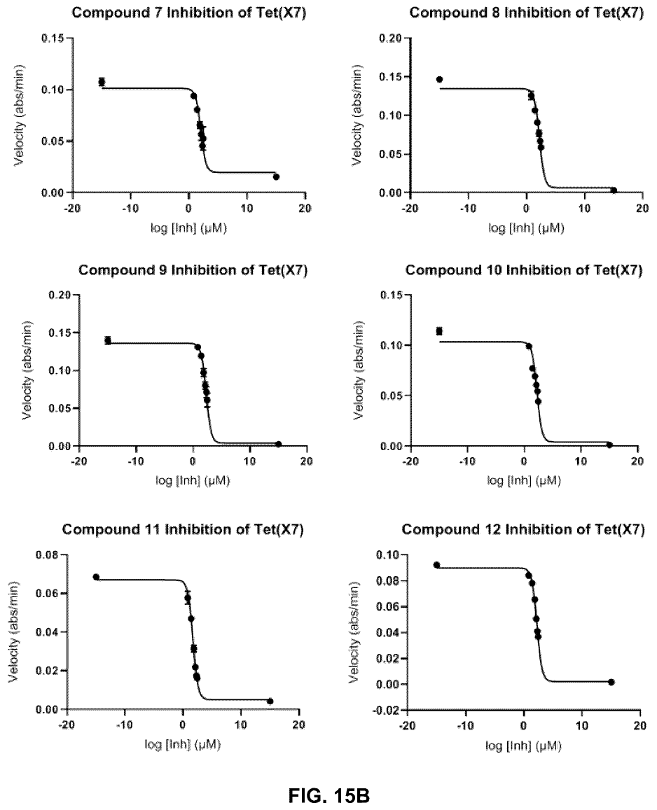

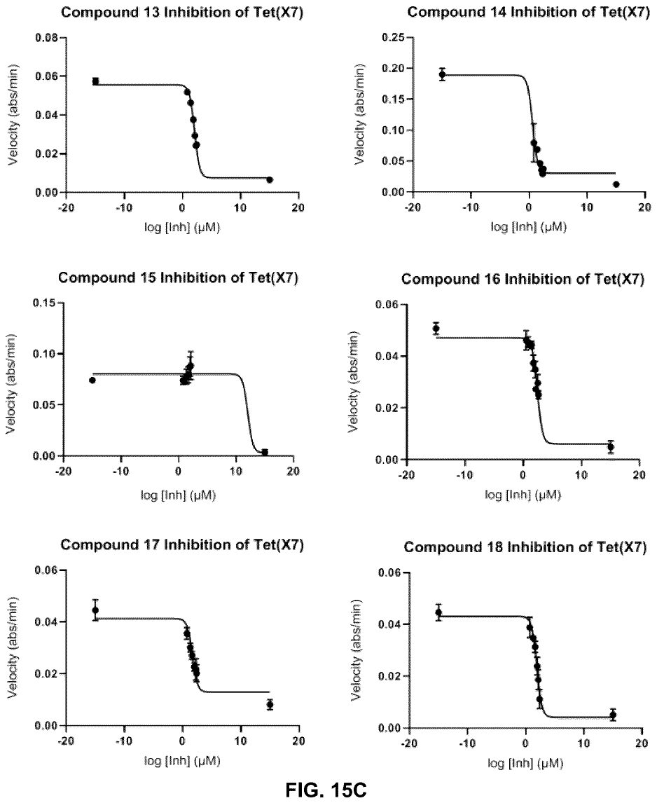

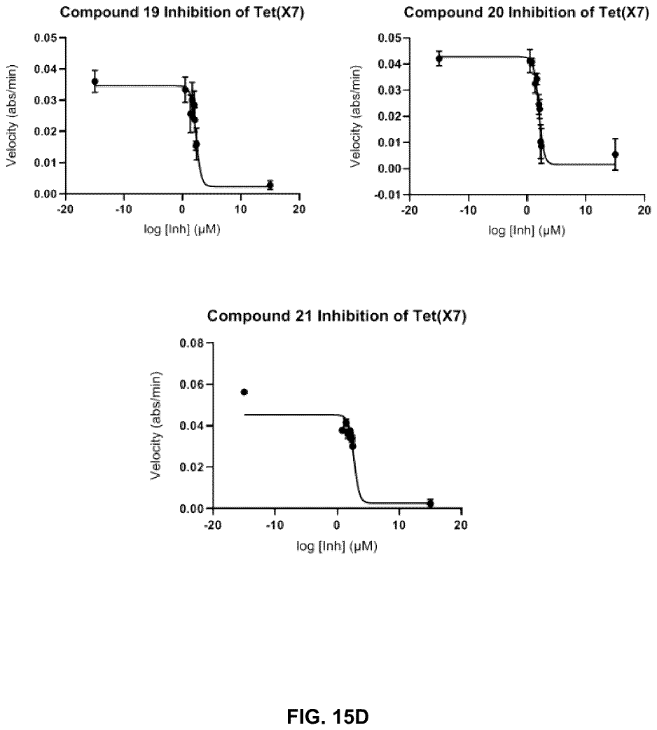

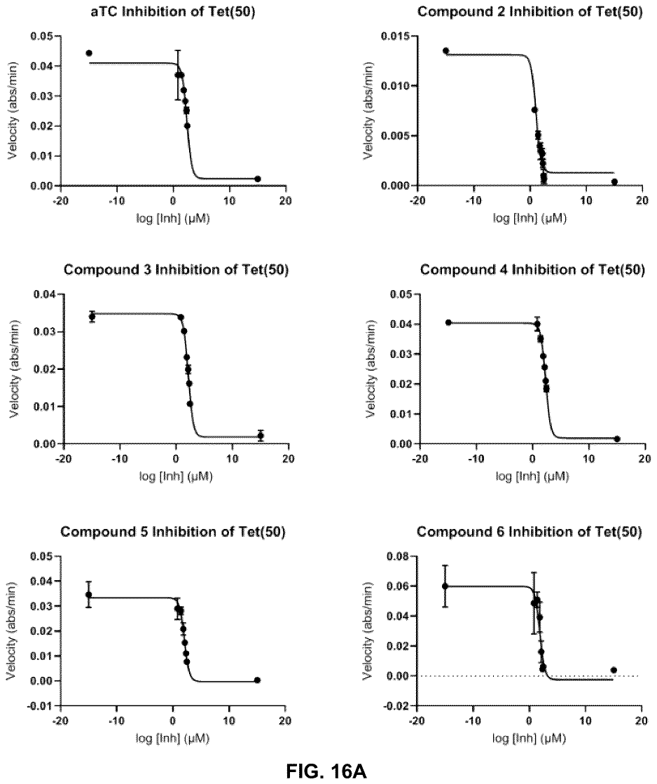

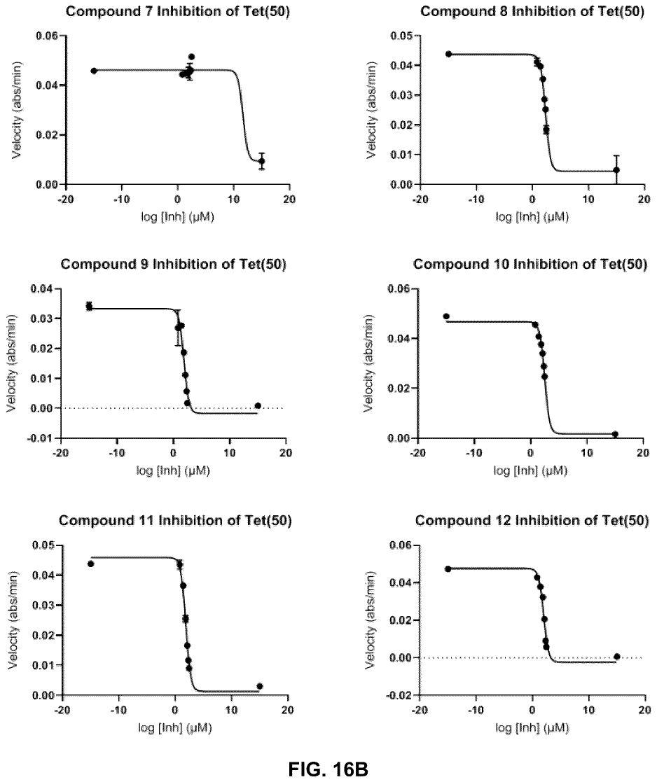

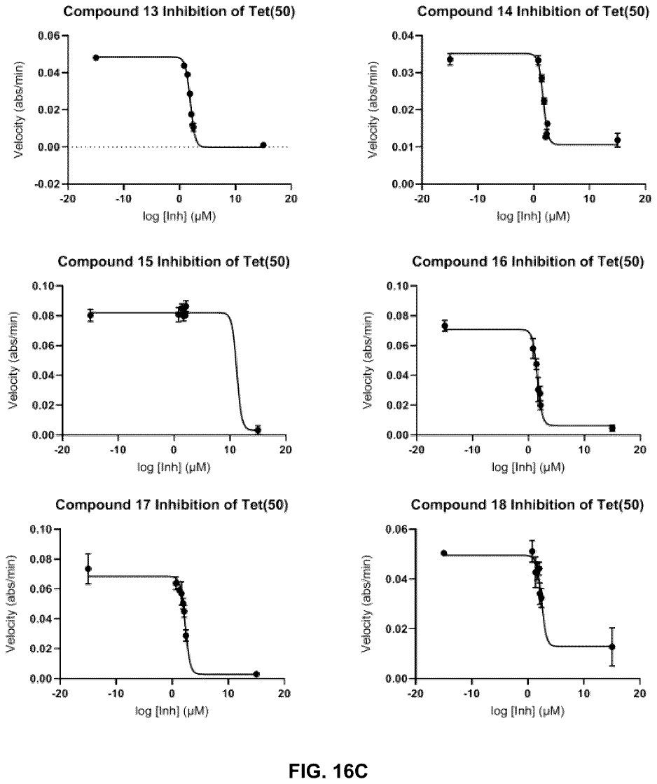

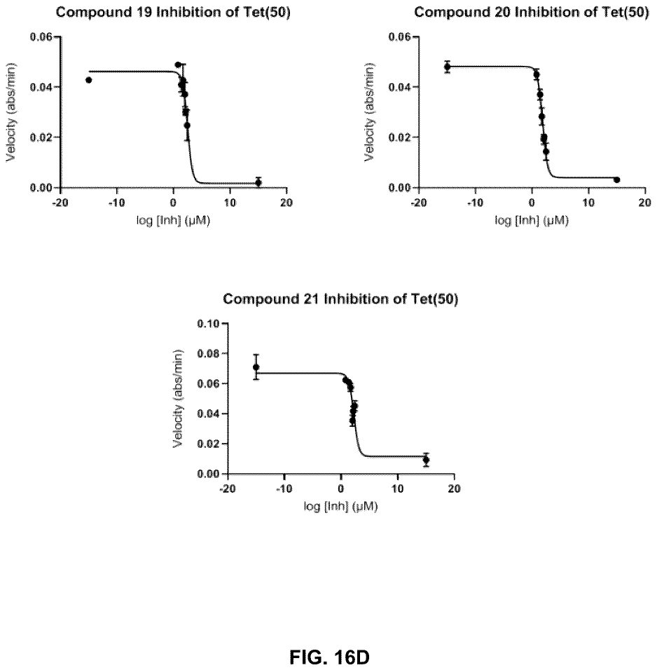

We determined apparent IC50 values for compounds 1-21 against the Type 1 TDase Tet(X7) and the Type 2 TDase Tet(50) under steady-state conditions using TC as the substrate and NADPH as a cosubstrate. Reactions were initiated by the addition of TDase, without pre-incubation with inhibitors, and were monitored by the continuous measurement of optical absorbance at −400 nm (ATC monitoring for direct TC consumption) to determine linear velocities for each reaction. Apparent IC50 values were determined by plotting the observed reaction velocity vs log[inhibitor](FIG. 2) and the convenient comparison of SAR for each enzyme type (FIG. 15A, FIG. 15B, FIG. 15C, FIG. 15D, FIG. 16A, FIG. 16B, FIG. 16C, FIG. 16D). We observed inhibition of Tet(X7) for 20 inhibitors (95% hit rate) with apparent IC50 values ranging from 3-600 μM adjusted for standard deviations. Similarly, 19 of the inhibitors were active against Tet(50) (90% hit rate) with a statistically significant range of apparent IC₅₀ values from 38-370 μM.

Some notable SAR patterns emerged from the apparent IC₅₀ values determined from the in vitro TDase inhibition assays (FIG. 2). aTC (1) represents the parent inhibitor structure and provides a convenient reference point for comparing inhibitor potencies. The apparent IC₅₀ values of aTC were 10±2 μM and 260±40 μM against Tet(X7) and Tet(50), respectively. These values are in good agreement with a previous report from our group (3±1 μM and 210±25 μM against Tet(X7) and Tet(50), respectively). The reason for preferential inhibition of Type 1 (Tet(X7)) over Type 2 (Tet(50)) TDases by aTC (1) was not immediately clear, but we note that simple C9 substitutions including nitroso (2) and amino (3) groups reduced the inhibition of Tet(X7) (30±10 μM and 70±10 μM, respectively) and improved the inhibition of Tet(50) (40±2 μM and 140±10 μM, respectively) compared to the parent aTC scaffold. Amido substituents at C9 were generally well tolerated with the simplest C9-acetamido (4) showing preference for inhibiting Tet(X7) (50±10 μM) over Tet(50) (200±10 μM). The bulkier C9-5-aminopentamide derivative (5) was also well tolerated with apparent IC₅₀ values of 40±10 μM (Tet(X7)) and 110±20 μM (Tet(50)).

The C9-benzamides 6-14 revealed a preference for meta- and para-substitution over orthosubstitution for both TDase types. Unsubstituted C9-benzamide 6 establishes a baseline for this series with apparent IC₅₀ values of 100±10 μM (Tet(X7)) and 70±30 μM (Tet(50)), which is reduced inhibitory activity against Tet(X7) and enhanced inhibitory activity against Tet(50) compared to the parent aTC scaffold. Substitution of the phenyl ring with an ortho-NO2 group (7) was well tolerated for Tet(X7) (110±20 μM) but abolished inhibitory activity against Tet(50) (>1000 μM). Halogens in the ortho-position of C9-benzamides were better tolerated than ortho-nitration with a relatively tight range of apparent IC₅₀ values of 140-220 μM (Tet(X7)) and 100-320 μM (Tet(50)) for ortho-F (8), ortho-Cl (9), and ortho-I (10). There is a slight preference for meta- and para-halogenation of C9-benzamides with apparent IC₅₀ values ranging from 48-180 μM (Tet(X7)) and 70-110 μM (Tet(50)) for metaF (11), meta-Cl (12), and para-Br (13). The para-NO2 C9-benzamide 14 produced the lowest apparent IC₅₀ value from the set against Tet(X7) (4±1 μM) and showed a striking increase in activity against Tet(50) (50±10 μM) compared to the ortho-NO2 derivative (>1000 μM). Simple homologation from C9-benzamide to C9-phenylacetamide (15) abolished all TDase inhibitory activity which deterred us from further pursuing phenyl substituted analogs in this series.

We next explored a series of C9-benzylamine derivatives (16-20) designed to maintain the same spacing as C9-benzamides between the C9-position of the aTC scaffold to the phenyl substituent (FIG. 2). The C9-benzylamines allowed us to evaluate a more flexible C9-linkage relative to the rigid amide linkage in the C9-benzamide inhibitor series. We only investigated para-substitutions on the phenyl ring for the C9-benzylamine derivatives given the observed preference for this position in the benzamide series. Overall, inhibitor potency in the benzylamine series (apparent IC₅₀ values ranging 30-500 μM) was in line with that observed for the benzamide series with a slight preference for inhibiting Tet(X7) over Tet(50) (except for compound 16). Electron withdrawing groups (NO2, CF3) on the phenyl ring of the benzylamine analogs were favored over donating groups (Me, Br) against Tet(X7) while the unsubstituted phenyl ring (16) was best against Tet(50). A homologated C9-phenethylamine derivative (21) showed moderate activity against Tet(50) (180±50 μM) and diminished activity against Tet(X7) (500±100 μM) similar to the homologated C9-phenylacetamide analog 15. The para-CF3 analog 20 was the most effective broad spectrum inhibitor of Type 1 and 2 TDases in the benzylamine series with apparent IC₅₀ values of 100±30 μM and 70±10 μM against Tet(X7) and Tet(50), respectively.

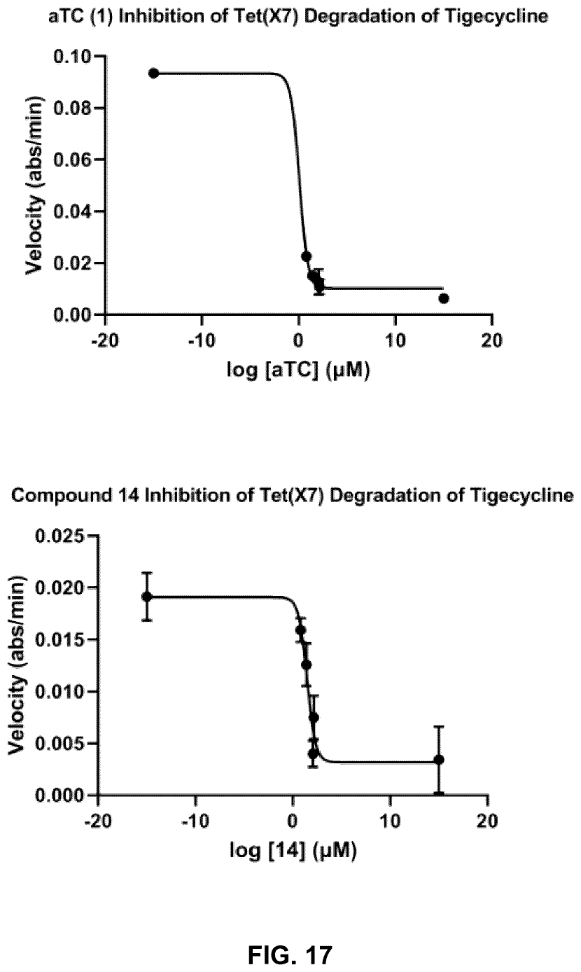

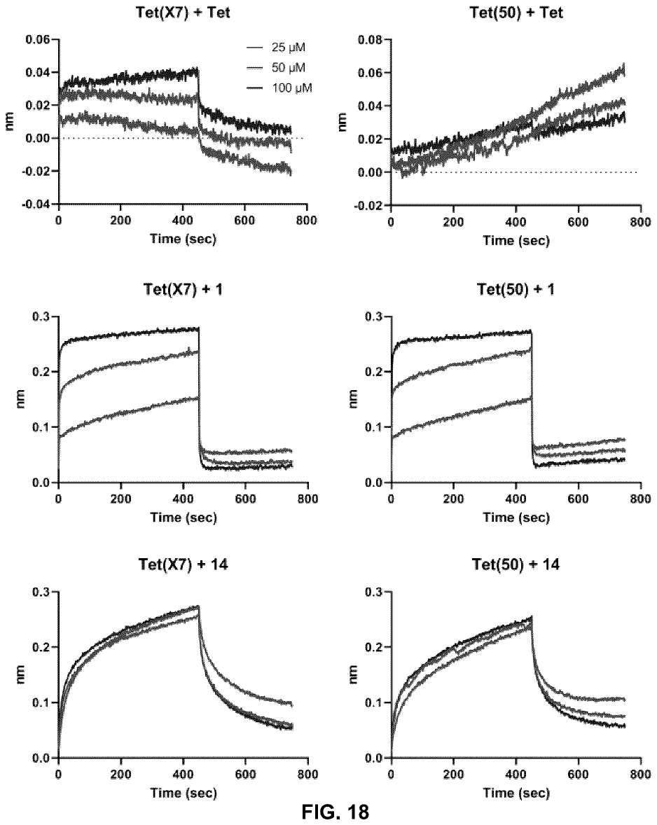

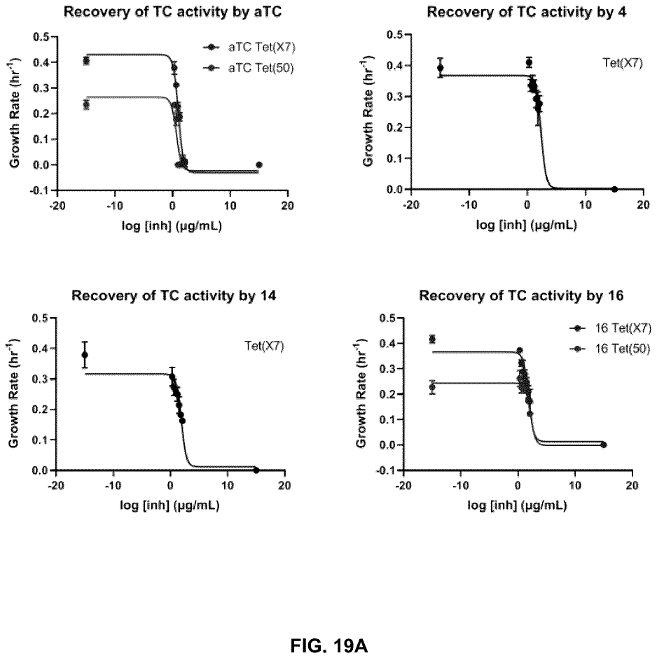

To validate that the compounds can inhibit degradation of more clinically relevant substrates, we investigated the ability of aTC (1) and compound 14 to inhibit Tet(X7) degradation of tigecycline, a third-generation glycylcycline. Compounds 1 and 14 inhibited tigecycline degradation with apparent IC₅₀ values of 1.2±0.2 μM and 30±11 μM, respectively (FIG. 17). Independent biophysical validation of inhibitor binding was carried out using Bio-Layer Interferometry (BLI). Treatment of TDases with EZ-Link NHS-PEG4-Biotin (ThermoFisher) resulted in biotinylation of 7.4 mol biotin/mol Tet(X7) (22 Lys residues) and 6.1 mol biotin/mol Tet(50) (23 Lys residues). We compared Tet(X7) and Tet(50) binding of TC, 1, and 14 (FIG. 18) using BLI with super streptavidin tips (Sartorius). While we were unable to quantitatively fit the kinetic binding data, the qualitative binding curves consistently show that inhibitor 14 displays slower binding kinetics (kₒₙ and k_{off}) compared to aTC which is consistent with a bisubstrate mode of inhibition.

Rescue of TC Antibacterial Activity with Bisubstrate TDase Inhibitors

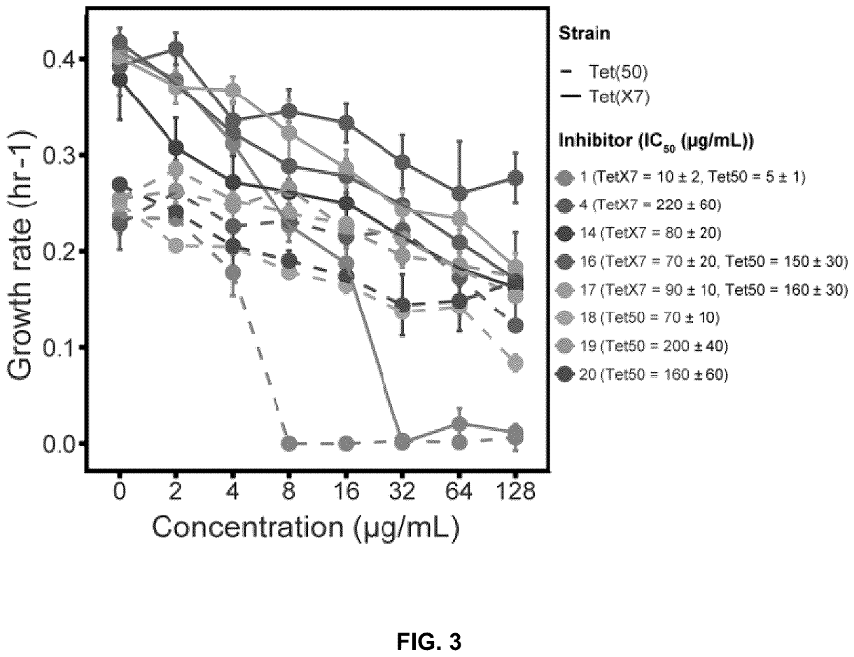

We selected a subset of C9-amide (compounds 4 and 14) and C9-amine inhibitors (compounds 16-20) for direct comparison with the parent inhibitor aTC (1) in the ability of TDase inhibitors to recover the antibacterial activity of TC in whole-cell bacterial growth experiments (FIG. 3). We selected more compounds from the amine series due to the similarity in apparent IC₅₀ values from the in vitro TDase inhibition assays and the improved water solubility of these compounds compared to the amides. We used E. coli MegaX harboring pZE21 plasmid constructs for constitutive expression of Tet(X7) and tet(50). Our group previously reported and validated this bacterial system for antibiotic susceptibility testing with TDase inhibitor and TC antibiotic combinations. An important consideration for the interpretation of antibacterial combinations is the inherent antibacterial activity of each compound alone. The MIC values for TC against E. coli MegaX harboring pZE21-Tet(X7) and pZE21-Tet(50) were recorded as 512 μg/mL and 128 μg/mL, respectively, using the standard broth microdilution method in MHII medium. The MIC values for aTC (1) against the same strains were recorded as 64 μg/mL (Tet(X7)) and 16 μg/mL (Tet(50)). In contrast, we found that compounds 4, 14, and 16-20 all lacked inherent antibacterial activity, with cell growth observed at concentrations up to 128 μg/mL.

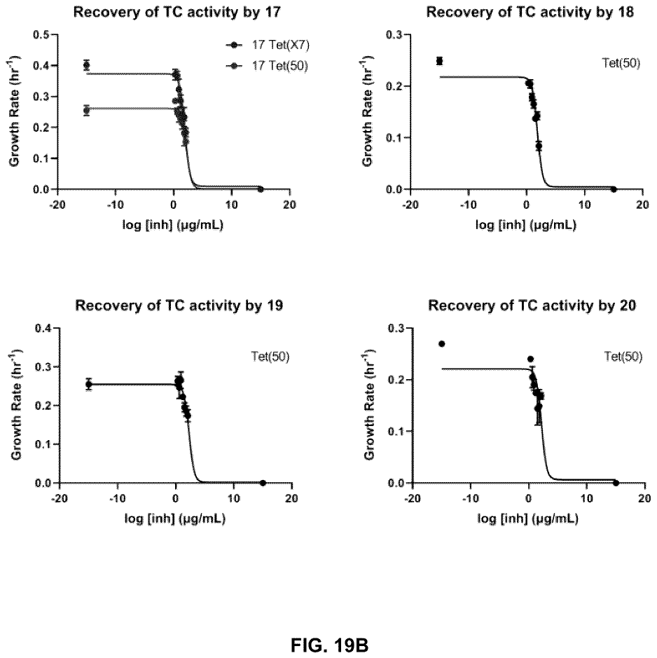

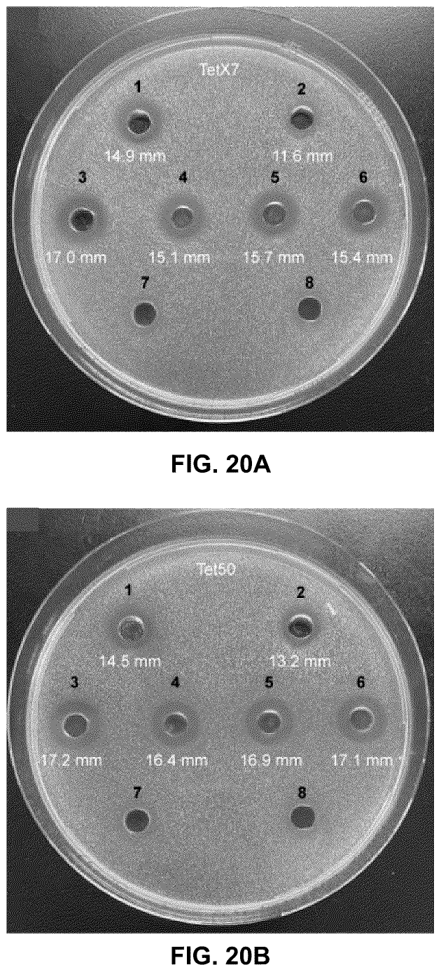

We designed a combination study using TC antibiotic fixed at 16 μg/mL, severalfold lower than the recorded MIC values, with variable concentrations of TDase inhibitor (1, 4, 14, or 16-20) ranging from 0-128 μg/mL. We determined full bacterial growth curves during incubation at 37° C. with optical density at 600 nm measured over a 20-hour window. The growth rate (reported as hr-1) was determined for each condition to compare the effects of TDase inhibitors in an unbiased manner (FIG. 3, Table 2). We observed dose-dependent rescue of TC activity by all the TDase inhibitors with apparent IC₅₀ values that agree with the in vitro potencies (FIG. 19A, FIG. 19B). The parent inhibitor aTC (1) appears to give the most potent rescue of TC activity with complete inhibition of bacterial growth observed at 32 μg/mL and 8 μg/mL for Tet(X7)- and Tet(50)-producing strains, respectively, with apparent IC₅₀ values of 10±2 μg/mL and 5±1 μg/mL. For aTC it is difficult to separate contributions of TDase inhibition and inherent antibacterial activity at this concentration range. Hence, it is appreciated that while compounds 4, 14, and 16-20 fall short of providing complete growth inhibition at 16 μg/mL TC there is a clear dose-dependent decrease in growth rates, consistent with TDase inhibition and TC rescue as the driver of this effect given these compounds independently possess no inherent growth inhibitory activity. All compounds tested resulted in significantly lower growth rates at concentrations 64 μg/mL, with significant decreases for compounds 14 and 18 with just 2 μg/mL (Table 2). We confirmed these results on solid medium using the Kirby-Bauer agar diffusion antibacterial susceptibility assay for tigecycline against the Tet(X7)-expressing strain and for TC against the Tet(50)-producing strain (FIG. 20A, FIG. 20B). This modest, yet promising bioactivity suggests that non-antibacterial TDase inhibitors can be developed to rescue TC antibiotic activity against bacterial pathogens expressing TC inactivating enzymes.

### Bisubstrate TDase Inhibitors Block FAD Reduction by NADPH

Our prior biochemical characterization of aTC (1) inhibition of Type 1 and 2 TDases using Lineweaver-Burke analyses support a model of competitive mechanism-based inhibition that is in agreement with the observed aTC binding mode in the Tet(50) co-crystal structure. The new C9-substituted aTC analogs in this study were designed to be bisubstrate inhibitors that engage both the TC and NADPH binding sites. We hypothesized that a bisubstrate TDase inhibitor will competitively bind in both the TC and NADPH binding sites. To test this hypothesis we used TC, aTC (1), and the para-N02-substituted C9-benzamide aTC analog (14) to investigate the impact of TC antibiotics and TDase inhibitors on NADPH consumption. FMOs including TDases consume NADPH by reduction of FAD to FADH2 that leads to the O2-dependent formation of a reactive C4a-peroxy-flavin intermediate. FAD reduction by NADPH can be coupled to substrate oxidation or uncoupled leading to the formation of H2O2. Detection of H2O2 has been used in diagnostic applications for TDase resistance in bacterial cultures40. Both the coupled and uncoupled pathways must be considered unless substrate is omitted where uncoupled H₂O₂ formation is the only viable path for enzyme turnover. Thus, H2O2 formation can serve as a proxy for FAD reduction by NADPH in FMOs.

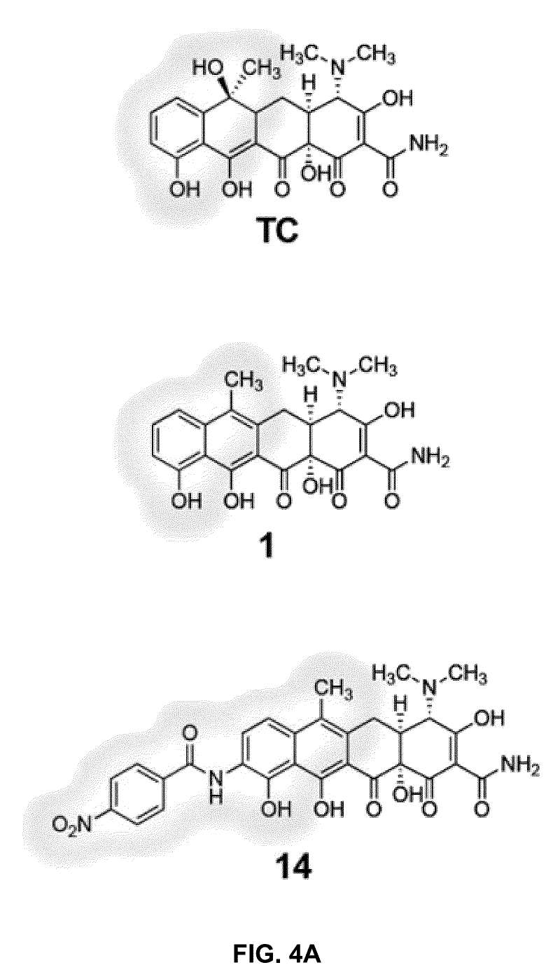

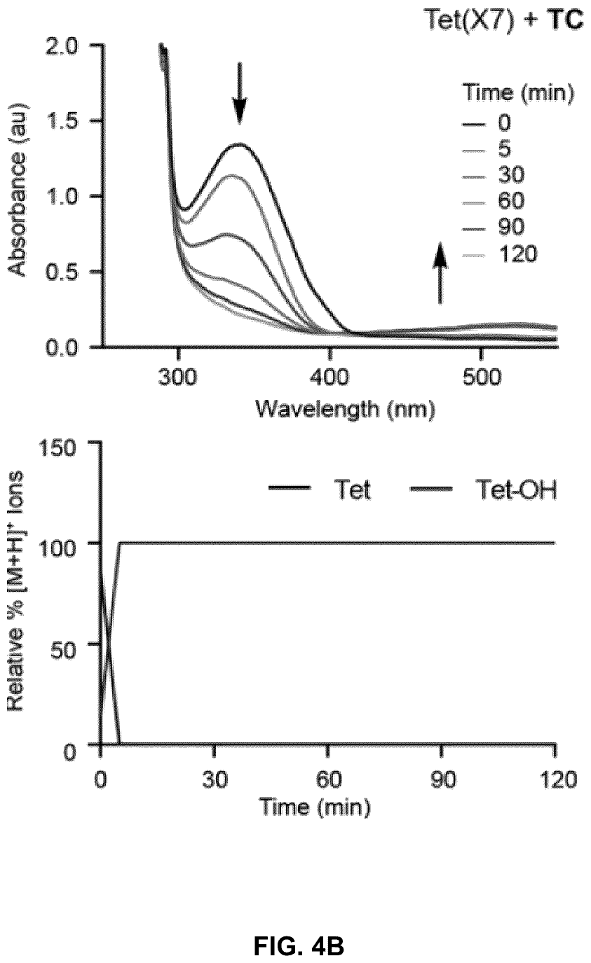

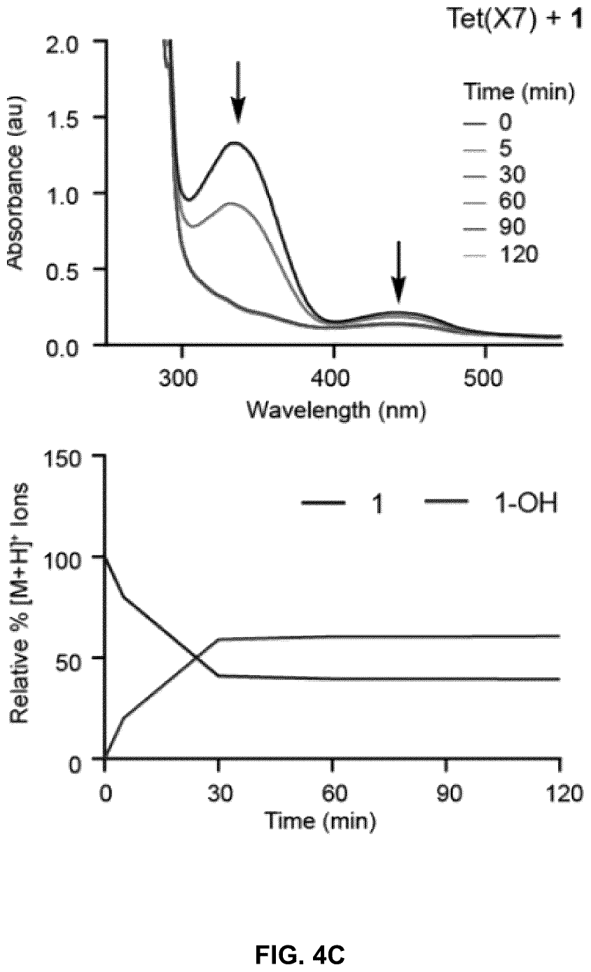

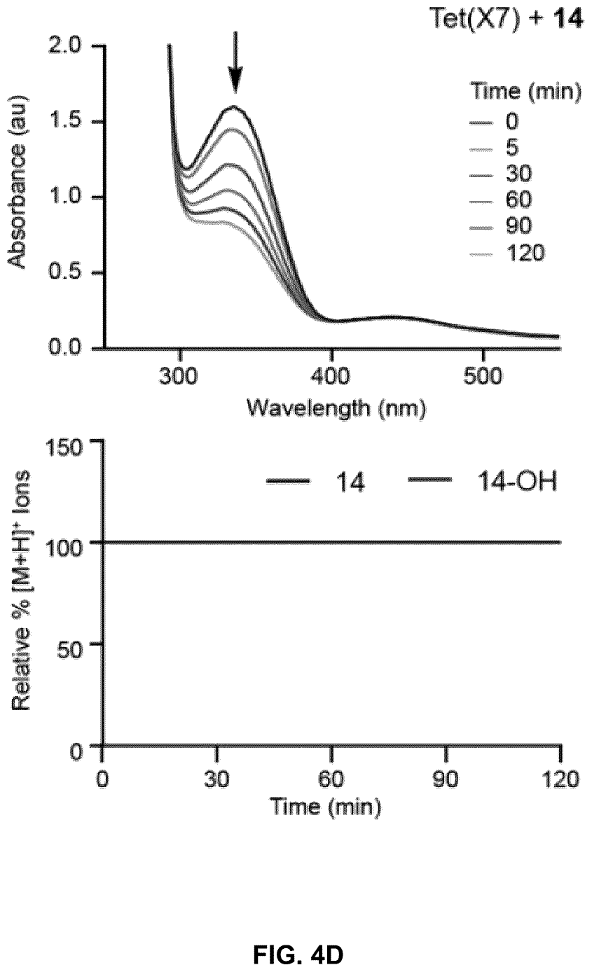

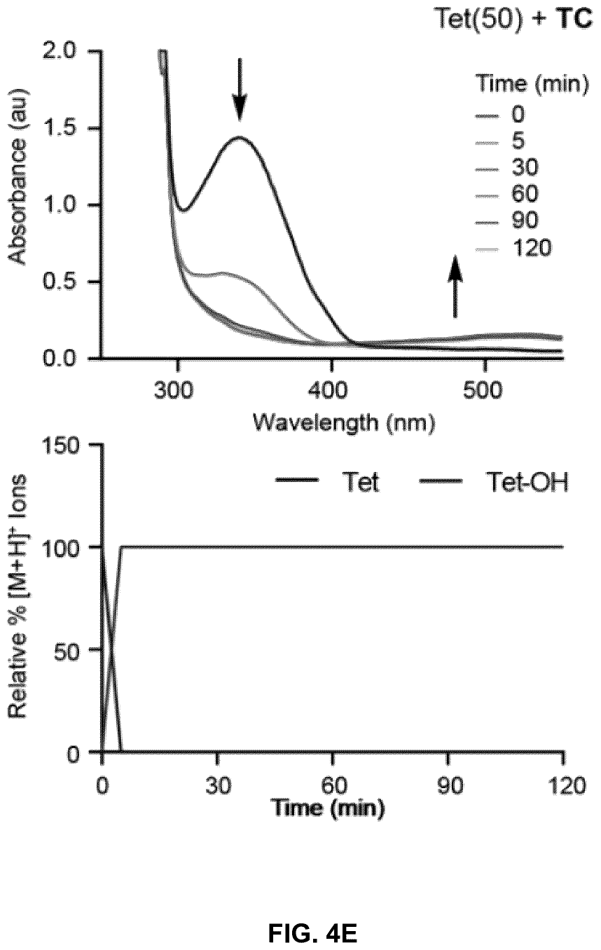

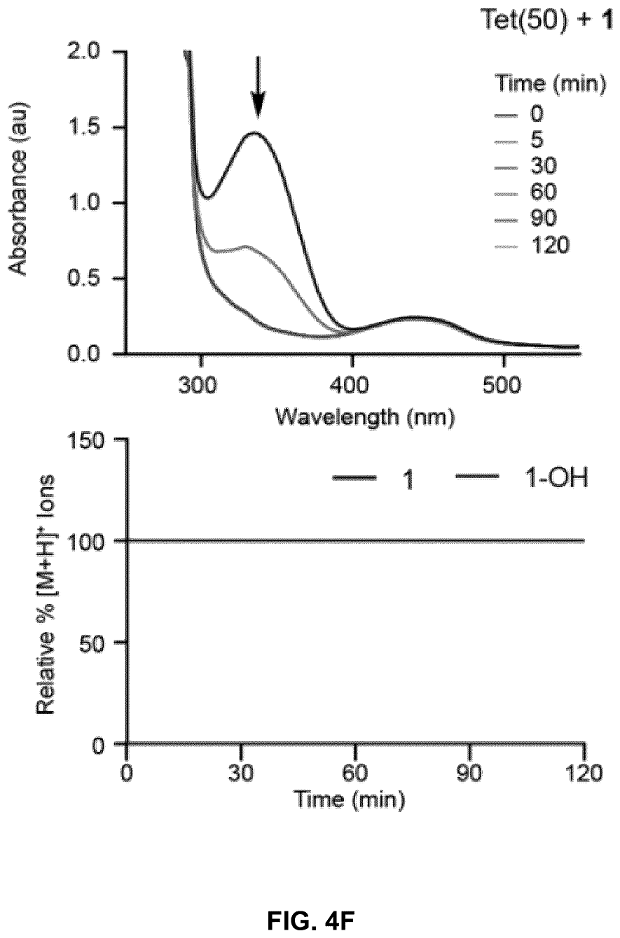

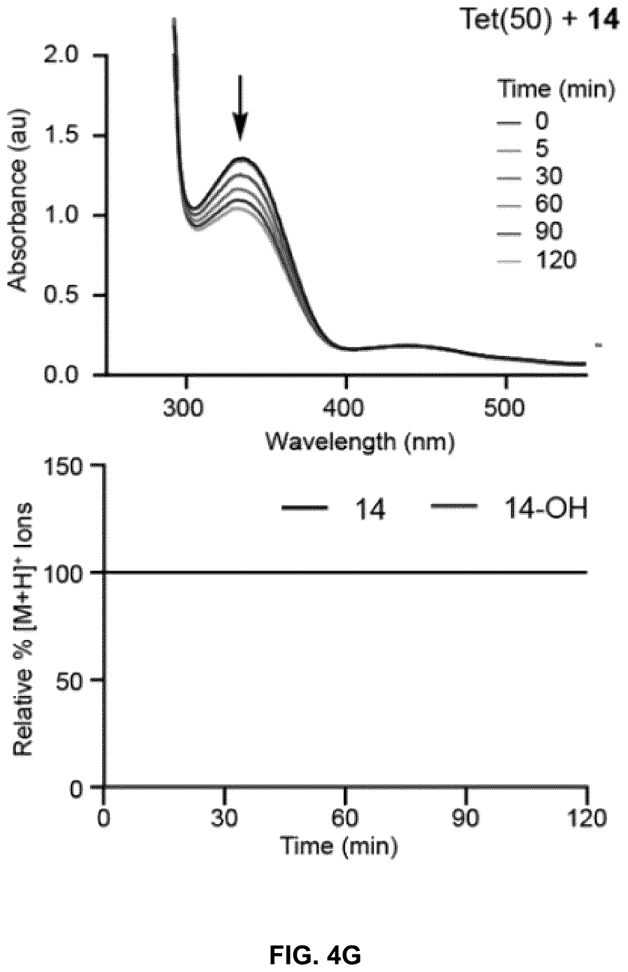

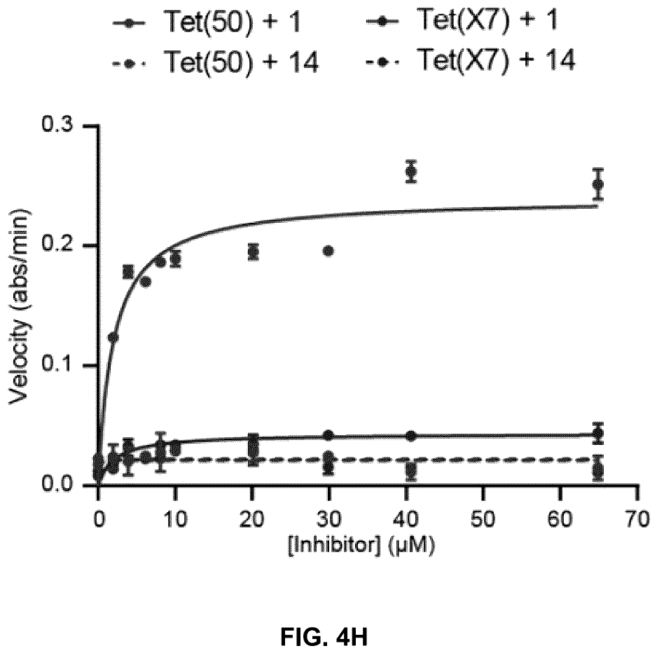

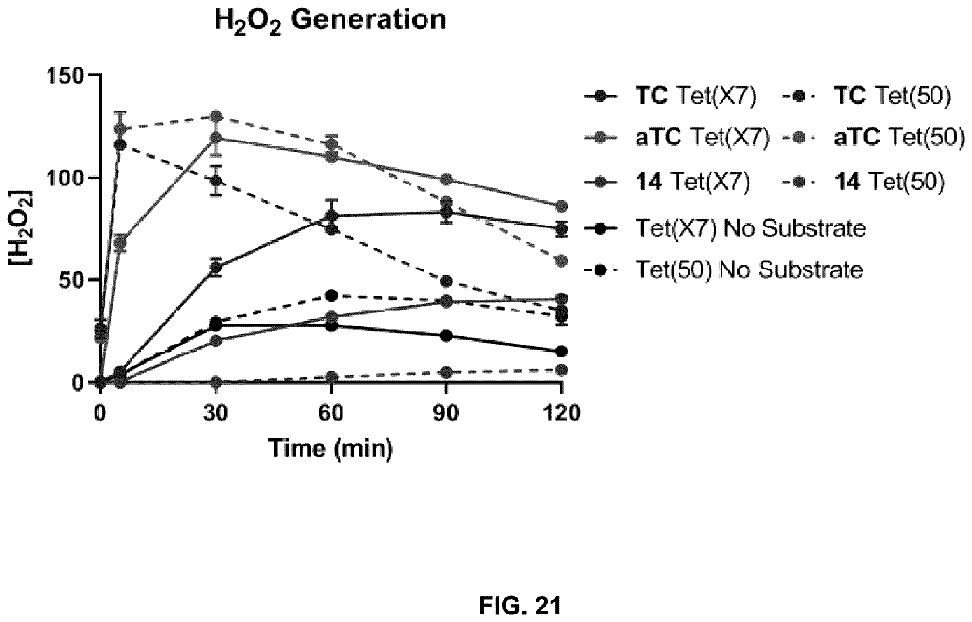

We analyzed TDase reactions with Tet(X7) and Tet(50) via optical absorbance (λ=250-500 nm), LC-MS, and H₂O₂ formation (FIG. 4A). We monitored the oxidation of Fe2+ to Fe3+ in the presence of xylenol orange to quantify H₂O₂ during TDase reactions. Xylenol orange provides a color change from orange to purple in the presence of H₂O₂ that can be monitored via optical absorbance at λ=595 nm (FIG. 21). Both TC (FIG. 4B) and aTC (FIG. 4C) enhanced the rate of NADPH consumption for Tet(X7) and generated H2O2 in excess of the control. LC-MS analysis of the reaction mixtures also showed that TC and aTC are oxidized by the addition of one 160 to the scaffold corresponding to the time and TDase-dependent formation of an observed [M+O+H]+ molecular ion for each product. Conversely, the para-NO2-C9-benzamide aTC analog (14) reduced the rate of NADPH consumption, blocked H2O2 formation, and was stable over the two-hour duration of the reaction with Tet(X7) (FIG. 4D, FIG. 4G). The results for TC with Tet(50) were similar to the Tet(X7) results where TC stimulated NADPH consumption, produced H2O2, and was rapidly oxidized (FIG. 4E). Like Tet(X7), aTC (1) stimulated NADPH consumption and produced H2O2 for the Tet(50) reaction. However, unlike the Tet(X7) reaction, aTC was stable over the reaction course with Tet(50) (FIG. 4E). The result for the para-NO2-C9-benzamide aTC analog (14) against Tet(50) showed an even more pronounced reduction in the rate of NADPH consumption and H2O2 generation (FIG. 4G).

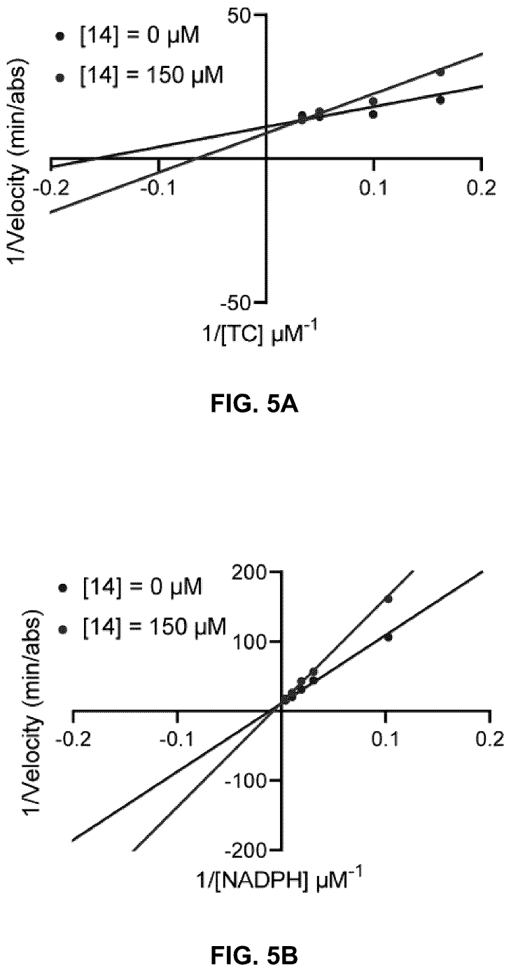

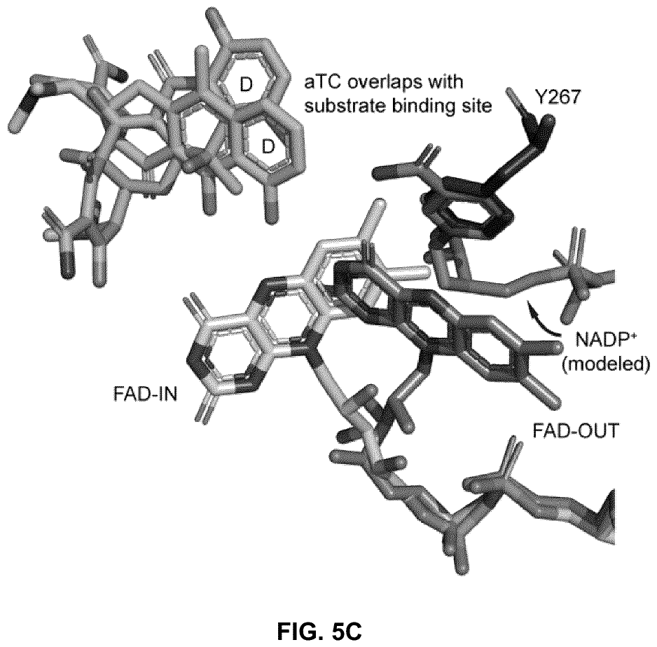

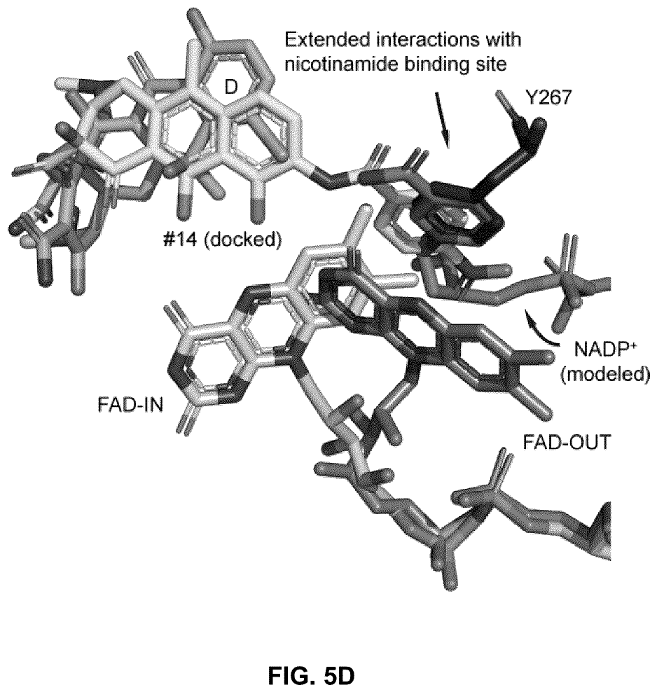

We also analyzed apparent steady-state kinetics NADPH consumption in the presence of variable TDase inhibitor, 1 or 14, to determine Kapp and vmax by fitting the observed reaction rates to the Michaelis-Menten equation (FIG. 4H). aTC (1) appeared to enhance the rate of NADPH consumption in a dose-dependent manner for Tet(X7) (Kapp=2.1±0.3 μM, vmax=0.043±0.002 min-1) and Tet(50) (Kapp=2.0±0.3 μM, vmax=0.24±0.01 min-1) (FIG. 4C, FIG. 4D). As expected, the para-NO2-C9-benzamide aTC analog (14) did not promote NADPH consumption at any concentration tested (FIG. 4D, FIG. 4G). Lineweaver-Burke plots for aTC (1) against Tet(X7) and Tet(50) suggest mixed competitive inhibition for variable TC. We were unable to obtain Lineweaver-Burke plots for aTC (1) with variable NADPH due to the dose-dependent enhancement of NADPH oxidation by aTC and assume this observed activity is due to an uncompetitive binding interaction (FIG. 4P). These findings support unique inhibition mechanisms for aTC (1) as a competitive slow substrate for Type 1 TDases and a mixed competitive inhibitor for Type 2 TDases. Lineweaver-Burke plots for para-NO2-C9-benzamide-aTC (14) against Tet(50) with variable TC (FIG. 5A) and variable NADPH (FIG. 5B) support a model for competitive inhibition with respect to both TC and NADPH. The same trend was true, although less pronounced, for compound 14 against Tet(X7) (FIG. 21). These findings along with molecular docking (FIG. 1E, FIG. 5C, FIG. 5D, FIG. 9E) validate the bisubstrate nature of C9-benzamide/benzylamine-aTC analogs as TDase inhibitors.

A variety of TDase inhibitors have been reported including AZT, bismuth salts, flavonoids, and aTC. The development of selective inhibitors for enzymes in the FMO superfamily has been challenging due in part to the mechanistic complexity, dynamic nature, and substrate flexibility observed for this enzyme class. aTC represents a cell permeable privileged scaffold from which to build better TDase inhibitors. Bisubstrate FMO inhibitors are promising given the potential to inhibit multiple steps of the catalytic cycle and to enhance binding affinity through extended ligand engagement across multiple binding sites. Bisubstrate inhibitors spanning the TC and NADPH binding sites of TDases are conceptually possible given these two ligands seem to have unique and adjacent binding sites near the dynamic FAD cofactor in class A FMOs. The NADPH binding site in class A FMOs has not been fully elucidated, however a co-crystal structure of a mutant version of para-hydroxybenzoate hydroxylase (PHBH) complexed to NADPH suggests that the NADPH binding site is located on a surface exposed groove between the substrate and FAD-binding domains. The crystallographically observed NADPH binding mode in PHBH appears to have the nicotinamide extended away from the active site, although molecular modeling by the GSK group with KMO supports an alternate binding mode that better supports a functional model for reduction of FAD adopting the solvent-exposed “OUT” conformation. Hence, bisubstrate inhibitors could in principle be constructed via fusion of the substrate and nicotinamide scaffolds (FIG. 5C. FIG. 5D). This concept has been demonstrated against the enzyme KMO and is validated in this work for TDases.

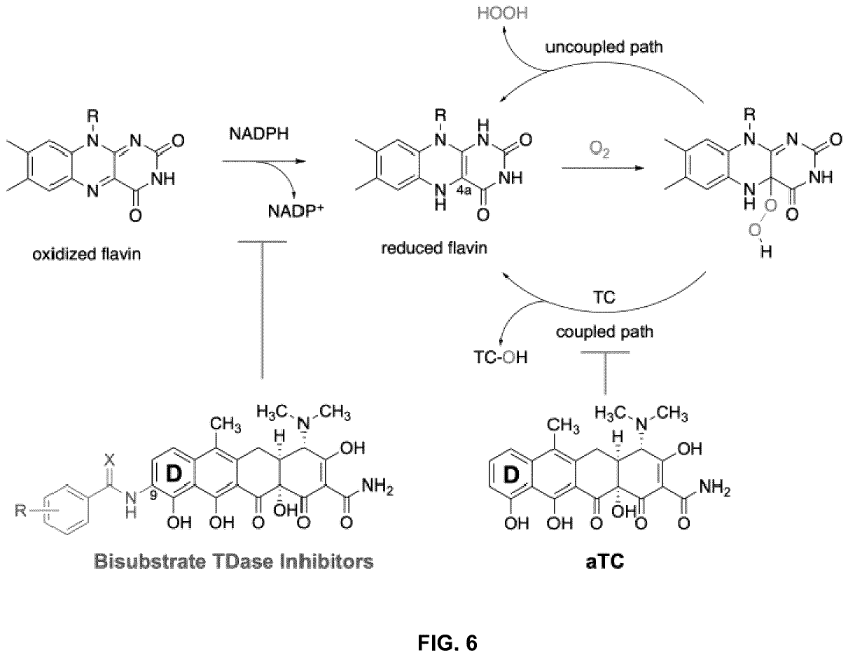

Our findings validate the promise of non-antibacterial TDase inhibitors to overcome TC resistance caused by enzymatic inactivation. Through the development of bisubstrate TDase inhibitors we have engineered out the inherent antibacterial activity of the parent aTC scaffold and improved selectivity and potency for TDase inhibition. We predict that this will widen the therapeutic window of TDase inhibitors and eliminate the potential for the TDase inhibitor to serve as a selection factor for resistance. aTC (1) acts as a competitive sacrificial substrate for Type 1 TDases (Tet(X7)) and a mixed competitive inhibitor for Type 2 TDases (Tet(50)). This suggests that aTC potentially samples multiple binding modes in the TDase active sites—TC substrate-like binding mode with FAD-‘IN’ for Type 1 TDases and aTC inhibitor binding mode with FAD-‘OUT’ for Type 2 TDases. The bisubstrate inhibitor 14 was competitive with both TC and NADPH for both Type 1 (Tet(X7)) and Type 2 (Tet(50)) TDases and was not turned over as a substrate by either enzyme type. This is consistent with our working model for bisubstrate inhibition where the C9-substituted aTC analogs bind in the TC site via the core aTC scaffold and the NADPH nicotinamide site via π-π stacking of the C9-benzamide/benzylamine aryl group with the isoalloxazine heterocycle of FAD in the ‘OUT’ conformation (FIG. 5D). Bisubstrate TDase inhibitors act upstream of the parent aTC inhibitor scaffold by inhibiting reduction of FAD to FADH2, which is a prerequisite for the formation of a reactive C4a-peroxy-flavin intermediate. This could explain why bisubstrate TDase inhibitors are not substrates for Type 1 TDases. The bisubstrate nature might also stabilize the inhibitor binding mode preventing sampling of the substrate binding mode that might explain turnover of aTC as a slow substrate by Type 1 TDases. aTC stimulates NADPH oxidation and FAD reduction leading to a futile cycle of the ‘uncoupled’ oxidative path resulting in accumulation of H2O2 (FIG. 6).

There is no deposited structure of a Type 1 TDase with the FAD cofactor in the ‘OUT’ conformation which limited our ability to pursue rigorous molecular docking to the Type 1 enzymes without a validated structural model. It is presumed that FAD dynamics are conserved across Type 1 and 2 enzymes, but it is noteworthy that Type 1 enzymes lack the conserved Y267 found to stabilize FAD in the ‘OUT’ conformation in Type 2 enzymes. However, the overall loop architecture is conserved across Type 1 and 2 enzymes with a Leu residue at the analogous 267 position in Type 1 enzymes. The inability of Leu267 to stabilize FAD in the ‘OUT’ conformation might be limiting the ability to capture this conformation in structural studies due to an increase in flavin and protein dynamics. Bisubstrate TDase inhibitors slow or block NADPH oxidation and FAD reduction for both Type 1 and 2 TDases (FIG. 4A) further supporting a shared model of bisubstrate TDase inhibition through competitive binding at the substrate and nicotinamide sites. Hence, we propose that the binding mode of bisubstrate TDase inhibitors can be controlled by extended interactions between aryl substitutions and the FAD isoalloxazine heterocycle in both Type 1 and 2 enzymes (FIG. 5D).

The SAR explored in this study revealed that the C9-benzamides are more potent than C9-benzylamines and that para-substitution of the phenyl ring with an electron withdrawing group (NO2 or CF3) was preferred. The more rigid amide linkage might better align the benzamide to facially interact with the FAD isoalloxazine. However, the water solubility of the C9-benzylamines was significantly better than the C9-benzamides which is critical for preclinical advancement. Hence, we propose that benzylic substitution of the C9-benzylamine series might help to rigidify the inhibitor to bias the binding conformation while preserving the water solubility and adding beneficial metabolic stability (given benzylic positions are often the site of oxidation via general metabolism). The modest whole cell activity of the bisubstrate inhibitors might also be associated to reduced cell permeability relative to aTC leading to low cell accumulation in E. coli. Despite the advancements made in this study, the bisubstrate TDase inhibitors require further optimization of solubility, potency, stability, selectivity, and cellular accumulation. This could potentially be achieved through systematic exploration of the C9-aryl substituent to leverage existing knowledge of favorable aromatic heterocycle π-π stacking interactions. These bisubstrate compounds present a new mechanism of TDase inhibition with the opportunity for further rational design to improve effectiveness of overcoming TDase resistance when used in combination with TC antibiotics.

### CONCLUSIONS

We synthesized and characterized bisubstrate inhibitors of TDase resistance enzymes via scaffold fusion of a known inhibitor, aTC (1), and benzamide/benzylamine structural mimics of nicotinamide (FIG. 5C, FIG. 5D). Inhibitor design was guided by molecular modeling using the co-crystal structure of aTC and the Type 1 TDase Tet(50). Modeling identified the C9-position of the aTC D-ring as the ideal attachment point for aryl nicotinamide isosteres to promote π-π stacking with the FAD isoalloxazine heterocycle in the solvent-exposed ‘OUT’ conformation to simulate the structural environment of the reductive step of the catalytic cycle. We validated the bisubstrate nature of the C9-functionalized TDase inhibitors against Type 1 (Tet(X7)) and Type 2 (Tet(50)) TDase enzymes and established important SARs via in vitro biochemical characterization. aTC (1) is a competitive inhibitor with respect to TC and is uncompetitive with respect to NADPH. aTC is a competitive sacrificial substrate against Type 1 TDases and promotes the uncoupled reduction of FAD by NADPH. However, para-NO2-C9-benzamide aTC inhibitor 14 is competitive with both TC and NADPH and blocks reduction of FAD by NADPH. This compound is stable towards Type 1 and 2 TDases consistent with a bisubstrate mode of TDase inhibition where the FAD ‘OUT’ conformation is stabilized by the inhibitor binding mode. aTC (1) rescues TC activity against resistant E. coli strains expressing TDases and also exhibits inherent antibacterial activity. Bisubstrate TDase inhibitors are non-antibacterial agents capable of rescuing TC antibacterial activity against E. coli expressing Type 1 or 2 TDases. Hence, TDase inhibition via combination therapy is a viable therapeutic approach for overcoming TC resistance via enzymatic inactivation.

### EXPERIMENTAL MATERIALS AND METHODS

### General Materials and Methods

All in vitro kinetic assays and whole cell bacterial growth assays were prepared open to air in non-degassed buffer solutions using sterile technique. All organic solvents including deuterated NMR solvents and reagent chemicals used in preparation or analysis of synthetic compounds were obtained commercially and used without further purification. TC (HCl salt), NADPH (tetrasodium salt), and G6P dehydrogenase Leuconostoc mesenteroides (recombinant, expressed in E. coli, suspension in (NH4)2SO4) were purchased from Millipore-Sigma (St. Louis, MO). aTC (HCl salt) was purchased from Chemodex (United Kingdom). NADP+(hydrate) was purchased from Carbosynth (Compton, England). G6P was purchased from Chem-Impex (Wood Dale, IL). NMR spectra were obtained on a Varian Unity-Inova 500 MHz or Agilent Premium Compact+600 MHz spectrometer in 5 mm type 1, class A borosilicate glass NMR tubes (Wilman LabGlass part No. 535-PP-8). All free induction decay files (FIDs) were processed using Mestrenova version 11.0.4 software. Chemical shifts (6) are reported in parts per million (ppm) and referenced to residual non-deuterated solvent. Coupling constants (J) are reported in hertz (Hz). TDase in vitro reactions were monitored by optical absorbance spectroscopy on an Agilent Cary 50 UV-visible spectrophotometer using polystyrene cuvettes and LC-MS using an Agilent 6130 single quadrupole instrument (ESI+) with G1313 autosampler, G1315 diode array detector, and 1200 series solvent module with separation on a Phenomenex Gemini C18 column, 50×2 mm (5 μm) fit with a guard column cassette. LC-MS solvents were 0.1% formic acid in H2O (A) and 0.1% formic acid in ACN (B). Solvent gradient was linear starting from 0% B to 95% B over 20 min at a flow rate of 0.5 mL/min. HPLC was performed on an HP1050 system using a Luna 10 mm C18(2) 100 Å column (250 mm×21.2 mm) from Phenomenex fit with a guard column of the same matrix (15 mm×21.2 mm). HPLC solvents were 0.1% formic acid in H2O (A) and 0.1% formic acid in ACN (B) with a gradient formed from 0% B to 95% B over 20 min at a flow rate of 9 mL/min. LC-MS and HPLC data were processed using ChemStation software version B.04.02 SP1. Liquid medium bacterial growth assays were performed using Difco BBL Mueller-Hinton broth in Costar 96-well plates at 37° C. End-point growth density was judged by OD600 measurement using a Synergy H1 plate reader (BioTek, Inc.). Solid medium bacterial growth assays were performed using Mueller-Hinton No. 2 (MHII) agar and inhibition zone sizes were measured using a Neiko electronic caliper. SDS-PAGE analysis was carried out using Bio-Rad Any kD precast polyacrylamide gels with staining by Coomassie brilliant blue and comparison to a Bio-Rad precision plus protein dual Xtra pre-stained protein standard ladder.

### Compound Purity Statement.

All compounds were fully characterized by NMR and HRMS with purity analysis by LC-MS conducted prior to biological testing. Some samples contained varying amounts of C4-epimers as reported in the Methods section. Equilibration of C4-epimers is common for TC, aTC, and related compounds and is pH dependent.

All compounds are >95% pure by HPLC analysis. Epimerization of some compounds, presumably at C4, was observed. The ‘epimeric purity’ of each test compound was determined by LCMS.

### Synthesis of C9-Substituted aTC Derivatives

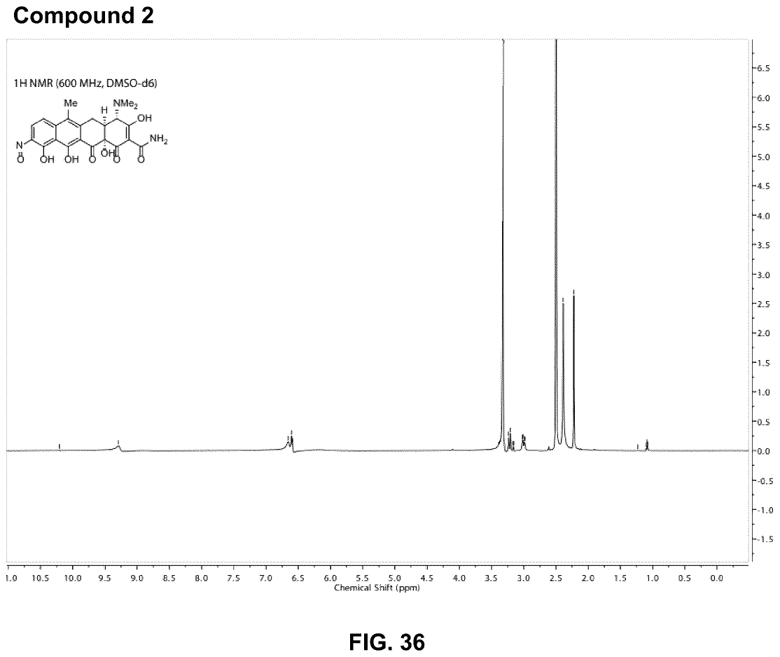

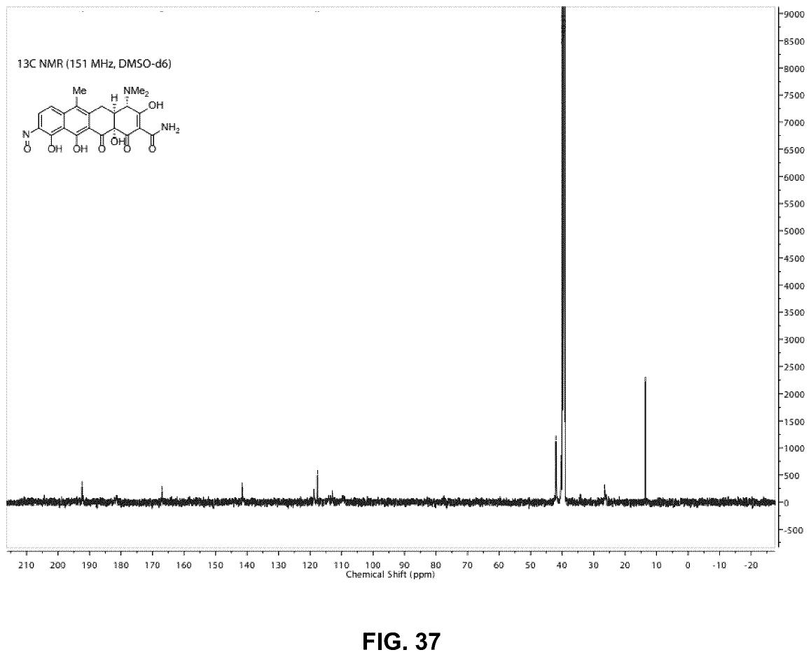

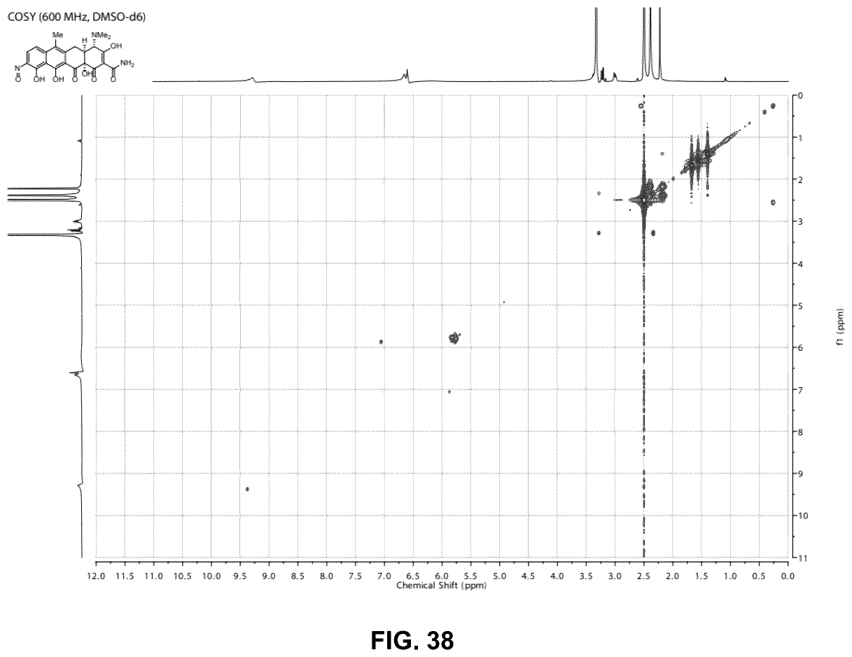

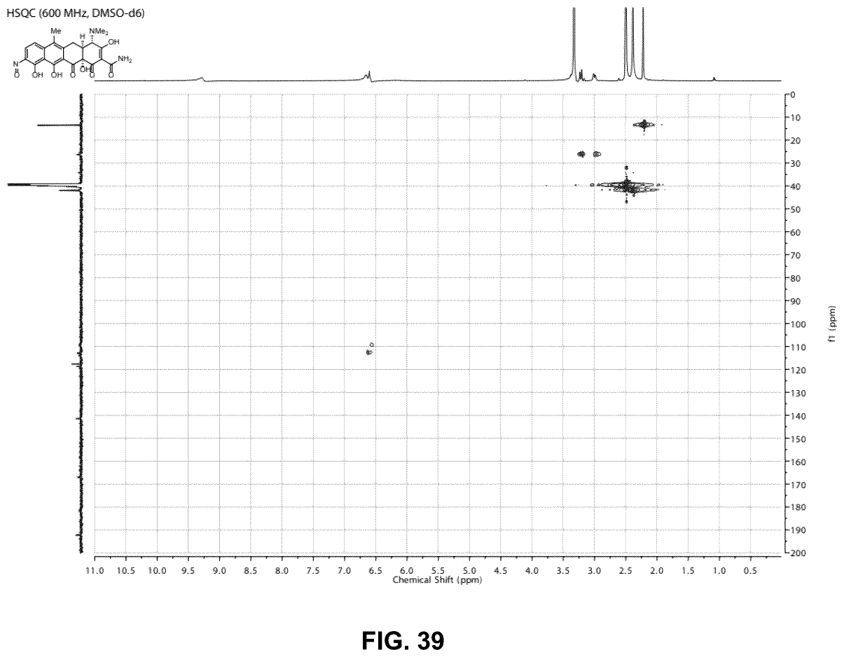

C9-NO-aTC (2).: A 25 mL Erlenmeyer flask was charged with aTC (0.1814 g, 0.392 mmol) and 9 mL of 0.1 M HCl (aq) and was cooled to 0° C. in an ice water bath to form a bright yellow suspension. Solid NaNO2 (0.0406 g, 0.588 mmol) was dissolved in 2.2 mL of 0.1 M HCl (aq) in a separate vial and was transferred dropwise over 15 min to the suspension of aTC at 0° C. The rusty orange reaction mixture was then filtered through a 0.45 μm PTFE syringe filter into a 100 mL round bottom flask containing 10 mL of MeOH to give a dark red filtrate. The original reaction flask was washed with 3×5 mL of 0.1 M HCl (aq), and the washes were subsequently filtered through the same syringe filter into the 100 mL round bottom flask. The solution was concentrated under reduced pressure via rotary evaporation to yield a reddish-orange residue. The residue was dissolved in minimal amounts of MeOH and diluted with 150 mL of Et2O and cooled to 4° C. to induce precipitation of the product. After ˜24 h, the solvent was removed via pipet and the orange solid was triturated with fresh Et2O. The solid was dried under a stream of N2 gas to yield compound 2 as the corresponding HCl salt (powdery, bright orange solid; 0.1659 g, 86% yield). The resulting solid was used for the next step without further purification. FIGS. 36, 37, 38, and 39 show the NMR characterization of compound 2. ¹H NMR (500 MHz, DMSO-d6) δ (ppm) 9.29 (s, 1H) 6.66-6.59 (m, 2H), 3.22 (d, J=17 Hz, 1H), 3.00 (dd, J=17 Hz, 5 Hz), 2.39 (s, 6H), 2.22 (s, 3H). ¹³C NMR (151 MHz, DMSO-d6) δ (ppm) 192.4, 167.0, 141.5, 118.8, 117.7, 42.0, 40.3, 26.5, 13.6. LCMS purity 98% (see FIG. 118). MS (ESI+) [M+H]+ found 455.9. High-resolution mass spectrometry (HRMS) (time-of-flight (TOF) MS ESI+) calculated for C22H21N3O8 [M+H]⁺ 456.1401; found 456.1387.

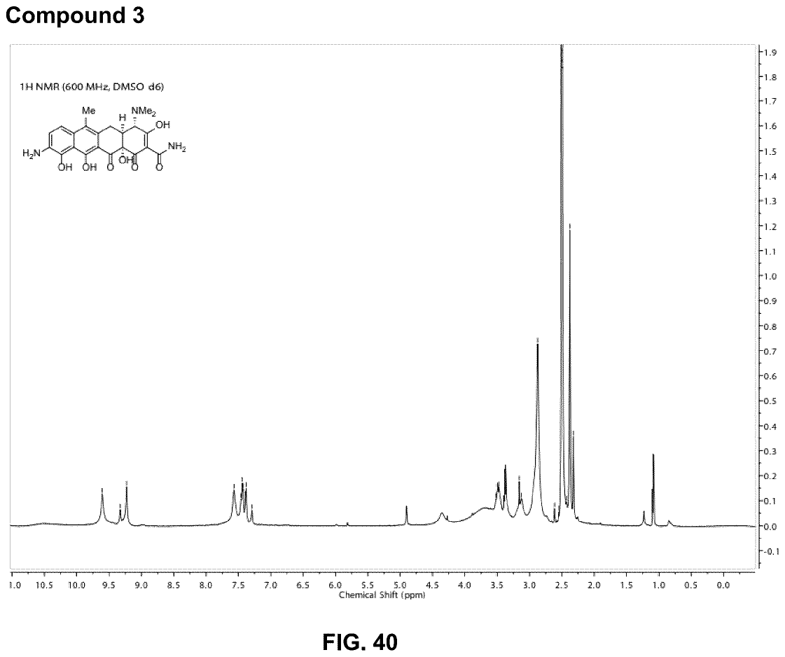

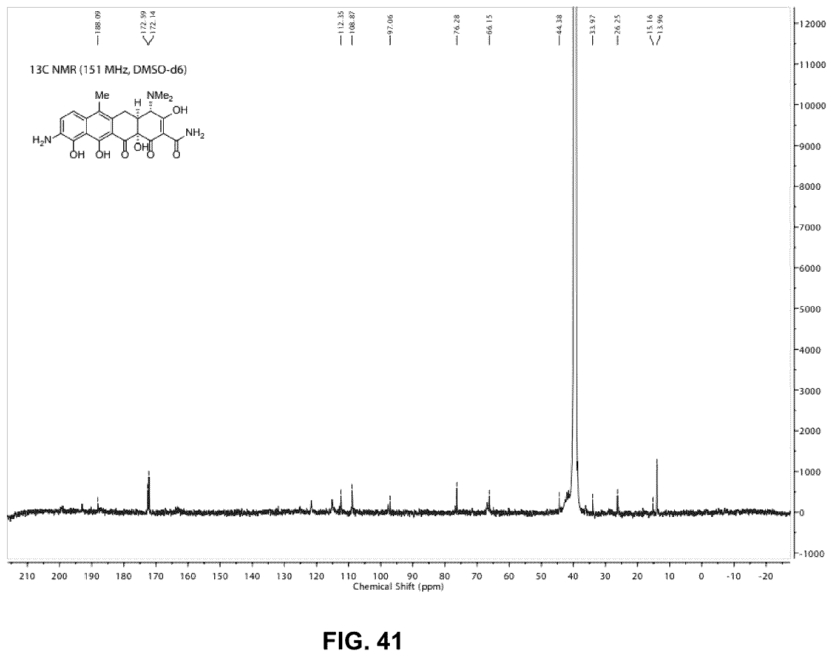

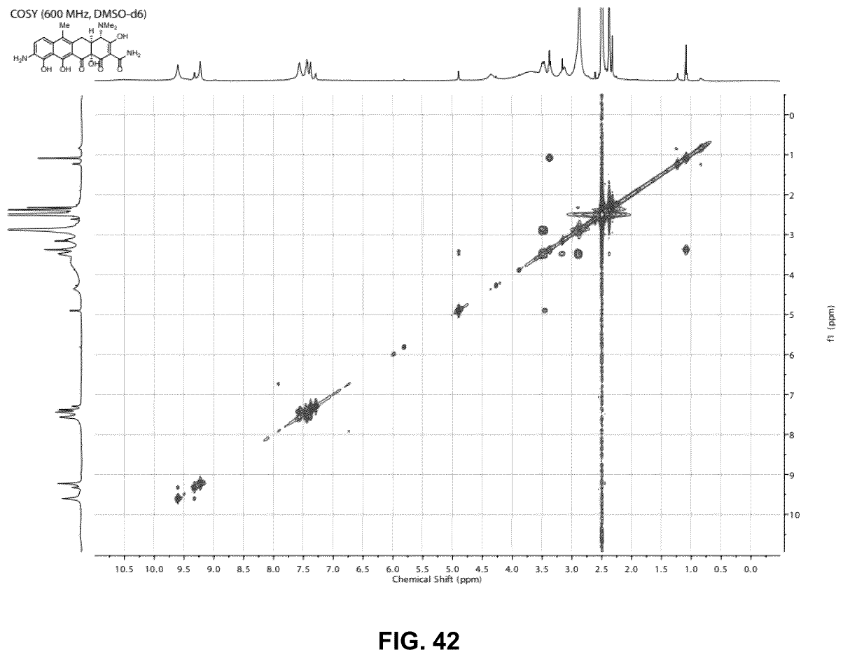

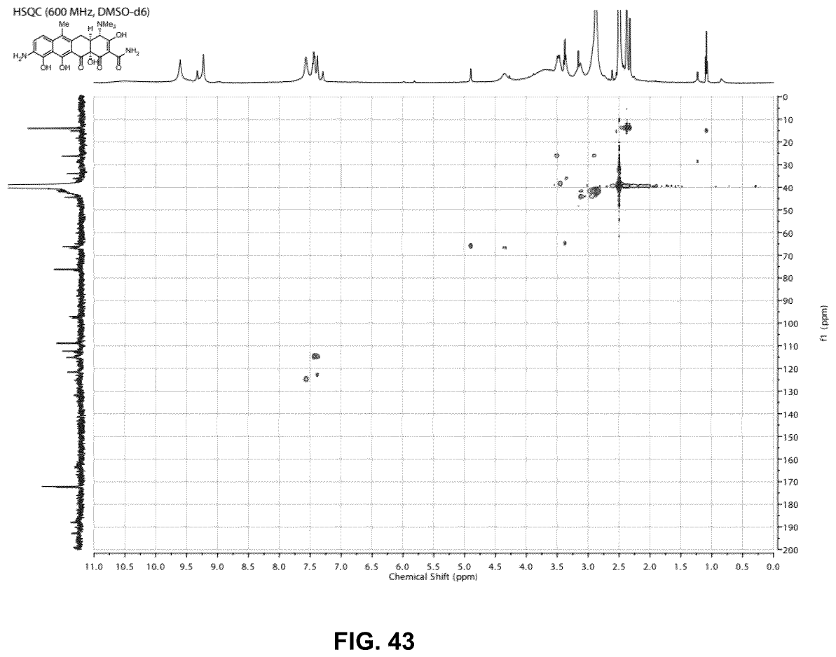

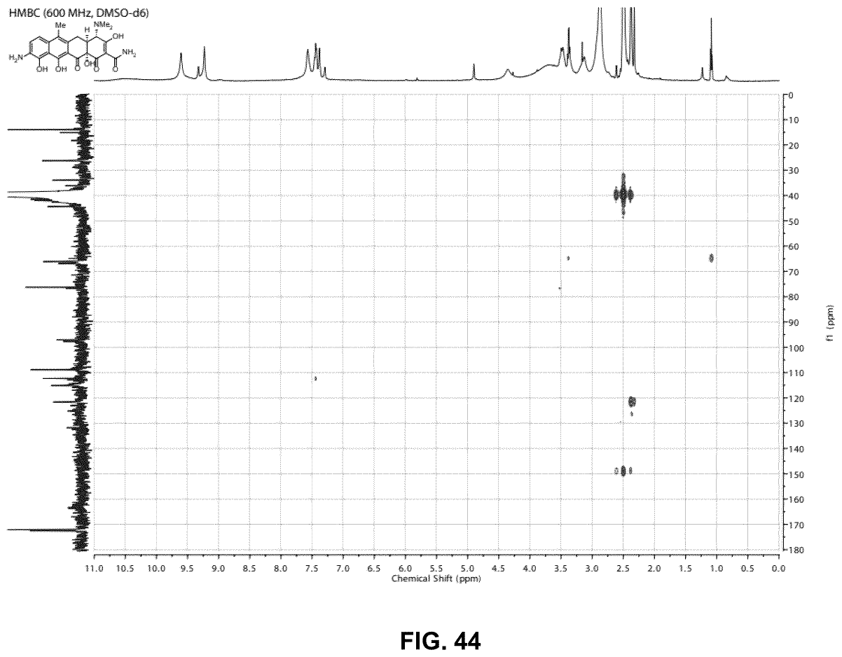

C9-NH2-aTC (3).: Compound 2 (0.1659 g, 0.337 mmol) was added as a solid to an oven-dried 100 mL round bottom flask. The solid was dissolved in a few mL of MeOH and 25 drops of 1 M NaOH (aq) were added while gently swirling the flask. The resulting dark reddish-purple solution was concentrated under reduced pressure via rotary evaporation and dried for ˜48 h under vacuum to yield a dark purple solid. After drying under vacuum, the flask was backfilled with Ar gas and charged with 17 mL of anhydrous MeOH (DriSolv). The flask was sonicated to help dissolve residual solids. The flask was charged with 10% Pd/C (0.008 g, 5% by mass) and exposed to H2 gas with direct bubbling into the MeOH solution (15 min). After 15 min, the vent needle was removed, and reaction mixture was left to stir under an H2 atmosphere (˜1 atm balloon) until complete (5 h as judged by LC-MS analysis). The reaction mixture was acidified by addition of 8 mL 1 M HCl (aq) prior to filtration through celite. The filtrate and combined MeOH washings were concentrated under reduced pressure via rotary evaporation to yield a brown residue. The residue was dissolved in a minimal amount of MeOH, diluted with 150 mL Et2O, and cooled to 4° C. to induce precipitation of the product. After ˜24 h, the solvent was removed via pipet and the light brown solid was triturated with fresh Et2O. The solid was dried under a stream of N2 gas to yield compound 3 as the corresponding HCl salt (powdery, light brown solid; 0.1264 g, 73% yield). The resulting solid was used for the next step without further purification. FIGS. 40, 41, 42, 43 and 44 show the NMR characterization of compound 3. ¹H NMR (600 MHz, DMSO-d6) δ (ppm) 9.60 (s, 1H), 9.23 (s, 1H), 7.63-7.51 (m, 1H), 7.44 (d, J=8.1 Hz, 1H), 7.38 (d, J=6.7 Hz, 1H), 3.49-3.45 (m, 3H), 2.97-2.91 (m), 2.38 (s, 3H). ¹³C NMR (151 MHz, DMSO-d6) δ (ppm) 188.1, 172.6, 172.1, 121.7, 115.2, 112.4, 108.9, 97.8, 97.1, 76.8, 76.3, 66.7, 66.2, 44.3, 34.0, 26.3, 15.2, 14.0. LCMS purity 99% (see FIG. 119). MS (ESI+) [M+H]⁺ found 442.0. HRMS (TOF MS ESI+) calcd for C22H23N3O7 [M+H]⁺ 442.1609; found 442.1594.

General procedure (A) for synthesis of C9-amido-aTC analogs (4-14).: Acetic acid (0.056 mL, 0.972 mmol) and DIPEA (0.271 mL, 1.556 mmol) were dissolved in 26 mL of anhydrous DMF (DriSolv) in a dry 100 mL round bottom flask charged with HATU (0.3696 g, 0.972 mmol). The clear solution turned pale yellow and was stirred at room temp for 10 min before the addition of compound 3 (HCl salt). The dark brown reaction solution was stirred for 5 min at room temp and then quenched with 5 mL of 1 M HCl (aq) and concentrated under reduced pressure via rotary evaporation to yield a dark brown residue. The residue was dissolved in MeOH, filtered through a 0.45 μm PTFE syringe filter, and purified by RP-C18 prep-HPLC to provide the desired products 4-15 as the corresponding formic acid salts.

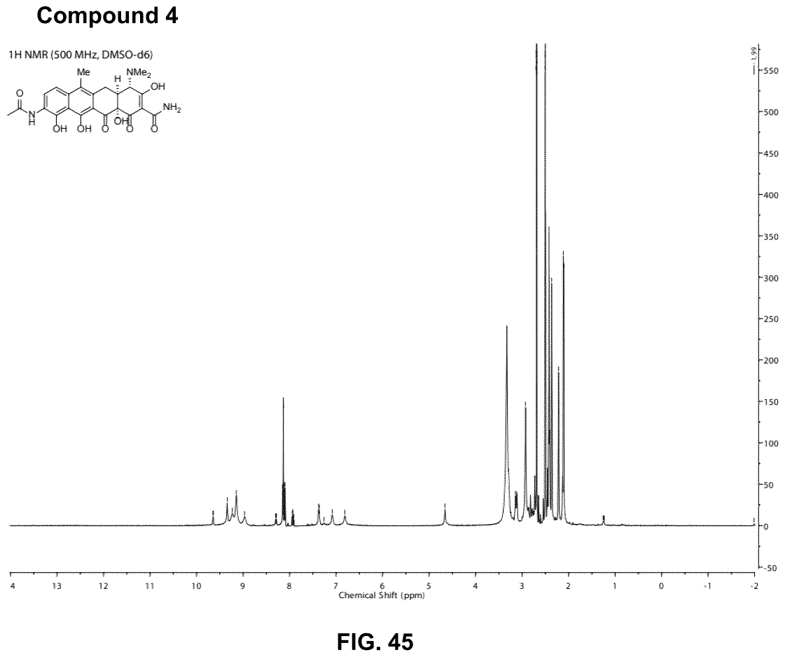

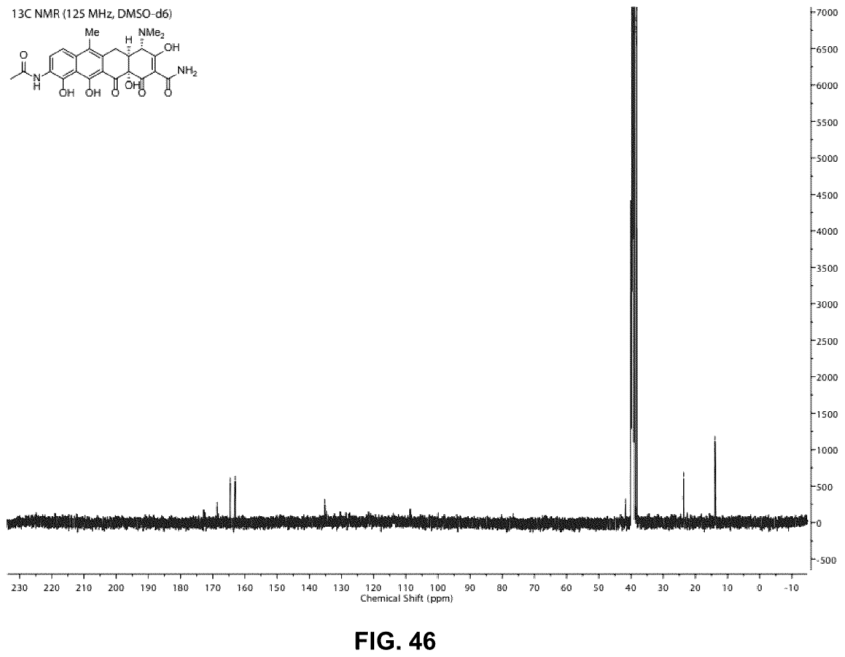

(4S,4aS,12aS)-9-acetamido-4-(dimethylamino)-3,10,11,12a-tetrahydroxy-6-methyl-1,12-dioxo-1,4,4a,5,12,12a-hexahydrotetracene-2-carboxamide (4).: Compound 4 was prepared according to general procedure A and was obtained in 25% yield as a brown solid. FIGS. 45 and 46 show the NMR characterization of compound 4. ¹H NMR (500 MHz, DMSO-d6) δ 9.64 (d, J=4.2 Hz, OH), 9.34 (s, 1H), 9.23 (s, 2H), 9.14 (s, 2H), 8.96 (s, 1H), 8.12 (t, J=10 Hz, 2H), 7.37 (d, J=8.8 Hz, 1H), 7.08 (s, 1H), 6.81 (s, 1H), 4.66 (s, 1H), 3.16-3.09 (m, 2H), 2.93 (s, 4H), 2.42 (s, 6H), 2.36 (s, 3H), 2.22 (s, 2H), 2.11 (d, J=7.8 Hz, 4H). ¹³C NMR (126 MHz, DMSO-d6) δ 168.6, 164.6, 163.0, 135.2, 41.7, 38.3, 23.8, 23.6, 13.9, 13.7. Prep-HPLC tr 13m. LCMS epimeric purity 82% (see FIG. 120). MS (ESI+) [M+H]⁺ found 483.8. HRMS (TOF MS ESI+) calculated for C₂₄H₂₅N₃O₈ [M+H]⁺ 484.1714; found 484.1697.

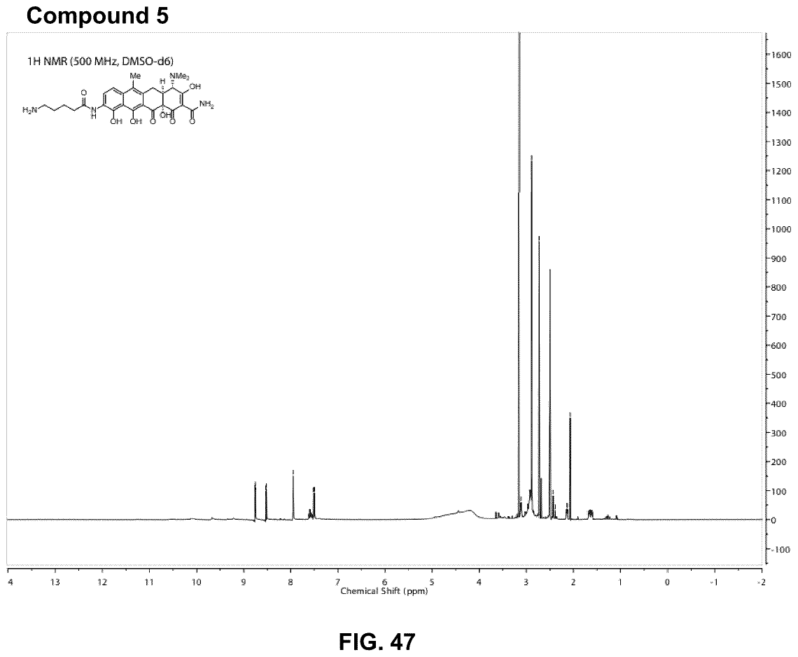

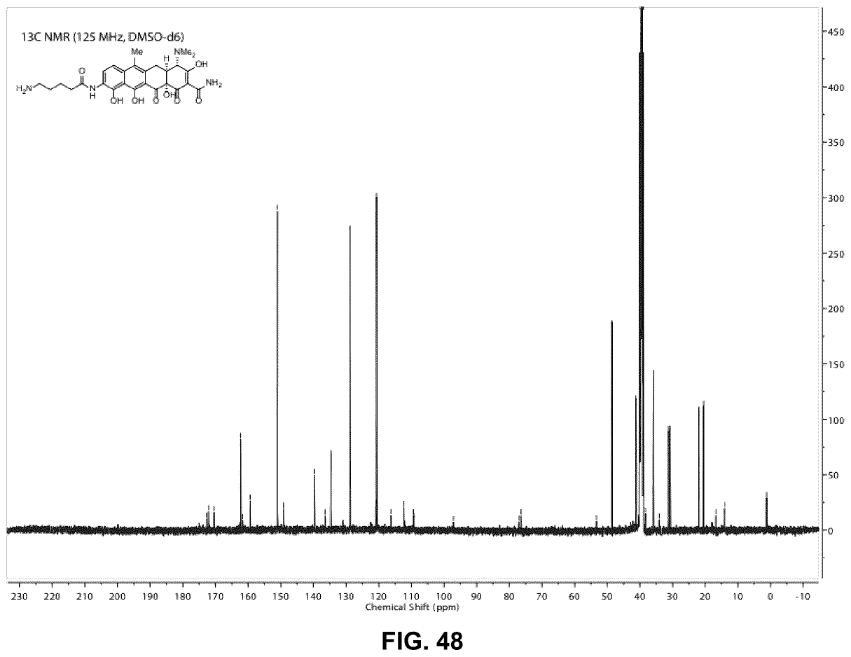

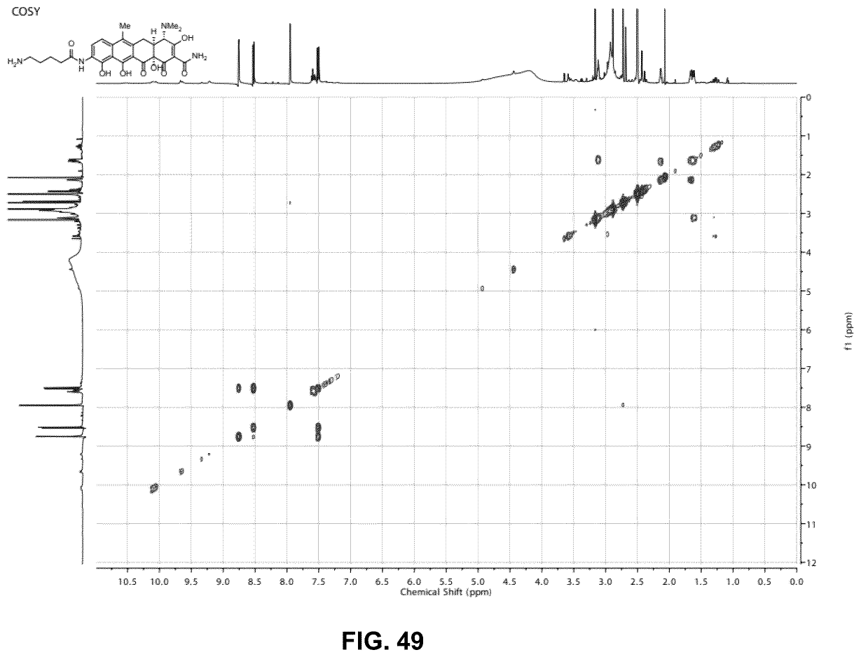

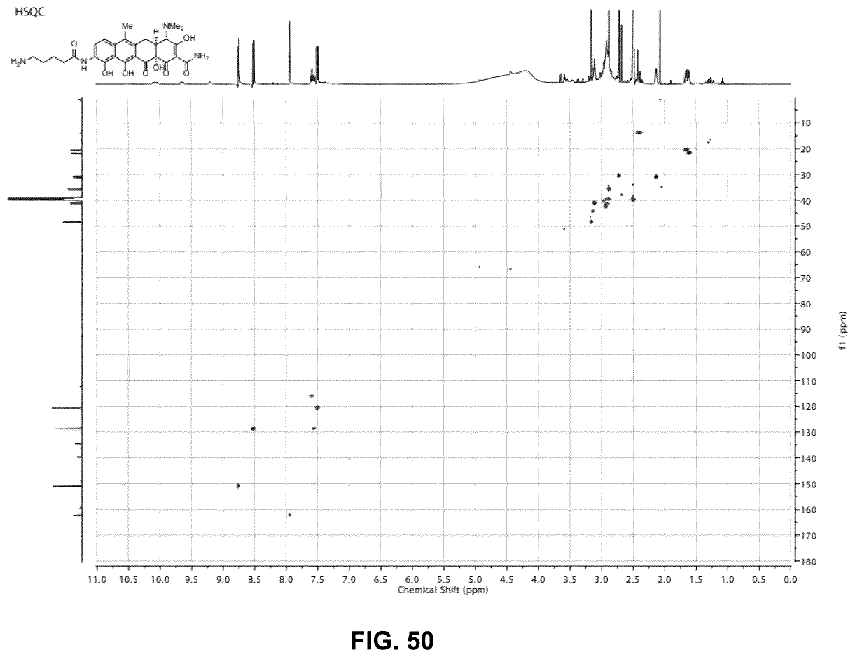

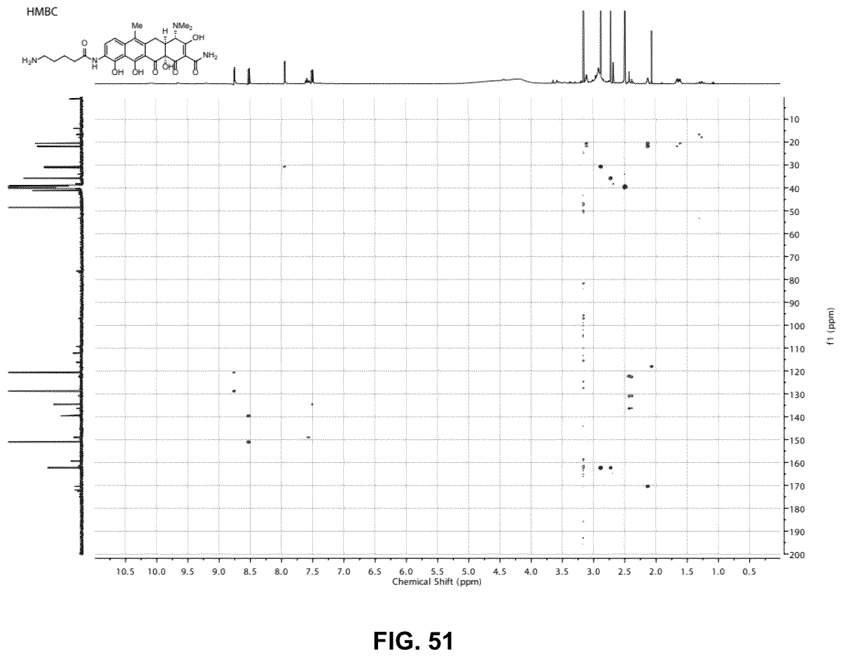

(4S,4aS,12aS)-9-(5-aminopentanamido)-4-(dimethylamino)-3,10,11,12a-tetrahydroxy-6-methyl-1,12-dioxo-1,4,4a,5,12,12a-hexahydrotetracene-2-carboxamide (5).: Compound 5 was prepared according to general procedure A and was obtained in 21% yield as a brown solid. FIGS. 47, 48, 49, 50, and 51 show the NMR characterization of compound 5. 1H NMR (500 MHz, DMSO-d6) δ 8.76 (d, J=4.4 Hz, 1H), 8.52 (d, J=8.4 Hz, 1H), 7.95 (s, 1H), 7.52 (d, J=4.4 Hz, 1H), 7.50 (d, J=4.4 Hz, 1H), 3.16 (s, 3H), 3.13-3.10 (m, 2H), 2.89 (s, 6H), 2.73 (s, 5H), 2.43 (s, 1H), 2.16-2.11 (m, 2H), 2.07 (s, 1H) 1.69-1.65 (m, 2H), 1.65-1.59 (m, 2H). ¹³C NMR (126 MHz, DMSO-d6) δ 172.60, 172.08, 170.44, 162.30, 161.68, 159.39, 151.07, 149.08, 139.66, 136.39, 134.60, 128.80, 120.87, 120.68, 116.23, 112.27, 109.31, 109.19, 97.12, 77.03, 76.35, 53.26, 48.58, 41.25, 38.25, 35.79, 34.06, 31.17, 30.77, 21.89, 20.55, 16.70, 14.08, 14.02, 1.16. Prep-HPLC tr 13m. LCMS epimeric purity 60% (see FIG. 121). MS (ESI+) [M+H]+ found 540.3. HRMS (TOF MS ESI+) calculated for C₂₇H₃₂N₄O₈ [M+H]⁺ 540.2215; found 540.2452.

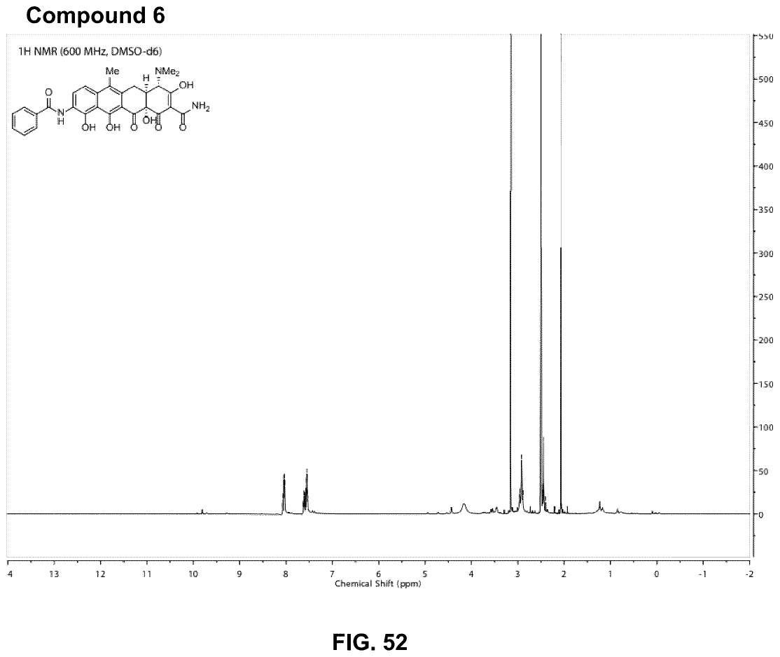

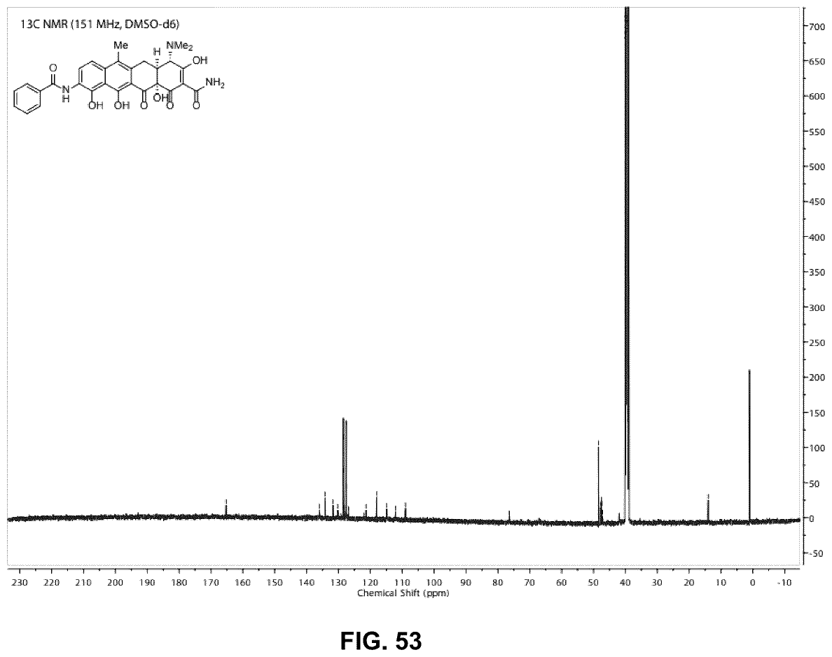

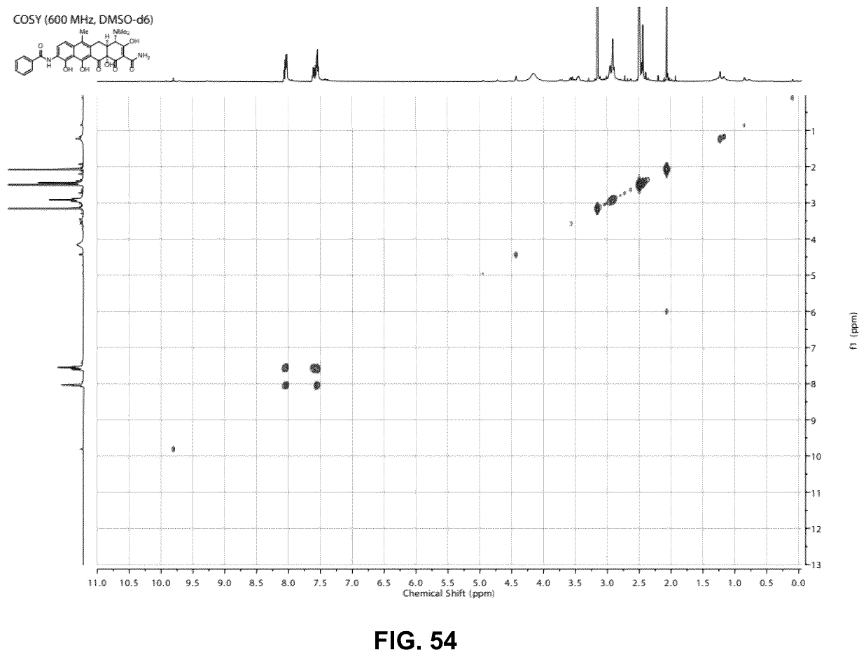

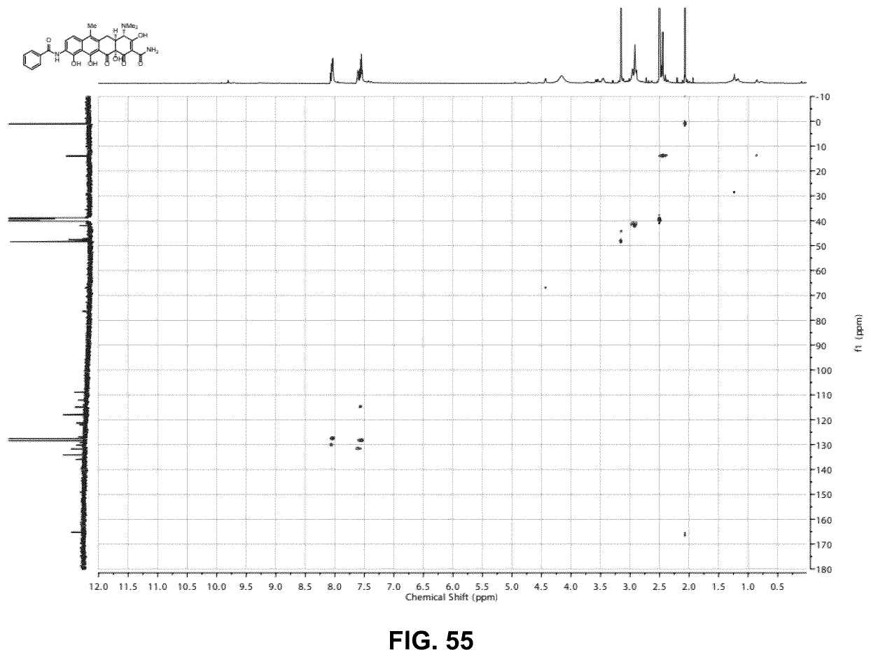

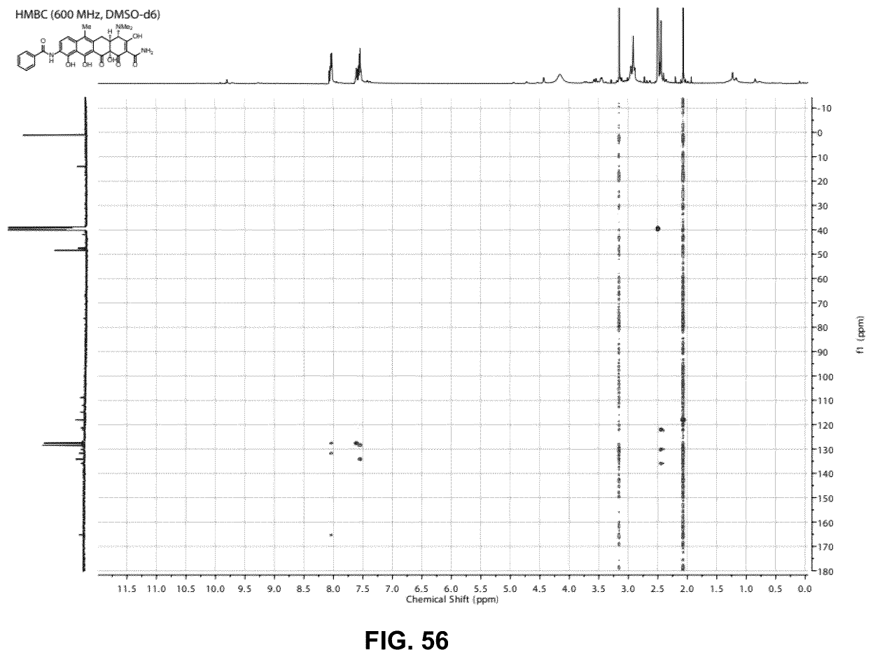

(4S,4aS,12aS)-9-benzamido-4-(dimethylamino)-3,10,11,12a-tetrahydroxy-6-methyl-1,12-dioxo-1,4,4a,5,12,12a-hexahydrotetracene-2-carboxamide (6).: Compound 6 was prepared according to general procedure A and was obtained in 21% yield as a brown solid. FIGS. 52, 53, 54, 55, and 56 show the NMR characterization of compound 6. ¹H NMR (500 MHz, DMSO-d6) δ (ppm) 8.08-8.02 (m, 3H), 7.64-7.53 (m, 4H) 3.16 (s, 3H), 2.98-2.94 (m, 1H), 2.90-2.88 (m, 1H), 2.44 (s, 1H), 2.40 (s, 1H), 2.07 (s, 6H). ¹³C NMR (125 MHz, DMSO-d6) δ (ppm) 165.2, 136.0, 134.2, 131.7, 130.2, 128.5, 128.2, 127.6, 126.9, 121.3, 118.0, 114.9, 112.1, 108.9, 76.4, 48.5, 47.9, 47.7, 47.6, 47.4, 47.2. Prep-HPLC tr 14m. LCMS epimeric purity 98% (see FIG. 122). MS (ESI+) calculated for C₂₉H₂₇N₃O₈ [M+H]⁺ 546.2; found 546.2.

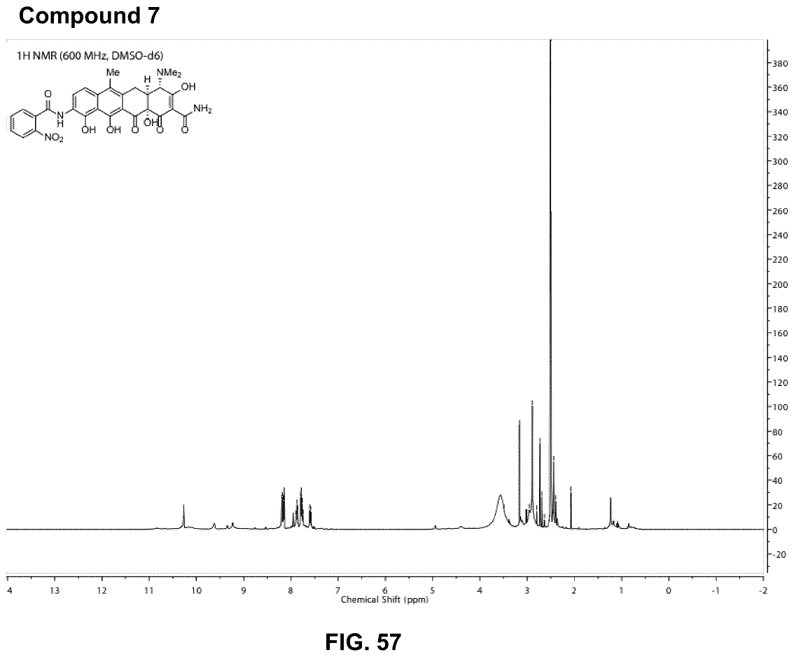

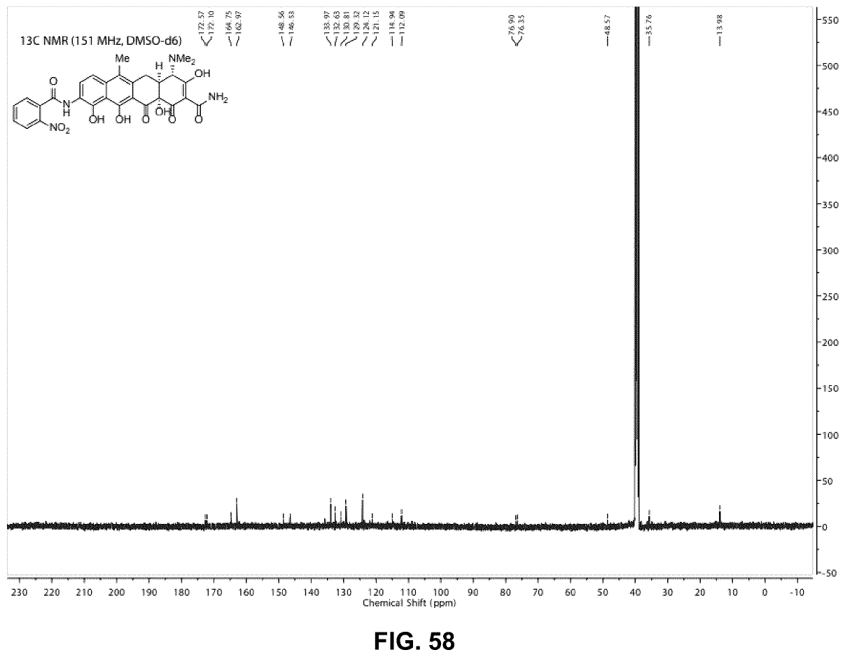

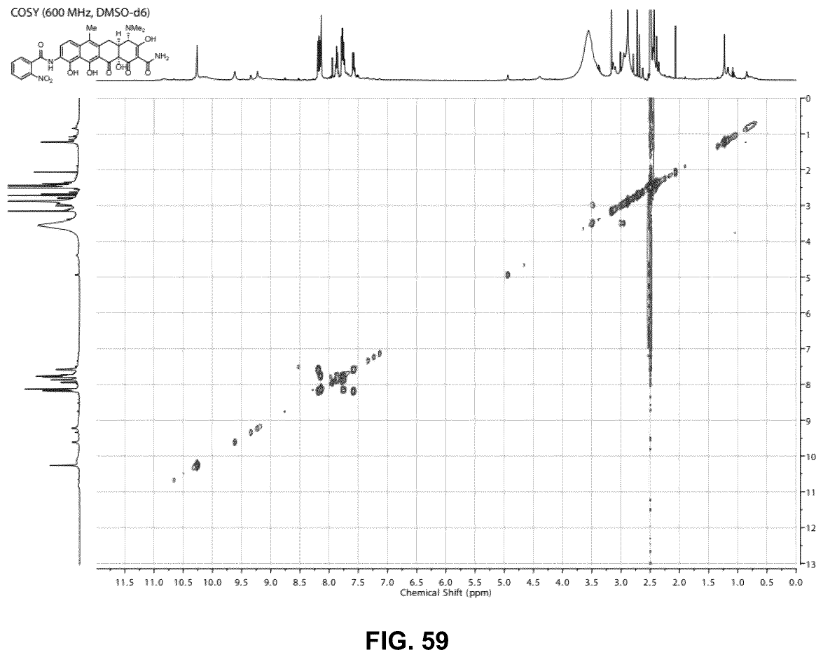

(4S,4aS,12aS)-9-(2-nitrobenzamido)-4-(dimethylamino)-3,10,11,12a-tetrahydroxy-6-methyl-1,12-dioxo-1,4,4a,5,12,12a-hexahydrotetracene-2-carboxamide (7).: Compound 7 was prepared according to general procedure A and was obtained in 5% yield as an orange solid. FIGS. 57, 58, and 59 show the NMR characterization of compound 7. ¹H NMR (500 MHz, DMSOd6) δ (ppm) 8.19 (d, J=9 Hz, 1H), 8.15 (d, J=9 Hz, 1H), 7.88 (t, J=7.5 Hz, 1H), 7.79 (d, J=8 Hz, 1H), 7.75 (d, J=8 Hz, 1H), 7.62-7.56 (m, J=5 Hz, 1H), 3.54-3.42 (m, 1H), 3.01 (s, 1H), 2.98-2.94 (m, 1H), 2.89 (s, 6H), 2.44 (s, 3H). ¹³C NMR (125 MHz, DMSOd6) δ (ppm) 172.6, 172.1, 164.7, 162.9, 148.6, 146.5, 134.0, 132.6, 130.8, 129.3, 124.1, 121.2, 114.9, 112.1, 76.9, 76.4, 48.6, 35.8, 14.0. Prep-HPLC tr 14m. LCMS epimeric purity 75% (see FIG. 123). MS (ESI+) [M+H]⁺ found 591.2. HRMS (TOF MS ESI+) calculated for C₂₉H₂₆N₄O₁₀ [M+H]⁺ 591.1722; found 591.1720.

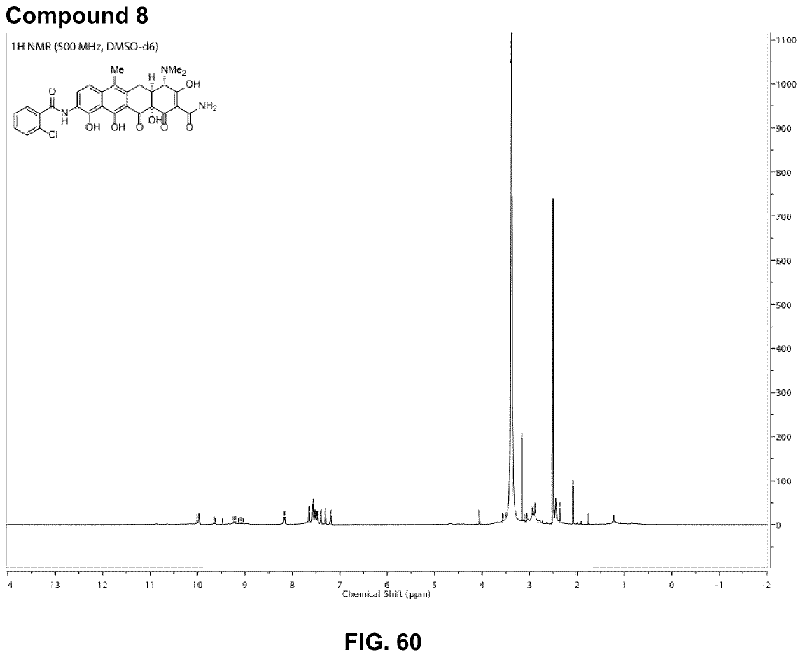

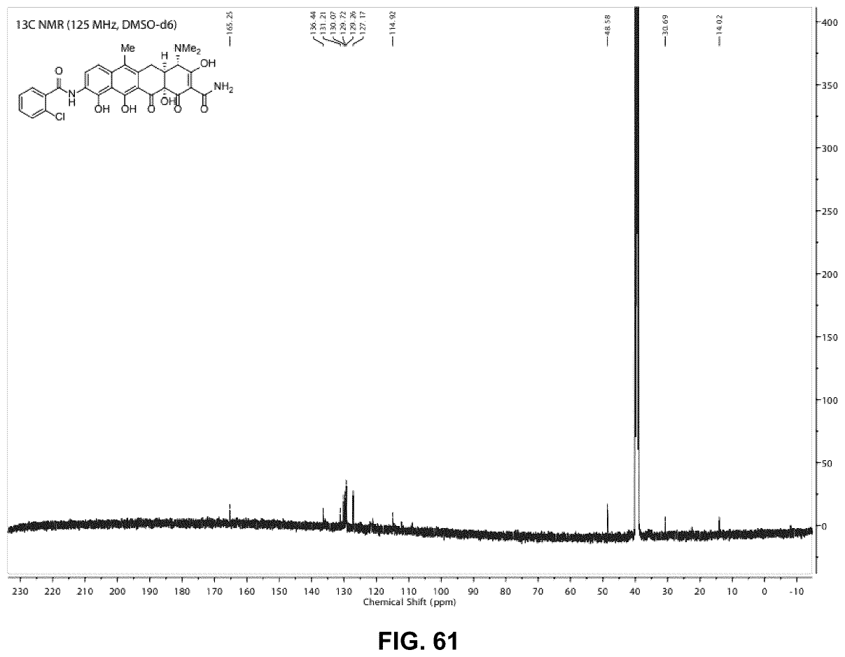

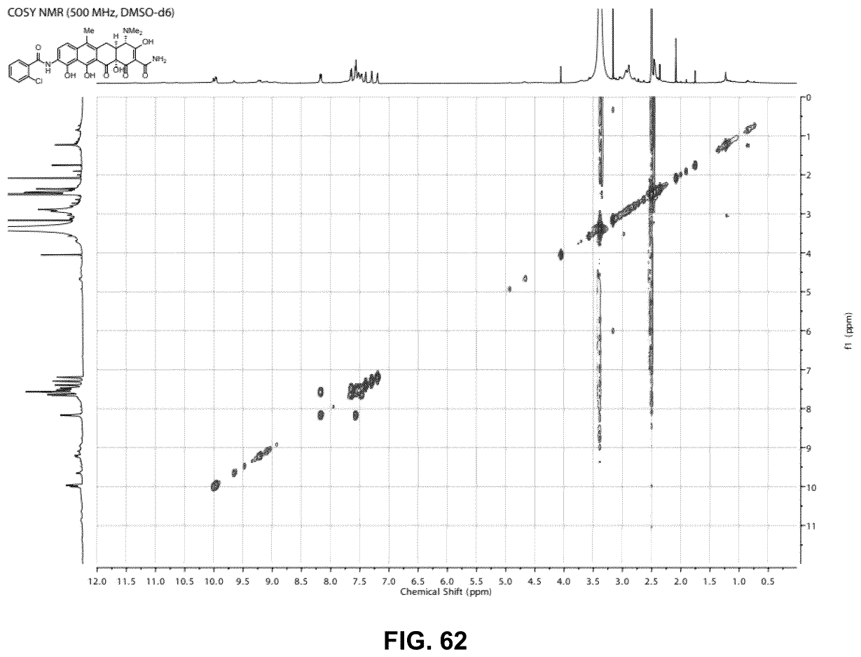

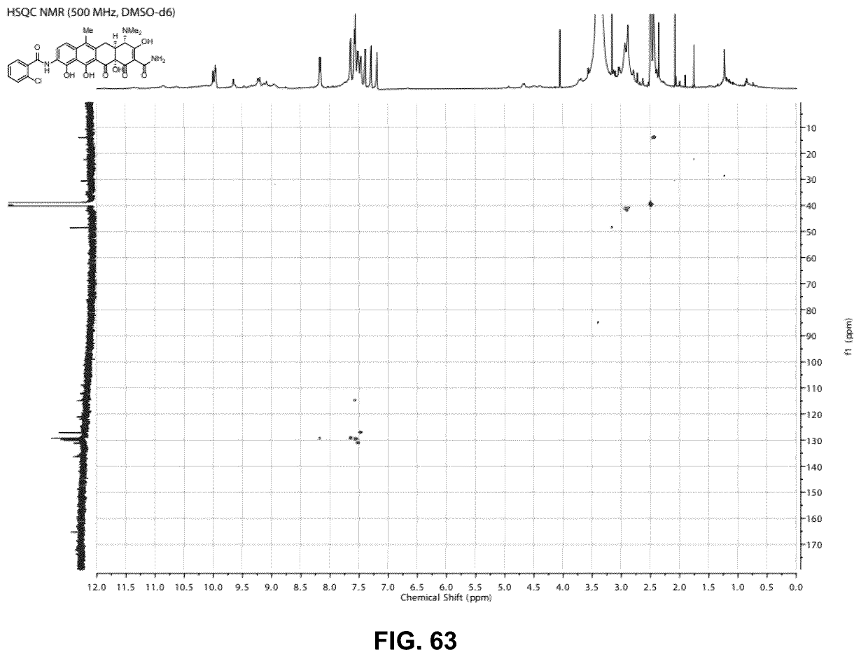

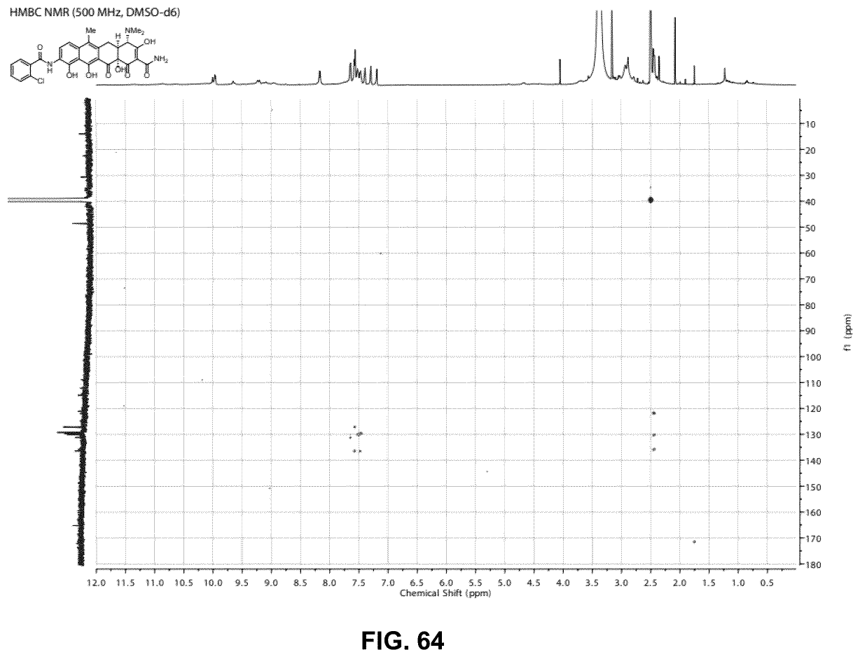

(4S,4aS,12aS)-9-(2-fluorobenzamido)-4-(dimethylamino)-3,10,11,12a-tetrahydroxy-6-methyl-1,12-dioxo-1,4,4a,5,12,12a-hexahydrotetracene-2-carboxamide (8).: Compound 8 was prepared according to general procedure A and was obtained in 8% yield as a dark orange solid. FIGS. 60, 61, 62, 63, and 64 show the NMR characterization of compound 8. ¹H NMR (500 MHz, DMSO-d6) δ 9.71 (d, J=7.6 Hz, 2H), 8.42 (d, J=8.9 Hz), 7.91 (t, J=7.2 Hz, 2H), 7.87 (td, J=7.9, 1.6 Hz, 1H), 7.67-7.62 (m, 3H), 7.58 (d, J=8.9 Hz), 7.41 (d, J=9.6 Hz, 2H), 7.38 (d, J=7.2 Hz, 2H), 7.32 (td, J=4.5, 1.2 Hz, 1H), 7.30 (d, J=7.9 Hz, 1H), 3.17 (s, 6H), 2.43 (s, 3H). ¹³C NMR (125 MHz, DMSO-d6) δ (ppm) 134.7, 134.6, 131.9, 130.8, 124.9, 124.5, 124.4, 117.0, 116.8, 116.3, 112.0, 76.4, 64.9, 48.6. Prep-HPLC tr 14m. LCMS epimeric purity 95% (see FIG. 124). MS (ESI+) [M+H]⁺ found 564.2. HRMS (TOF MS ESI+) calculated for C₂₉H₂₆FN₃O₈ [M+H]⁺ 564.1777; found 564.1775.

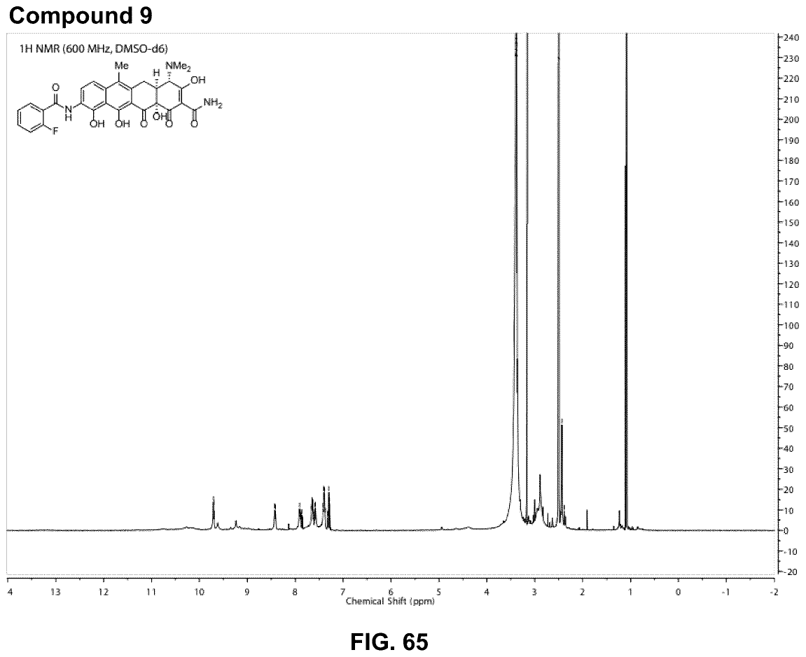

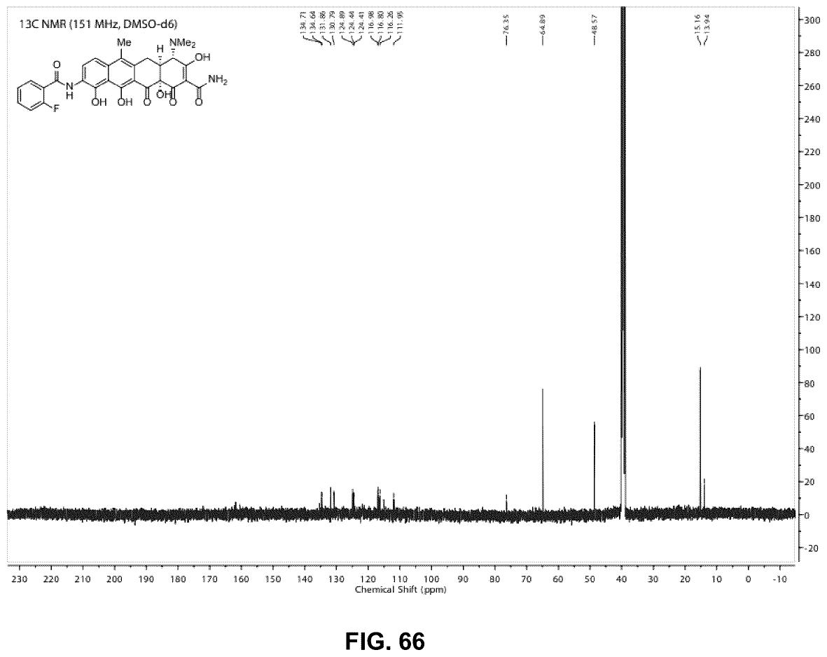

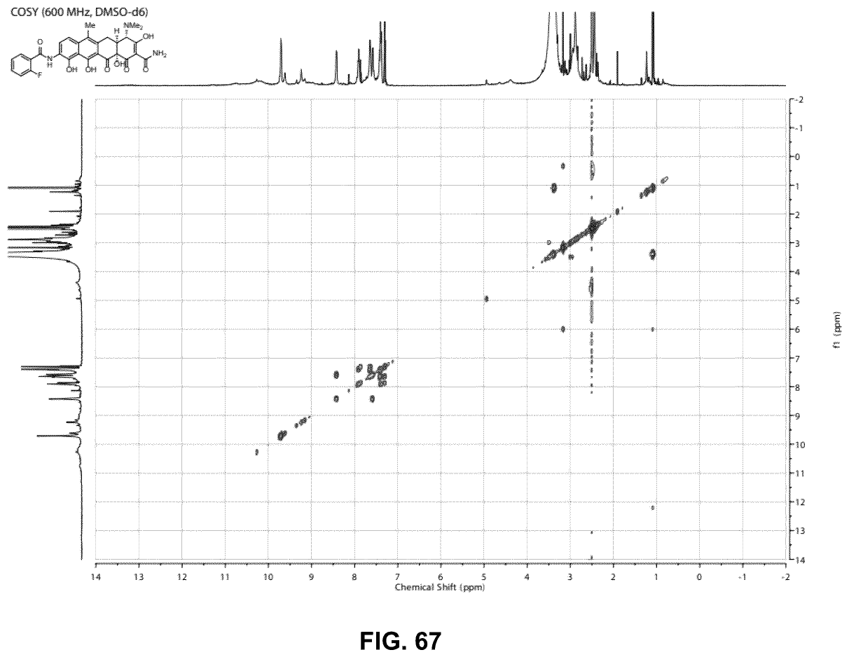

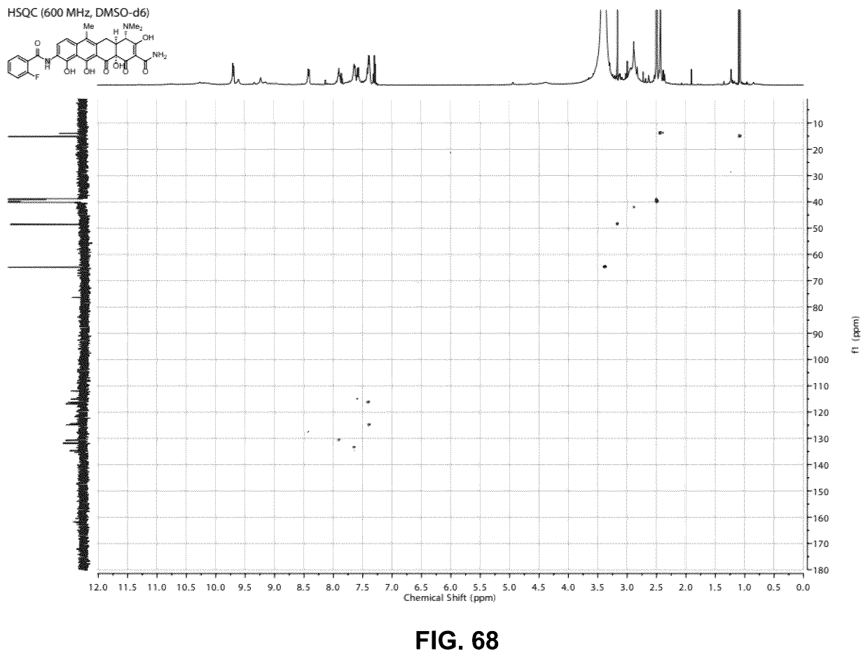

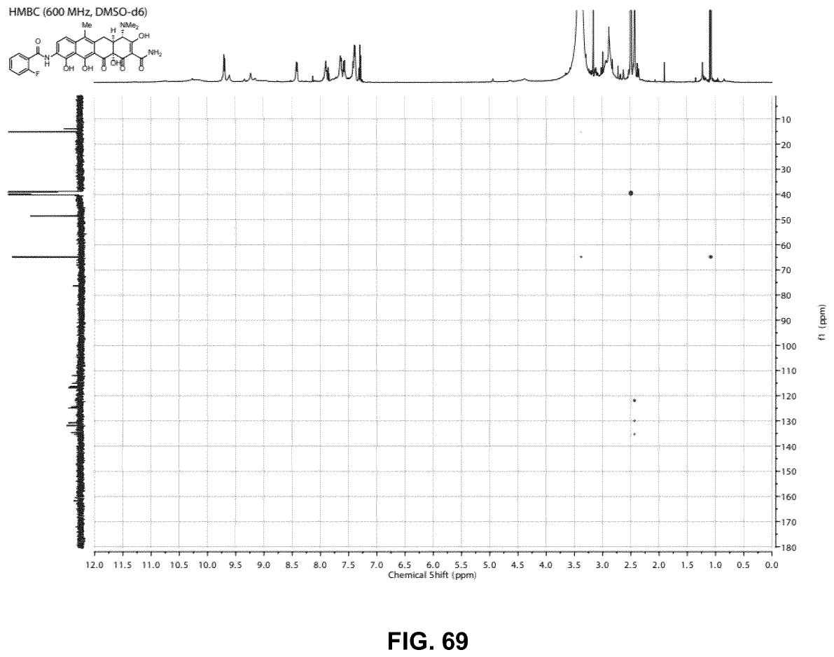

(4S,4aS,12aS)-9-(2-chlorobenzamido)-4-(dimethylamino)-3,10,11,12a-tetrahydroxy-6-methyl-1,12-dioxo-1,4,4a,5,12,12a-hexahydrotetracene-2-carboxamide (9).: Compound 9 was prepared according to general procedure A and was obtained in 10% yield as an orange solid. FIGS. 65, 66, 67, 68 and 69 show the NMR characterization of compound 9. ¹H NMR (500 MHz, DMSO-d6) δ (ppm) 8.17 (d, J=8.9, 1H) 7.67-7.63 (m, 1H), 7.60-7.57 (m, 1H), 7.57-7.55 (s, 1H), 7.55-7.50 (m, 1H), 7.50-7.45, (m, 1H), 3.55-3.50 (m, 1H), 3.16 (s, 3H), 2.99-2.95 (m, 1H), 2.89 (s, 1H), 2.47-2.42 (m, 1H), 2.36 (s, 6H). ¹³C NMR (125 MHz, DMSO-d6) δ (ppm) 165.3, 136.4, 131.2, 130.1, 129.7, 129.3, 127.2, 115.0, 48.6, 30.7, 14.0. Prep-HPLC tr 14m. LCMS epimeric purity 75% (see FIG. 125). MS (ESI+) [M+H]⁺ found 579.6. HRMS (TOF MS ESI+) calculated for C₂₉H₂₆ClN₃O₈ [M+H]⁺ 580.1481; found 580.1465.

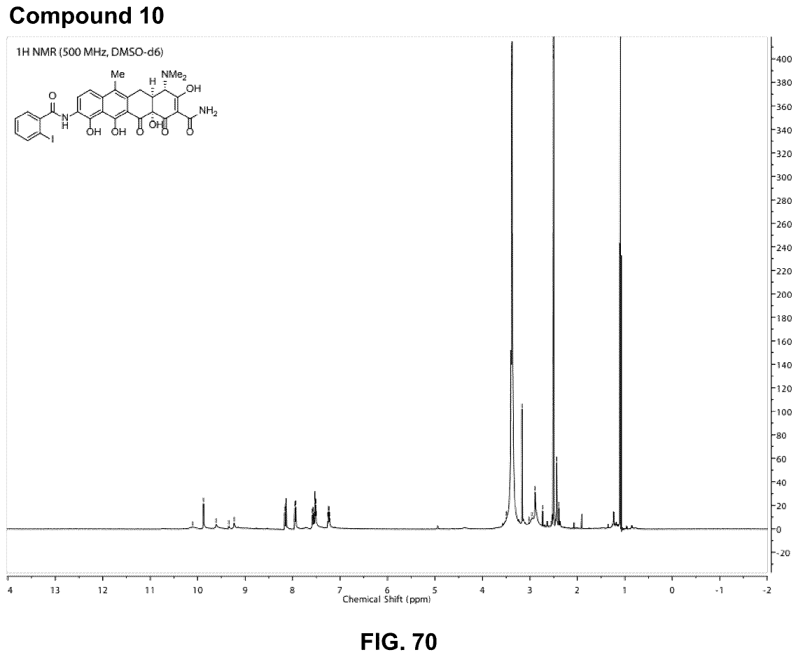

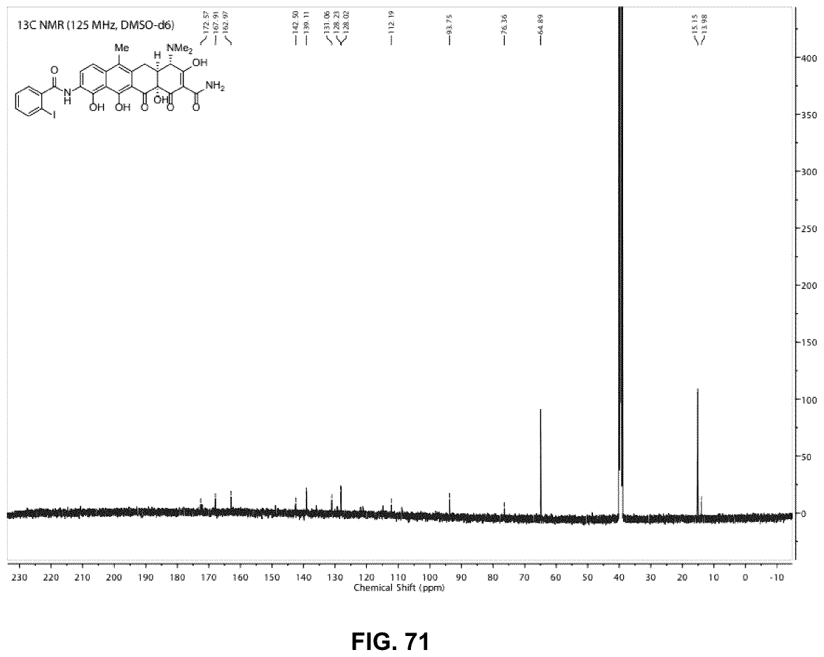

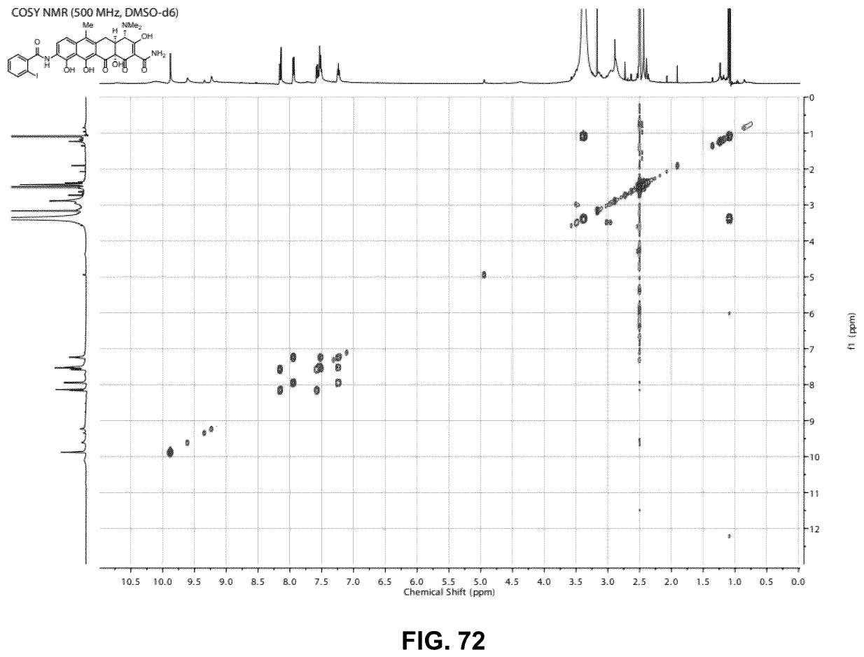

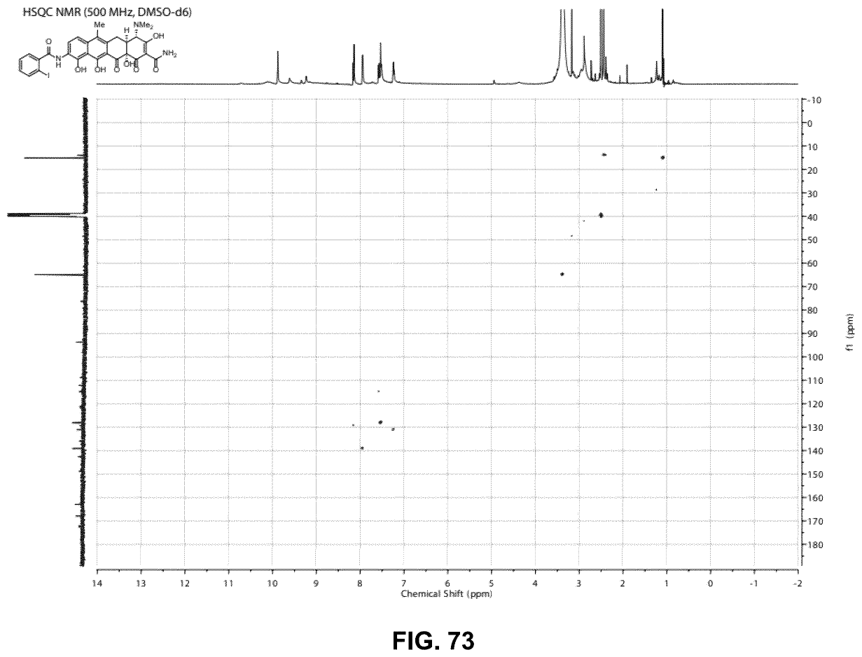

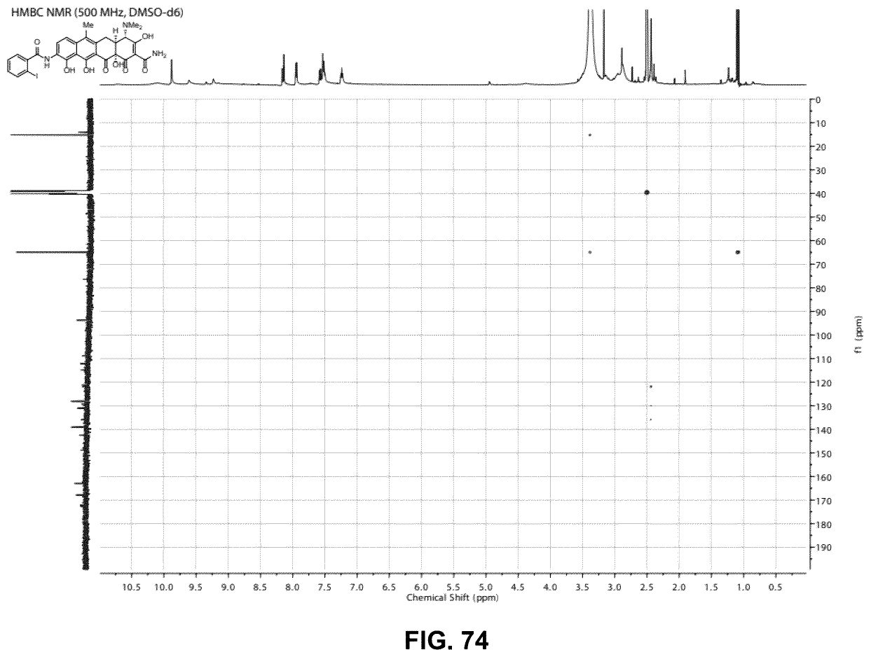

(4S,4aS,12aS)-9-(2-iodobenzamido)-4-(dimethylamino)-3,10,11,12a-tetrahydroxy-6-methyl-1,12-dioxo-1,4,4a,5,12,12a-hexahydrotetracene-2-carboxamide (10).: Compound 10 was prepared according to general procedure A and was obtained in 16% yield as a brown solid. FIGS. 70, 71, 72, 73, and 74 show the NMR characterization of compound 10. ¹H NMR (500 MHz, DMSO-d6) δ 8.16 (d, J=9 Hz, 1H), 7.94 (d, J=7.7, 1H), 7.59-7.50 (m, 3H), 7.24 (td, J=7.7, 2.0, 1H), 3.52-3.48 (m, 1H), 3.03-3.01 (m, 1H), 2.98-2.94 (m, 2H), 2.44 (s, 1H), 2.39 (s, 3H). ¹³C NMR (125 MHz, DMSO-d6) δ (ppm) 172.6, 167.9, 163.0, 142.5, 139.1, 131.1, 128.2, 128.0, 112.2, 93.8, 76.4, 64.9, 15.2, 14.0. LCMS epimeric purity 97% (see FIG. 126). MS (ESI+) M+H]⁺ found 671.6. HRMS (TOF MS ESI+) calculated for C₂₉H₂₆IN₃O₈ [M+H]⁺ 672.0837; found 672.0819.

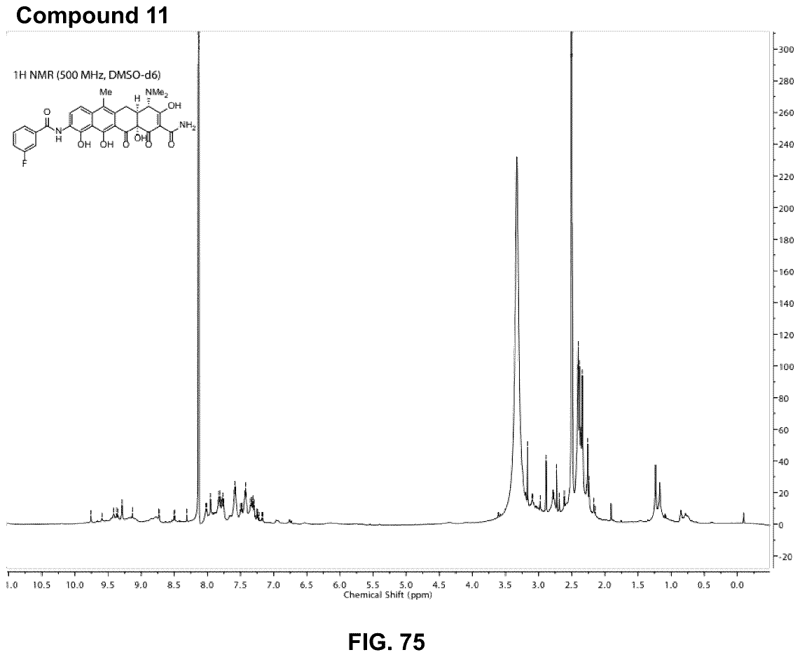

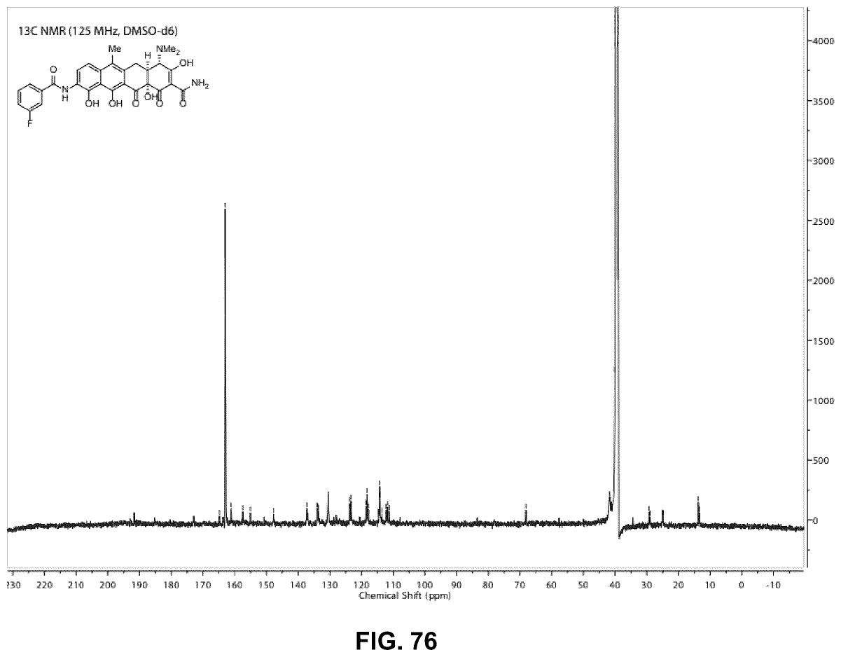

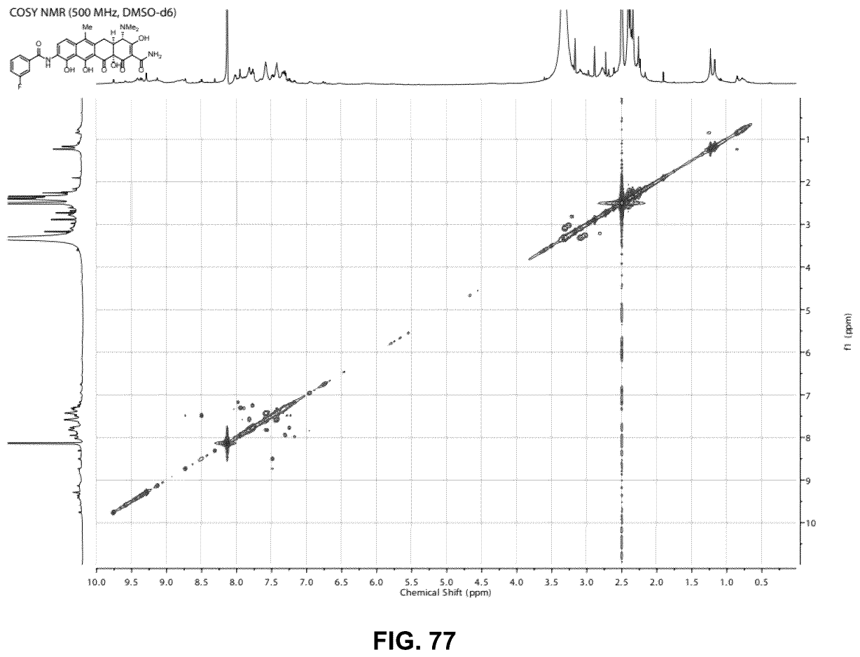

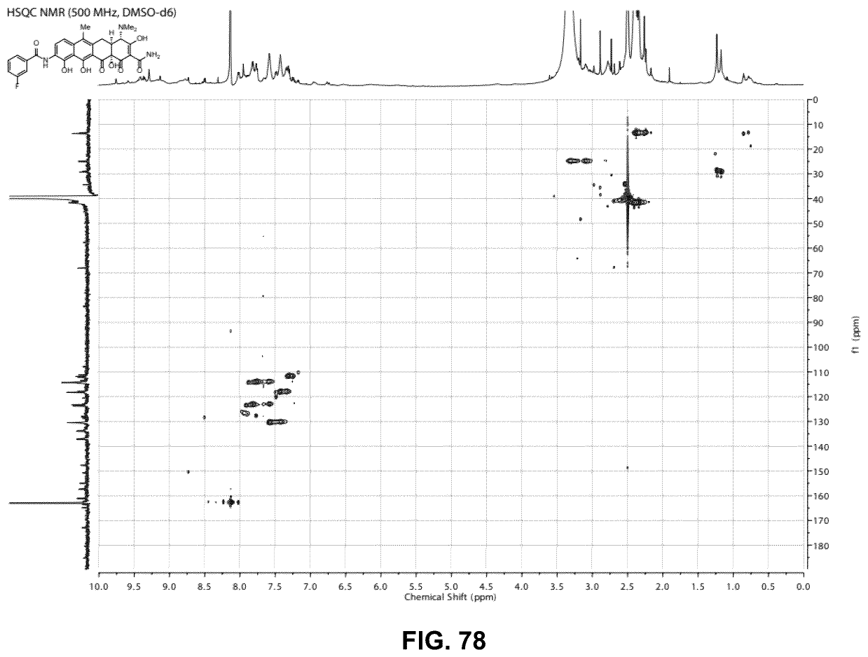

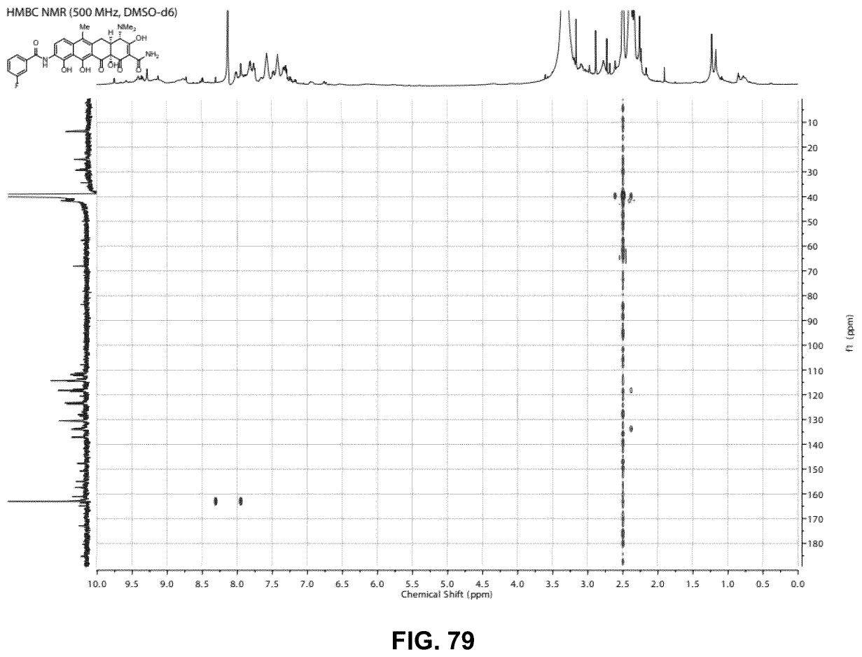

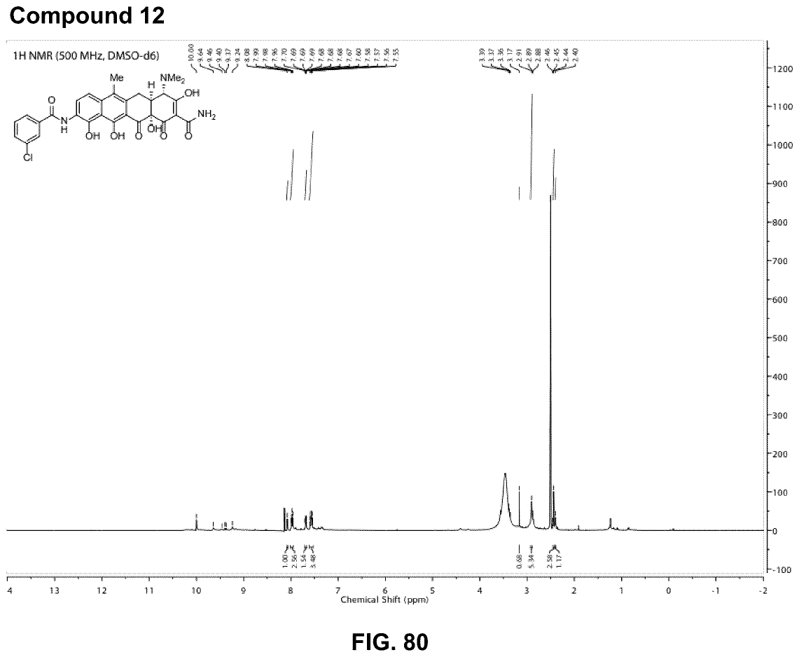

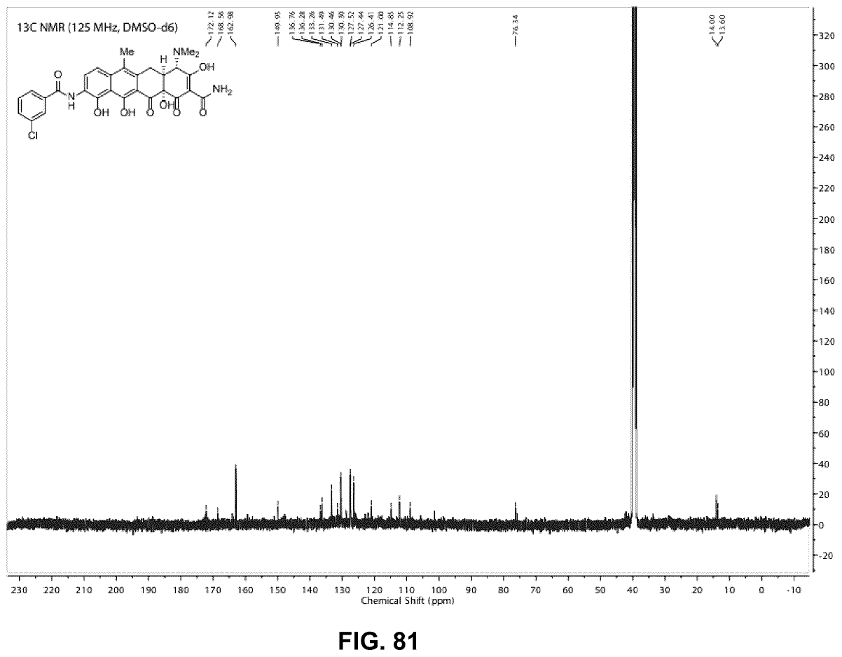

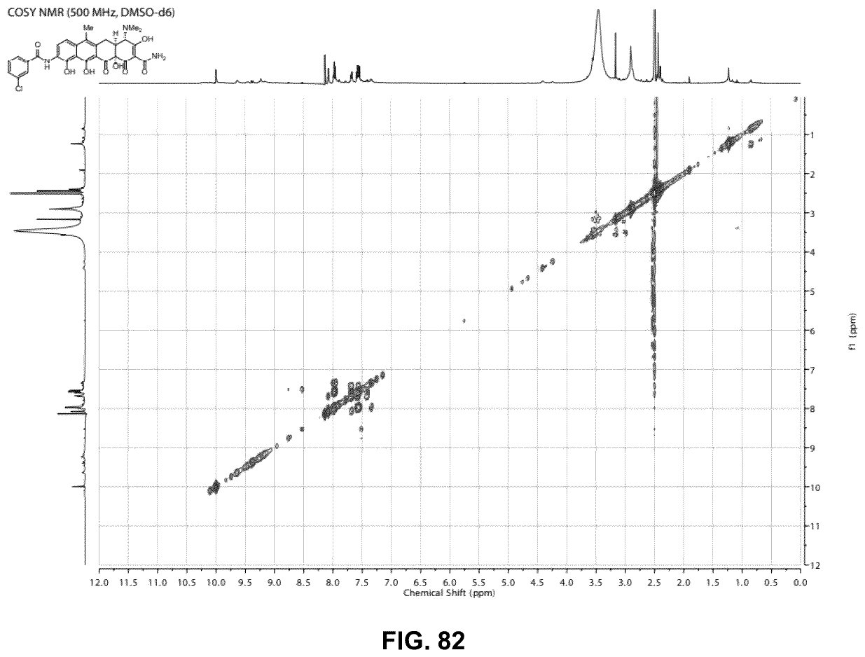

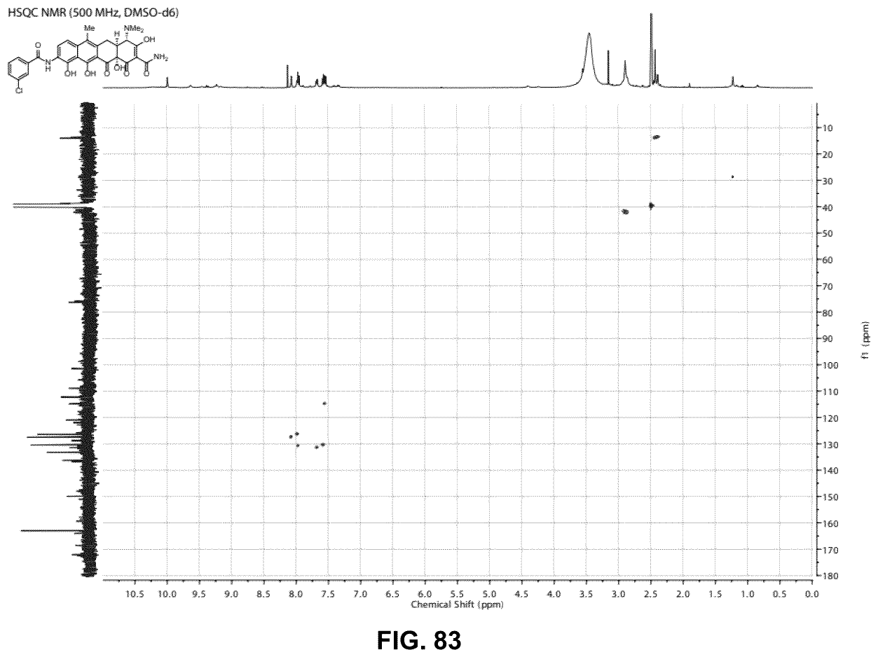

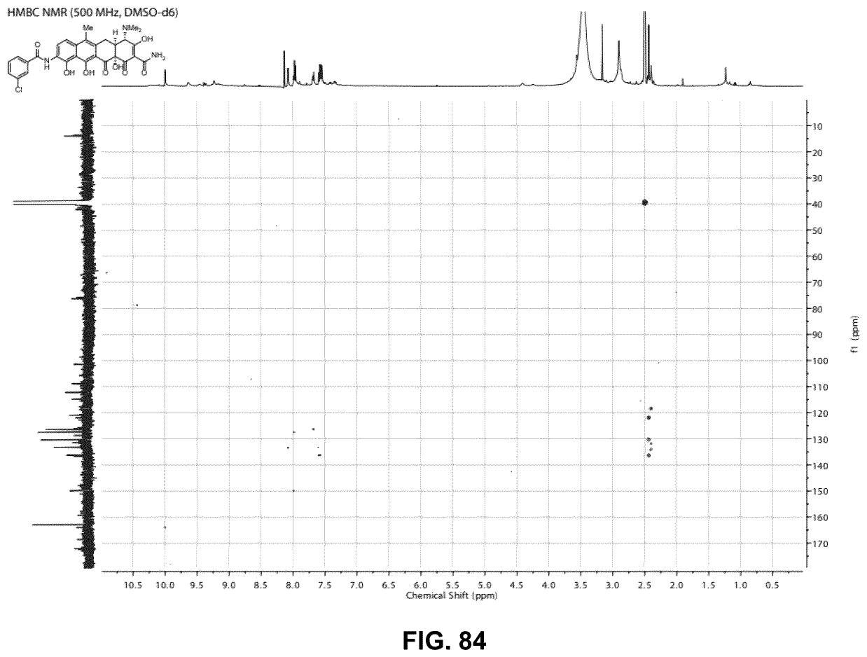

(4S,4aS,12aS)-9-(3-fluorobenzamido)-4-(dimethylamino)-3,10,11,12a-tetrahydroxy-6-methyl-1,12-dioxo-1,4,4a,5,12,12a-hexahydrotetracene-2-carboxamide (11).: Compound 11 was prepared according to general procedure A and was obtained in 6% yield as an orange solid. FIGS. 75, 76, 77, 78, and 79 show the NMR characterization of compound 11. ¹H NMR (500 MHz, DMSO-d6) δ 8.03-7.75 (m), 7.60-7.57 (m), 7.44-7.41 (m), 7.35-7.30 (m), 3.17 (s), 2.89 (s), 2.73 (s), 2.43-2.32 (m). Prep-HPLC tr 14m. LCMS epimeric purity 98% (see FIG. 127). MS (ESI+) [M+H]⁺ found 564.2. HRMS (TOF MS ESI+) calculated for C₂₉H₂₆FN₃O₈ [M+H]⁺ 564.1777; found 564.1761. [0354](4S,4aS,12aS)-9-(3-chlorobenzamido)-4-(dimethylamino)-3,10,11,12a-tetrahydroxy-6-methyl-1,12-dioxo-1,4,4a,5,12,12a-hexahydrotetracene-2-carboxamide (12).: Compound 12 was prepared according to general procedure A and was obtained in 5% yield as an orange solid. FIGS. 80, 81, 82, 83, and 84 show the NMR characterization of compound 12. ¹H NMR (500 MHz, DMSO-d6) δ 10.00 (s, 1H), 9.64 (s, 1H), 9.38 (d, J=13.4 Hz, 1H), 9.24 (s, 2H), 8.09-8.07 (m, 1H), 7.99-7.96 (m, 3H), 7.69 (ddd, J=8.0, 2.1, 0.9 Hz, 2H), 7.61-7.53 (m, 3H), 3.39-3.36 (m), 3.17 (s, 1H), 2.91 (s, 6H), 2.44 (s, 3H), 2.41-2.39 (m, 1H). ¹³C NMR (125 MHz, DMSOd6) 172.1, 168.6, 163.0, 150.0, 136.8, 136.3, 133.3, 131.5, 130.5, 130.3, 127.5, 127.4, 126.4, 121.0, 114.9, 112.3, 108.9, 76.34, 14.00, 13.60. Prep-HPLC tr 14m. LCMS epimeric purity 98% (see FIG. 128). MS (ESI+) [M+H]⁺ found 580.2. HRMS (TOF MS ESI+) calculated for C₂₉H₂₆ClN₃O₈ [M+H]⁺580.1481; found 580.1467.

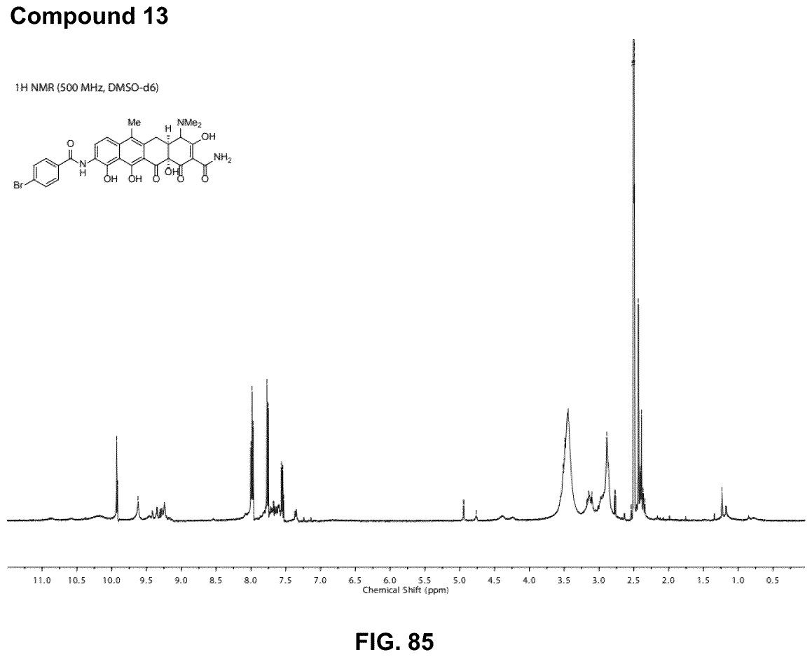

(4S,4aS,12aS)-9-(4-bromobenzamido)-4-(dimethylamino)-3,10,11,12a-tetrahydroxy-6-methyl-1,12-dioxo-1,4,4a,5,12,12a-hexahydrotetracene-2-carboxamide (13).: Compound 13 was prepared according to general procedure A and was obtained in 5% yield as a dark brown solid. FIG. 85 shows the NMR characterization of compound 13. ¹H NMR (500 MHz, DMSO-d6) δ 9.92 (d, J=6.2 Hz, 1H), 7.98 (t, J=8.0 Hz, 4H), 7.76 (d, J=8.5 Hz, 2H), 7.54 (dd, J=9.1, 5.8 Hz, 2H), 4.94 (d, J=3.0 Hz, 1H), 2.87 (s, 1H), 2.77 (d, J=4.6 Hz, 1H), 2.43 (s, 6H), 2.39 (s, 3H). Prep-HPLC tr 14m. LCMS epimeric purity 65% (see FIG. 129). MS (ESI+) [M+H]⁺ found 623.5. HRMS (TOF MS ESI+) calculated for C₂₉H₂₆BrN₃O₈ [M+H]⁺ 624.0976; found 624.0962.

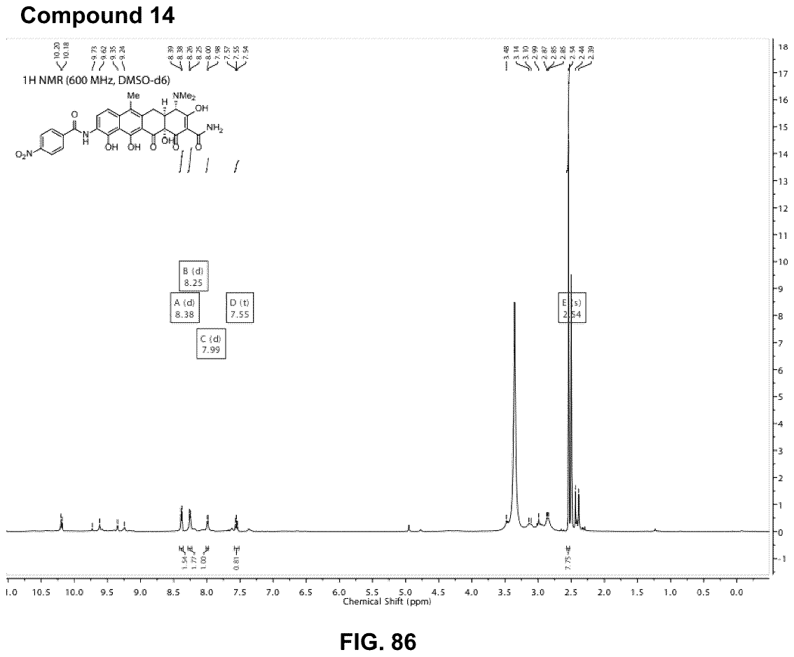

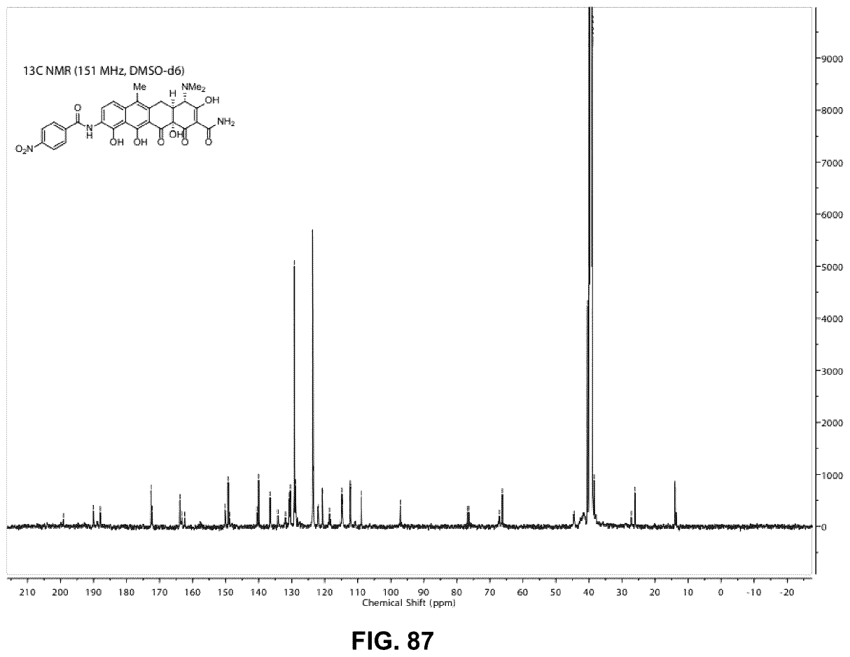

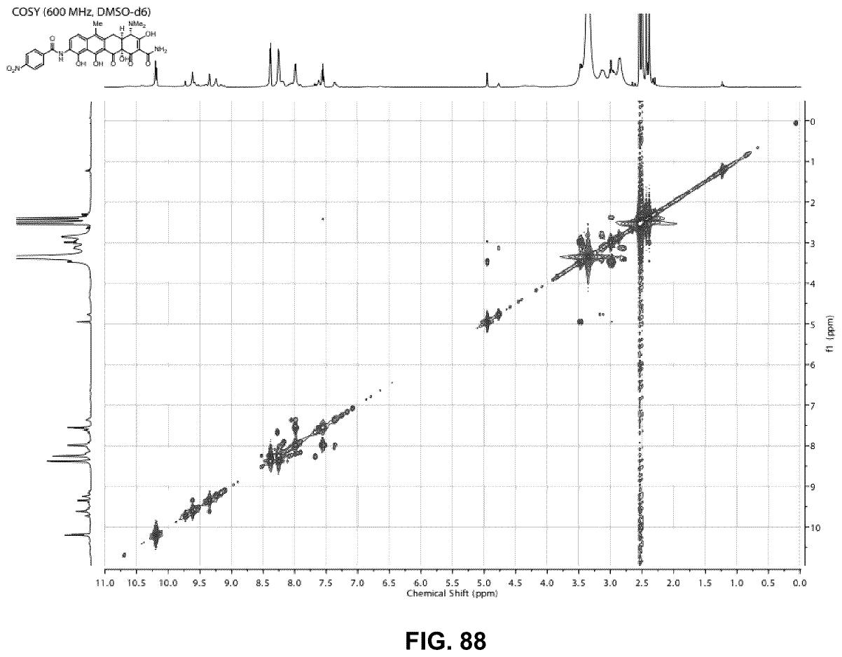

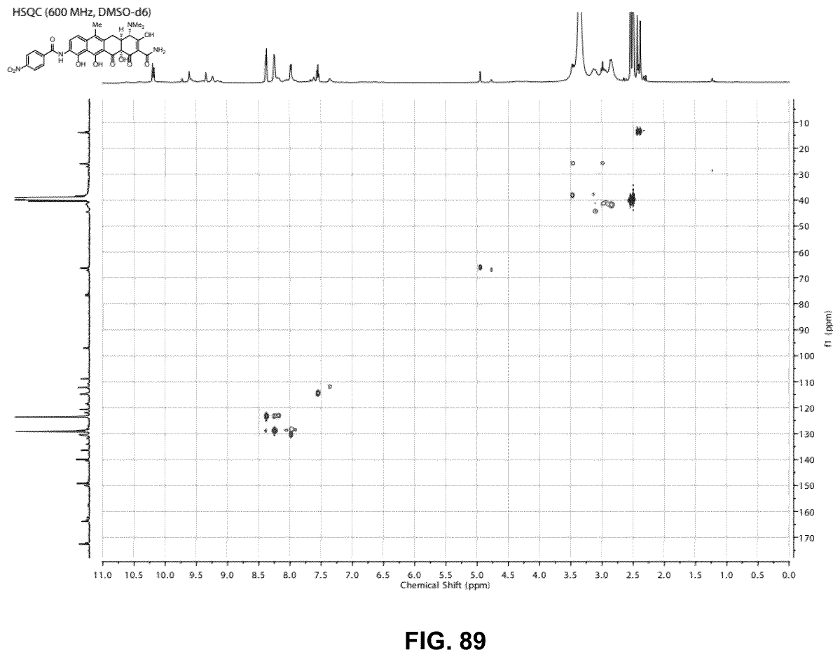

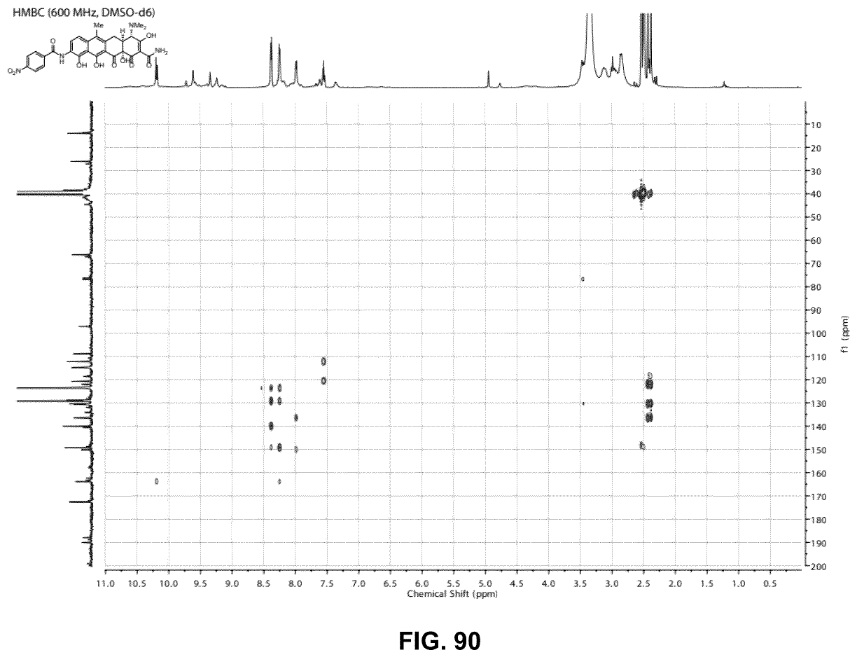

(4S,4aS,12aS)-9-(4-nitrobenzamido)-4-(dimethylamino)-3,10,11,12a-tetrahydroxy-6-methyl-1,12-dioxo-1,4,4a,5,12,12a-hexahydrotetracene-2-carboxamide (14).: Compound 14 was prepared according to general procedure A and was obtained in 15% yield as a rusty orange solid. FIGS. 86, 87, 88, 89, and 90 show the NMR characterization of compound 14. ¹H NMR (600 MHz, DMSO-d6) δ 8.38 (d, J=7.5, 2H), 8.25 (d, J=7.5, 2H), 7.99 (d, J=9, 1H), 7.57-7.54 (m, 1H), 3.50-3.44 (m, 1H), 3.17-3.07 (m, 1H), 3.00-2.97 (m, 1H), 2.86-2.81 (m, 1H), 2.54 (s, 6H)¹³C NMR (151 MHz, DMSO-d6) δ 199.19, 190.07, 187.99, 172.58, 172.21, 163.88, 163.22, 162.35, 150.16, 149.22, 148.87, 140.51, 140.01, 136.49, 134.15, 131.88, 130.73, 130.59, 130.40, 129.22, 128.82, 123.62, 123.44, 122.14, 121.93, 120.73, 120.68, 118.58, 114.84, 114.65, 112.26, 112.21, 108.93, 97.06, 76.86, 76.39, 67.10, 66.25, 44.49, 40.43, 38.43, 27.16, 26.08, 14.04, 14.00. Prep-HPLC tr 14m. LCMS epimeric purity 90% (see FIG. 130). MS (ESI+) [M+H]⁺ found 590.6. HRMS (TOF MS ESI+) calculated for C₂₉H₂₆N₄O₁₀ [M+H]⁺ 591.1722; found 591.1708.

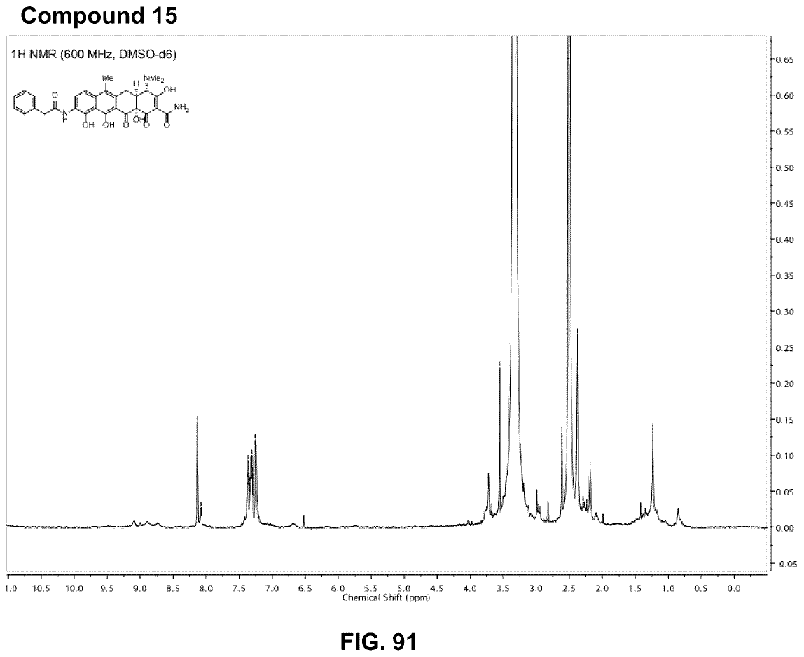

(4S,4aS,12aS)-4-(dimethylamino)-3,10,11,12a-tetrahydroxy-6-methyl-1,12-dioxo-9-(2-phenylacetamido)-1,4,4a,5,12,12a-hexahydrotetracene-2-carboxamide (15).: Compound 15 was prepared according to general procedure A and was obtained in 30% yield as a brown oil. FIG. 91 Shows the NMR characterization of compound 15. ¹H NMR (600 MHz, DMSO-d6) δ 8.08 (d, 1H), 7.38-7.23 (m, 7H), 3.56 (s, 2H), 2.99-2.94 (m, 2H) 2.61 (s, 2H), 2.37 (s, 6H), 2.32-2.24 (m, 2H), 2.18 (s, 3H). Prep-HPLC tr 16m. LCMS epimeric purity 95% (see FIG. 131). MS (ESI+) calculated for C₃₀H₂₉N₂O₈ [M+H]⁺ 559.2; found 559.9.

General procedure (B) for synthesis of C9-amino-aTC analogs (16-20).: To a clean, dry round-bottom flask, equipped with stir bar, was added C9-NH2-aTC-NH2 HCl salt (compound 3) (0.1069 mmol) as a solution in 5.0 mL of anhydrous MeOH (DriSolv) under an Ar atmosphere. A solution of Et3N (14.91 mL, 0.1069 mmol) in 1.5 mL anhydrous MeOH was prepared in a separate vial and transferred to the brown stirring solution of 3. The benzaldehyde substrate (0.1925 mmol) was dissolved in 1 mL of anhydrous MeOH in a separate vial and transferred to the reaction in one portion. Na(OAc)3BH (0.0793 g, 0.3742 mmol) was added in one portion to the reaction mixture as a solid. The reaction mixture was stirred for 3 h at room temp prior to the addition of a second portion of Na(OAc)3BH (0.0226 g, 0.1069 mmol). The reaction mixture was stirred for an additional 3 h prior to quenching with a solution of saturated NaHCO3(aq). The quenched reaction mixture was concentrated under reduced pressure via rotary evaporation. The dark red solid was dissolved in MeOH and filtered through a 0.45 μM PTFE syringe filter prior to purification by RP-C18 prep-HPLC to provide the desired products 16-20 as the corresponding formic acid salts.

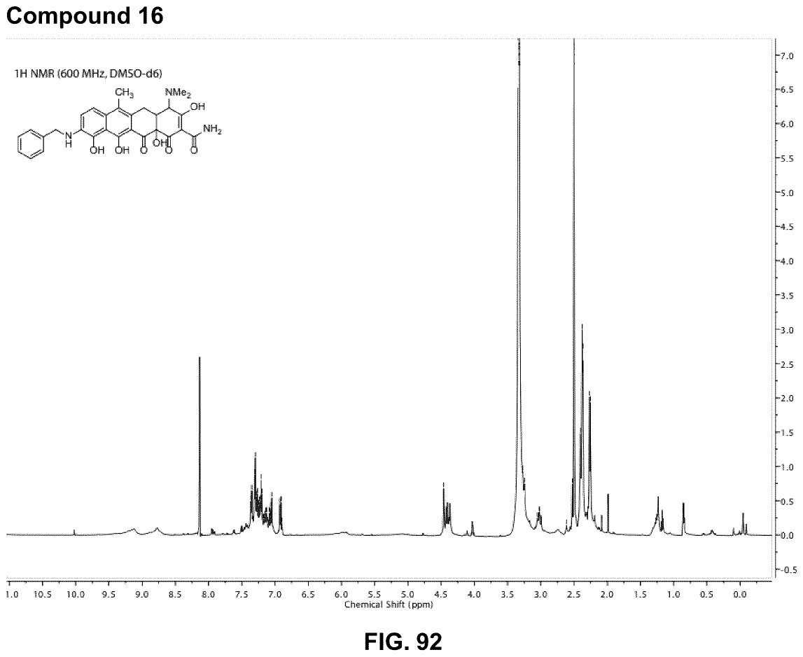

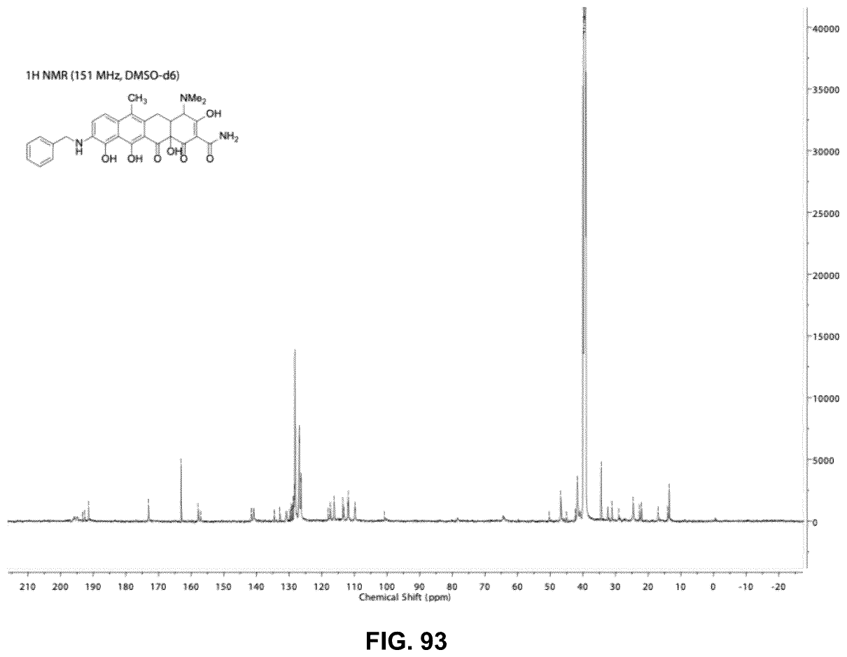

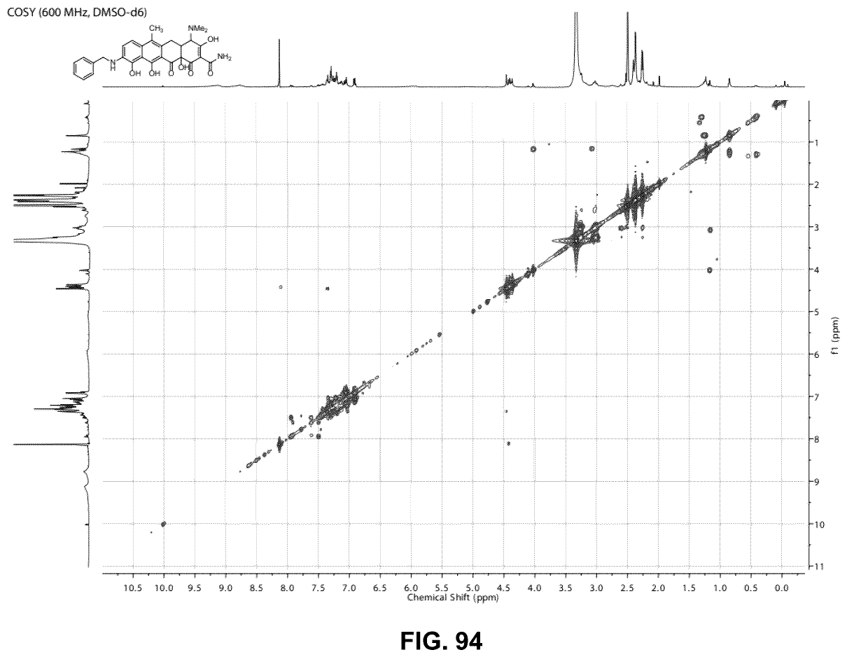

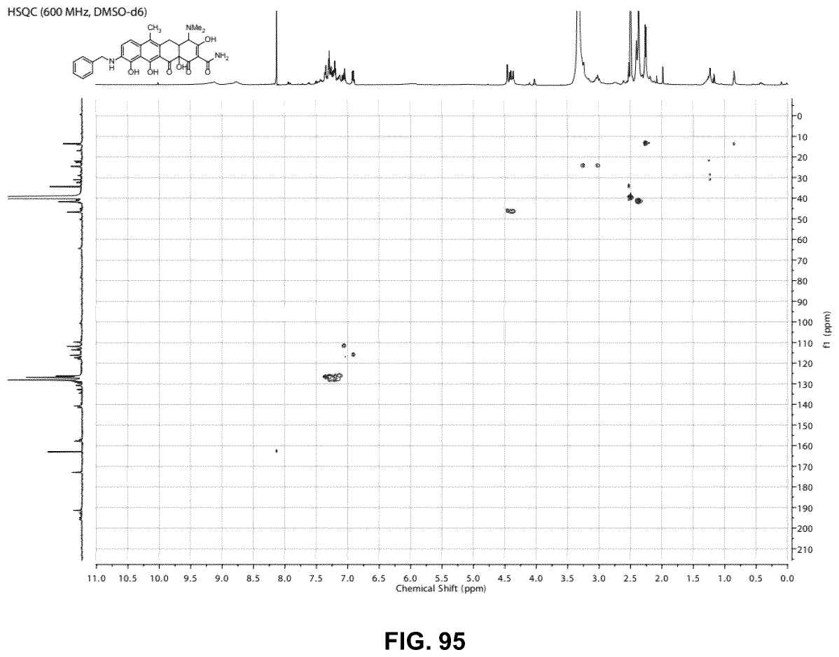

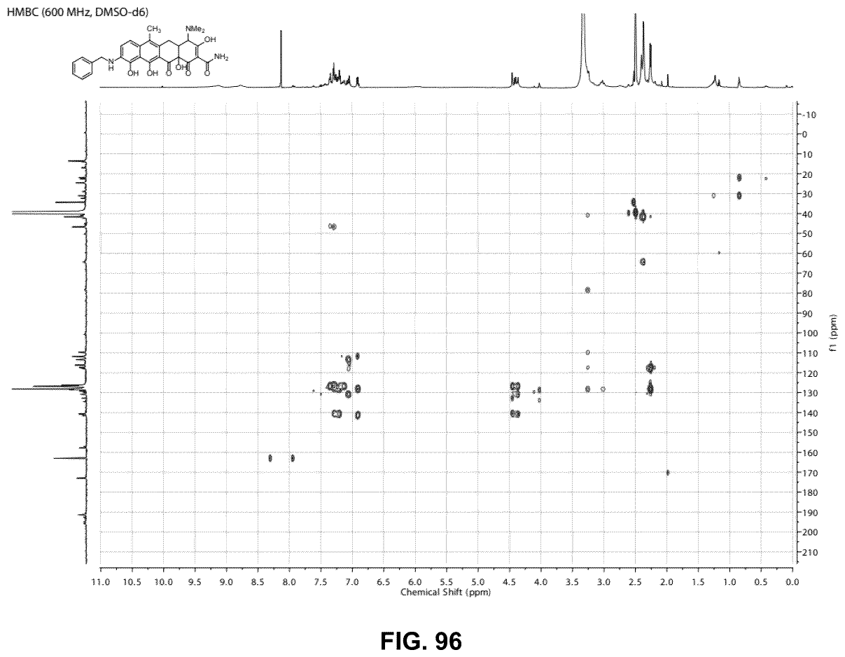

(4S,4aS,12aS)-9-benzylamino-4-(dimethylamino)-3,10,11,12a-tetrahydroxy-6-methyl-1,12-dioxo-1,4,4a,5,12,12a-hexahydrotetracene-2-carboxamide (16).: Compound 16 was prepared according to general procedure B and was obtained in 22% yield as a red solid. FIGS. 92, 93, 94, 95, and 96 show the NMR characterization of compound 16. ¹H NMR (600 MHz, DMSO-d6) δ (ppm) 7.36 (d, J=7.5 Hz, 1H), 7.30 (d, J=7.4 Hz, 2H), 7.28 (d, 1H), 7.20 (d, J=7.4 Hz, 2H), 6.93-6.89 (m, 1H), 3.28-3.23 (m, 1H), 3.05-2.99 (m, 1H), 2.63-2.59 (m, 1H), 2.53 (s, 3H), 2.41 (s, 6H), 2.26 (d, 1H) 13C NMR (151 MHz, DMSO-d6) δ 193.25, 192.62, 191.41, 173.03, 163.04, 157.84, 157.05, 141.57, 134.59, 132.87, 129.84, 129.49, 129.25, 129.17, 128.83, 128.65, 128.57, 128.47, 128.21, 126.89, 126.84, 126.36, 126.30, 117.40, 116.23, 113.53, 113.22, 112.01, 111.85, 109.73, 46.76, 42.36, 41.73, 34.39, 32.41, 31.10, 29.02, 24.59, 22.64, 22.03, 17.02, 13.99, 13.59. PrepHPLC tr 16m. LCMS epimeric purity 92% (see FIG. 132). MS (ESI+) [M+H]⁺ found 531.8. HRMS (TOF MS ESI+) calculated for C₂₉H₂₉N₃O₇ [M+H]⁺ 532.2078; found 532.2079.

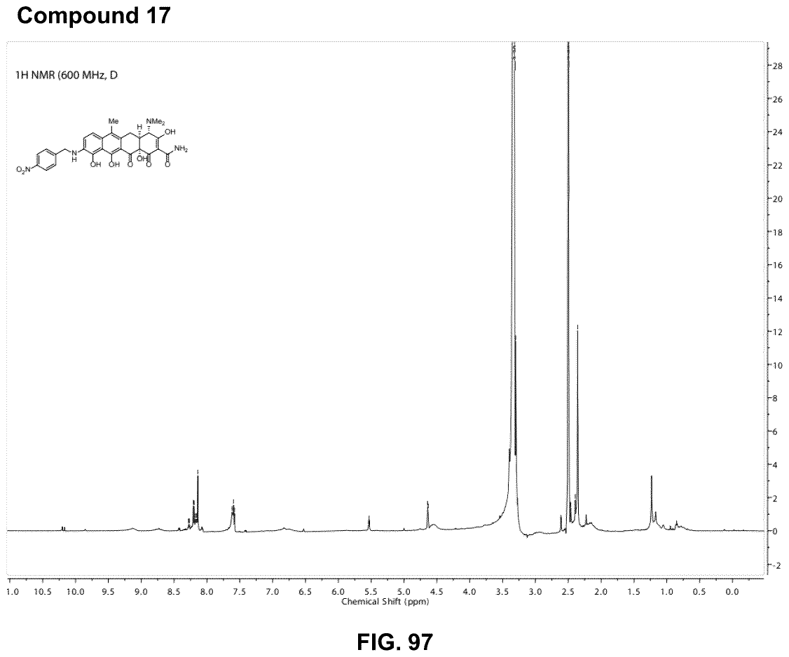

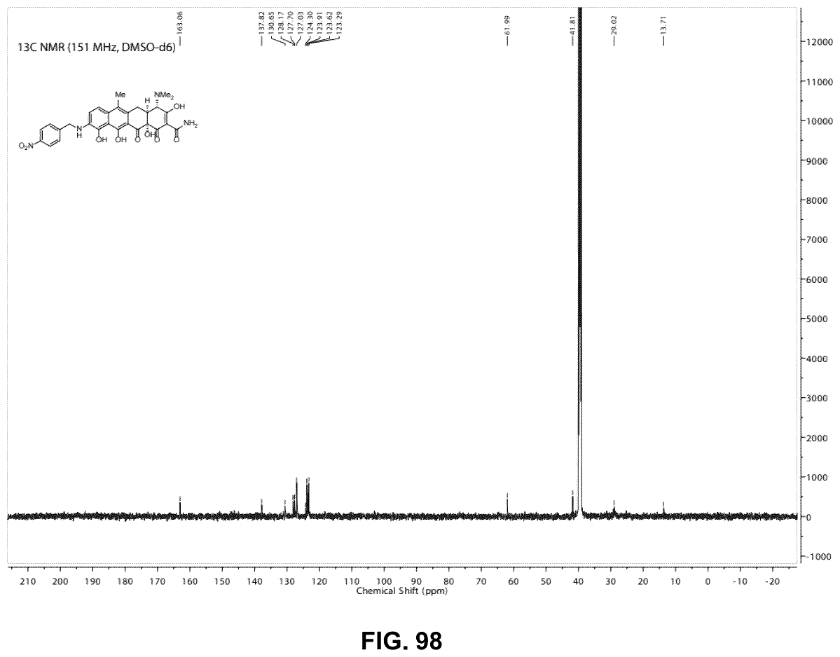

(4S,4aS,12aS)-9-(4-nitrobenzylamino)-4-(dimethylamino)-3,10,11,12a-tetrahydroxy-6-methyl-1,12-dioxo-1,4,4a,5,12,12a-hexahydrotetracene-2-carboxamide (17).: Compound 17 was prepared according to general procedure B and was obtained in 5% yield as a red solid. FIGS. 97 and 98 show the NMR characterization of compound 17. ¹H NMR (600 MHz, DMSO-d6) δ 8.27 (d, J=8.7 Hz, 1H), 8.20 (d, J=8.7 Hz, 2H), 8.14 (s, 1H), 7.59 (d, J=8.6 Hz, 2H), 5.53 (t, J=5.5 Hz, 1H), 4.64 (d, J=5.1 Hz, 2H), 3.30 (s, 20H), 2.62-2.60 (m, 1H), 2.48-2.45 (m, 3H), 2.40 (s, 3H), 2.36 (s, 13H). ¹³C NMR (151 MHz, DMSO-d6) δ 163.06, 137.82, 130.65, 128.17, 127.70, 127.03, 124.30, 123.91, 123.62, 123.29, 61.99, 41.81, 29.02, 13.71. Prep-HPLC tr 16m. LCMS epimeric purity 90% (see FIG. 133). MS (ESI+) calculated for C₂₉H₂₈N₄O₉ [M+H]⁺ 577.2; found 576.7.

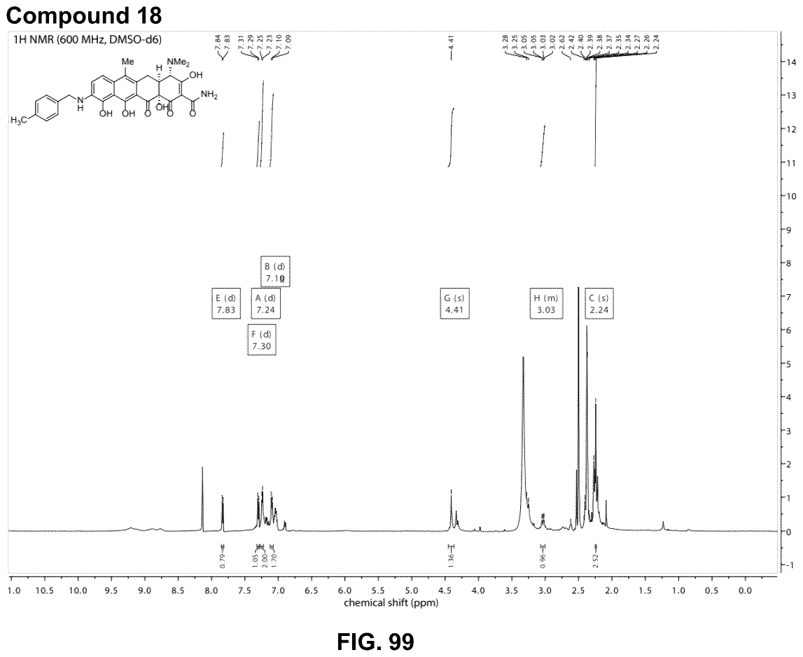

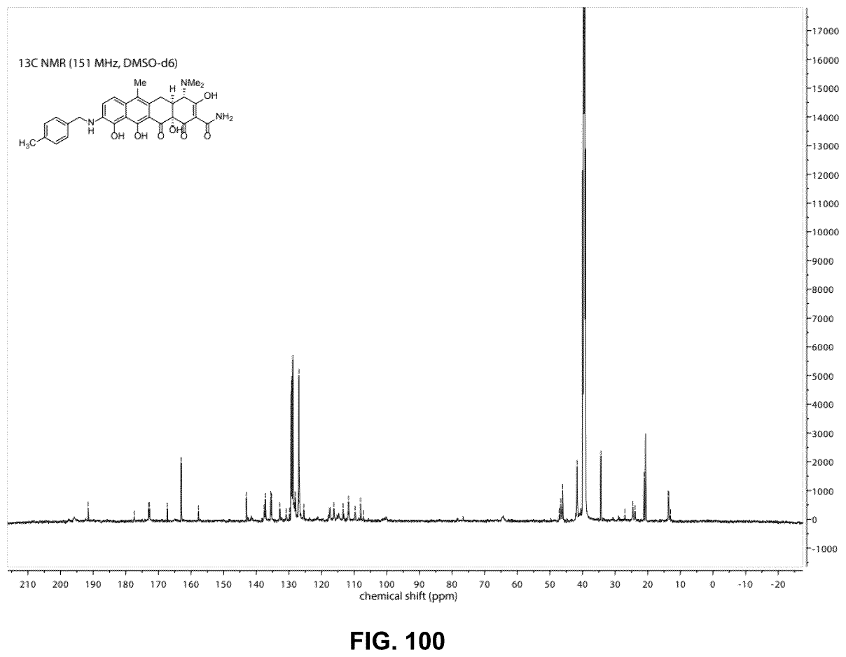

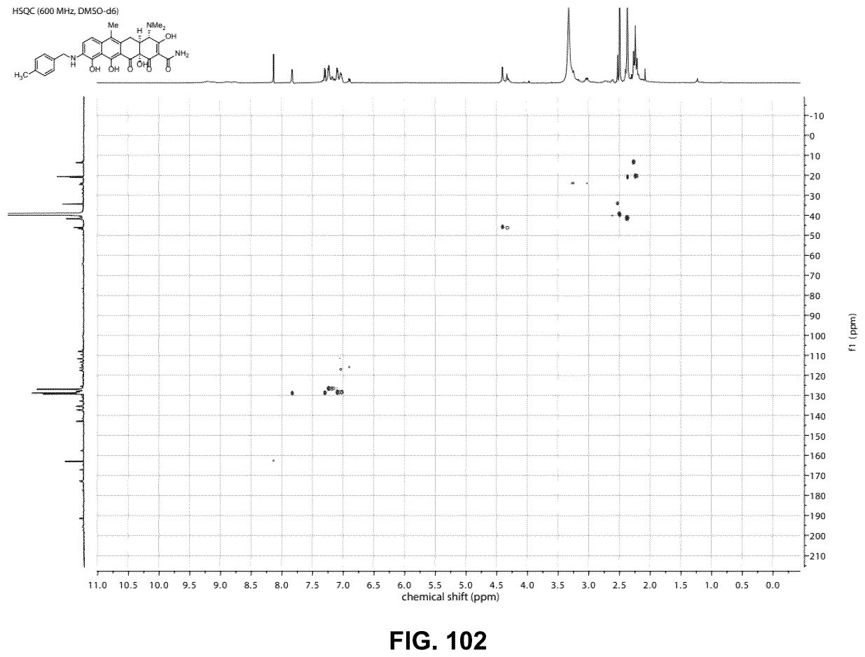

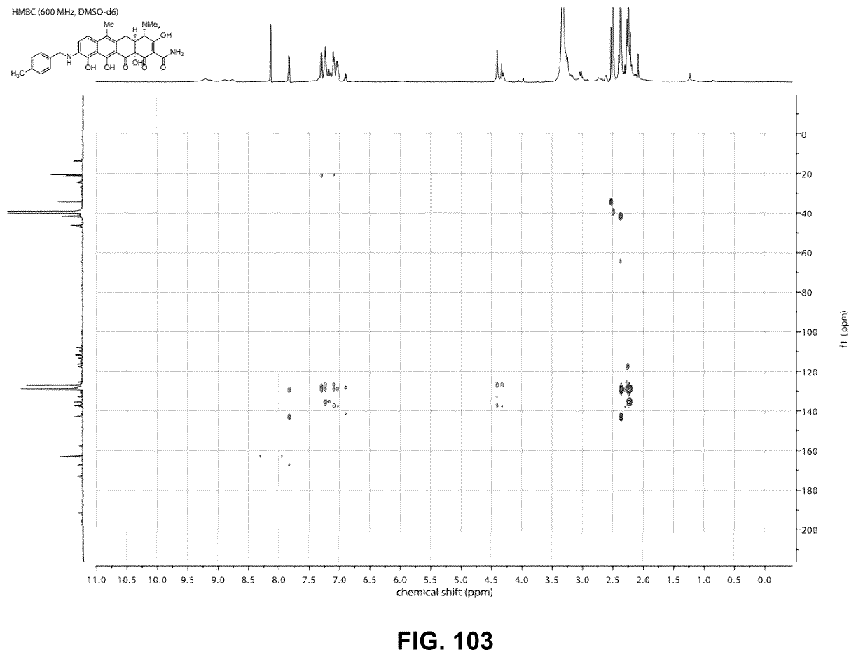

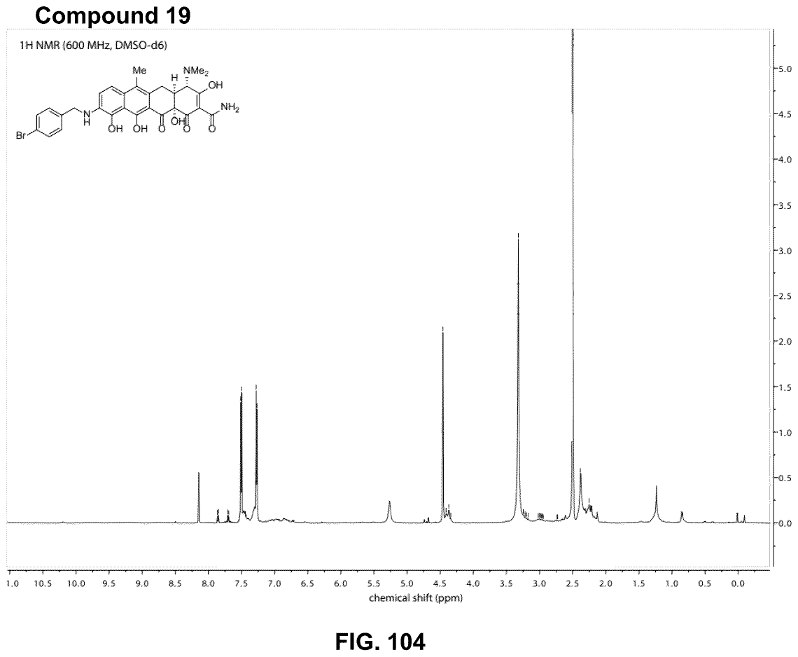

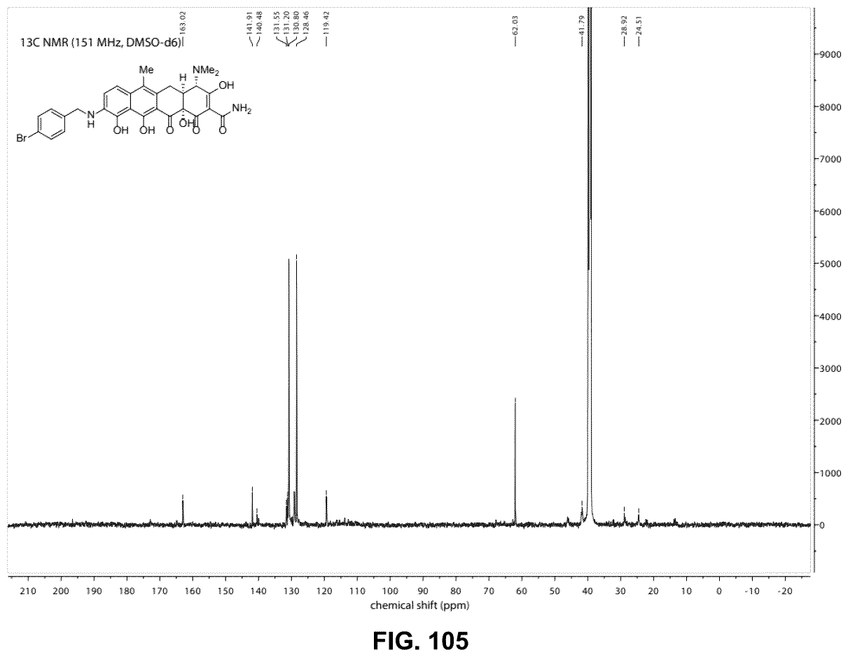

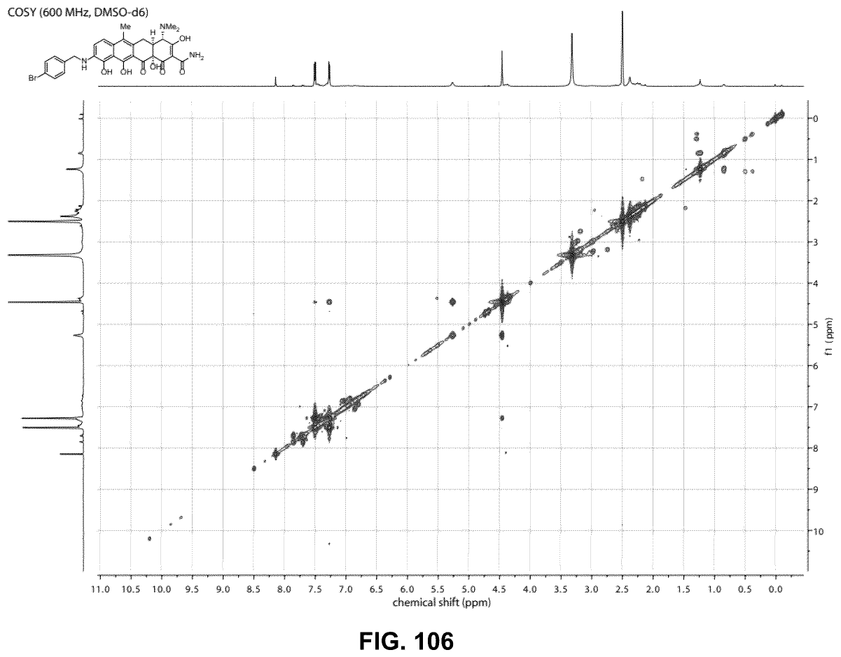

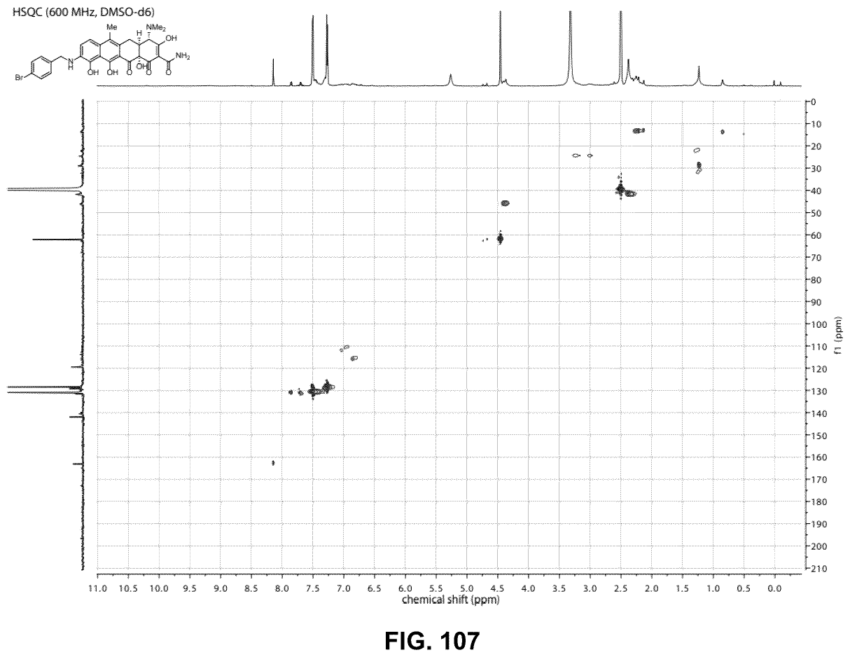

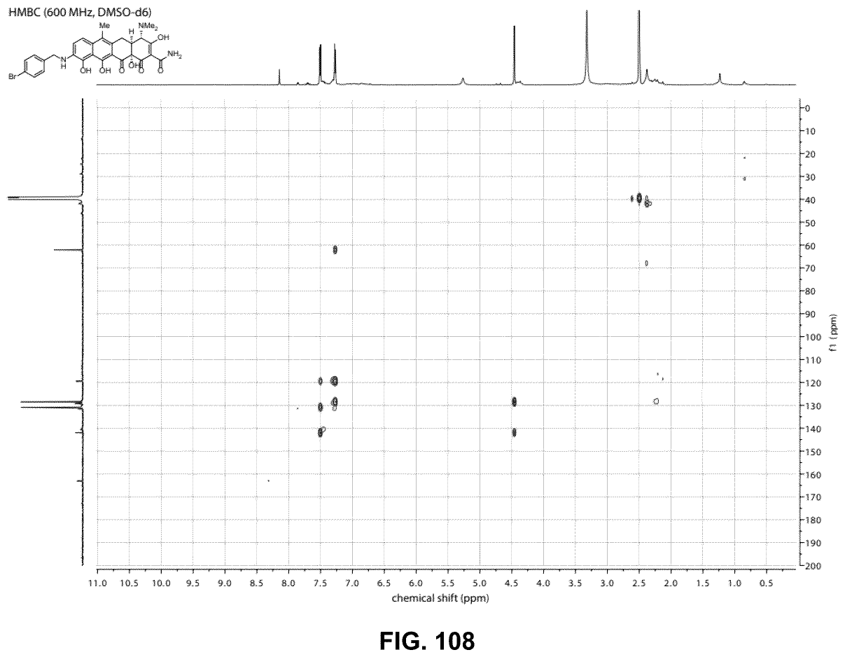

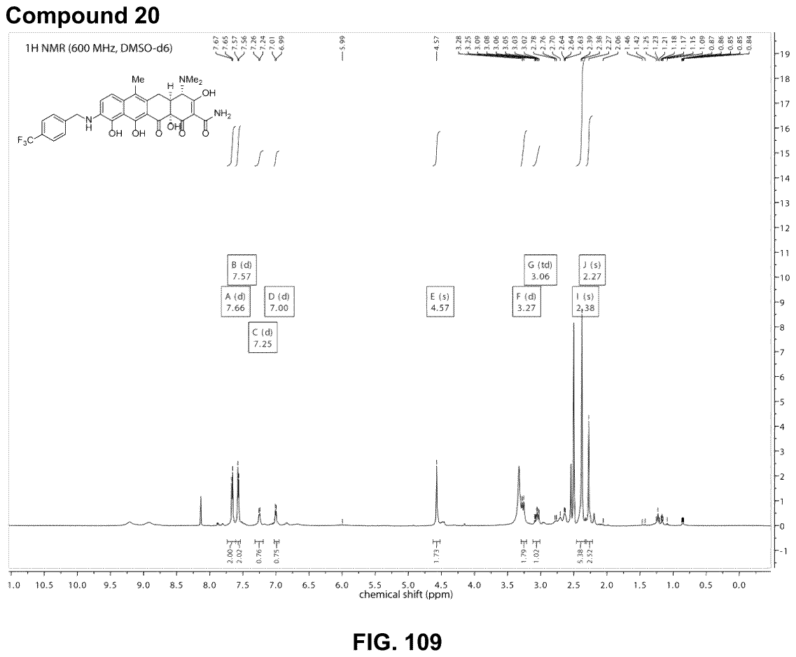

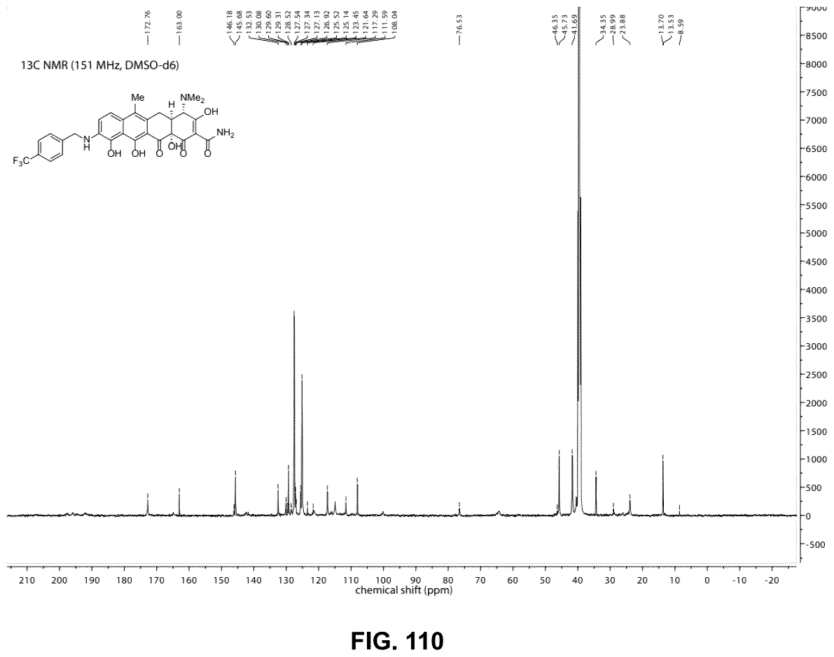

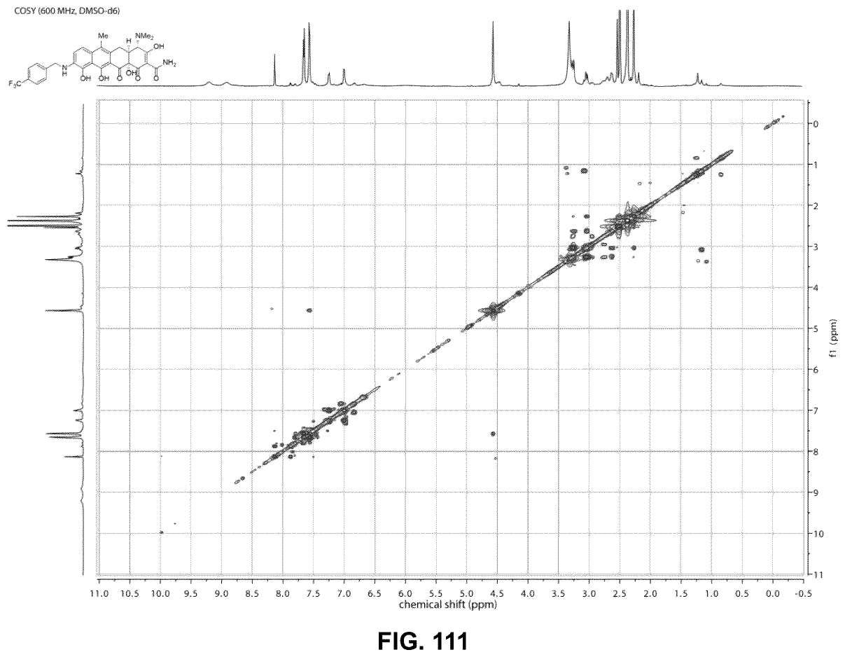

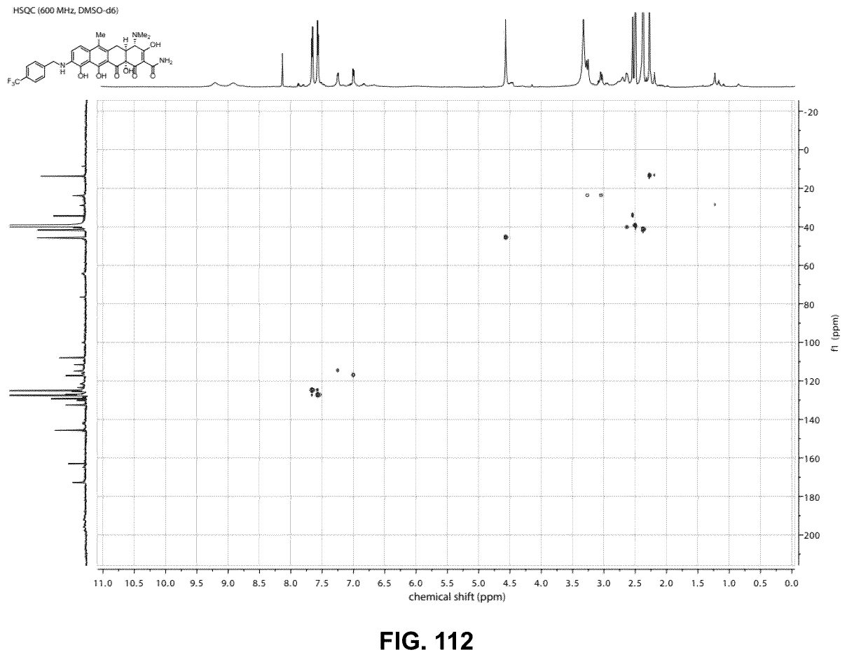

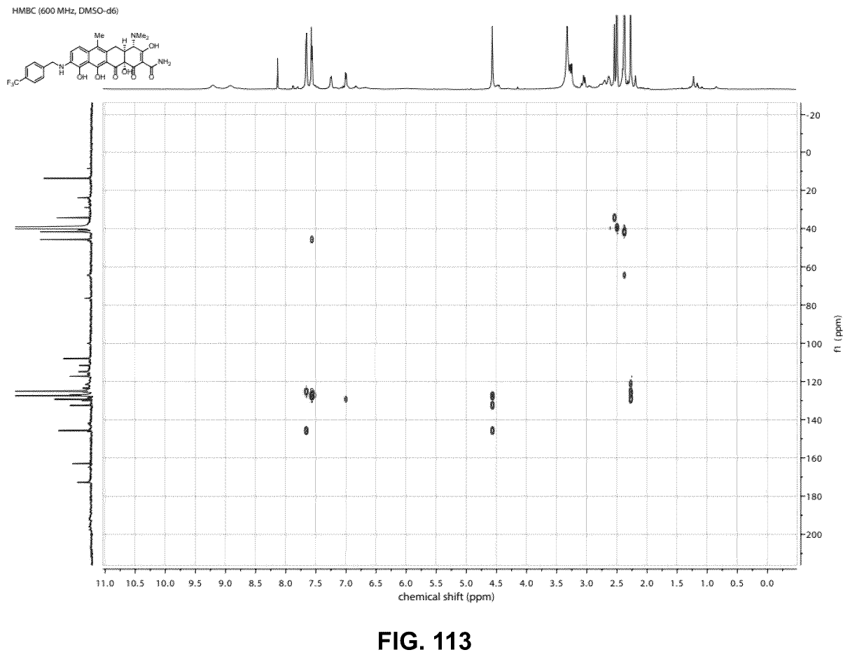

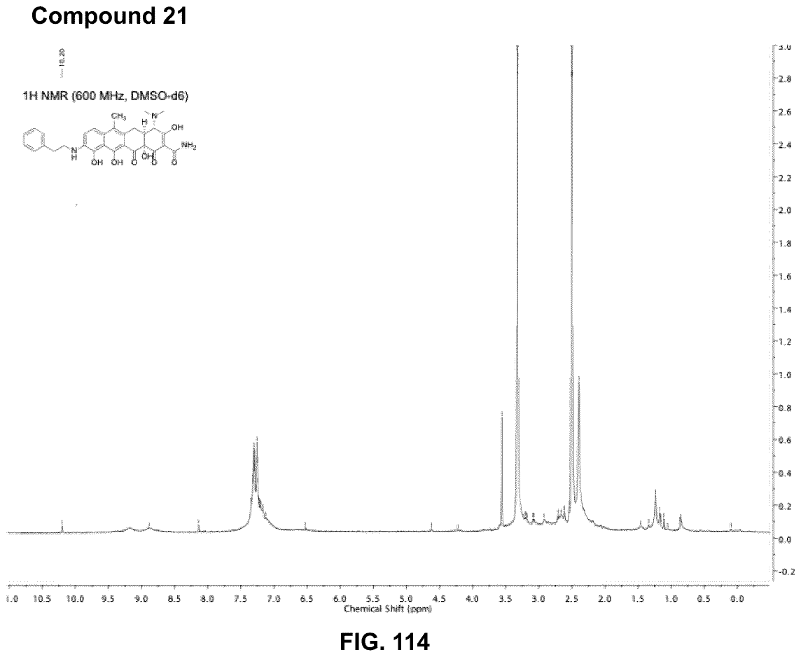

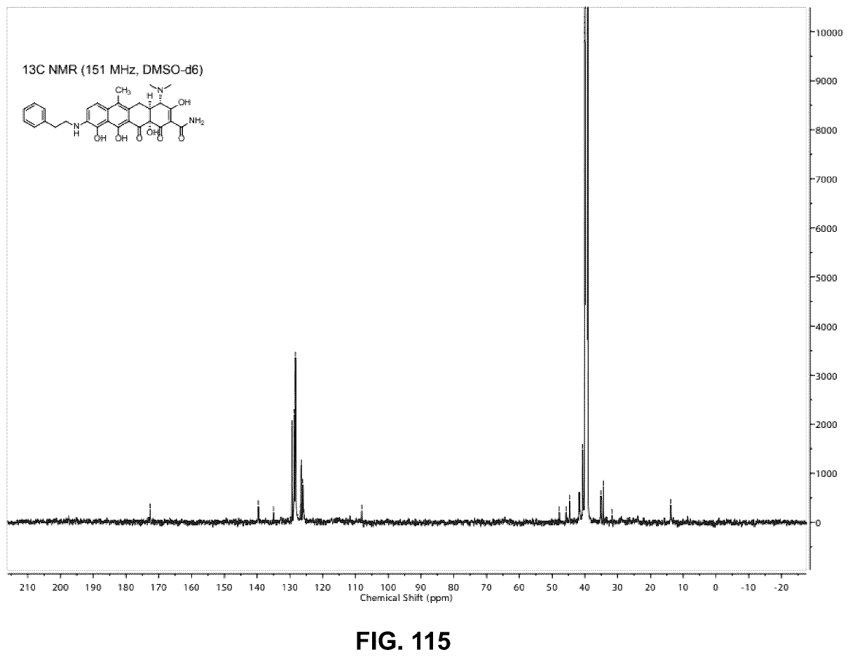

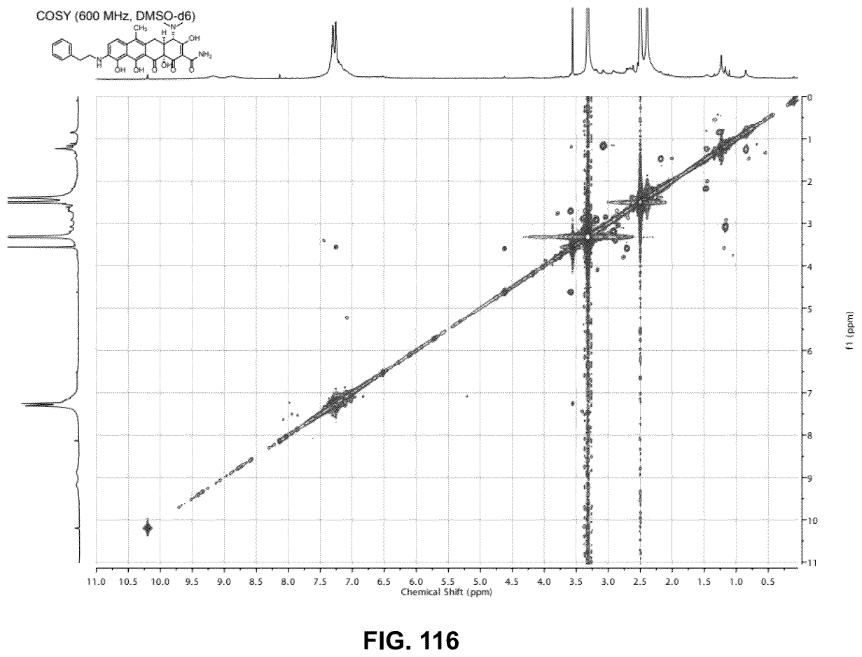

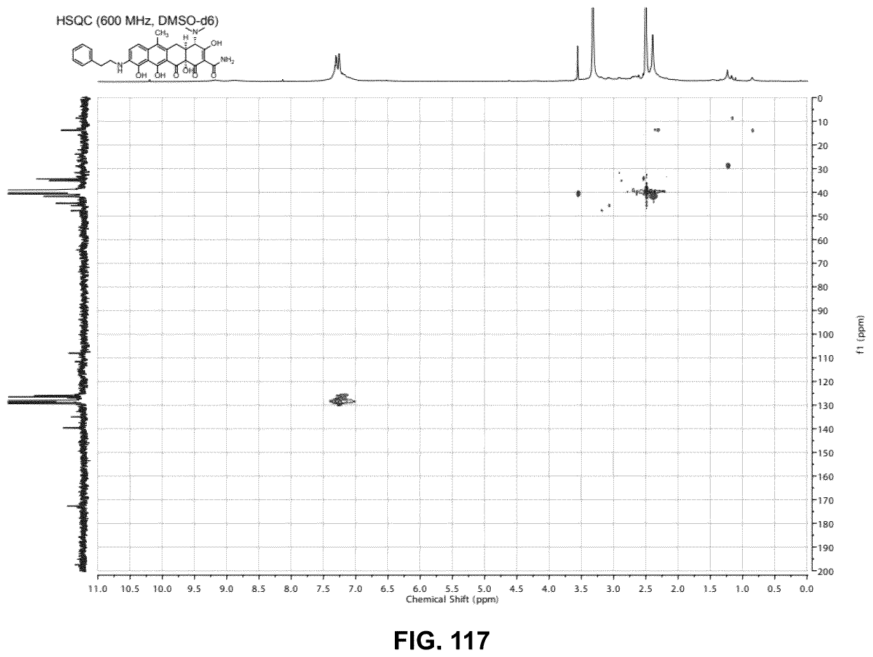

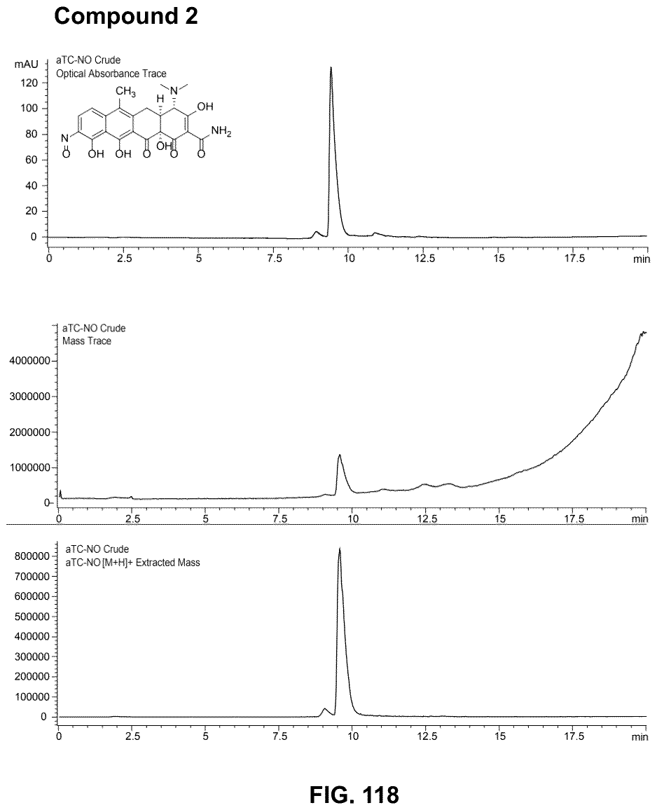

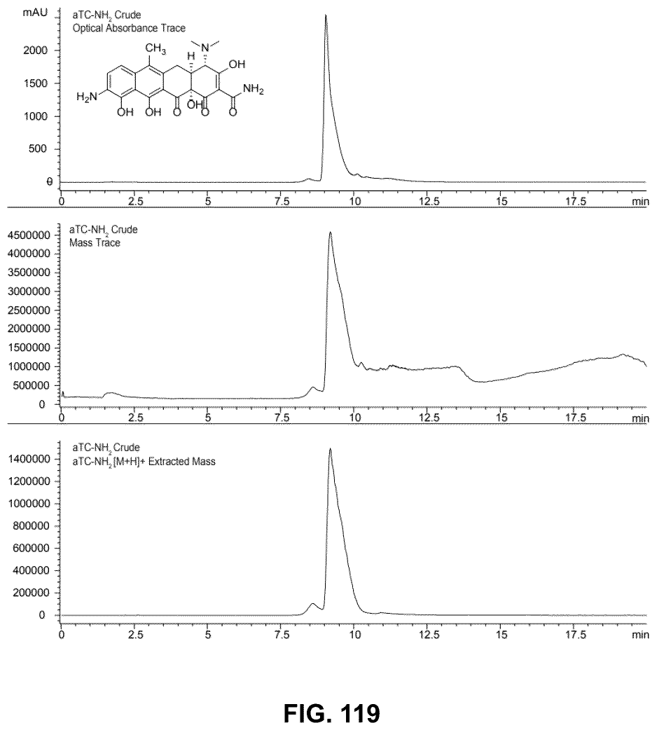

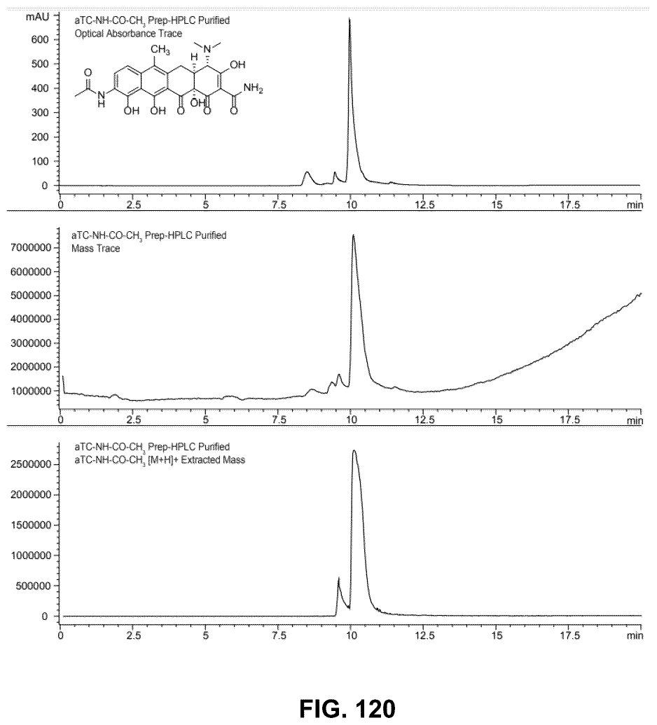

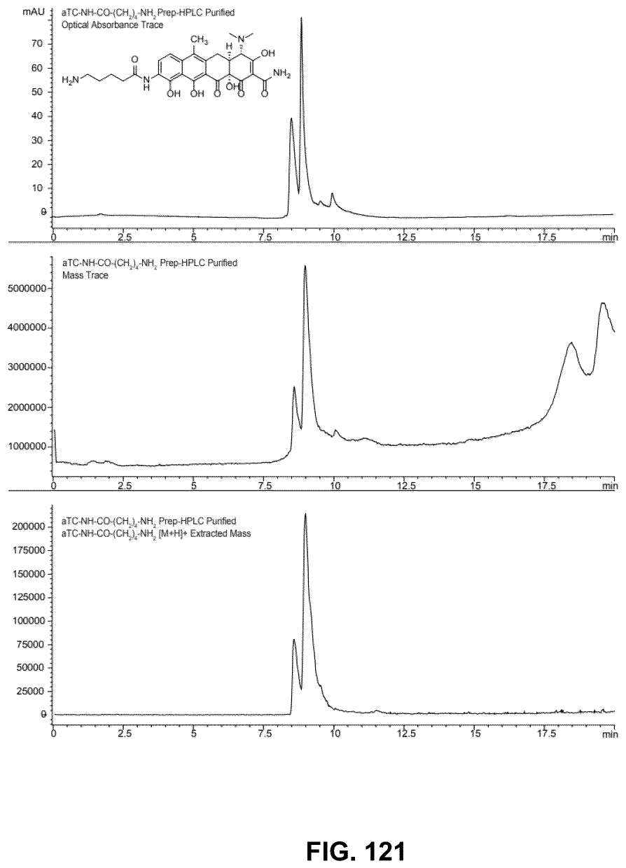

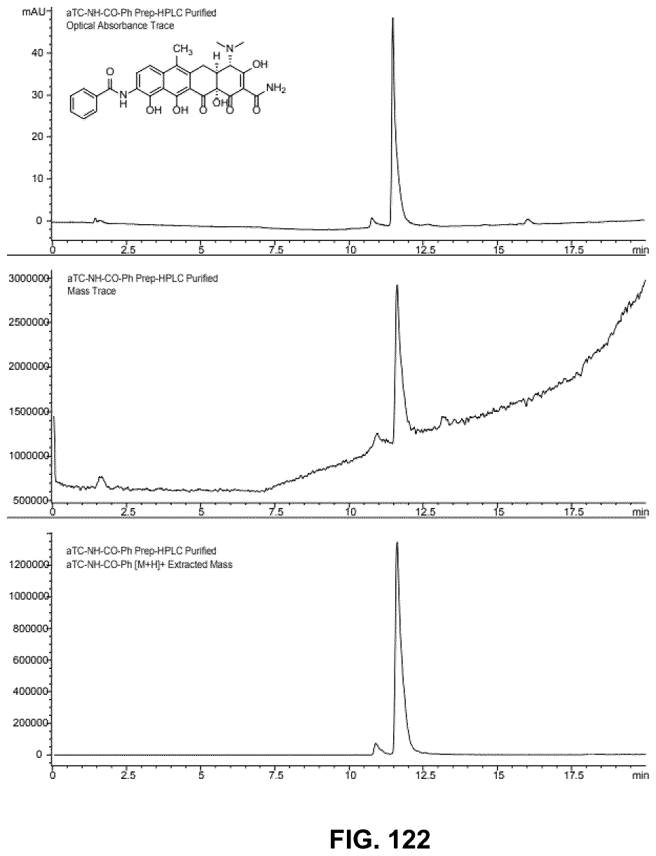

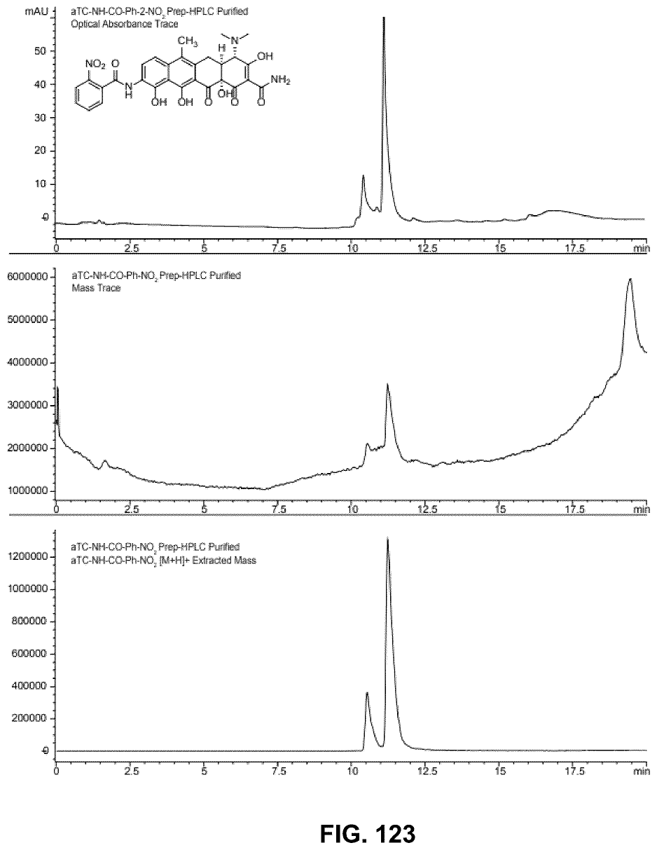

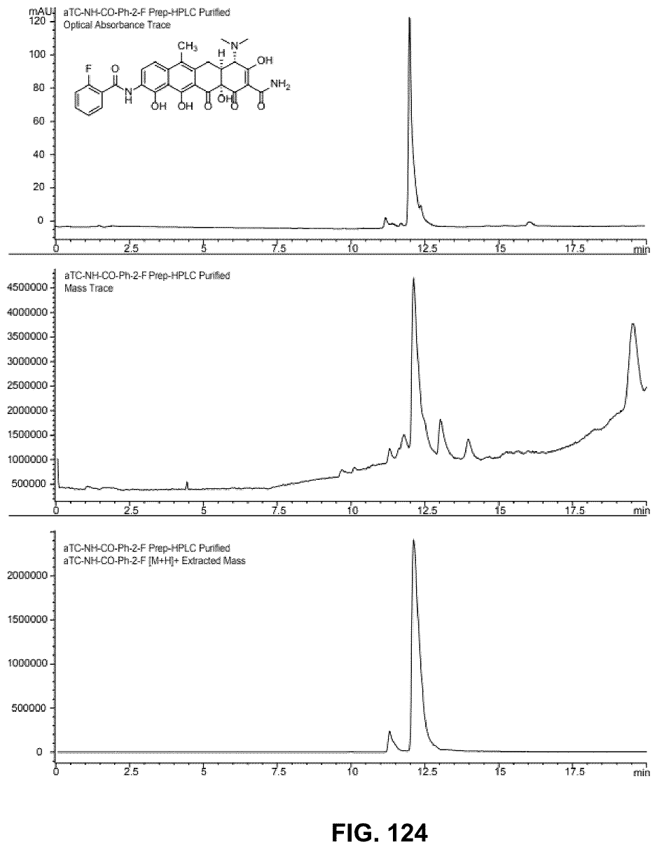

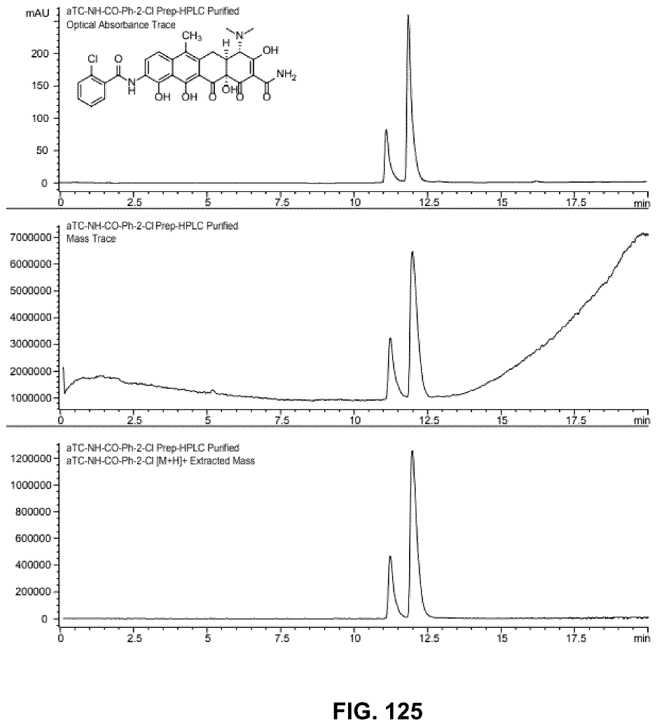

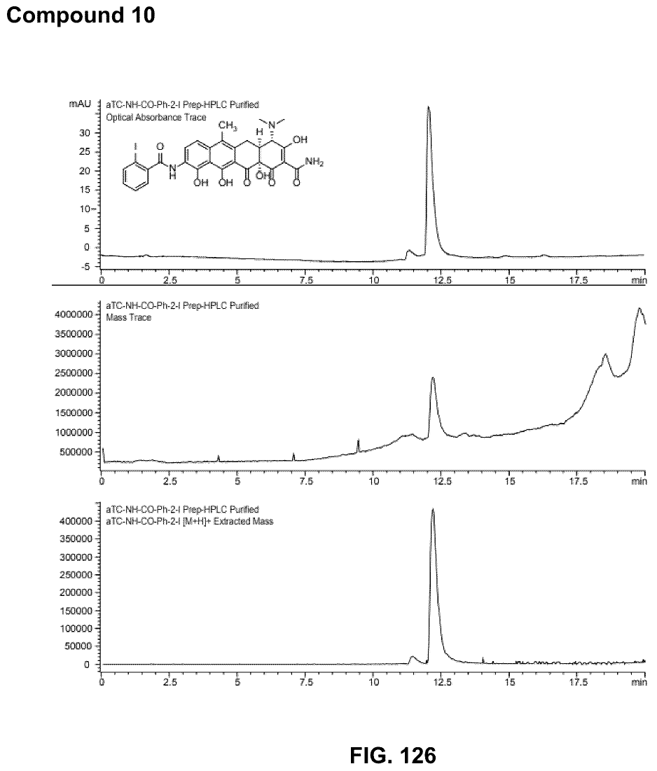

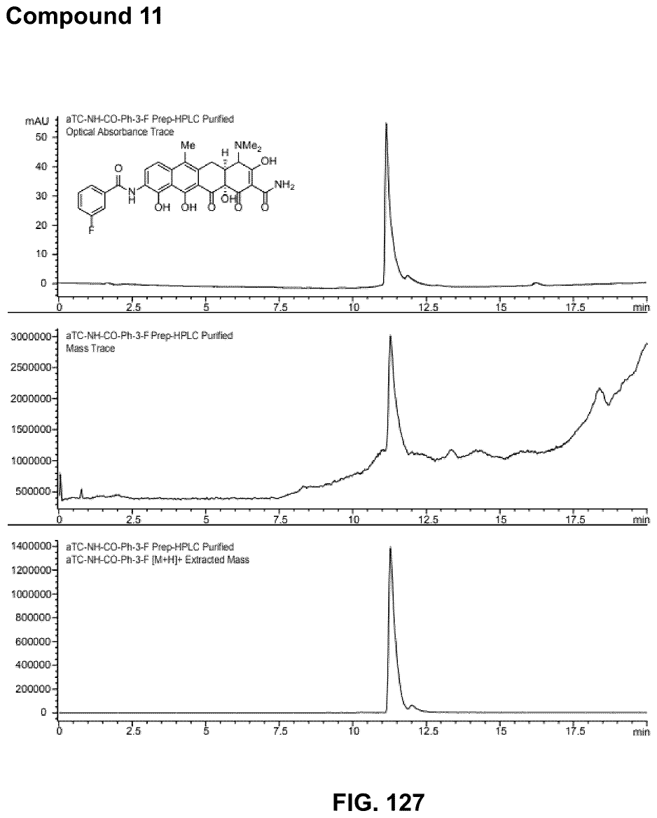

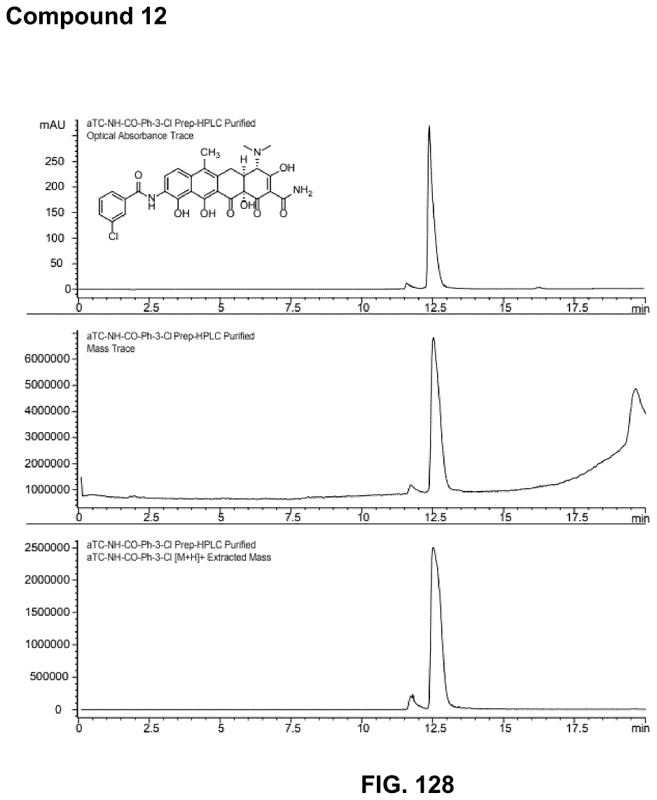

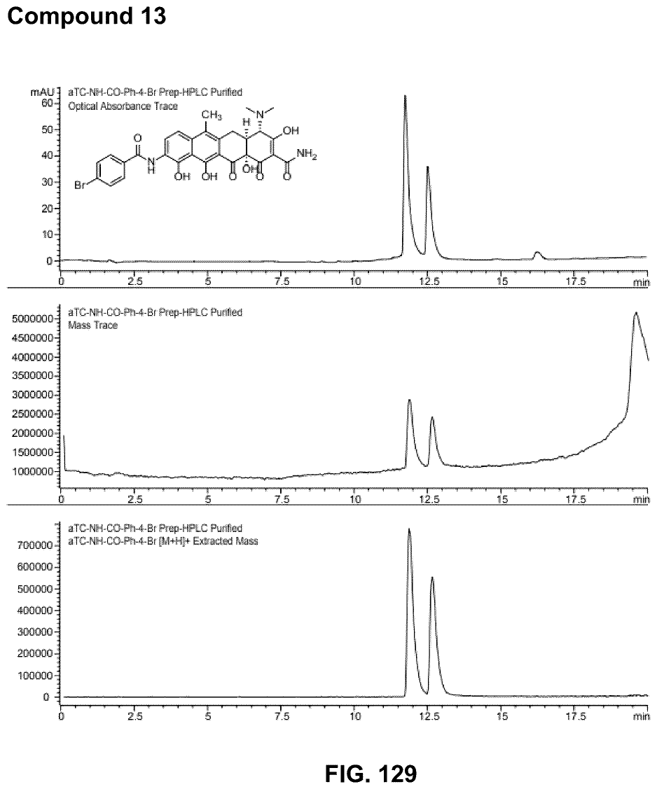

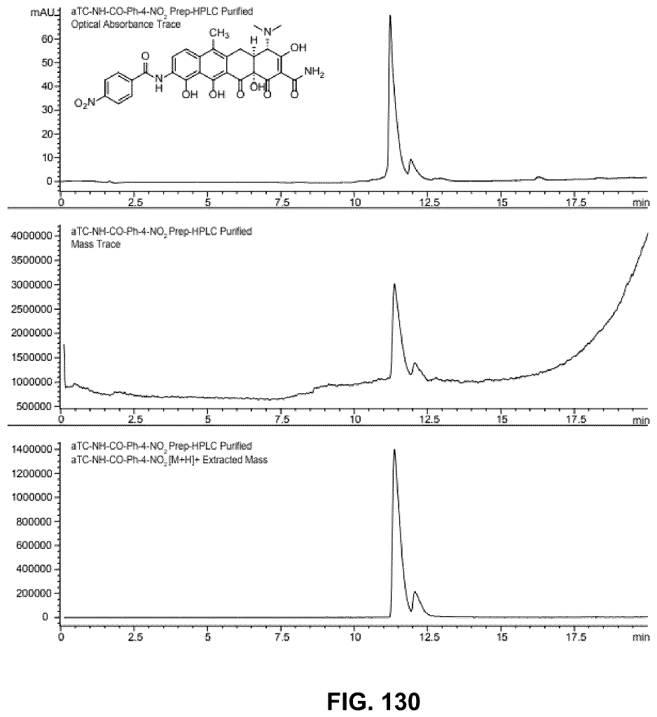

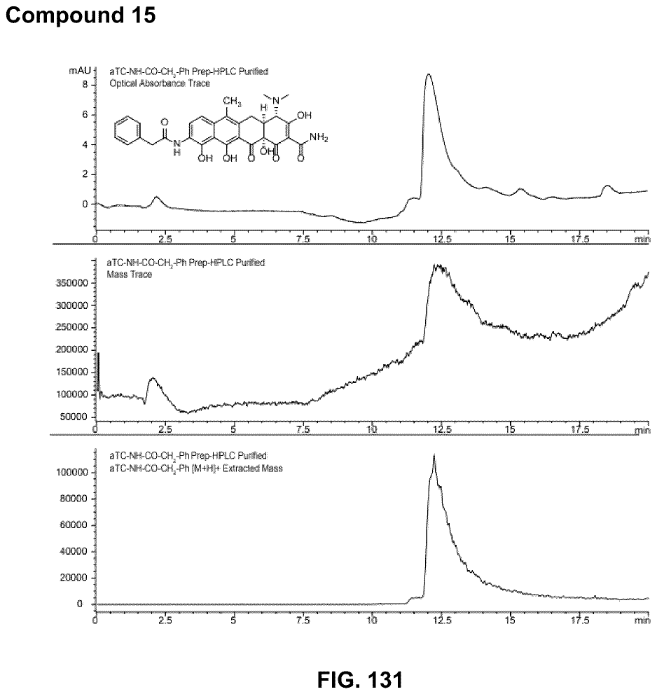

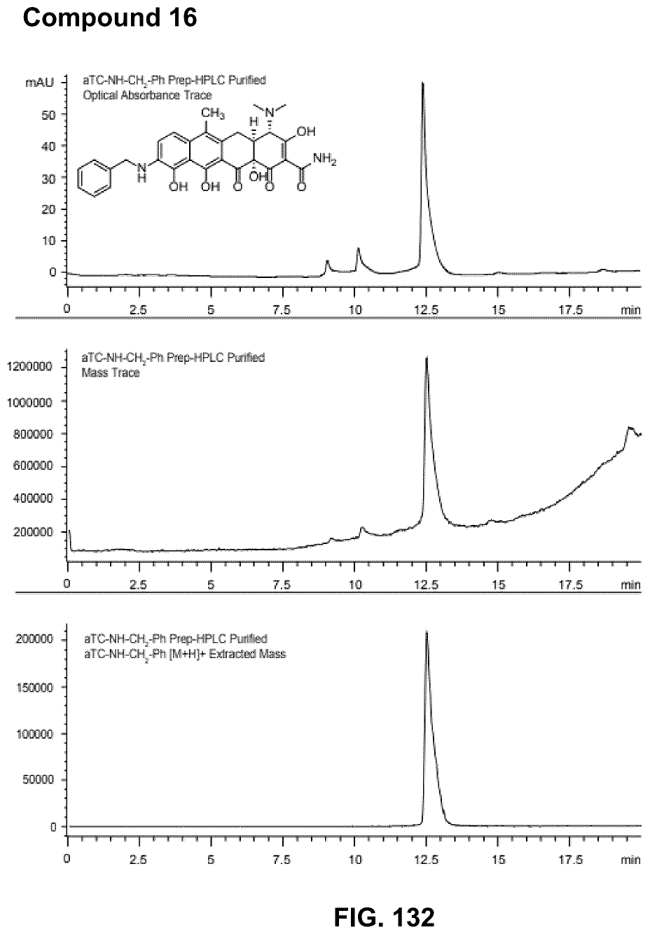

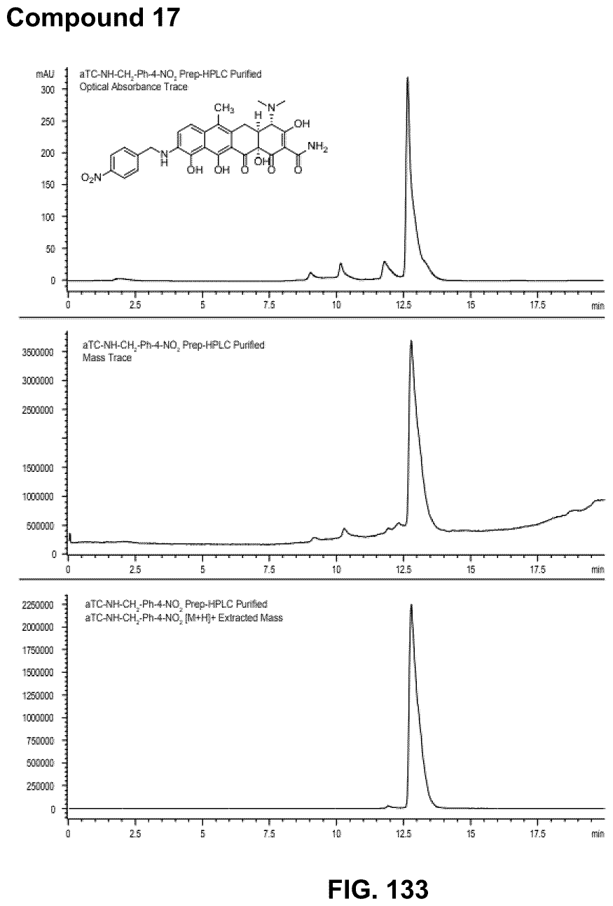

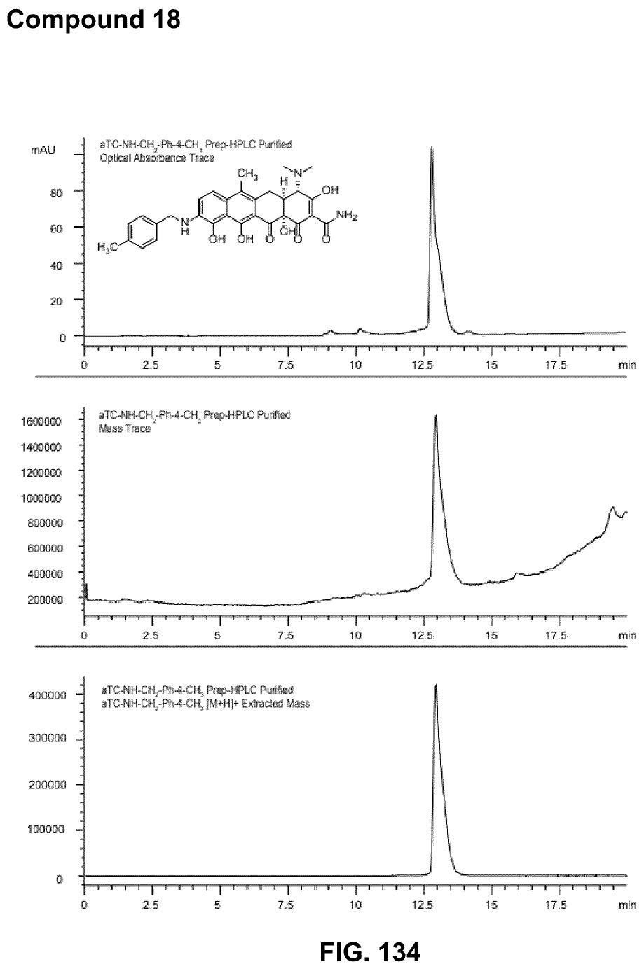

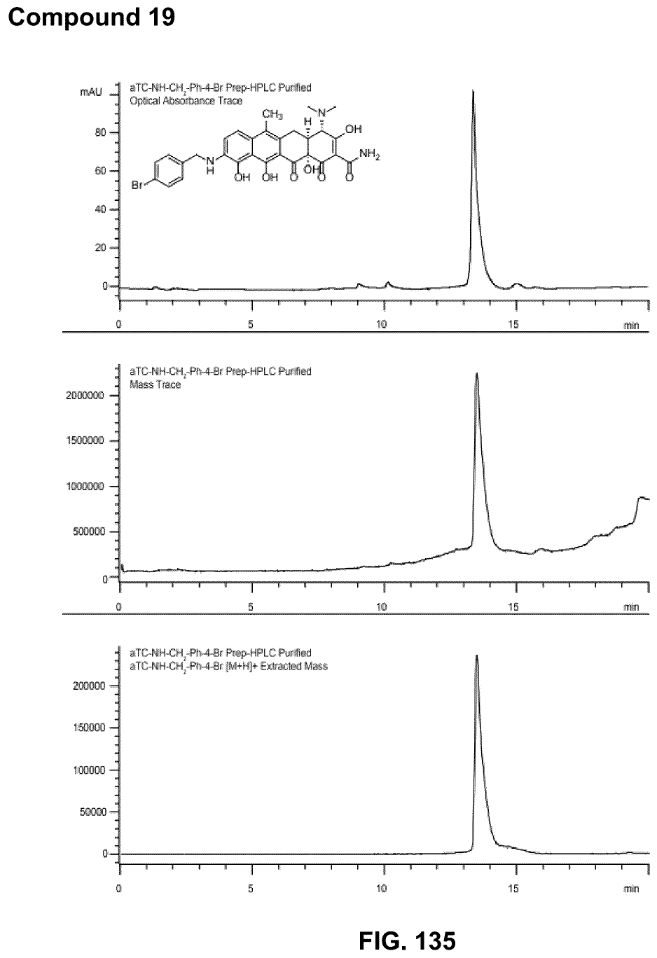

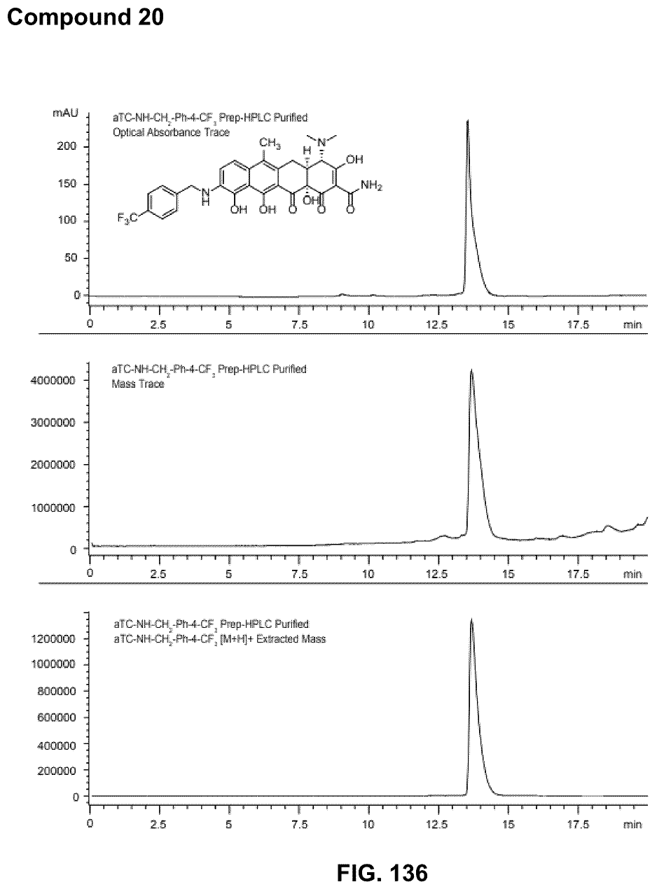

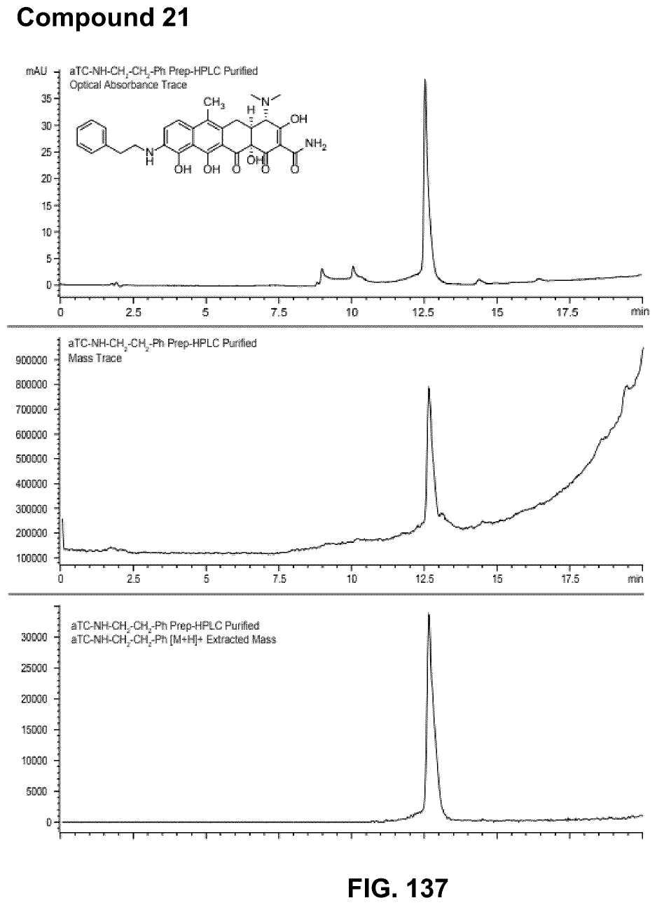

(4S,4aS,12aS)-9-(4-methylbenzylamino)-4-(dimethylamino)-3,10,11,12a-tetrahydroxy-6-methyl-1,12-dioxo-1,4,4a,5,12,12a-hexahydrotetracene-2-carboxamide (18).: Compound 18 was prepared according to general procedure B and was obtained in 20% yield as a red solid. FIGS. 99, 100, 101, 102, and 103 show the NMR characterization of compound 18. ¹H NMR (600 MHz, DMSO-d6) δ (ppm) 7.83 (d, J=7.9 Hz, 1H), 7.30 (d, J=7.9 Hz, 1H), 7.24 (d, J=7.4 Hz, 2H), 7.09 (d, J=7.4 Hz, 2H), 4.41 (s, 2H), 3.28-3.25 (m, 1H), 3.05-3.02 (m, 1H), 2.63-2.60 (m, 1H), 2.39-2.37 (m, 1H) 2.38 (s, 6H), 2.37 (s, 3H), 2.24 (s, 3H). ¹³C NMR (151 MHz, DMSO-d6) δ (ppm) 191.55, 177.41, 173.03, 172.76, 167.28, 163.03, 157.73, 143.01, 137.58, 137.21, 135.58, 135.32, 132.81, 130.88, 129.83, 129.31, 129.21, 129.10, 128.87, 128.60, 128.00, 127.14, 126.92, 126.83, 125.44, 117.65, 116.24, 115.25, 113.39, 113.18, 111.73, 109.71, 108.01, 76.59, 47.14, 46.70, 45.70, 41.73, 40.29, 34.36, 26.99, 24.58, 23.90, 21.11, 20.63, 13.71, 13.56. Prep-HPLC tr 16m. LCMS epimeric purity 98% (see FIG. 134). MS (ESI+) [M+H]⁺ 545.8. HRMS (TOF MS ESI+) calculated for C₃₀H₃₁N₃O₇ [M+H]⁺ 546.2235; found 546.2235.

(4S,4aS,12aS)-9-(4-bromobenzylamino)-4-(dimethylamino)-3,10,11,12a-tetrahydroxy-6-methyl-1,12-dioxo-1,4,4a,5,12,12a-hexahydrotetracene-2-carboxamide (19).: Compound 19 was prepared according to general procedure B and was obtained in 30% yield as a bright red solid. FIGS. 104, 105, 106, 107, and 108 show the NMR characterization of compound 19. ¹H NMR (600 MHz, DMSO-d6) δ (ppm) 7.86 (d, J=8.3 Hz, 1H), 7.70 (d, J=8.3, 1H), 7.51 (d, J=8.1 Hz, 2H), 7.27 (d, J=8.1 Hz, 2H), 4.46 (s, 2H), 3.32 (s, 6H), 3.25-3.15 (m, 1H), 3.02-2.94 (m, 1H), 2.38 (s, 3H), 2.25-2.23 (m, 1H)¹³C NMR (151 MHz, DMSO-d6) δ (ppm) 163.02, 141.91, 140.48, 131.55, 131.20, 130.80, 128.46, 119.42, 62.03, 41.79, 28.92, 24.51. Prep-HPLC tr 16m. LCMS epimeric purity 98% (see FIG. 135). MS (ESI+) [M+H]⁺ 609.9. HRMS (FT-ICR APCI+) calculated for C₂₉H₂₈BrN₃O₇ [M+H]⁺ 610.1183; found 610.1163.

(4S,4aS,12aS)-9-(4-trifluoromethylbenzylamino)-4-(dimethylamino)-3,10,11,12atetrahydroxy-6-methyl-1,12-dioxo-1,4,4a,5,12,12a-hexahydrotetracene-2-carboxamide (20).: Compound 20 was prepared according to general procedure B and was obtained in 25% yield as a red solid. FIGS. 109, 110, 111, 112, and 113 show the NMR characterization of compound 20. ¹H NMR (600 MHz, DMSO-d6) δ (ppm) 7.66 (d, J=8.1 Hz, 2H), 7.57 (d, J=8.1 Hz, 2H), 7.25 (d, J=8.9 Hz, 1H), 7.00 (d, J=8.9 Hz, 1H), 4.57 (s, 2H), 3.27 (d, J=16 Hz, 2H), 3.09-3.02 (m, J=17, 16, 5.9 Hz, 1H), 2.54 (s, 1H), 2.38 (s, 5H), 2.27 (s, 3H). ¹³C NMR (151 MHz, DMSO-d6) δ (ppm) 172.76, 163.02, 146.19, 145.69, 132.53, 130.09, 129.61, 129.32, 128.53, 127.55, 127.35, 127.14, 126.93, 125.52, 125.15, 123.45, 121.65, 117.29, 111.59, 108.04, 76.54, 45.74, 41.69, 39.92, 39.78, 39.64, 39.50, 39.36, 39.22, 39.08, 34.36, 28.99, 23.89, 13.70, 13.54, 8.59. Prep-HPLC tr 16m. LCMS epimeric purity 99% (see FIG. 136). MS (ESI+) calculated for C₃₀H₂₈F₃N₃O₇ [M+H]⁺600.2; found 600.2.

(4S,4aS,12aS)-9-phenethylamino-4-(dimethylamino)-3,10,11,12a-tetrahydroxy-6-methyl-1,12-dioxo-1,4,4a,5,12,12a-hexahydrotetracene-2-carboxamide (21).: To a clean, dry round-bottom flask, equipped with stir bar, was added aTC-NH2 hydrochloride (compound 3) (63.8 mg, 0.124 mmol) and DriSolv methanol (6.0 mL) under argon atmosphere. Et3N (18.7 uL, 0.1341 mmol) was dissolved in 1.2 mL of methanol in a separate vial and transferred to the brown stirring solution. The dark red reaction mixture was cooled to 0° C. Phenylacetaldehyde, synthesized via Dess-Martin oxidation of phenethyl alcohol, was dissolved in 1 mL of methanol in a separate vial and transferred to the reaction. The reaction was stirred for 5 minutes. Na(OAc)3BH (0.0788 g, 0.3720 mmol) was added in one portion and the reaction was stirred for 1 hour. The reaction was quenched with a saturated sodium bicarbonate solution and concentrated under reduced pressure. The dark red solid was filtered through a 0.45 μM PTFE syringe filter and purified by preparative HPLC (tR=15m) to provide the formate salt. (pink solid, 0.0110 g, 15% yield). FIGS. 114, 115, 116, and 117 show the NMR characterization of compound 21. 1H NMR (600 MHz, DMSO-d6) δ (ppm) 7.34-7.23 (m, 10H), 3.56 (s, 2H), 3.21-3.18 (m, 2H), 3.10-3.06 (m, 1H), 2.94-2.90 (m, 1H), 2.73-7.69 (m, 1H), 2.39 (s, 8H) ¹³C NMR (151 MHz, DMSO) δ 172.66, 139.62, 135.00, 129.34, 128.72, 128.65, 128.37, 128.33, 128.21, 126.81, 126.55, 126.08, 108.05, 47.83, 45.71, 44.68, 40.67, 39.94, 39.80, 39.66, 39.52, 39.38, 39.24, 39.10, 35.10, 34.37, 31.69, 13.78. LCMS epimeric purity 90% (see FIG. 137). MS (ESI+) [M+H]⁺ 546.0. HRMS (TOF MS ESI+) calculated for C₃₀H₃₁N₃O₇ [M+H]⁺ 546.2235; found 546.2242.

### Cloning, Expression, and Purification of TDases.

All genes encoding TDases used in this study were cloned into pET28b(+) vectors (Novagen) as previously described (BamHI and NdeI restriction sites) and transformed into BL21-Star (DE3) competent cells (Life Technologies). Cells were cultured at 37° C. in lysogeny broth (LB) containing kanamycin (Kan) at 0.03 mg/mL (final concentration); once the culture reached an OD600 of -0.6, the cells were cooled to 0° C. in an ice water bath. Protein expression was induced by addition of 1 mM IPTG (final concentration), and cells were grown at 15° C. for 12-15 h. To harvest protein, the induced cells were pelleted by centrifugation at 4000 rpm for 15 min (4° C.) and resuspended in cold 40 mL of lysis buffer (50 mM K2HPO4, 500 mM NaCl, 20 mM imidazole, 10% glycerol, 5 mM BME, pH 8.0) containing SIGMAFAST© protease inhibitor (Millipore-Sigma). Cell suspensions were flash frozen in liquid nitrogen and stored at −80° C. Frozen cell suspensions were thawed and mechanically lysed using an Avestin EmulsiFlex-C5 cell disruptor, and the resultant lysate was clarified via ultra-centrifugation at 45,000 rpm for 35 min at 4° C. The clarified supernatant was transferred to a fritted column containing washed and equilibrated Ni-NTA resin and incubated for 30-45 min with gentle rocking. Resin was then washed with lysis buffer (2×40 mL), and the protein was eluted from the resin with elution buffer (5×10 mL elutions, 50 mM K2HPO4, 500 mM NaCl, 5 mM BME, 300 mM imidazole, 10% glycerol, pH 8.0). Fractions containing the desired proteins (as judged by SDS-PAGE analysis) were combined and transferred to a 10,000 molecular weight cutoff (MWCO) Snakeskin dialysis tubing (ThermoScientific) and equilibrated in dialysis buffer (50 mM K2HPO4 pH 8.0, 150 mM NaCl, 1 mM DTT) overnight. Dialyzed protein solutions were concentrated using a 30,000 MWCO Amicon centrifugal filter (Millipore-Sigma), and concentrated protein solution was flash frozen as beads in liquid nitrogen (50 μL portions) and stored at −80° C. The % FAD for each batch of protein varies and is determined by back calculation from a denaturated aliquot of enzyme using optical absorbance with the published extinction coefficient of 11,300 M-1 cm-1 for FAD. On average, batches of Tet(50) and Tet(X7) ranged from 20-60% FAD with an average of 30% FAd for batches of Tet(50) and 50% FAD for batches of Tet(X7).

### Characterization of Substrate and Inhibitor Degradation and Hydrogen Peroxide Detection by In Vitro TDase Reaction.

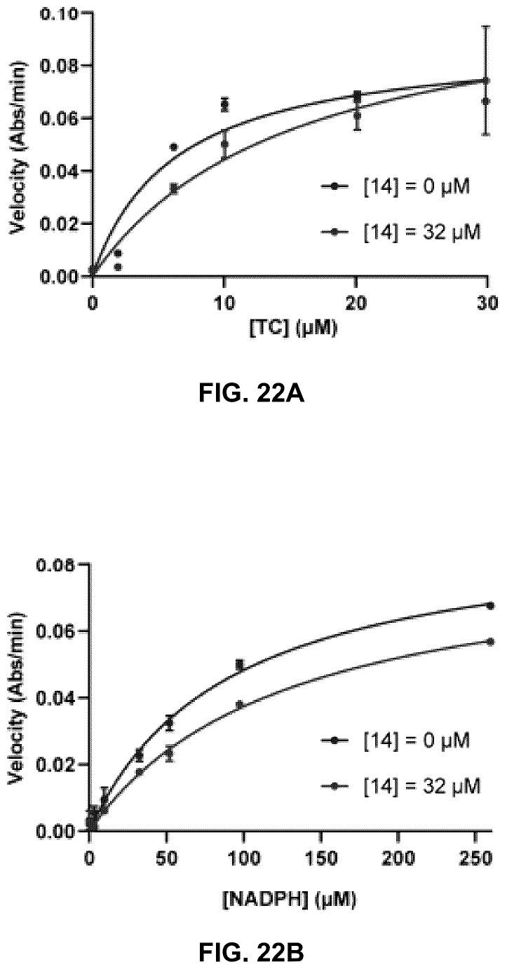

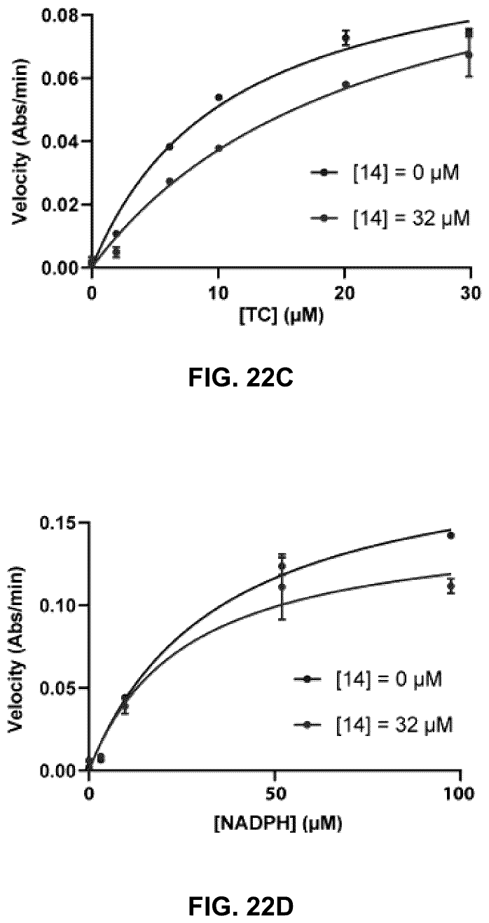

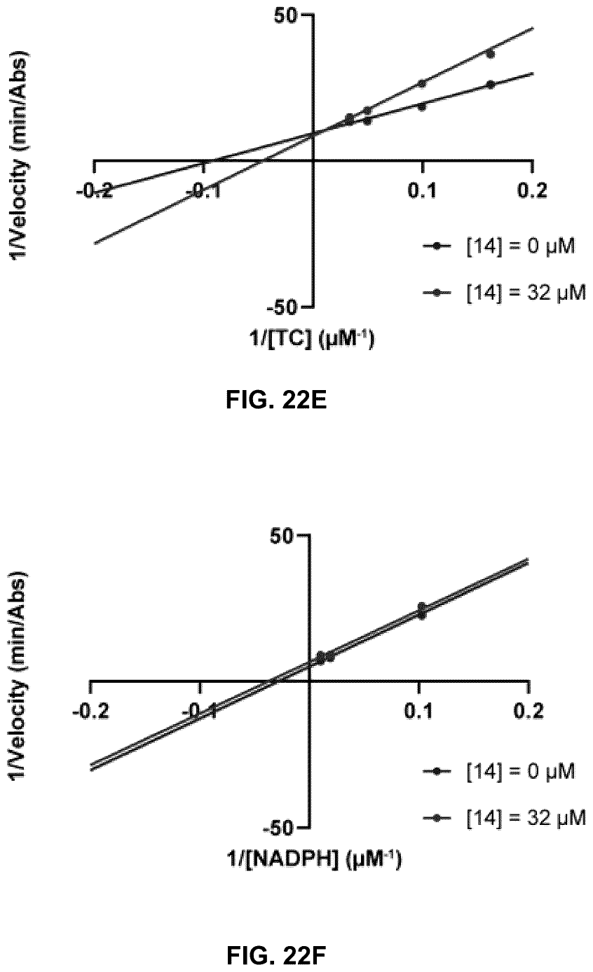

TDase reactions were prepared in 100 mM TAPS buffer (pH 8.5) with an NADPH regenerating system (40 mM G6P, 4 mM NADP+, 1 mM MgCl2, 4 U/mL G6P dehydrogenase) or 252 μM NADPH and 5.04 mM MgCl2, 20 μM substrate (15 μM substrate and 30 μM inhibitor in mixed assays), and 0.24 μM TDase enzyme (Tet(X7) and Tet(50)) (all concentrations represent final protein concentrations based on absorbance at 280 nm with % FAD-bound protein ranging from 20-60% batch-to-batch). Reaction progress was monitored by optical absorbance spectroscopy (280-550 nm, 1 nm and 5 min intervals) over 2 h. Aliquots of reaction sample (150 μL) were removed and quenched (600 μL of 1:1 acetonitrile/0.25 M aqueous HCl) immediately after enzyme was added (0 min) and at 5, 30, 60, 90, and 120 min intervals. The quenched samples were centrifuged (5000 rpm, 4° C.) for 5 min, and 600 μL of the resulting supernatant was mixed with an Fmoc-Ala internal standard (3.12 μM final concentration) and analyzed by LC-MS in positive ion mode (single trial). Extracted ion chromatograms (EICs) for the expected [M+H]⁺ molecular ions corresponding to substrate and mono-hydroxylated product were normalized to the Fmoc-Ala internal standard [M+H]⁺ counts. A separate 20 μL aliquot of reaction mixture was removed at 0, 5, 30, 60, 90, and 120 min timepoints and used for colorimetric detection of hydrogen peroxide formation performed using an aqueous Pierce Quantitative Peroxide Assay Kit (ThermoScientific). Each 20 μL aliquot (performed in triplicate on three separate aliquots for each timepoint) was added to a 96-well plate containing 200 μL of working reagent (prepared according to specifications for Pierce Quantitative Peroxide Assay kit). The plate was incubated for at least 20 min at room temp and optical absorbance was measured at 595 nm. Control reactions containing no TDase enzyme or no compound were also performed to determine background signals and no significant substrate/inhibitor degradation or H₂O₂ formation were observed (FIG. 22A).

### Apparent Steady-State Kinetics of aTC Inactivation and Inhibitor Acceleration of NADPH Consumption.

Reactions were prepared in 100 mM TAPS buffer at pH 8.5 with varying amounts of anhydrotetracycline (0-60 μM), 504 μM NADPH, 5.04 mM MgCl2, and 0.4 μM TDase (final protein concentration; working concentration of active enzyme is predicted to be 0.08-0.24 μM based on FAD content). Reactions were initiated by the addition of TDase and were monitored continuously via optical absorbance spectroscopy at 440 nm for aTC degradation or 360 nm for NADPH consumption for 2 min (performed in triplicate as independent trials). Initial enzyme velocities were determined by linear regression using Agilent Cary WinUV Software over the linear range of the reaction (typically between 0 to 1 min), plotted against the concentration of the substrate, and fitted to the Michaelis-Menten equation using GraphPad Prism 6.

### Lineweaver-Burke Kinetics of Inhibition of NADPH and TC Consumption.

Reactions were prepared in 100 mM TAPS buffer at pH 8.5 with varying amounts of NADPH (3-65 μM for Tet(X7); 3-260 μM for Tet(50)) or TC (2-30 μM for both Tet(X7) and Tet(50)), 5.04 mM MgCl2, and 0.4 μM TDase (final protein concentration; working concentration of active enzyme is predicted to be 0.08-0.24 μM based on FAD content). Reactions were initiated by the addition of TDase and were monitored continuously via optical absorbance spectroscopy at 400 nm for up to 2 min (performed in triplicate as independent trials). Initial enzyme velocities were determined by linear regression using Agilent Cary WinUV Software over the linear range of the reaction (typically between 0 to 1 min), plotted against the concentration of the substrate, and fitted to the Michaelis-Menten equation using GraphPad Prism 6.

### Determination of Apparent TDase Inhibitor IC₅₀ Values.

Half-maximal inhibitory concentrations (IC₅₀) for the inhibition of Tet(X7) and Tet(50) were determined from the velocities of TC degradation in the presence of varying concentrations of inhibitor. Reaction samples were prepared in 100 mM TAPS buffer (pH 8.5) with 504 μM NADPH, 5.04 mM MgCl2, 25.3 μM TC, varying concentrations of inhibitor (typically 2-262 μM), and 0.4 μM TDase (final protein concentration; working concentration of active enzyme is predicted to be 0.08-0.24 μM based on FAD content). Reactions were initiated by the addition of TDase and were monitored continuously via optical absorbance spectroscopy at 400 nm for 3 min (performed in triplicate as independent trials). Initial enzyme velocities were determined by linear regression using Agilent Cary WinUV Software over the linear range of the reaction (typically between 0 to 1 min). The velocities were plotted against the logarithm of inhibitor concentration, and apparent IC50 values were determined using nonlinear regression analysis in GraphPad Prism v6. Each set of experiments included a noTDase control reaction which was used as the full enzyme inhibition velocity and assigned to inhibitor concentration of 1×10¹⁵, and a no-inhibitor control which was assigned an inhibitor concentration of 1×10⁻¹⁵. A no-TC control was also performed to search for potentially competitive background signals generated from the enzymatic degradation of the inhibitor itself. For all inhibitor-enzyme combinations, the initial velocities of the no-TC controls were negligible.

### Antibiotic Susceptibility Testing—Broth Microdilution Method.

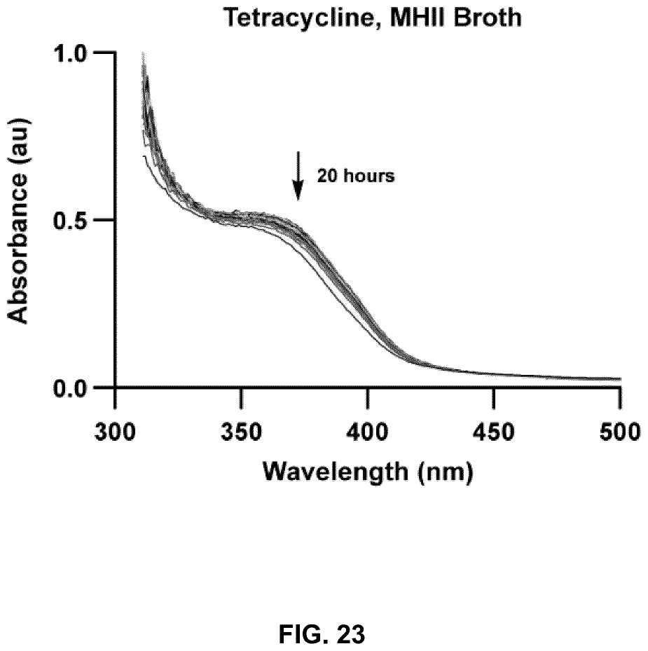

Inhibitors were diluted to 20 mg/mL in DMSO then diluted to 256 μg/mL in cation adjusted MH-II broth supplemented with 50 μg/mL Kan. Inhibition panels were prepared by performing a two-fold dilution series of each inhibitor, including a no-drug control, resulting in a concentration gradient of 0-128 μg/mL in MH-II broth containing a constant concentration of TC (16 μg/mL; final working concentration). E. coli MegaX (Invitrogen) expressing a TDase (Tet(X7) or Tet(50)) in the pZE21 plasmid vector were cultured to an OD600 of 0.3-0.8, then inoculated into the inhibition panel for a final concentration of ˜5×106 CFU/mL, including three replicates per inhibitor and a no-inocula control column. Plates were sealed with Breathe-Easy membranes (Sigma-Aldrich) and incubated at 37° C. For end-point growth assays, plates were shaken at 225 rpm and OD600 was measured at 20 h using a Synergy H1 plate reader (BioTek). For growth rate assays, inhibition panels were placed in a BioStack plate stacker (BioTek) and every 10-20 min panels were shaken for 30 s followed by OD600 measured using the Synergy H1 plate reader. The resulting growth curves were log-transformed and a rolling regression with a shifting window of 1 h was applied, such that the maximum slope of any of the regressions is the exponential growth rate53. If one of the three technical replicates showed no growth while the other two had growth (or vice-versa), or droplets on the Breathe-Easy membrane were observed, those wells were masked. For each strain-[inhibitor] combination, growth rates were compared to the no-inhibitor control using a one-way ANOVA with p-values corrected for multiple hypotheses using Benjamini-Hochberg method (FDR) in Prism. The growth rates were plotted against the logarithm of inhibitor concentration, and apparent IC₅₀ values were determined using nonlinear regression analysis in GraphPad Prism v6. A control reaction containing only TC in MH-II broth at 37° C. was performed to determine background TC degradation, and no significant non-enzymatic degradation was observed (FIG. 23).

### Antibiotic Susceptibility Testing—Agar Diffusion Method.

Antibacterial activity of the compounds was determined by a modified Kirby-Bauer agar diffusion assay. Overnight cultures of E. coli MegaX (Invitrogen) expressing a TDase (Tet(X7) or Tet(50)) from pZE21 plasmid vector were grown in LB broth containing 4 μg/mL TC and 50 μg/mL Kan for 18-24 h. Standardized cell suspensions of a 0.5 McFarland standard (OD600-0.08-0.1) were prepared in MH-II broth. Each standardized cell suspension (0.1 mL) was added to 35 mL of sterile, melted MH-II agar containing 4 μg/mL TC and 50 μg/mL Kan tempered to 47-50° C. After gentle mixing, the inoculated agar media was poured into a sterile plastic petri dish (145 mm×20 mm) and allowed to solidify. Wells of 9.0 mm diameter were cut from the petri dish agar and filled with 50 μL of the test sample solution. Substrate alone wells contained 4 mg/mL TC or 0.5 mg/mL Tigecycline. All wells with inhibitor contained 0.25 mg/mL of the corresponding inhibitor and all wells contained 80% DMSO v/v. Controls for 80% DMSO v/v were performed and resulted in no observable growth inhibition. The petri dish was incubated at 37° C. for -24 h and the inhibition zone diameters were measured (mm) with an electronic caliper.

### Bio-Layer Interferometry.

BLI experiments were performed on an Octet RED384 system (Forté Bio) with super streptavidin pins (Sartorius). Tet(50) and Tet(X7) were biotinylated using EZ-Link NHSPEG4-Biotin No-Weigh Format (ThermoScientific) and Pierce Biotin Quantitation kit to quantify biotinylation. Experiments were conducted in a running buffer of HEPES (pH=7.5) supplemented with 150 mM NaCl, 0.005% tween 20, 1 mM MgCl2, and 5% DMSO. Data were processed using double-reference subtraction with protein-binding signal corrected by subtracting signal of both immobilized protein into buffer and biotin-loaded pin into ligand (FIG. 18).

### Preparation of Receptor and Ligands for Molecular Docking.

All computations were performed using the Schrödinger platform (release 2021-1) accessed through the SBGrid consortium. Ligands and protein were prepared as previously described. In brief, ligands were obtained using 2D sketcher in maestro (version 12.7.156). Different 3D structural and chemical possibilities of ligands were enumerated using LigPrep. Epik was used to generate ionization states at pH 7.0±2.0. The receptor was prepared from chain B of PDB ID 5TUF using the protein preparation wizard. The protein was pre-processed to add hydrogens, to fill in missing sidechains and to delete water molecules. The bound inhibitor, aTC (TDC in PDB ID 5TUF) was deleted. An alternate rotamer was selected for Tyr267 such that the hydroxyphenyl-side chain flips away from the FAD interacting region (FIGS. 1B, 1C, 1D, and 1E). Different tautomeric states of the bound co-factor, FAD, were generated at pH 7.0±2.0 and the conformation with the least clash score was chosen. H-bonds were assigned using PROPKA at pH 7.0 and thus prepared protein was restrained minimized.

### Generation of Receptor Grid for Molecular Docking.

OPLS_2005 forcefield was used to generate the receptor grid. The grid center was manually adjusted to include the entire active site region. The bound cofactor, FAD, was included in the receptor while generating the grid. An inner box of 10 Å×10 Å×10 Å and an outer box of 30 Å×30 Å×30 Å was set and the grid center was fixed at cartesian coordinates of 18.7, 61.7 and 60.0.

### Molecular Docking and Analyses.

Molecular docking was performed using Glide with following parameters: precision mode=standard precision (SP); ligand sampling=flexible; added Epik state penalties to the docking scores; no constrains; and perform post-docking minimization. The docking results were visualized using the pose viewer in maestro. PyMOL v2.3.2 was used to generate images. To validate the docking protocol, the co-crystallized ligand, aTC, was extracted from chain B of Tet(50) (PDB ID: 5TUF) and re-docked into the prepared receptor. aTC binds with a docking score of −7.21 kcal/mol and the docked conformation overlaps with the co-crystallized conformation (FIG. 9B). The validated docking protocol was used to predict the binding modes of aTC derivatives (compounds 14 and 20) in Tet(50).

TABLE 2
Inhibitor growth rate statistical comparisons.
Non-significant comparisons (FDR p > 0.05).

| | | | | Indi- |
|---|---|---|---|---|
| Inhibitor | | Mean | q | vidual |
| strain | Original FDR method | Diff. | value | P Value |
| aTC_tetX7 | 0 ug/mL vs. 2 ug/mL | 0.0284 | 0.0313 | 0.0313 |
| | 0 ug/mL vs. 4 ug/mL | 0.09513 | <0.0001 | <0.0001 |
| | 0 ug/mL vs. 8 ug/mL | 0.1803 | <0.0001 | <0.0001 |
| | 0 ug/mL vs. 16 ug/mL | 0.2194 | <0.0001 | <0.0001 |
| | 0 ug/mL vs. 32 ug/mL | 0.4043 | <0.0001 | <0.0001 |
| | 0 ug/mL vs. 64 ug/mL | 0.386 | <0.0001 | <0.0001 |
| | 0 ug/mL vs. 128 ug/mL | 0.3954 | <0.0001 | <0.0001 |
| aTC_tet50 | 0 ug/mL vs. 2 ug/mL | 0.001261 | 0.9031 | 0.9031 |
| | 0 ug/mL vs. 4 ug/mL | 0.05675 | <0.0001 | <0.0001 |
| | 0 ug/mL vs. 8 ug/mL | 0.2348 | <0.0001 | <0.0001 |
| | 0 ug/mL vs. 16 ug/mL | 0.2348 | <0.0001 | <0.0001 |
| | 0 ug/mL vs. 32 ug/mL | 0.2317 | <0.0001 | <0.0001 |
| | 0 ug/mL vs. 64 ug/mL | 0.2306 | <0.0001 | <0.0001 |
| | 0 ug/mL vs. 128 ug/mL | 0.2223 | <0.0001 | <0.0001 |
| 14_tetX7 | 0 ug/mL vs. 2 ug/mL | 0.07127 | 0.0028 | 0.0028 |
| | 0 ug/mL vs. 4 ug/mL | 0.1067 | <0.0001 | <0.0001 |
| | 0 ug/mL vs. 8 ug/mL | 0.1169 | <0.0001 | <0.0001 |
| | 0 ug/mL vs. 16 ug/mL | 0.129 | <0.0001 | <0.0001 |
| | 0 ug/mL vs. 32 ug/mL | 0.1644 | <0.0001 | <0.0001 |
| | 0 ug/mL vs. 64 ug/mL | 0.1967 | <0.0001 | <0.0001 |
| | 0 ug/mL vs. 128 ug/mL | 0.2161 | <0.0001 | <0.0001 |
| 4_tetX7 | 0 ug/mL vs. 2 ug/mL | −0.01758 | 0.5195 | 0.5195 |
| | 0 ug/mL vs. 4 ug/mL | 0.05719 | 0.0682 | 0.0487 |
| | 0 ug/mL vs. 8 ug/mL | 0.04768 | 0.1096 | 0.0939 |
| | 0 ug/mL vs. 16 ug/mL | 0.05974 | 0.0682 | 0.0406 |
| | 0 ug/mL vs. 32 ug/mL | 0.1002 | 0.0044 | 0.0019 |
| | 0 ug/mL vs. 64 ug/mL | 0.1323 | 0.0012 | 0.0002 |
| | 0 ug/mL vs. 128 ug/mL | 0.1163 | 0.0019 | 0.0006 |
| 16_tet50 | 0 ug/mL vs. 2 ug/mL | −0.03437 | 0.0774 | 0.0332 |
| | 0 ug/mL vs. 4 ug/mL | 0.002257 | 0.8803 | 0.8803 |
| | 0 ug/mL vs. 8 ug/mL | −0.003518 | 0.8803 | 0.8145 |
| | 0 ug/mL vs. 16 ug/mL | 0.01304 | 0.6819 | 0.3897 |
| | 0 ug/mL vs. 32 ug/mL | 0.006279 | 0.8803 | 0.6759 |
| | 0 ug/mL vs. 64 ug/mL | 0.05494 | 0.0064 | 0.0018 |
| | 0 ug/mL vs. 128 ug/mL | 0.1054 | <0.0001 | <0.0001 |
| 16_tetX7 | 0 ug/mL vs. 2 ug/mL | 0.0435 | 0.07 | 0.07 |
| | 0 ug/mL vs. 4 ug/mL | 0.09439 | 0.0008 | 0.0007 |
| | 0 ug/mL vs. 8 ug/mL | 0.1282 | <0.0001 | <0.0001 |
| | 0 ug/mL vs. 16 ug/mL | 0.1386 | <0.0001 | <0.0001 |
| | 0 ug/mL vs. 32 ug/mL | 0.1685 | <0.0001 | <0.0001 |
| | 0 ug/mL vs. 64 ug/mL | 0.2079 | <0.0001 | <0.0001 |
| | 0 ug/mL vs. 128 ug/mL | 0.2457 | <0.0001 | <0.0001 |
| 17_tet50 | 0 ug/mL vs. 2 ug/mL | −0.03053 | 0.1168 | 0.0667 |
| | 0 ug/mL vs. 4 ug/mL | 0.00171 | 0.9136 | 0.9136 |
| | 0 ug/mL vs. 8 ug/mL | 0.01601 | 0.3705 | 0.3176 |
| | 0 ug/mL vs. 16 ug/mL | 0.02688 | 0.1435 | 0.1025 |
| | 0 ug/mL vs. 32 ug/mL | 0.04204 | 0.0361 | 0.0155 |
| | 0 ug/mL vs. 64 ug/mL | 0.07332 | 0.0008 | 0.0002 |
| | 0 ug/mL vs. 128 ug/mL | 0.1012 | <0.0001 | <0.0001 |
| 17_tetX7 | 0 ug/mL vs. 2 ug/mL | 0.03064 | 0.1044 | 0.1044 |
| | 0 ug/mL vs. 4 ug/mL | 0.03398 | 0.0867 | 0.0743 |
| | 0 ug/mL vs. 8 ug/mL | 0.07843 | 0.0006 | 0.0004 |
| | 0 ug/mL vs. 16 ug/mL | 0.115 | <0.0001 | <0.0001 |
| | 0 ug/mL vs. 32 ug/mL | 0.1582 | <0.0001 | <0.0001 |
| | 0 ug/mL vs. 64 ug/mL | 0.1676 | <0.0001 | <0.0001 |
| | 0 ug/mL vs. 128 ug/mL | 0.2176 | <0.0001 | <0.0001 |
| 18_tet50 | 0 ug/mL vs. 2 ug/mL | 0.04308 | <0.0001 | <0.0001 |
| | 0 ug/mL vs. 4 ug/mL | 0.04448 | <0.0001 | <0.0001 |
| | 0 ug/mL vs. 8 ug/mL | 0.07033 | <0.0001 | <0.0001 |
| | 0 ug/mL vs. 16 ug/mL | 0.08355 | <0.0001 | <0.0001 |
| | 0 ug/mL vs. 32 ug/mL | 0.1117 | <0.0001 | <0.0001 |
| | 0 ug/mL vs. 64 ug/mL | 0.1062 | <0.0001 | <0.0001 |
| | 0 ug/mL vs. 128 ug/mL | 0.1648 | <0.0001 | <0.0001 |
| 19_tet50 | 0 ug/mL vs. 2 ug/mL | −0.008313 | 0.5434 | 0.5434 |
| | 0 ug/mL vs. 4 ug/mL | 0.008482 | 0.5434 | 0.5353 |
| | 0 ug/mL vs. 8 ug/mL | −0.0105 | 0.5434 | 0.4445 |
| | 0 ug/mL vs. 16 ug/mL | 0.0327 | 0.0465 | 0.0265 |
| | 0 ug/mL vs. 32 ug/mL | 0.05974 | 0.0009 | 0.0004 |
| | 0 ug/mL vs. 64 ug/mL | 0.06931 | 0.0003 | <0.0001 |
| | 0 ug/mL vs. 128 ug/mL | 0.08142 | 0.0001 | <0.0001 |
| 20_tet50 | 0 ug/mL vs. 2 ug/mL | 0.02938 | 0.0661 | 0.0661 |
| | 0 ug/mL vs. 4 ug/mL | 0.06455 | 0.0006 | 0.0005 |
| | 0 ug/mL vs. 8 ug/mL | 0.07942 | <0.0001 | <0.0001 |
| | 0 ug/mL vs. 16 ug/mL | 0.0955 | <0.0001 | <0.0001 |
| | 0 ug/mL vs. 32 ug/mL | 0.1254 | <0.0001 | <0.0001 |
| | 0 ug/mL vs. 64 ug/mL | 0.1209 | <0.0001 | <0.0001 |
| | 0 ug/mL vs. 128 ug/mL | 0.1011 | <0.0001 | <0.0001 |

What is claimed is:

1. A bisubstrate tetracycline destructase inhibitor compound comprising a C9-substituted anhydrotetracycline (aTC) analog, the C9-substituted anhydrotetracycline (aTC) analog comprising the structure: 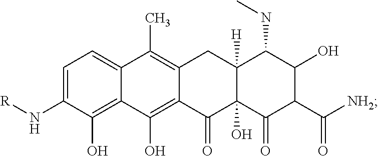
wherein R is selected from

| R Group | |
|---|---|
| 4 | 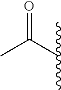 [SMILES: *C(C)=O] |
| 5 | 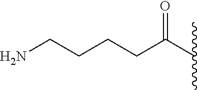 [SMILES: *C(=O)CCCCN] |
| 6 | 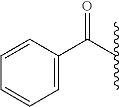 [SMILES: *C(=O)c1ccccc1] |
| 7 | 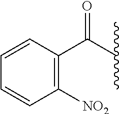 [SMILES: *C(=O)c1ccccc1[N+](=O)[O-]] |
| 8 | 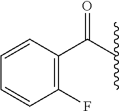 [SMILES: *C(=O)c1ccccc1F] |
| 9 | 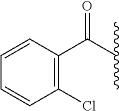 [SMILES: *C(=O)c1ccccc1Cl] |
| 10 | 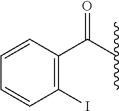 [SMILES: *C(=O)c1ccccc1I] |
| 11 | 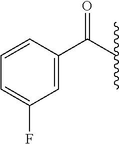 [SMILES: *C(=O)c1cccc(F)c1] |
| 12 | 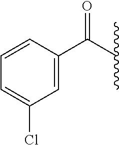 [SMILES: *C(=O)c1cccc(Cl)c1] |
| 13 | 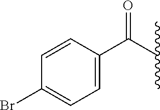 [SMILES: *C(=O)c1ccc(Br)cc1] |
| 14 | 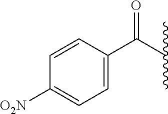 [SMILES: *C(=O)c1ccc([N+](=O)[O-])cc1] |
| 15 | 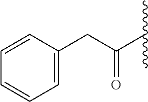 [SMILES: *C(=O)Cc1ccccc1] |
| 16 | 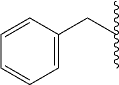 [SMILES: *Cc1ccccc1] |
| 17 | 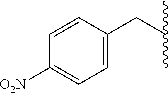 [SMILES: *Cc1ccc([N+](=O)[O-])cc1] |
| 18 | 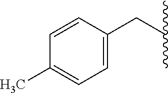 [SMILES: *Cc1ccc(C)cc1] |
| 19 | 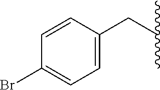 [SMILES: *Cc1ccc(Br)cc1] |
| 20 | 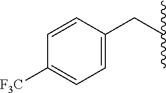 [SMILES: *Cc1ccc(C(F)(F)F)cc1] |
| 21 | 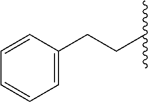 [SMILES: *CCc1ccccc1] . |

2. The compound of claim 1, wherein the compound inhibits both type 1 and type 2 tetracycline destructases (TDases).

3. The compound of claim 1, wherein the compound is a competitive inhibitor of TDases.

4. The compound of claim 1, wherein the compound binds sites comprising a substrate site and a nicotinamide site.

5. The compound of claim 4, wherein the substrate site is a tetracycline (TO) binding site.

6. The compound of claim 4, wherein the nicotinamide site is an NADPH binding site.

7. The compound of claim 1, wherein the compound inhibits TDase reactions comprising a NADPH oxidation and a FAD reduction.

8. A method of treating an infection with at least one of multidrug resistant (MDR) Gram-negative bacteria, extensively drug-resistant (XDR) Enterobacteriaceae species, and XDR Acinetobacter species, the method comprising administering, to a patient, a third-generation tetracycline in combination with a bisubstrate tetracycline destructase inhibitor compound.

9. The method of claim 8, wherein the bisubstrate tetracycline destructase inhibitor compound comprises a C9-substituted anhydrotetracycline (aTC) analog, the C9-substituted anhydrotetracycline (aTC) analog comprising the structure: 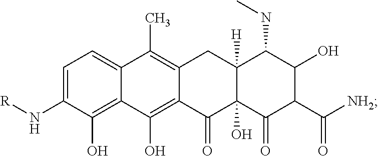
wherein R is selected from

| R Group | |
|---|---|
| 4 | 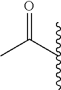 [SMILES: *C(C)=O] |
| 5 | 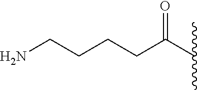 [SMILES: *C(=O)CCCCN] |
| 6 | 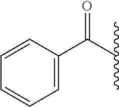 [SMILES: *C(=O)c1ccccc1] |
| 7 | 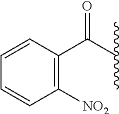 [SMILES: *C(=O)c1ccccc1[N+](=O)[O-]] |
| 8 | 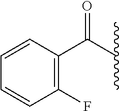 [SMILES: *C(=O)c1ccccc1F] |
| 9 | 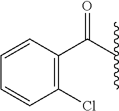 [SMILES: *C(=O)c1ccccc1Cl] |
| 10 | 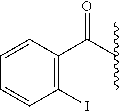 [SMILES: *C(=O)c1ccccc1I] |
| 11 | 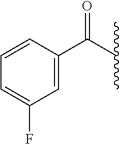 [SMILES: *C(=O)c1cccc(F)c1] |
| 12 | 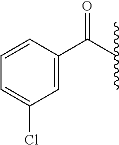 [SMILES: *C(=O)c1cccc(Cl)c1] |
| 13 | 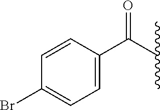 [SMILES: *C(=O)c1ccc(Br)cc1] |
| 14 | 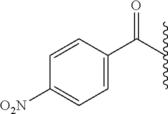 [SMILES: *C(=O)c1ccc([N+](=O)[O-])cc1] |
| 15 | 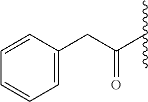 [SMILES: *C(=O)Cc1ccccc1] |
| 16 | 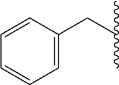 [SMILES: *Cc1ccccc1] |
| 17 | 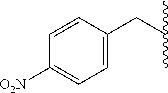 [SMILES: *Cc1ccc([N+](=O)[O-])cc1] |
| 18 | 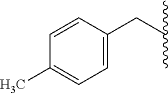 [SMILES: *Cc1ccc(C)cc1] |
| 19 | 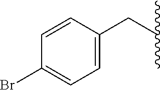 [SMILES: *Cc1ccc(Br)cc1] |
| 20 | 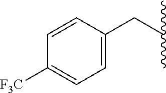 [SMILES: *Cc1ccc(C(F)(F)F)cc1] |
| 21 | 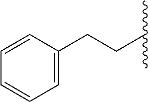 [SMILES: *CCc1ccccc1] . |

10. The method of claim 8, wherein the bisubstrate tetracycline destructase inhibitor compound inhibits both type 1 and type 2 tetracycline destructases (TDases).

11. The method of claim 8, wherein the bisubstrate tetracycline destructase inhibitor compound is a competitive inhibitor of TDases.

12. The method of claim 8, wherein the bisubstrate tetracycline destructase inhibitor compound binds sites on TDases comprising a substrate site and a nicotinamide site.

13. The method of claim 12, wherein the substrate site is a tetracycline (TC) binding site.

14. The method of claim 12, wherein the nicotinamide site is an NADPH binding site.

15. The method of claim 8, wherein the bisubstrate tetracycline destructase inhibitor compound inhibits TDase reactions comprising a NADPH oxidation and a FAD reduction.

16. The method of claim 9, wherein the C9-substituted aTC analog comprises a TDase inhibitory activity and an antibacterial activity.
